(19) United States
(12) Patent Application Publication
(10) Pub. No.: US 2026/0176333 A1
(43) Pub. Date: Jun. 25, 2026

(54) E3 LIGASE FAMILY FUNCTIONS AND INTERACTIONS

(71) Applicants: Genentech, Inc., South San Francisco, CA (US); The Broad Institute, Inc., Cambridge, MA (US); Massachusetts Institute of Technology, Cambridge, MA (US)

(72) Inventors: Aviv REGEV, Hillsborough, CA (US); Orit ROZENBLATT-ROSEN, Riverside, RI (US); Basak ERASLAN, San Francisco, CA (US); Kathryn Rachelle GEIGER-SCHULLER, San Francisco, CA (US)

(21) Appl. No.: 19/274,023

(22) Filed: Jul. 18, 2025

Related U.S. Application Data
(63) Continuation of application No. PCT/US2024/012180, filed on Jan. 19, 2024.
(60) Provisional application No. 63/440,365, filed on Jan. 20, 2023.

(30) Foreign Application Priority Data
Oct. 31, 2023 (JP) 2023-186191

(51) Int. Cl. C07K 16/00; C07K 16/28

(52) U.S. Cl. CPC C07K 16/00; C07K 16/2896; C07K 2317/73

(57) ABSTRACT

Provided herein are methods of treating cancers, inflammatory diseases, and autoimmune diseases and methods of modulating related phenotypes and expression levels by targeting interactions among E3 ligases, E3-like proteins, and their interacting partners. Methods of identifying modulators of such interactions are also provided. Also provided herein are cell therapies comprising alterations in at least two members of a co-functional gene module.

### CROSS-REFERENCE TO RELATED APPLICATIONS

This application is a continuation of International Patent Application No. PCT/US2024/012180, filed on Jan. 19, 2024, which claims benefit to U.S. Provisional Patent Application No. 63/440,365, filed on Jan. 20, 2023, and Japanese Patent Application No. 2023-186191, filed on Oct. 31, 2023, the entire contents of each of which are incorporated herein by reference in their entirety.

### STATEMENT AS TO FEDERALLY FUNDED RESEARCH

This invention was made with government support provided under Grant No. 5RM1 HGP706193-09 awarded by the National Human Genome Research Institute (NHGRI) Centers of Excellence in Genome Science (CEGS) and under Grant No. 5F32A1138458 awarded by the National Institutes of Health (NIH) Ruth L. Kirschstein National Research Service Award (NRSA) for Individual Postdoctoral Fellows (F32). The U.S. government has certain rights in the invention.

### FIELD OF THE INVENTION

Provided herein are methods of treating cancers, inflammatory diseases, and autoimmune diseases and methods of modulating related phenotypes and expression levels by targeting interactions among E3 ligases, E3-like proteins, and their interacting partners. Methods of identifying modulators of such interactions are also provided. Also provided herein are cell therapies comprising alterations in at least two members of a co-functional gene module.

### BACKGROUND

The human genome encodes more than 600 E3 ubiquitin ligases, which are responsible for catalyzing the ligation of ubiquitin to substrates in almost every biochemical pathway. Genome-wide association studies (GWAS) have implicated variants in E3 ligase genes in many diseases, including inflammatory and autoimmune diseases. While previous studies have implicated certain E3 ligases in the dendritic cell inflammatory response to lipopolysaccharide, relatively little is known about the roles of E3 ligases, E3-like proteins and interacting partners, and their substrates in dendritic cells or other primary immune cells. Thus, there is a need in the art for elucidation of novel roles of and relationships among E3 ligases and related genes in primary immune cells, as well as methods of modulating the newly discovered roles and relationships (e.g., to treat a cancer, an inflammatory disease, or an autoimmune disease).

### SUMMARY OF THE INVENTION

In one aspect, the invention provides a method for treating a cancer, an inflammatory disease, or an autoimmune disease in an individual, the method comprising administering to the individual an effective amount of a modulator of the interaction between (a) one, two, or all three of LIM domain-binding protein 2 (Ldb2), Ring finger protein 165 (Rnf165), and TNF receptor-associated factor 2 (Traf2) and (b) chemokine receptor type 7 (CCR7).

In some aspects, the individual has a cancer and the modulator is an agent that decreases the expression and/or activity of one, two, or all three of Ldb2, Rnf165, and Traf2.

In other aspects, the individual has an inflammatory disease or an autoimmune disease and the modulator is an agent that increases the expression and/or activity of one, two, or all three of Ldb2, Rnf165, and Traf2.

In another aspect, the invention provides a method for increasing expression of chemokine receptor type 7 (CCR7) in an antigen-presenting cell (APC), the method comprising contacting the APC with an effective amount of an agent that decreases the expression and/or activity of one, two, or all three of LIM domain-binding protein 2 (Ldb2), Ring finger protein 165 (Rnf165), and TNF receptor-associated factor 2 (Traf2).

In some aspects, the APC is in an individual. In some aspects, the individual has a cancer.

In some aspects, CCR7 expression in the APC is increased by at least 10% relative to expression in the absence of the agent.

In another aspect, the invention provides a method for increasing APC migration to a tumor and/or a lymph node in an individual, the method comprising administering to the individual an effective amount of an agent that decreases the expression and/or activity of one, two, or all three of LIM domain-binding protein 2 (Ldb2), Ring finger protein 165 (Rnf165), and TNF receptor-associated factor 2 (Traf2).

In some aspects, the individual has a cancer.

In some aspects, APC migration to the tumor and/or lymph node in the individual is increased by at least 10% relative to migration in the absence of the agent.

In some aspects, the APC is a dendritic cell (DC), a macrophage, or a glial cell. In some aspects, the glial cell is a microglial cell, an astrocyte, or an oligodendrocyte. In some aspects, the APC is a DC.

In another aspect, the invention provides a method for increasing T cell homing to a tumor in an individual, the method comprising administering to the individual an effective amount of an agent that decreases the expression and/or activity of one, two, or all three of LIM domain-binding protein 2 (Ldb2), Ring finger protein 165 (Rnf165), and TNF receptor-associated factor 2 (Traf2).

In some aspects, T cell homing to the tumor in the individual is increased by at least 10% relative to T cell homing in the absence of the agent.

In some aspects, the inflammatory disease or autoimmune disease is a neurodegenerative disease, arthritis, allergy, eczema, fibrosis, asthma, lupus erythematosus, an inflammatory bowel disease, ulcerative colitis, or Crohn's disease. In some aspects, the neurodegenerative disease is multiple sclerosis (MS), Alzheimer's disease (AD), amyotrophic lateral sclerosis (ALS), or Parkinson's disease (PD). In some aspects, the inflammatory disease or autoimmune disease is Crohn's disease.

In some aspects, the agent is a proteolysis targeting chimera (PROTAC), a small molecule, an antibody or antigen-binding fragment thereof, a peptide, a mimic, or an inhibitory nucleic acid.

In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain. In some aspects, the antibody or antigen-binding fragment thereof binds Ldb2, Rnf165, or Traf2. In some aspects, the antibody or antigen-binding fragment thereof binds CCR7. In some aspects, the agent is a bispecific antibody comprising an antigen-binding domain that targets the tumor microenvironment.

In some aspects, the method further comprises administering to the individual or contacting the APC with one or more additional agents.

In some aspects, the method further comprises administering to the individual or contacting the APC with one or more agents that modulate the expression of one or more of Akt1, Ankfy1, Apc, Arpc1 b, Birc2, Bmi1, Bub3, Cacybp, Cebpb, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnfll3a1, Rnf135, Rnf216, Rptor, Scap, Sec13, Sec31a, Smad2, Syvn1, Taf51, Traf2, Traf3, Traf7, Trim24, Trp53, Ube2e1, Ube2e3, Ube3c, Ufm1, Wdfy3, Wdr1, Wdr82, Whsc1, and Zbtb11.

In another aspect, the invention provides a kit comprising a modulator of the interaction between (a) one, two, or all three of Ldb2, Rnf165, and Traf2 and (b) CCR7 for treating an individual having a cancer, an inflammatory disease, or an autoimmune disease according to any one of the methods provided herein. In some aspects, the kit comprises a package insert comprising instructions to administer the modulator to an individual having a cancer, an inflammatory disease, or an autoimmune disease.

In another aspect, the invention provides a method of monitoring the response of an individual having a cancer, an inflammatory disease, or an autoimmune disease to treatment with a modulator of the interaction between (a) one, two, or all three of Ldb2, Rnf165, and Traf2 and (b) CCR7, the method comprising (i) determining, in a biological sample obtained from the individual at a time point following administration of the modulator, the expression level of one or more of Ldb2, Rnf165, and Traf2; and (ii) comparing the expression level of the one or more genes in the biological sample with a reference level, thereby monitoring the response in the individual to treatment with the modulator.

In some aspects, the reference level is selected from the group consisting of (i) the expression level of the one or more genes in a biological sample from the individual obtained prior to administration of the modulator; (ii) the expression level of the one or more genes in a reference population; (iii) a pre-assigned expression level for the one or more genes; or (iv) the expression level of the one or more genes in a biological sample obtained from the individual at a previous time point, wherein the previous time point is following administration of the modulator.

In some aspects, the individual has a cancer, the expression level of the one or more genes is increased in the biological sample obtained from the individual relative to the reference level, and the method further comprises administering to the individual one or more additional doses of the modulator, wherein the modulator is an agent that decreases the expression and/or activity of one, two, or all three of Ldb2, Rnf165, and Traf2.

In some aspects, the individual has an inflammatory disease or an autoimmune disease, the expression level of the one or more genes is decreased in the biological sample obtained from the individual relative to the reference level, and the method further comprises administering to the individual one or more additional doses of the modulator; wherein the modulator is an agent that increases the expression and/or activity of one, two, or all three of Ldb2, Rnf165, and Traf2.

In another aspect, the invention provides a method for treating a cancer, an inflammatory disease, an autoimmune disease, or an infectious disease in an individual, the method comprising administering to the individual an effective amount of (a) an agent that decreases the expression and/or activity of CCAAT/enhancer-binding protein beta (Cebpb); (b) an agent that decreases the expression and/or activity of TNF receptor-associated factor 2 (Traf2); and/or (c) an agent that increases the expression and/or activity of Death-inducer obliterator 1 (Dido1).

In some aspects, the autoimmune disease is associated with a reduced proportion of migratory dendritic cells (mDCs).

In some aspects, the individual has a loss-of-function mutation in Dido1.

In another aspect, the invention provides a method for treating an inflammatory disease, an autoimmune disease, or an infectious disease in an individual, the method comprising administering to the individual an effective amount of (a) an agent that increases the expression and/or activity of CCAAT/enhancer-binding protein beta (Cebpb); (b) an agent that increases the expression and/or activity of TNF receptor-associated factor 2 (Traf2); and/or (c) an agent that decreases the expression and/or activity of Death-inducer obliterator 1 (Dido1).

In another aspect, the invention provides a method for increasing the proportion of migratory dendritic cells (mDCs) in an individual, the method comprising administering to the individual an effective amount of (a) an agent that decreases the expression and/or activity of CCAAT/enhancer-binding protein beta (Cebpb); (b) an agent that decreases the expression and/or activity of TNF receptor-associated factor 2 (Traf2); and/or (c) an agent that increases the expression and/or activity of Death-inducer obliterator 1 (Dido1).

In some aspects, the proportion is a proportion in a tumor or a tissue of the individual.

In some aspects, the proportion of mDCs in the individual is increased by at least 10% relative to the proportion in the absence of the agent.

In another aspect, the invention provides a method for increasing anti-tumor immunity in an individual, the method comprising administering to the individual an effective amount of (a) an agent that decreases the expression and/or activity of CCAAT/enhancer-binding protein beta (Cebpb); (b) an agent that decreases the expression and/or activity of TNF receptor-associated factor 2 (Traf2); and/or (c) an agent that increases the expression and/or activity of Death-inducer obliterator 1 (Dido1).

In some aspects, anti-tumor immunity in the individual is increased by at least 10% relative to anti-tumor immunity in the absence of the agent.

In another aspect, the invention provides a method for decreasing the proportion of migratory dendritic cells (mDCs) in an individual, the method comprising administering to the individual an effective amount of (a) an agent that increases the expression and/or activity of CCAAT/enhancer-binding protein beta (Cebpb); (b) an agent that increases the expression and/or activity of TNF receptor-associated factor 2 (Traf2); and/or (c) an agent that decreases the expression and/or activity of Death-inducer obliterator 1 (Dido1).

In some aspects, the proportion is a proportion in a tumor or a tissue of the individual.

In some aspects, the proportion of mDCs in the individual is decreased by at least 10% relative to the proportion in the absence of the agent.

In another aspect, the invention provides a method for decreasing autoimmune activity in an individual, the method comprising administering to the individual an effective amount of (a) an agent that increases the expression and/or activity of CCAAT/enhancer-binding protein beta (Cebpb); (b) an agent that increases the expression and/or activity of TNF receptor-associated factor 2 (Traf2); and/or (c) an agent that decreases the expression and/or activity of Death-inducer obliterator 1 (Dido1).

In some aspects, autoimmune activity in the individual is decreased by at least 10% relative to anti-tumor immunity in the absence of the agent.

In some aspects, the inflammatory disease or autoimmune disease is a neurodegenerative disease, arthritis, allergy, eczema, fibrosis, asthma, lupus erythematosus, an inflammatory bowel disease, ulcerative colitis, or Crohn's disease. In some aspects, the neurodegenerative disease is MS, AD, ALS, or PD.

In some aspects, the agent is a proteolysis targeting chimera (PROTAC), a small molecule, an antibody or antigen-binding fragment thereof, a peptide, a mimic, or an inhibitory nucleic acid.

In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody or antigen-binding fragment thereof binds Cebpb, Traf2; and/or Dido1.

In some aspects, the method further comprises administering to the individual one or more additional agents.

In some aspects, the method further comprises administering to the individual one or more agents that modulate the expression of one or more of (a) Ago2, Ahr, Anapc13, Bach1, Baz1 a, Bid, Bptf, Brca1, Brwd3, Btbd1, Cblc, Ccnf, Cdc27, Cntn4, Copa, Copb2, Coro1 a, Cpne9, Cul4b, Ddb1, E4f1, Ecel1, Fbxl14, Fbxl5, Fbxo11, Fbxo42, Fzr1, Gemin5, Gm10697, Gm9117, Gtf2 h2, Gtf3c1, Hdac4, Hectdl, Ift122, Ikbkg, Ing2, Jun, Katnbl, Kbtbdl3, Kdm2a, Klhl23, Klhl3, Kmt2b, LOC100861784, Lrr1, Lrrc41, Map3k7, Mdm4, Mib1, Mkrn1, Mnat1, Naca, Nsmaf, Ogt, Pa2g4, Pcif1, Ppp1 r11, Prc1, Ring1, Rnf128, Rnf20, Rnf225, Rnf40, Siah1a, Siah2, Taf3, Tdpoz2, Tmem183a, Tnfsf11, Tradd, Traf3ip2, Trim35, Trim7, Tssc1, Ttc3, Ube2n, Ufl1, Unk1, Upf1, Vdr, Wdhd1, Wdr48, Wdr95, Wwp1, Ybx1, Zbtb14, Zbtb49, Zbtb7a, and Zmiz1; and/or (b) Akt1, Ankfy1, Apc, Arpc1 b, Birc2, Bmi1, Bub3, Cacybp, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31 a, Smad2, Syvn1, Taf51, Traf3, Traf7, Trim24, Trp53, Ube2e1, Ube2e3, Ube3c, Ufm1, Wdfy3, Wdr1, Wdr82, Whsc1, and Zbtb11.

In another aspect, the invention provides a kit comprising (a) an agent that decreases the expression and/or activity of Cebpb; (b) an agent that decreases the expression and/or activity of Traf2; and/or (c) an agent that increases the expression and/or activity of Dido1 for treating an individual having a cancer, an inflammatory disease, or an autoimmune disease according to any one of the methods provided herein. In some aspects, the kit comprises a package insert comprising instructions to administer the agent to an individual having a cancer, an inflammatory disease, or an autoimmune disease.

In another aspect, the invention provides a kit comprising (a) an agent that increases the expression and/or activity of Cebpb; (b) an agent that increases the expression and/or activity of Traf2; and/or (c) an agent that decreases the expression and/or activity of Dido1 for treating an individual having an inflammatory disease or an autoimmune disease according to any one of the methods provided herein. In some aspects, the kit comprises a package insert comprising instructions to administer the agent to an individual having an inflammatory disease or an autoimmune disease.

In another aspect, the invention provides a method of monitoring the response of an individual having a cancer, an inflammatory disease, an autoimmune disease, or an infectious disease to treatment with (a) an agent that decreases the expression and/or activity of Cebpb; (b) an agent that decreases the expression and/or activity of Traf2; and/or (c) an agent that increases the expression and/or activity of Dido1, the method comprising (i) determining, in a biological sample obtained from the individual at a time point following administration of the agent, the expression level of one or more of Cebpb, Traf2, and Dido1; and (ii) comparing the expression level of the one or more genes in the biological sample with a reference level, thereby monitoring the response in the individual to treatment with the agent.

In another aspect, the invention provides a method of monitoring the response of an individual having an inflammatory disease, an autoimmune disease, or an infectious disease to treatment with (a) an agent that increases the expression and/or activity of Cebpb; (b) an agent that increases the expression and/or activity of Traf2; and/or (c) an agent that decreases the expression and/or activity of Dido1, the method comprising (i) determining, in a biological sample obtained from the individual at a time point following administration of the agent, the expression level of one or more of Cebpb, Traf2, and Dido1; and (ii) comparing the expression level of the one or more genes in the biological sample with a reference level, thereby monitoring the response in the individual to treatment with the agent.

In some aspects, the reference level is selected from the group consisting of (i) the expression level of the one or more genes in a biological sample from the individual obtained prior to administration of the agent; (ii) the expression level of the one or more genes in a reference population; (iii) a pre-assigned expression level for the one or more genes; or (iv) the expression level of the one or more genes in a biological sample obtained from the individual at a previous time point, wherein the previous time point is following administration of the agent.

In some aspects, (a) the expression and/or activity of Cebpb is increased in the biological sample obtained from the individual relative to the reference level; (b) the expression and/or activity of Traf2 is increased in the biological sample obtained from the individual relative to the reference level; and/or (c) the expression and/or activity of Dido1 is decreased in the biological sample obtained from the individual relative to the reference level, and the method further comprises administering to the individual one or more additional doses of the agent, wherein the agent decreases the expression and/or activity of Cebpb; decreases the expression and/or activity of Traf2; and/or increases the expression and/or activity of Dido1.

In some aspects, (a) the expression and/or activity of Cebpb is decreased in the biological sample obtained from the individual relative to the reference level; (b) the expression and/or activity of Traf2 is decreased in the biological sample obtained from the individual relative to the reference level; and/or (c) the expression and/or activity of Dido1 is increased in the biological sample obtained from the individual relative to the reference level, and the method further comprises administering to the individual one or more additional doses of the agent, wherein the agent increases the expression and/or activity of Cebpb; increases the expression and/or activity of Traf2; and/or decreases the expression and/or activity of Dido1.

In another aspect, the invention provides a method for treating a cancer, an inflammatory disease, or an autoimmune disease in an individual, the method comprising administering to the individual an effective amount of a modulator of the interaction between (a) F-box and WD repeat domain containing 11 (Fbxw11) and (b) nuclear factor kappa B subunit 1 (Nfkb1) or nuclear factor kappa B subunit 2 (Nfkb2).

In some aspects, the individual has a cancer and the modulator is an agent that increases the expression and/or activity of Fbxw11.

In some aspects, the individual has an inflammatory disease or an autoimmune disease and the modulator is an agent that decreases the expression and/or activity of Fbxw11.

In another aspect, the invention provides a method for increasing processing of Nfkb1 and/or Nfkb2 into an active form, the method comprising contacting a cell capable of expressing Fbxw11 with an agent that increases expression and/or activity of Fbxw11.

In some aspects, the cell capable of expressing Fbxw11 is in an individual. In some aspects, the individual has a cancer.

In some aspects, the level of Nfkb1 and/or Nfkb2 in an active form is increased by at least 10% relative to the level in the absence of the agent.

In another aspect, the invention provides a method for decreasing processing of Nfkb1 and/or Nfkb2 into an active form, the method comprising contacting a cell capable of expressing Fbxw11 with an agent that decreases expression and/or activity of Fbxw11.

In some aspects, the cell capable of expressing Fbxw11 is in an individual.

In some aspects, the individual has an inflammatory disease or an autoimmune disease.

In some aspects, the level of Nfkb1 and/or Nfkb2 in an active form is decreased by at least 10% relative to the level in the absence of the agent.

In another aspect, the invention provides a method for increasing an immune response directed by Nfkb1 and/or Nfkb2 in an individual, the method comprising administering to the individual an effective amount of an agent that increases expression and/or activity of Fbxw11.

In some aspects, the individual has a cancer.

In another aspect, the invention provides a method for decreasing an immune response directed by Nfkb1 and/or Nfkb2 in an individual, the method comprising administering to the individual an effective amount of an agent that decreases expression and/or activity of Fbxw11.

In some aspects, the individual has an inflammatory disease or an autoimmune disease.

In some aspects, the inflammatory disease or autoimmune disease is a neurogenerative disease, arthritis, allergy, eczema, fibrosis, asthma, lupus erythematosus, an inflammatory bowel disease, ulcerative colitis, or Crohn's disease. In some aspects, the neurodegenerative disease is MS, AD, ALS, or PD.

In some aspects, the agent is a proteolysis targeting chimera (PROTAC), a small molecule, an antibody or antigen-binding fragment thereof, a peptide, a mimic, or an inhibitory nucleic acid. In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody or antigen-binding fragment thereof binds Fbxw11.

In some aspects, the method further comprises administering to the individual one or more additional agents.

In some aspects, the method further comprises administering to the individual one or more agents that modulate the expression of one or more of (a) Acaca, Ambra1, Amfr, Arih1, Cbll1, Cfap57, Cnot4, Cyld, Dcaf7, Det1, Dpf2, Eed, Efcab8, Egr2, Fasn, Fbxw7, Foxo3, Gsk3b, Hectd3, Hira, Icos, Ifnarl, Ikbke, Ints12, Junb, Kat6a, Kctd10, Kctd13, Kctd21, Kctd5, Klhl30, Klhl6, Lztr1, March6, Msl2, Nf1, Nsd1, Patz1, Pias1, Prdm1, Pten, Rfwd2, Rnf139, Socs3, Spag16, Strap, Stub1, Syk, Tab1, Tank, Tbk1, Tnf, Trim45, Trip12, Ube2j2, Wdfy2, Wdr61, Wdr81, Wdr91, Zbtb25, Zfp106, Zfp91, and Zmiz2; and/or (b) Ahctfl, Anapcl 1, Arih2, Arnt, Bcl6, Brap, Cbl, Cd28, Cstf1, Cul1, Cul3, Cul5, Dda1, Fbxo33, Fus, Gm9840, Hif1 a, Huwe1, Ing3, Kcmf1, Kdm5c, Keap1, Maea, Mycbp2, Nbeall, Nedd8, Nup43, Nup62, Phf8, Ptpn1, Rae1, Ranbp2, Rbbp6, Rbck1, Rbx1, Rc3 h1, Rela, Rlim, Rnf144a, Rnf31, Rnf7, Seh1I, Skp1a, Spop, Ssr3, Tbl1xr1, Tceb1, Tceb2, Tceb3, Tdpoz5, Thoc3, Tlr4, Traf6, Trim28, Trim33, Ube2d3, Ube2f, Ube2h, Ube2i, Ubr4, Ubr5, Vhl, Wdr20, Wdr26, Wdr33, Zbtb17, and Zbtb7b.

In another aspect, the invention provides a kit comprising a modulator of the interaction between (a) Fbxw11 and (b) Nfkb1 or Nfkb2 for treating an individual having a cancer, an inflammatory disease, or an autoimmune disease according to any one of the methods provided herein. In some aspects, the kit comprises a package insert comprising instructions to administer the modulator to an individual having a cancer, an inflammatory disease, or an autoimmune disease.

In another aspect, the invention provides a method of monitoring the response of an individual having a cancer, an inflammatory disease, or an autoimmune disease to treatment with a modulator of the interaction between (a) Fbxw11 and (b) Nfkb1 or Nfkb2, the method comprising (i) determining, in a biological sample obtained from the individual at a time point following administration of the modulator, the expression level of an active form of one or both of Nfkb1 and Nfkb2; and (ii) comparing the expression level of the active form of one or both of Nfkb1 and Nfkb2 in the biological sample with a reference level, thereby monitoring the response in the individual to treatment with the modulator.

In some aspects, the reference level is selected from the group consisting of (i) the expression level of the one or both genes in a biological sample from the individual obtained prior to administration of the modulator; (ii) the expression level of the one or both genes in a reference population; (iii) a pre-assigned expression level for the one or both genes; or (iv) the expression level of the one or both genes in a biological sample obtained from the individual at a previous time point, wherein the previous time point is following administration of the modulator.

In some aspects, the individual has a cancer, the expression level of the active form of one or both of Nfkb1 and Nfkb2 in is decreased in the biological sample obtained from the individual relative to the reference level, and the method further comprises administering to the individual one or more additional doses of the modulator, wherein the modulator is an agent that increases the expression and/or activity of Fbxw11.

In some aspects, the individual has an inflammatory disease or an autoimmune disease, the expression level of the active form of one or both of Nfkb1 and Nfkb2 is increased in the biological sample obtained from the individual relative to the reference level, and the method further comprises administering to the individual one or more additional doses of the modulator, wherein the modulator is an agent that decreases the expression and/or activity of Fbxw11.

In another aspect, the invention provides a method for treating a cancer, an inflammatory disease, or an autoimmune disease in an individual, the method comprising administering to the individual an effective amount of a cell therapy comprising a cell comprising alterations in at least two of the genes in one or more of the following co-functional gene modules: (a) Module M1 comprising Aamp, Bop1, Cirh1a, Dcaf13, Grb2, Myc, Nle1, Noll0, Pak1ip1, Ptpn11, Rack1, Raf1, Rrp9, Taf5, Tbl3, Uhrf1, Utp15, Utp18, Vprbp, Wdr3, Wdr36, Wdr43, Wdr5, Wdr74, and Wdr75; (b) Module M2 comprising Ago2, Ahr, Anapc13, Bach1, Baz1a, Bid, Bptf, Brca1, Brwd3, Btbd1, Cblc, Ccnf, Cdc27, Cntn4, Copa, Copb2, Coro1a, Cpne9, Cul4b, Ddb1, Dido1, E4f1, Ecel1, Fbxl14, Fbxl5, Fbxo11, Fbxo42, Fzr1, Gemin5, Gml0697, Gm9117, Gtf2 h2, Gtf3c1, Hdac4, Hectdl, Ift122, Ikbkg, Ing2, Jun, Katnbl, Kbtbdl3, Kdm2a, Klhl23, Klhl3, Kmt2b, LOC100861784, Lrr1, Lrrc41, Map3k7, Mdm4, Mib1, Mkrn1, Mnat1, Naca, Nsmaf, Ogt, Pa2g4, Pcif1, Ppp1r11, Prc1, Ring1, Rnf128, Rnf20, Rnf225, Rnf40, Siah1a, Siah2, Taf3, Tdpoz2, Tmem183a, Tnfsf11, Tradd, Traf3ip2, Trim35, Trim7, Tssc1, Ttc3, Ube2n, Ufl1, Unk1, Upf1, Vdr, Wdhd1, Wdr48, Wdr95, Wwp1, Ybx1, Zbtb14, Zbtb49, Zbtb7a, and Zmiz1; (c) Module M3 comprising Akt1, Ankfy1, Apc, Arpc1 b, Birc2, Bmi1, Bub3, Cacybp, Cebpb, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31 a, Smad2, Syvn1, Taf51, Traf2, Traf3, Traf7, Trim24, Trp53, Ube2e1, Ube2e3, Ube3c, Ufm1, Wdfy3, Wdr1, Wdr82, Whsc1, and Zbtb11; (d) Module M4 comprising Cdc40, Ddx41, Plrg1, Ppil2, Ppwd1, Prpf19, Prpf4, Sart1, Smu1, Snrnp40, and Wdr70; (e) Module M5 comprising Acaca, Ambra1, Amfr, Arih1, Cbl1, Cfap57, Cnot4, Cyld, Dcaf7, Det1, Dpf2, Eed, Efcab8, Egr2, Fasn, Fbxw7, Foxo3, Gsk3b, Hectd3, Hira, Icos, Ifnarl, Ikbke, Ints12, Junb, Kat6a, Kctd10, Kctd13, Kctd21, Kctd5, Klhl30, Klhl6, Lztr1, March6, Msl2, Nf1, Nfkb1, Nsd1, Patz1, Pias1, Prdm1, Pten, Rfwd2, Rnf139, Socs3, Spag16, Strap, Stub1, Syk, Tab1, Tank, Tbk1, Tnf, Trim45, Trip12, Ube2j2, Wdfy2, Wdr61, Wdr81, Wdr91, Zbtb25, Zfp106, Zfp91, and Zmiz2; and (f) Module M6 comprising Ahctfl, Anapcl 1, Arih2, Arnt, Bcl6, Brap, Cbl, Cd28, Cstf1, Cul1, Cul3, Cul5, Dda1, Fbxo33, Fbxw11, Fus, Gm9840, Hif1a, Huwe1, Ing3, Kcmf1, Kdm5c, Keap1, Maea, Mycbp2, Nbeall, Nedd8, Nup43, Nup62, Phf8, Ptpn1, Rae1, Ranbp2, Rbbp6, Rbck1, Rbx1, Rc3 h1, Rela, Rlim, Rnf144a, Rnf31, Rnf7, Seh11, Skp1a, Spop, Ssr3, Tbllxr1, Tceb1, Tceb2, Tceb3, Tdpoz5, Thoc3, Tlr4, Traf6, Trim28, Trim33, Ube2d3, Ube2f, Ube2h, Ube2i, Ubr4, Ubr5, Vhl, Wdr20, Wdr26, Wdr33, Zbtb17, and Zbtb7b.

In another aspect, the invention provides a method for treating a cancer, an inflammatory disease, or an autoimmune disease in an individual, the method comprising administering to the individual an effective amount of a cell therapy comprising a cell comprising alterations in at least two of the genes in one or more of the following gene sets: (a) Gene Set 1 comprising Aamp, Actb, Alcam, Ambra1, Anxa2, Aprt, Atp5e, B2m, Btf3, Ccdc88a, Cdh1, Chd4, Cirh1 a, Cox4i1, Cox7a21, Crebbp, Ctsb, Dcaf13, Ddx41, Eef1a1, Eef1b2, Eef1g, Eef2, Eif1, Eif3e, Eif3f, Eif3i, Eif3k, Fau, Gapdh, H2-D1, H2-K1, H2-M2, Hsp90ab1, Hspa5, Hspa8, Ill rn, Laptm5, Lhfpl2, March6, Ms4a7, Mtor, Myc, Naca, Ncl, Nf1, Noll0, Npm1, Ogt, Pabpc1, Paf1, Plrg1, Pparg, Psap, Rack1, Raf1, Rheb, Rpl10, Rpl10a, Rpll1, Rpl12, Rpl13, Rpl13a, Rpl14, Rpl15, Rpl17, Rpl18, Rpl18a, Rpl19, Rpl21, Rpl22, Rpl2211, Rpl23, Rpl23a, Rpl24, Rpl26, Rpl27a, Rpl28, Rpl29, Rpl3, Rpl30, Rpl31, Rpl32, Rpl34, Rpl35, Rpl35a, Rpl36, Rpl36a, Rpl37, Rpl37a, Rpl38, Rpl39, Rpl4, Rpl41, Rpl5, Rpl6, Rpl7, Rpl7a, Rpl8, Rpl9, Rplp0, Rplp1, Rplp2, Rps10, Rps11, Rps12, Rps13, Rps14, Rps15, Rps15a, Rps16, Rps17, Rps18, Rps19, Rps2, Rps20, Rps21, Rps23, Rps24, Rps25, Rps26, Rps27, Rps27a, Rps28, Rps29, Rps3, Rps3a1, Rps4x, Rps5, Rps6, Rps7, Rps8, Rps9, Rpsa, Rptor, Sgk1, Ssr4, Tab1, Taf5, Tpt1, Uhrf1, Uqcrh, Utp15, Wdr3, Wdr36, Wdr43, Wdr5, and Zbtb25; (b) Gene Set 2 comprising A1314180, Abcc1, Acod1, Akr1 a1, Alas1, Alox5ap, Ampd3, Arih1, Ass1, B430306N03Rik, Bach1, Blvrb, Bmi1, Brca1, Btbd1, Btg1, Cat, Ccr5, Cd36, Cd52, Cd53, Cd81, Chd4, Chpf2, Clec4n, Crebbp, Creg1, Cul3, Cxcl3, Cyb5a, Dap, Dars, Dck, Ddb1, Ddit3, Egr2, Eif3f, Eif3i, Ep300, Esd, Fbxl5, Fbxw11, Gbe1, Gclm, Gdap10, Gm9840, Gss, Gstm1, H3f3b, Hmox1, Hvcn1, 111f9, Inhba, Keap1, Lipa, Lmo4, Map3k7, Mcli, Mcoln2, Met, Mgst2, Mmp12, Mmp19, Mmp8, Mylip, Nampt, Nedd8, Nf1, Npy, Nrp1, Nup43, Nupr1, Paf1, Pf4, Pgd, Phldal, Pla2g7, Plet1, Ppfibp2, Prdx1, Prdx6, Preb, Prkcb, Procr, Ptgr1, Ptpn1, Raf1, Rbx1, Rhob, Rnasel, Rnf128, Runx2, Sdc4, Sec13, Seh11, Skp1 a, Slc43a2, Slc48a1, Slc7a11, Slpi, Smad2, Srxn1, Taldo1, Tarm1, Thbs1, Tlr2, Tlr4, Tma16, Tpm4, Traf2, Traf5, Traf6, Trip12, Tubb2a, Txnrd1, Ube2d3, Ube2n, Ubr5, Uchl1, Upf1, Wdr43, Wdr61, Zbtb17, and Zyx; (c) Gene Set 3 comprising Acp5, Ankfy1, Arpc1 b, Atp6v0d2, Bptf, Brap, C5ar1, Ccdc88a, Cd14, Cd36, Cd63, Cebpb, Chd4, Clec4d, Clec5a, Cpd, Creg1, Ctsb, Ctsz, Cul3, Ddhd1, Dnmt3a, Egr2, Emb, F630028010Rik, Fabp5, Fam46c, Fbxo42, Fcgr2b, Fn1, Foxo3, Fpr1, Ftl1, Gadd45a, Glrx, Gpnmb, Gpr84, Huwe1, Icam1, Id1, Il1f9, Kctd10, Keap1, Klhl6, Lcn2, Lgals1, Lgals3, Lgmn, Lipa, Lpcat2, Ly6c2, March6, Metrnl, Mgll, Mt1, Mtor, Myof, Naaa, Naca, Nf1, Paf1, Phldal, Pid1, Pik3r4, Pld3, Plet1, Plk2, Pou2f2, Pparg, Prdx5, Psap, Ptpn11, Rab3il1, Rela, Rfwd2, Rnase2a, S100a1, S100a11, S100a8, Saa3, Sdc3, Serpinb2, Slamf7, Snx18, Sod2, Spatal3, Stap1, Strap, Tab1, Tceb2, Tgfbi, Thbs1, Trem1, Upf1, Upp1, Vat1, Wdfy3, Wfdc21, 2010005H15Rik, and Zbtb25; (d) Gene Set 4 comprising AC160336.1, Actb, Actg1, Ankfy1, Arhgdib, Bptf, Brap, Bri3, Ccr2, Ccr5, Cd274, Cdkn1a, Cfl1, Chd4, Clec4a2, Copa, Coro1 a, Cotl1, Crip1, Cul1, Cul3, Dars, Ddhd1, Ddit3, Ear2, Eif3f, Eif3i, Ep300, Fbxw11, FIna, Gbp2, Gbp5, Grb2, Gtf3c1, H2-D1, H2-K1, Huwe1, Ifi2712a, Ill rn, Keap1, Klk1 b1, Lcp1, Lgals1, Lpl, Lrr1, Lsp1, Malat1, Marcksl1, Med8, Mgll, Mndal, Mtor, Naca, Nedd8, Nf1, Paf1, Pfn1, Pik3r4, Pten, Ptma, Ptpn11, Rack1, Rela, Rnf20, Sdc4, Skp1 a, Taf3, Taf5, Tlr4, Tmsb4x, Ubb, Ube2i, Upf1, Vhl, Wdfy3, Wdr43, Wdr82, and Wfdc17; (e) Gene Set 5 comprising AA467197, AW112010, Abcg1, Acod1, Bcl2a1b, Bcl2a1d, Cav1, Ccll7, Ccl3, Ccl4, Cd14, Cd200r1, Cd300lf, Cdkn1a, Cebpb, Cflar, Chd4, Clec4e, Clic4, Copa, Cpd, Cpeb4, Cul1, Cul3, Cxcl1, Cxcl2, Cxcl3, Ehd1, Ep300, Fam102b, Fam20c, Fbxw11, Gda, Gpr84, Hist1h1c, Hivep3, Ikbke, Ikbkg, Il12b, Il1a, Il1b, 116, Ing3, Inhba, Kctd21, Klf4, Laptm5, Mafb, Malati, Malt1, Marcks, Marcksl1, Marco, Met, Mtpn, Nabp1, Nedd8, Nfkb1, Nfkbiz, Nlrp3, Nrp2, Nup62, Ogt, Paf1, Plek, Plrg1, Ppfia3, Prpf19, Ptgs2, Ptx3, Rassf4, Rbx1, Rela, Rfwd2, Rnf31, Serpinb2, Sh3bp5, Skp1a, Slc7a11, Slc7a2, Slco3a1, Slfn2, Smad2, Smu1, Socs6, Sod2, Spop, Stub1, Tank, Tbk1, Tceb3, TIr4, Tnf, Tnfaip3, Tnfsf15, Tradd, Traf6, Trip12, Txnip, Ube2d3, Ube2i, Ube2n, Wdr82, Zbtb17, and Zc3h12c; (f) Gene Set 6 comprising AA467197, Ahr, Akt1, Ankfy1, Axl, Bhlhe40, Bhlhe41, Btg1, Cc117, Cc122, Ccr2, Cd40, Cd52, Cd74, Cebpb, Chd4, Clec4e, Clec4n, Clic4, Cst3, Cstf1, Ctsb, Ctsd, Cxcl16, Dcstamp, Egr2, Etv3, Fabp4, Fabp5, Fam20c, Fbxw7, Fbxw9, Foxo3, Fpr1, Fth1, Ftli, Gbp2, Gbp5, Gm2a, Gnb1, Gnb2, Grb2, Grk3, Gsk3b, H2-Aa, H2-Ab1, Hmox1, Igf1, Il4i1, Irf4, Itgax, Jak2, Jund, Kcmf1, Klhl6, Kmt2d, Lgals1, Lyz2, March6, Mg12, Mmp12, Mtor, Myc, Ndufa4, Nectin2, Nf1, Nfkb1, Pfkp, Pid1, Pik3r4, Plet1, Pmp22, Pten, Ptpn1, Ptpn11, Rheb, Rilpl2, Rptor, S100a8, Sart1, Scimp, Sdcbp, Sema4a, Sgk1, Slamf9, Smad2, Srgn, Stat5a, Tab1, Taf51, Tank, Tceb1, Tceb2, Tlr2, TIr4, Traf2, Traf3, Ube2n, Vcan, Wdfy3, Wdr26, Wdr61, and Zfp3611; (g) Gene Set 7 comprising Abca1, Actb, Ambra1, Atf4, Atp5g1, Atp5j, Atp5j2, Bcl2a1b, Calm1, Cfli, Chd4, Copa, Copb2, Cotli, Cox8a, Cul3, Cybb, Dbi, Ddit3, Eef1a1, Eif3f, Eif3i, Fbxo28, Fcer1g, Gpx1, Grb2, H2-M2, H2afz, H3f3a, Ilrn, Inhba, Keap1, Kmt2d, Lhfpl2, Ly6e, March6, Med8, Mtor, Nedd8, Nf1, Nme1, Ogt, Paf1, Plrg1, Pnp, Pparg, Rack1, S100a10, S100a4, S100a6, Sdc4, Sec13, Serf2, Sgk1, Smad2, Smu1, Sqstmi, Tab1, Taf3, Trp53, Uhrf1, Wdr43, Wdr61, and Zbtb25; (h) Gene Set 8 comprising Aamp, Acsli, Ambrai, Arf4, Arih2, Atf4, Bop1, Clqb, Calr, Canx, Ccng1, Cdkn1a, Chd4, Cirh1a, Clec2d, Copa, Copb2, Cope, Cpd, Ctss, Cul3, Dad1, Dap, Dcaf13, Ddit3, Ddx41, Dstn, Eif3f, Eif3i, Erp29, Fbxw7, Fth1, Ftli, Gm9840, Grb2, Gtf3c1, Herpudi, Hif1a, Hnrnpa3, Hsp90bi, Hspa5, Ift20, Keap1, Kmt2d, Krtcap2, Lgals3, Lrr1, Lyz2, Manf, Map3k7, Mthfd2, Mtor, Myc, Naca, Nedd8, Nf1, Nol10, Ostc, P4 hb, Pdia3, Pdia4, Pdia6, Phgdh, Plrg1, Preb, Prpf19, Pten, Ptpn1, Rack1, Rbx1, Rela, Rp12211, Rpn1, Rps19, Rrp9, Sdf2li, Sec11c, Sec13, Sec22b, Sec31a, Sec61b, Sec61g, Selenos, Serf2, Serp1, Sf3b5, Spcs2, Ssr3, Surf4, Syvn1, Tceal9, Tceb1, Tceb2, Timm13, Tpt1, Tram1, Trp53, Ube2f, Ufm1, Uqcrq, Utp15, Vcp, Vhl, Vprbp, Wdr36, Wdr43, Wdr5, Wdr74, Wdr75, and Xbp1; (i) Gene Set 9 comprising Acod1, Adam8, Atp5g3, Brap, C3ar1, Ccl2, Ccl3, Ccl4, Ccl7, Ccnd1, Cd3001d, Cd63, Ch25h, Chd4, Chil3, Crip1, Ctsb, Ctsl, Cul1, Cul3, Cxcl1, Cyp51, Det1, Ear2, Egr2, F10, Fbxo42, Fbxw11, Ffar2, Fpr2, Fyb, Gas7, Gm9840, Gnb2, Gpnmb, Grb2, Gsk3b, Hmgcsi, Huwe1, Ifitm3, Il1f9, Itgam, Jun, Kctd12, Kctd5, Keap1, Klhdc4, Kmt2c, Kmt2d, Lgals1, Lgals3, Lmna, Lmo4, Lrpapi, Ly6c2, Lztr1, Maf, March6, Mcempi, Mmp12, Mmp13, Mmp8, Msr1, Mtor, Naaa, Naca, Nf1, Nfkbiz, Npc2, Npy, Paf1, Pdpn, Pf4, Plet1, Pparg, Prkcd, Pten, Ptgs2, Ptpn1, Ptpn11, Ptprc, Ptx3, Rbbp5, Rela, Rfwd2, Rheb, Rptor, S100a6, Saa3, Scap, Scd2, Serpinb2, Serpinb6a, Sgk1, Slc7a11, Smad2, Srgn, Syk, Syngri, Timp2, Trem2, Ube2h, Ube2i, Ucp2, Vasp, Vhl, Wdr26, Wfdc21, Ybx1, Zbtb7a, and Zfp3612; (j) Gene Set 10 comprising Acaca, Ak4, Aldoa, Aldoc, Anapc13, Anxa2, Arih2, Arnt, Basp1, Bnip31, Bsg, C3ar1, Cc19, Cd52, Chil3, Copa, Cul2, Cul3, Cul5, Egr2, Eif3i, Eif4ebpi, Emilin2, Eno1, Ep300, Fam162a, Gapdh, Gbe1, Gpi1, Gsn, Herpudi, Hif1a, Higd1a, Hilpda, Hk1, Hk2, Hmox1, Huwe1, ler3, Kctd10, Klk1b1, Ldha, Lgals3, Lipa, Lmo4, Lpcat2, Lyz2, March6, Mif, Mt1, Mt2, Mtor, Myc, Ndufv3, Nf1, Pdk1, Pfkl, Pgam1, Pgk1, Pgm2, Pkm, Prdx1, Prelidi, Ptpn1, Ptpn11, Rbpj, Rfwd2, Rilpl2, Rnase2a, Sacs, Scd2, Sdc3, Sdc4, Sec13, Slamf9, Slc16a3, Slc2a1, Slc7a2, Smu1, Socs3, Strap, Tarm1, Tceb1, Tceb2, Tgm2, TIr4, Tpi1, Trf, Ube2f, Vhl, Vim, Wdr43, Wdr82, Wfdc17, and 2010005H1i5Rik; (k) Gene Set 11 comprising AA467197, Apobeci, Apoe, Ciqa, Clqb, Clqc, C3, Car4, Ccl22, Ccl3, Ccl4, Ccl6, Cc19, Cd83, Cdc40, Cebpb, Ch25h, Chd4, Copa, Crebbp, Cull, Ddhd1, Ddx41, Egr2, Eif3f, Eif3i, Ep300, Fam49a, Fbxw11, Fn1, Fnbp11, Gadd45b, Hdac4, Icam1, Icosl, Id2, Ikbkg, Il1a, 114i1, Inhba, Itgax, Itgb2, Kctd10, Klk1b11, Lpl, Maf, Marcks, Marcksll, Med8, Met, Mmp12, Ms4a6c, Ms4a7, Mt2, Mycbp2, Naca, Nedd8, Nfkbia, Phldal, Plaur, Plrg1, Pparg, Ppfibp2, Prpf19, Ptpn1, Rassf4, Rfwd2, Ring1, Rnase2a, Rpl12, Scimp, Sec13, Skp1a, Slc43a2, Smu1, Sqstml, Syk, Syvn1, Taf51, Tceb2, Tmem176a, Tmem176b, Tnfaip2, Traf3, Ufm1, Upp1, Wdr5, Wdr70, Wdr82, Wfdc17, Wfdc21, 0610012G03Rik, Zbtb7a, and Zyx; (I) Gene Set 12 comprising Ambra1, Aplp2, Atp5g1, Atpif1, B2m, Ccdc88a, Chd4, Copa, Cyba, Ddit3, Ear2, Egr2, Eif3f, Eif3i, Eif5, Fcgrt, Grn, H2-M2, H2-Q6, Hint1, Id1, Ifi204, Itgal, Kctd12, Laptm5, Lgals3, Ly6e, Mgst1, Mpeg1, Mtdh, Nf1, Nfe212, Nupr1, Paf1, Pparg, Prpf19, Psmb5, Psmb6, Pycard, Rack1, Rnase4, Rpl2211, Rpl37a, Rplp0, Sart1, Sdc3, Sec61b, Smad2, Smdt1, Smu1, Spp1, Syvn1, Tab1, Taf5, Taf51, Tagln2, Tmsb10, Traf2, Traf3, Trf, Trp53, Upf1, and Wdr5; (m) Gene Set 13 comprising Ankfy1, Anxa1, Anxa5, Aph1c, Brap, C3ar1, Ccnd2, Ccr1, Cd3001f, Cd38, Cd68, Cd9, Cdc27, Cdc40, Cebpb, Chd4, Chst11, Clec4e, Creb5, Cul1, Cul3, Cxcl3, Cyba, Dstn, Eif3f, Eif3i, Emp1, Epha4, Fam102b, Fam46a, Fbxw11, Fn1, Foxo3, Ft|l, Furin, Gas7, Gdf15, Grb2, H2-K1, Huwe1, Icam1, 117r, Inhba, Keap1, Klhdc4, Klk1bll, Lgals3, Lpl, Ly6c2, Lyz2, March6, Mbnll, Mmp14, Mmp8, Ms4a7, Naca, Neat1, Nf1, Nrp2, Plin2, Plk2, Plrg1, Polr21, Prdx1, Pten, Ptpn1, Rack1, Rasgeflb, Rasgrpl, Rela, Rnf20, Rnh1, Rpl2211, Rrp9, Saa3, Scd2, Sdc4, Sec13, Selenoh, Serp1, Skp1 a, Slamf7, Slc7a2, Smu1, Spp1, Tab1, Taf5, Ube2d3, Ubr4, Upf1, Vim, Wdr43, Wdr5, Wdr70, Wdr82, and Zbtb25; (n) Gene Set 14 comprising AC160336.1, Adgrel, Adgre4, Adgrl2, Anxa1, B2m, Clqb, C3, Car4, Ccdc88a, Ccl6, Cd52, Cdc40, Chd4, Chil3, Crip1, Ctsk, Ddx41, Dpf2, Egr2, Eif3i, Ep300, F7, Fcer1g, Fn1, Foxo3, Gpx3, H2-D1, H2-K1, H2-Q6, H2-Q7, H3f3b, Hira, Hsp90aal, Hvcn1, Id2, Ifi203, 1118, 111f9, Kdm5c, Klhl6, Lgals1, Lgals3, Ly6e, Malt1, March6, Marcks, Mcub, Med8, Mpc1, Ms4a6d, Msrb1, Mt1, Mt2, Nedd8, Nfe212, Nov, Npc2, Paf1, Pdzk1ip1, Phgdh, Pias1, Pla2g7, Plrg1, Ppic, Ppil2, Ppwd1, Prkcd, Prpf19, Ptges, Rab32, Rbx1, Rela, Rps20, S100a11, Sart1, Selenow, Smu1, St8sia4, Tab1, Taf51, Tceb2, Tmem176a, Tmem176b, Tnip3, Traf2, Tyrobp, Ube2i, Uchl1, Wdr5, Wdr70, Wdr82, Zbtb25, and Zfp106; and (o) Gene Set 15 comprising AC160336.1, Adgrel, Ahnak, Alcam, Aprt, Bcl2ll1, Blvrb, Brap, Bub3, Clqb, Clqc, C3ar1, Cd300c2, Cd33, Cd68, Cdc40, Cebpb, Chchd2, Clec12a, Clec4n, Copa, Csf1r, Ctsz, Cul3, Cul5, Cyba, Ddx41, Dstn, Egr2, Ep300, F7, Fbxw7, Fcer1g, Fcgr2b, Gmfg, Gngt2, Gpr84, Hsp90aal, Huwe1, Igf1, Kat6a, Kctdl2b, Kdm5c, Keap1, Kmt2d, Lst1, Mmp14, Mpeg1, Myc, Naca, P2ry14, Paf1, Pirb, Plrg1, Pou2f2, Pparg, Ppil2, Ppwd1, Prkcd, Prpf19, Prpf4, Ptpn1, Ptpn18, Rack1, Rbbp5, Rnf20, Rnf40, Rnf7, Rps271, Sat1, Serpinb2, Smu1, Socs3, Spp1, Taf5, Tank, Tceb1, Tceb2, Tgm2, Tnfsfl 5, Traf2, Trem2, Tyrobp, Ufm1, Vcan, Wdr1, Wdr33, Wdr43, Wdr5, Wdr61, Wdr70, Wdr82, Wfdc21, and Ybx1.

In some aspects, the cell therapy is a dendritic cell therapy, a macrophage cell therapy, an adoptive T cell therapy (ACT), a tumor-infiltrating lymphocyte (TIL) therapy, an engineered T cell receptor (TCR) therapy, a chimeric antigen receptor T cell (CAR-T) therapy, a CAR-Treg therapy, or a natural killer (NK) cell therapy.

In another aspect, the invention provides a kit comprising a cell therapy comprising a cell comprising alterations in at least two of the genes in one or more of the co-functional gene modules provided above for treating an individual having a cancer, an inflammatory disease, or an autoimmune disease according to a method provided herein.

In another aspect, the invention provides a kit comprising reagents for modifying a cell to comprise alterations in at least two of the genes in one or more of the co-functional gene modules provided above for treating an individual having a cancer, an inflammatory disease, or an autoimmune disease according to a method provided herein.

In some aspects, the kit comprises a package insert comprising instructions to administer the agent to an individual having a cancer, an inflammatory disease, or an autoimmune disease.

In another aspect, the invention provides a kit comprising a cell therapy comprising a cell comprising alterations in at least two of the genes in one or more of the gene sets provided above for treating an individual having a cancer, an inflammatory disease, or an autoimmune disease according to a method provided herein.

In another aspect, the invention provides a kit comprising reagents for modifying a cell to comprise alterations in at least two of the genes in one or more of the gene sets provided above for treating an individual having a cancer, an inflammatory disease, or an autoimmune disease according to a method provided herein.

In some aspects, the kit comprises a package insert comprising instructions to administer the agent to an individual having a cancer, an inflammatory disease, or an autoimmune disease.

In another aspect, the invention provides a genetically modified isolated cell comprising alterations in at least two of the genes in one or more of the following co-functional gene modules: (a) Module M1 comprising Aamp, Bop1, Cirh1a, Dcaf13, Grb2, Myc, Nle1, Noll0, Pak1ip1, Ptpn11, Rack1, Raf1, Rrp9, Taf5, Tbl3, Uhrf1, Utp15, Utp18, Vprbp, Wdr3, Wdr36, Wdr43, Wdr5, Wdr74, and Wdr75; (b) Module M2 comprising Ago2, Ahr, Anapc13, Bach1, Baz1 a, Bid, Bptf, Brca1, Brwd3, Btbd1, Cblc, Ccnf, Cdc27, Cntn4, Copa, Copb2, Coro1a, Cpne9, Cul4b, Ddb1, Dido1, E4f1, Ecel1, Fbxl14, Fbxl5, Fbxo11, Fbxo42, Fzr1, Gemin5, Gm10697, Gm9117, Gtf2 h2, Gtf3c1, Hdac4, Hectdl, Ift122, Ikbkg, Ing2, Jun, Katnbl, Kbtbdl3, Kdm2a, Klhl23, Klhl3, Kmt2b, LOC100861784, Lrr1, Lrrc41, Map3k7, Mdm4, Mib1, Mkrn1, Mnat1, Naca, Nsmaf, Ogt, Pa2g4, Pcif1, Ppp1r11, Prc1, Ring1, Rnf128, Rnf20, Rnf225, Rnf40, Siah1 a, Siah2, Taf3, Tdpoz2, Tmem183a, Tnfsfl 1, Tradd, Traf3ip2, Trim35, Trim7, Tssc1, Ttc3, Ube2n, Ufl1, Unk1, Upf1, Vdr, Wdhd1, Wdr48, Wdr95, Wwp1, Ybx1, Zbtb14, Zbtb49, Zbtb7a, and Zmiz1; (c) Module M3 comprising Akt1, Ankfy1, Apc, Arpc1 b, Birc2, Bmi1, Bub3, Cacybp, Cebpb, Chd4, Crebbp, Cul2, Dars, Dcafl0, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31 a, Smad2, Syvn1, Taf5l, Traf2, Traf3, Traf7, Trim24, Trp53, Ube2e1, Ube2e3, Ube3c, Ufm1, Wdfy3, Wdr1, Wdr82, Whsc1, and Zbtb11; (d) Module M4 comprising Cdc40, Ddx41, Plrg1, Ppil2, Ppwd1, Prpf19, Prpf4, Sart1, Smu1, Snrnp40, and Wdr70; (e) Module M5 comprising Acaca, Ambra1, Amfr, Arih1, Cbll1, Cfap57, Cnot4, Cyld, Dcaf7, Det1, Dpf2, Eed, Efcab8, Egr2, Fasn, Fbxw7, Foxo3, Gsk3b, Hectd3, Hira, Icos, Ifnarl, Ikbke, Ints12, Junb, Kat6a, Kctd10, Kctd13, Kctd21, Kctd5, Klhl30, Klhl6, Lztr1, March6, Msl2, Nf1, Nfkb1, Nsd1, Patz1, Pias1, Prdm1, Pten, Rfwd2, Rnf139, Socs3, Spag16, Strap, Stub1, Syk, Tab1, Tank, Tbk1, Tnf, Trim45, Trip12, Ube2j2, Wdfy2, Wdr61, Wdr81, Wdr91, Zbtb25, Zfp106, Zfp91, and Zmiz2; and (f) Module M6 comprising Ahctf1, Anapc11, Arih2, Arnt, Bcl6, Brap, Cbl, Cd28, Cstf1, Cul1, Cul3, Cul5, Dda1, Fbxo33, Fbxw11, Fus, Gm9840, Hif1a, Huwe1, Ing3, Kcmf1, Kdm5c, Keap1, Maea, Mycbp2, Nbeal1, Nedd8, Nup43, Nup62, Phf8, Ptpn1, Rae1, Ranbp2, Rbbp6, Rbck1, Rbx1, Rc3 h1, Rela, Rlim, Rnf144a, Rnf31, Rnf7, Seh11, Skp1a, Spop, Ssr3, Tbl1xr1, Tceb1, Tceb2, Tceb3, Tdpoz5, Thoc3, Tlr4, Traf6, Trim28, Trim33, Ube2d3, Ube2f, Ube2h, Ube2i, Ubr4, Ubr5, Vhl, Wdr20, Wdr26, Wdr33, Zbtb17, and Zbtb7b.

In another aspect, the invention provides a genetically modified isolated cell comprising alterations in at least two of the genes in one or more of the following gene sets: (a) Gene Set 1 comprising Aamp, Actb, Alcam, Ambra1, Anxa2, Aprt, Atp5e, B2m, Btf3, Ccdc88a, Cdh1, Chd4, Cirh1a, Cox4i1, Cox7a21, Crebbp, Ctsb, Dcaf13, Ddx41, Eef1a1, Eef1b2, Eef1g, Eef2, Eif1, Eif3e, Eif3f, Eif3i, Eif3k, Fau, Gapdh, H2-D1, H2-K1, H2-M2, Hsp90ab1, Hspa5, Hspa8, Ill rn, Laptm5, Lhfpl2, March6, Ms4a7, Mtor, Myc, Naca, Ncl, Nf1, Noll0, Npm1, Ogt, Pabpc1, Paf1, Plrg1, Pparg, Psap, Rack1, Raf1, Rheb, Rpl10, Rpl10a, Rpll1, Rpl12, Rpl13, Rpl13a, Rpl14, Rpl15, Rpl17, Rpl18, Rpl18a, Rpl19, Rpl21, Rpl22, Rpl2211, Rpl23, Rpl23a, Rpl24, Rpl26, Rpl27a, Rpl28, Rpl29, Rpl3, Rpl30, Rpl31, Rpl32, Rpl34, Rpl35, Rpl35a, Rpl36, Rpl36a, Rpl37, Rpl37a, Rpl38, Rpl39, Rpl4, Rpl41, Rpl5, Rpl6, Rpl7, Rpl7a, Rpl8, Rpl9, Rplp0, Rplp1, Rplp2, Rps10, Rps11, Rps12, Rps13, Rps14, Rps15, Rps15a, Rps16, Rps17, Rps18, Rps19, Rps2, Rps20, Rps21, Rps23, Rps24, Rps25, Rps26, Rps27, Rps27a, Rps28, Rps29, Rps3, Rps3a1, Rps4x, Rps5, Rps6, Rps7, Rps8, Rps9, Rpsa, Rptor, Sgk1, Ssr4, Tab1, Taf5, Tpt1, Uhrf1, Uqcrh, Utp15, Wdr3, Wdr36, Wdr43, Wdr5, and Zbtb25; (b) Gene Set 2 comprising A1314180, Abcc1, Acod1, Akr1 a1, Alas1, Alox5ap, Ampd3, Arih1, Ass1, B430306N03Rik, Bach1, Blvrb, Bmi1, Brca1, Btbd1, Btg1, Cat, Ccr5, Cd36, Cd52, Cd53, Cd81, Chd4, Chpf2, Clec4n, Crebbp, Creg1, Cul3, Cxcl3, Cyb5a, Dap, Dars, Dck, Ddb1, Ddit3, Egr2, Eif3f, Eif3i, Ep300, Esd, Fbxl5, Fbxw11, Gbe1, Gclm, Gdap10, Gm9840, Gss, Gstm1, H3f3b, Hmox1, Hvcn1, Il1f9, Inhba, Keap1, Lipa, Lmo4, Map3k7, Mcli, Mcoln2, Met, Mgst2, Mmp12, Mmp19, Mmp8, Mylip, Nampt, Nedd8, Nf1, Npy, Nrp1, Nup43, Nupr1, Paf1, Pf4, Pgd, Phldal, Pla2g7, Plet1, Ppfibp2, Prdx1, Prdx6, Preb, Prkcb, Procr, Ptgr1, Ptpn1, Raf1, Rbx1, Rhob, Rnasel, Rnf128, Runx2, Sdc4, Sec13, Seh11, Skp1a, Slc43a2, Slc48a1, Slc7a11, Slpi, Smad2, Srxn1, Taldo1, Tarm1, Thbs1, Tlr2, Tlr4, Tma16, Tpm4, Traf2, Traf5, Traf6, Trip12, Tubb2a, Txnrd1, Ube2d3, Ube2n, Ubr5, Uchll, Upf1, Wdr43, Wdr61, Zbtb17, and Zyx; (c) Gene Set 3 comprising Acp5, Ankfy1, Arpc1b, Atp6vOd2, Bptf, Brap, C5ar1, Ccdc88a, Cd14, Cd36, Cd63, Cebpb, Chd4, Clec4d, Clec5a, Cpd, Creg1, Ctsb, Ctsz, Cul3, Ddhd1, Dnmt3a, Egr2, Emb, F630028010Rik, Fabp5, Fam46c, Fbxo42, Fcgr2b, Fn1, Foxo3, Fpr1, Ftl1, Gadd45a, Glrx, Gpnmb, Gpr84, Huwe1, Icam1, Id1, Il1f9, Kctd10, Keap1, Klhl6, Lcn2, Lgals1, Lgals3, Lgmn, Lipa, Lpcat2, Ly6c2, March6, Metrnl, Mgll, Mt1, Mtor, Myof, Naaa, Naca, Nf1, Paf1, Phldal, Pid1, Pik3r4, Pld3, Plet1, Plk2, Pou2f2, Pparg, Prdx5, Psap, Ptpn11, Rab3il1, Rela, Rfwd2, Rnase2a, S100a1, S100a11, S100a8, Saa3, Sdc3, Serpinb2, Slamf7, Snx18, Sod2, Spata13, Stap1, Strap, Tab1, Tceb2, Tgfbi, Thbs1, Trem1, Upf1, Upp1, Vat1, Wdfy3, Wfdc21, 2010005H15Rik, and Zbtb25; (d) Gene Set 4 comprising AC160336.1, Actb, Actg1, Ankfy1, Arhgdib, Bptf, Brap, Bri3, Ccr2, Ccr5, Cd274, Cdkn1a, Cfl1, Chd4, Clec4a2, Copa, Coro1a, Cotl1, Crip1, Cul1, Cul3, Dars, Ddhd1, Ddit3, Ear2, Eif3f, Eif3i, Ep300, Fbxw11, FIna, Gbp2, Gbp5, Grb2, Gtf3c1, H2-D1, H2-K1, Huwe1, Ifi2712a, Ill rn, Keap1, Klk1 b1, Lcp1, Lgals1, Lpl, Lrr1, Lsp1, Malat1, Marcksl1, Med8, Mgll, Mndal, Mtor, Naca, Nedd8, Nf1, Paf1, Pfn1, Pik3r4, Pten, Ptma, Ptpn11, Rack1, Rela, Rnf20, Sdc4, Skp1 a, Taf3, Taf5, Tlr4, Tmsb4x, Ubb, Ube2i, Upf1, Vhl, Wdfy3, Wdr43, Wdr82, and Wfdc17; (e) Gene Set 5 comprising AA467197, AW112010, Abcg1, Acod1, Bcl2a1b, Bcl2a1d, Cav1, Ccll7, Ccl3, Cc14, Cd14, Cd200r1, Cd3001f, Cdkn1a, Cebpb, Cflar, Chd4, Clec4e, Ciic4, Copa, Cpd, Cpeb4, Cul1, Cul3, Cxcl1, Cxcl2, Cxcl3, Ehd1, Ep300, Fam102b, Fam20c, Fbxw11, Gda, Gpr84, Hist1h1c, Hivep3, Ikbke, Ikbkg, Il12b, Il1a, Il1b, 116, Ing3, Inhba, Kctd21, Klf4, Laptm5, Mafb, Malat1, Malt1, Marcks, Marcksl1, Marco, Met, Mtpn, Nabp1, Nedd8, Nfkb1, Nfkbiz, Nlrp3, Nrp2, Nup62, Ogt, Paf1, Plek, Plrg1, Ppfia3, Prpf19, Ptgs2, Ptx3, Rassf4, Rbx1, Rela, Rfwd2, Rnf31, Serpinb2, Sh3bp5, Skp1a, Slc7a11, Slc7a2, Slco3a1, Slfn2, Smad2, Smu1, Socs6, Sod2, Spop, Stub1, Tank, Tbk1, Tceb3, Tir4, Tnf, Tnfaip3, Tnfsf15, Tradd, Traf6, Trip12, Txnip, Ube2d3, Ube2i, Ube2n, Wdr82, Zbtb17, and Zc3h12c; (f) Gene Set 6 comprising AA467197, Ahr, Akt1, Ankfyi, Axl, Bhlhe40, Bhlhe41, Btg1, Cc117, Cc122, Ccr2, Cd40, Cd52, Cd74, Cebpb, Chd4, Clec4e, Clec4n, Ciic4, Cst3, Cstf1, Ctsb, Ctsd, Cxcl16, Dcstamp, Egr2, Etv3, Fabp4, Fabp5, Fam20c, Fbxw7, Fbxw9, Foxo3, Fpr1, Fth1, Ftl1, Gbp2, Gbp5, Gm2a, Gnb1, Gnb2, Grb2, Grk3, Gsk3b, H2-Aa, H2-Ab1, Hmox1, Igf1, Ii4i1, Irf4, Itgax, Jak2, Jund, Kcmf1, Klhl6, Kmt2d, Lgals1, Lyz2, March6, Mgi2, Mmp12, Mtor, Myc, Ndufa4, Nectin2, Nf1, Nfkb1, Pfkp, Pid1, Pik3r4, Plet1, Pmp22, Pten, Ptpn1, Ptpn11, Rheb, Rilpl2, Rptor, S100a8, Sart1, Scimp, Sdcbp, Sema4a, Sgk1, Slamf9, Smad2, Srgn, Stat5a, Tab1, Taf51, Tank, Tceb1, Tceb2, Tlr2, Tir4, Traf2, Traf3, Ube2n, Vcan, Wdfy3, Wdr26, Wdr61, and Zfp3611; (g) Gene Set 7 comprising Abca1, Actb, Ambra1, Atf4, Atp5g1, Atp5j, Atp5j2, Bcl2a1 b, Calm1, Cfl1, Chd4, Copa, Copb2, Cotl1, Cox8a, Cul3, Cybb, Dbi, Ddit3, Eef1a1, Eif3f, Eif3i, Fbxo28, Fcer1g, Gpx1, Grb2, H2-M2, H2afz, H3f3a, Ilrn, Inhba, Keap1, Kmt2d, Lhfpl2, Ly6e, March6, Med8, Mtor, Nedd8, Nf1, Nme1, Ogt, Paf1, Plrg1, Pnp, Pparg, Rack1, S100a10, S100a4, S100a6, Sdc4, Sec13, Serf2, Sgk1, Smad2, Smu1, Sqstm1, Tab1, Taf3, Trp53, Uhrf1, Wdr43, Wdr61, and Zbtb25; (h) Gene Set 8 comprising Aamp, Acsi1, Ambra1, Arf4, Arih2, Atf4, Bop1, C1 qb, Calr, Canx, Ccng1, Cdkn1 a, Chd4, Cirh1 a, Clec2d, Copa, Copb2, Cope, Cpd, Ctss, Cul3, Dad1, Dap, Dcaf13, Ddit3, Ddx41, Dstn, Eif3f, Eif3i, Erp29, Fbxw7, Fth1, Ftil, Gm9840, Grb2, Gtf3c1, Herpud1, Hif1 a, Hnrnpa3, Hsp90b1, Hspa5, Ift20, Keap1, Kmt2d, Krtcap2, Lgals3, Lrr1, Lyz2, Manf, Map3k7, Mthfd2, Mtor, Myc, Naca, Nedd8, Nf1, Nol10, Ostc, P4 hb, Pdia3, Pdia4, Pdia6, Phgdh, Plrg1, Preb, Prpf19, Pten, Ptpn1, Rack1, Rbx1, Rela, Rpi2211, Rpn1, Rps19, Rrp9, Sdf2i1, Sec11c, Sec13, Sec22b, Sec31a, Sec61b, Sec61g, Selenos, Serf2, Serp1, Sf3b5, Spcs2, Ssr3, Surf4, Syvn1, Tceal9, Tceb1, Tceb2, Timm13, Tpt1, Tram1, Trp53, Ube2f, Ufm1, Uqcrq, Utp15, Vcp, Vhl, Vprbp, Wdr36, Wdr43, Wdr5, Wdr74, Wdr75, and Xbp1; (i) Gene Set 9 comprising Acod1, Adam8, Atp5g3, Brap, C3ar1, Cci2, Ccl3, Cci4, Ccl7, Ccnd1, Cd300id, Cd63, Ch25h, Chd4, Chil3, Crip1, Ctsb, Ctsl, Cul1, Cul3, Cxcl1, Cyp51, Det1, Ear2, Egr2, F10, Fbxo42, Fbxw11, Ffar2, Fpr2, Fyb, Gas7, Gm9840, Gnb2, Gpnmb, Grb2, Gsk3b, Hmgcs1, Huwe1, Ifitm3, Ilif9, Itgam, Jun, Kctd12, Kctd5, Keap1, Klhdc4, Kmt2c, Kmt2d, Lgals1, Lgals3, Lmna, Lmo4, Lrpap1, Ly6c2, Lztr1, Maf, March6, Mcemp1, Mmp12, Mmp13, Mmp8, Msr1, Mtor, Naaa, Naca, Nf1, Nfkbiz, Npc2, Npy, Paf1, Pdpn, Pf4, Plet1, Pparg, Prkcd, Pten, Ptgs2, Ptpn1, Ptpn11, Ptprc, Ptx3, Rbbp5, Rela, Rfwd2, Rheb, Rptor, S100a6, Saa3, Scap, Scd2, Serpinb2, Serpinb6a, Sgk1, Slc7a11, Smad2, Srgn, Syk, Syngr1, Timp2, Trem2, Ube2h, Ube2i, Ucp2, Vasp, Vhl, Wdr26, Wfdc21, Ybx1, Zbtb7a, and Zfp3612; (j) Gene Set 10 comprising Acaca, Ak4, Aldoa, Aldoc, Anapc13, Anxa2, Arih2, Arnt, Basp1, Bnip31, Bsg, C3ar1, Cc19, Cd52, Chil3, Copa, Cui2, Cul3, Cui5, Egr2, Eif3i, Eif4ebp1, Emilin2, Eno1, Ep300, Fam162a, Gapdh, Gbe1, Gpi1, Gsn, Herpud1, Hif1a, Higd1a, Hilpda, Hk1, Hk2, Hmox1, Huwe1, ler3, Kctd10, Klk1b1, Ldha, Lgals3, Lipa, Lmo4, Lpcat2, Lyz2, March6, Mif, Mt1, Mt2, Mtor, Myc, Ndufv3, Nf1, Pdk1, Pfkl, Pgam1, Pgk1, Pgm2, Pkm, Prdx1, Prelid1, Ptpn1, Ptpn11, Rbpj, Rfwd2, Rilpl2, Rnase2a, Sacs, Scd2, Sdc3, Sdc4, Sec13, Slamf9, Slc16a3, Slc2a1, Slc7a2, Smu1, Socs3, Strap, Tarm1, Tceb1, Tceb2, Tgm2, Tlr4, Tpi1, Trf, Ube2f, Vhl, Vim, Wdr43, Wdr82, Wfdc17, and 2010005H15Rik; (k) Gene Set 11 comprising AA467197, Apobec1, Apoe, Clqa, Clqb, Clqc, C3, Car4, Ccl22, Ccl3, Ccl4, Ccl6, Cc19, Cd83, Cdc40, Cebpb, Ch25h, Chd4, Copa, Crebbp, Cul1, Ddhd1, Ddx41, Egr2, Eif3f, Eif3i, Ep300, Fam49a, Fbxw11, Fn1, Fnbp11, Gadd45b, Hdac4, Icam1, Icosl, Id2, Ikbkg, Il1a, 114i1, Inhba, Itgax, Itgb2, Kctd10, Klk1b11, Lpl, Maf, Marcks, Marcksll, Med8, Met, Mmp12, Ms4a6c, Ms4a7, Mt2, Mycbp2, Naca, Nedd8, Nfkbia, Phldal, Plaur, Plrg1, Pparg, Ppfibp2, Prpf19, Ptpn1, Rassf4, Rfwd2, Ring1, Rnase2a, Rpll2, Scimp, Sec13, Skp1a, Slc43a2, Smu1, Sqstml, Syk, Syvn1, Taf51, Tceb2, Tmem176a, Tmem176b, Tnfaip2, Traf3, Ufm1, Upp1, Wdr5, Wdr70, Wdr82, Wfdc17, Wfdc21, 0610012G03Rik, Zbtb7a, and Zyx; (I) Gene Set 12 comprising Ambra1, Aplp2, Atp5g1, Atpif1, B2m, Ccdc88a, Chd4, Copa, Cyba, Ddit3, Ear2, Egr2, Eif3f, Eif3i, Eif5, Fcgrt, Grn, H2-M2, H2-Q6, Hint1, Id1, Ifi204, Itgal, Kctd12, Laptm5, Lgals3, Ly6e, Mgst1, Mpeg1, Mtdh, Nf1, Nfe212, Nupr1, Paf1, Pparg, Prpf19, Psmb5, Psmb6, Pycard, Rack1, Rnase4, Rpl2211, Rpl37a, Rplp0, Sart1, Sdc3, Sec61 b, Smad2, Smdt1, Smu1, Spp1, Syvn1, Tab1, Taf5, Taf51, Tagln2, Tmsb10, Traf2, Traf3, Trf, Trp53, Upf1, and Wdr5; (m) Gene Set 13 comprising Ankfy1, Anxa1, Anxa5, Aph1 c, Brap, C3ar1, Ccnd2, Ccr1, Cd3001f, Cd38, Cd68, Cd9, Cdc27, Cdc40, Cebpb, Chd4, Chst11, Clec4e, Creb5, Cul1, Cul3, Cxcl3, Cyba, Dstn, Eif3f, Eif3i, Emp1, Epha4, Fam102b, Fam46a, Fbxw11, Fn1, Foxo3, Ft|l, Furin, Gas7, Gdf15, Grb2, H2-K1, Huwe1, Icam1, 117r, Inhba, Keap1, Klhdc4, Klk1bll, Lgals3, Lpl, Ly6c2, Lyz2, March6, Mbnll, Mmp14, Mmp8, Ms4a7, Naca, Neat1, Nf1, Nrp2, Plin2, Plk2, Plrg1, Polr21, Prdx1, Pten, Ptpn1, Rack1, Rasgeflb, Rasgrpl, Rela, Rnf20, Rnh1, Rpl2211, Rrp9, Saa3, Scd2, Sdc4, Sec13, Selenoh, Serp1, Skp1 a, Slamf7, Slc7a2, Smu1, Spp1, Tab1, Taf5, Ube2d3, Ubr4, Upf1, Vim, Wdr43, Wdr5, Wdr70, Wdr82, and Zbtb25; (n) Gene Set 14 comprising AC160336.1, Adgrel, Adgre4, Adgrl2, Anxa1, B2m, Clqb, C3, Car4, Ccdc88a, Ccl6, Cd52, Cdc40, Chd4, Chil3, Crip1, Ctsk, Ddx41, Dpf2, Egr2, Eif3i, Ep300, F7, Fcer1g, Fn1, Foxo3, Gpx3, H2-D1, H2-K1, H2-Q6, H2-Q7, H3f3b, Hira, Hsp90aal, Hvcn1, Id2, Ifi203, 1118, 111f9, Kdm5c, Klhl6, Lgals1, Lgals3, Ly6e, Malt1, March6, Marcks, Mcub, Med8, Mpc1, Ms4a6d, Msrb1, Mt1, Mt2, Nedd8, Nfe212, Nov, Npc2, Paf1, Pdzk1ip1, Phgdh, Pias1, Pla2g7, Plrg1, Ppic, Ppil2, Ppwd1, Prkcd, Prpf19, Ptges, Rab32, Rbx1, Rela, Rps20, S100a11, Sart1, Selenow, Smu1, St8sia4, Tab1, Taf51, Tceb2, Tmem176a, Tmem176b, Tnip3, Traf2, Tyrobp, Ube2i, Uchl1, Wdr5, Wdr70, Wdr82, Zbtb25, and Zfp106; and (o) Gene Set 15 comprising AC160336.1, Adgrel, Ahnak, Alcam, Aprt, Bcl2ll1, Blvrb, Brap, Bub3, Clqb, Clqc, C3ar1, Cd300c2, Cd33, Cd68, Cdc40, Cebpb, Chchd2, Clec12a, Clec4n, Copa, Csf1r, Ctsz, Cul3, Cul5, Cyba, Ddx41, Dstn, Egr2, Ep300, F7, Fbxw7, Fcer1g, Fcgr2b, Gmfg, Gngt2, Gpr84, Hsp90aal, Huwe1, Igf1, Kat6a, Kctdl2b, Kdm5c, Keap1, Kmt2d, Lst1, Mmp14, Mpeg1, Myc, Naca, P2ry14, Paf1, Pirb, Plrg1, Pou2f2, Pparg, Ppil2, Ppwd1, Prkcd, Prpf19, Prpf4, Ptpn1, Ptpn18, Rack1, Rbbp5, Rnf20, Rnf40, Rnf7, Rps271, Sat1, Serpinb2, Smu1, Socs3, Spp1, Taf5, Tank, Tceb1, Tceb2, Tgm2, Tnfsfl 5, Traf2, Trem2, Tyrobp, Ufm1, Vcan, Wdr1, Wdr33, Wdr43, Wdr5, Wdr61, Wdr70, Wdr82, Wfdc21, and Ybx1.

In some aspects, at least one of the alterations is a loss-of-function alteration.

In some aspects, at least one of the alterations is a gain-of-function alteration.

In some aspects, the genetically modified isolated cell comprises loss-of-function alterations in one, two, or all three of Ldb2, Rnf165, and Traf2. In some aspects, the loss-of-function alteration is a knockout (KO) mutation.

In some aspects, the genetically modified isolated cell comprises a gain-of-function alteration in CCR7. In some aspects, the gain-of-function alteration is overexpression.

In another aspect, the invention provides a method of identifying a modulator of the interaction between F-box and WD repeat domain containing 11 (Fbxw11) and nuclear factor kappa B subunit 1 (Nfkb1) or nuclear factor kappa B subunit 2 (Nfkb2), the method comprising (a) providing a candidate modulator; (b) contacting Fbxw11 with Nfkb1 or Nfkb2 in the presence or absence of the candidate modulator under conditions permitting the binding of Fbxw11 to Nfkb1 or Nfkb2; and (c) measuring the binding of Fbxw11 to Nfkb1 or Nfkb2, wherein an increase or decrease in binding in the presence of the candidate modulator relative to binding in the absence of the candidate modulator identifies the candidate modulator as a modulator of the interaction between Fbxw11 and Nfkb1 or Nfkb2.

In another aspect, the invention provides a method of identifying a modulator of a downstream activity of Fbxw11, the method comprising (a) providing a candidate modulator; (b) contacting Fbxw11 with Nfkb1 or Nfkb2 in the presence or absence of the candidate modulator under conditions permitting the binding of Fbxw11 to Nfkb1 or Nfkb2; and (c) measuring a downstream activity of Fbxw11, wherein a change in the downstream activity in the presence of the candidate modulator relative to the downstream activity in the absence of the candidate modulator identifies the candidate modulator as a modulator of the downstream activity of Fbxw11.

In another aspect, the invention provides a method of identifying a modulator of a downstream activity of Nfkb1 or Nfkb2, the method comprising (a) providing a candidate modulator; (b) contacting Nfkb1 or Nfkb2 with Fbxw11 in the presence or absence of the candidate modulator under conditions permitting the binding of Nfkb1 or Nfkb2 to Fbxw11; and (c) measuring a downstream activity of Nfkb1 or Nfkb2, wherein a change in the downstream activity in the presence of the candidate modulator relative to the downstream activity in the absence of the candidate modulator identifies the candidate modulator as a modulator of the downstream activity of Nfkb1 or Nfkb2.

In some aspects, the increase or decrease in binding is at least 50%, as measured by surface plasmon resonance, biolayer interferometry, or an enzyme-linked immunosorbent assay (ELISA).

In some aspects, the modulator is an inhibitor of the downstream activity of Fbxw11 or Nfkb1 or Nfkb2.

In some aspects, the change in the downstream activity is an increase in the amount, strength, or duration of the downstream activity.

In some aspects, the change in the downstream activity is a decrease in the amount, strength, or duration of the downstream activity.

In some aspects, the modulator is a proteolysis targeting chimera (PROTAC), a small molecule, an antibody or antigen-binding fragment thereof, a peptide, a mimic, or an inhibitory nucleic acid. In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody or antigen-binding fragment thereof binds Fbxw11.

In some aspects, the antibody or antigen-binding fragment thereof binds Nfkb1 or Nfkb2.

In some aspects, the downstream activity is immune response activation.

In another aspect, the invention provides a method for preventing or treating a cancer, an inflammatory disease, or an autoimmune disease in an individual, the method comprising administering to the individual an effective amount of a modulator identified by any one of the methods provided herein, thereby treating the individual.

In another aspect, the invention provides a method for treating a cancer in an individual, the method comprising administering to the individual an effective amount of a modulator of the interaction between Fbxw11 and one or both of Nfkb1 and Nfkb2, wherein immune response activation is increased in the presence of the modulator.

In another aspect, the invention provides a method for treating an inflammatory disease or an autoimmune disease in an individual, the method comprising administering to the individual an effective amount of a modulator of the interaction between Fbxw11 and one or both of Nfkb1 and Nfkb2, wherein immune response activation is decreased in the presence of the modulator.

In another aspect, the invention provides a method of identifying a modulator of the interaction between Ring finger and WD repeat domain 2 (Rfwd2) and a query protein selected from Forkhead box L2 (Foxl2), JunD, WD repeat domain 82 (Wdr82); ElA binding protein p300 (Ep300); Anaphase promoting complex subunit 13 (Anapc13); Cullin 2 (Cul2); Cullin 5 (Cul5); HECT, UBA and WWE domain containing E3 ubiquitin protein ligase 1 (Huwe1); CREB binding protein (Crebbp); S-phase kinase associated protein 1 (Skp1 a); Neural precursor cell expressed, developmentally down-regulated gene 8 (Nedd8); Cullin 1 (Cul1); and WD repeat domain 5 (Wdr5), the method comprising (a) providing a candidate modulator; (b) contacting Rfwd2 with the query protein in the presence or absence of the candidate modulator under conditions permitting the binding of Rfwd2 to the query protein; and (c) measuring the binding of Rfwd2 to the query protein, wherein an increase or decrease in binding in the presence of the candidate modulator relative to binding in the absence of the candidate modulator identifies the candidate modulator as a modulator of the interaction between Rfwd2 and the query protein.

In another aspect, the invention provides a method of identifying a modulator of a downstream activity of Rfwd2, the method comprising (a) providing a candidate modulator; (b) contacting Rfwd2 with a query protein selected Wdr82, Ep300, Anapc13, Cul2, Cul5, Huwe1, Crebbp, Skp1 a, Nedd8, Cul1, and Wdr5 in the presence or absence of the candidate modulator under conditions permitting the binding of Rfwd2 to the query protein; and (c) measuring a downstream activity of Rfwd2, wherein a change in the downstream activity in the presence of the candidate modulator relative to the downstream activity in the absence of the candidate modulator identifies the candidate modulator as a modulator of the downstream activity of Rfwd2.

In another aspect, the invention provides a method of identifying a modulator of a downstream activity of a query protein selected from Wdr82, Ep300, Anapc13, Cul2, Cul5, Huwe1, Crebbp, Skp1 a, Nedd8, Cul1, and Wdr5, the method comprising (a) providing a candidate modulator; (b) contacting the query protein with Rfwd2 in the presence or absence of the candidate modulator under conditions permitting the binding of the query protein to Rfwd2; and (c) measuring a downstream activity of the query protein, wherein a change in the downstream activity in the presence of the candidate modulator relative to the downstream activity in the absence of the candidate modulator identifies the candidate modulator as a modulator of the downstream activity of the query protein.

In some aspects, the increase or decrease in binding is at least 50%, as measured by surface plasmon resonance, biolayer interferometry, or an enzyme-linked immunosorbent assay (ELISA).

In some aspects, the modulator is an inhibitor of the downstream activity of Rfwd2 or the query protein.

In some aspects, the change in the downstream activity is an increase in the amount, strength, or duration of the downstream activity.

In some aspects, the change in the downstream activity is a decrease in the amount, strength, or duration of the downstream activity.

In some aspects, the modulator is a proteolysis targeting chimera (PROTAC), a small molecule, an antibody or antigen-binding fragment thereof, a peptide, a mimic, or an inhibitory nucleic acid. In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody or antigen-binding fragment thereof binds Rfwd2.

In some aspects, the antibody or antigen-binding fragment thereof binds the query protein.

In some aspects, the downstream activity is dendritic cell or macrophage migration.

In another aspect, the invention provides a method for preventing or treating a cancer, an inflammatory disease, or an autoimmune disease in an individual, the method comprising administering to the individual an effective amount of a modulator identified by a method provided herein, thereby treating the individual.

In another aspect, the invention provides a method for treating an inflammatory disease or an autoimmune disease in an individual, the method comprising administering to the individual an effective amount of a modulator of the interaction between Rfwd2 and one or more of Wdr82, Ep300, Anapc13, Cul2, Cul5, Huwe1, Crebbp, Skp1 a, Nedd8, Cul1, and Wdr5, wherein dendritic cell or macrophage migration is decreased in the presence of the modulator.

In another aspect, the invention provides a method for treating a cancer in an individual, the method comprising administering to the individual an effective amount of a modulator of the interaction between Rfwd2 and one or more of Wdr82, Ep300, Anapc13, Cul2, Cul5, Huwe1, Crebbp, Skp1 a, Nedd8, Cull, and Wdr5, wherein dendritic cell or macrophage migration is increased in the presence of the modulator.

In another aspect, the invention provides a kit comprising a modulator of the interaction between Rfwd2 and one or more of Wdr82, Ep300, Anapc13, Cul2, Cul5, Huwe1, Crebbp, Skp1 a, Nedd8, Cul1, and Wdr5 for treating an individual having a cancer, an inflammatory disease, or an autoimmune disease according to a method provided herein. In some aspects, the kit comprises a package insert comprising instructions to administer the modulator to an individual having a cancer, an inflammatory disease, or an autoimmune disease.

In another aspect, the invention provides a method of identifying a modulator of the interaction between a protein complex comprising Tyrosine-protein phosphatase non-receptor type 11 (Ptpn11) and Ring finger and WD repeat domain 2 (Rfwd2) and a CCAAT enhancer-binding protein (Cebp) family transcription factor, the method comprising (a) providing a candidate modulator; (b) contacting the protein complex with the Cebp family transcription factor in the presence or absence of the candidate modulator under conditions permitting the binding of Rfwd2 to the query protein; and (c) measuring the binding of the protein complex to the Cebp family transcription factor, wherein an increase or decrease in binding in the presence of the candidate modulator relative to binding in the absence of the candidate modulator identifies the candidate modulator as a modulator of the interaction between the protein complex and the Cebp family transcription factor.

In another aspect, the invention provides a method of identifying a modulator of a downstream activity of a protein complex comprising Ptpn11 and Rfwd2, the method comprising (a) providing a candidate modulator; (b) contacting the protein complex with a Cebp family transcription factor in the presence or absence of the candidate modulator under conditions permitting the binding of the protein complex to the Cebp family transcription factor; and (c) measuring a downstream activity of the protein complex, wherein a change in the downstream activity in the presence of the candidate modulator relative to the downstream activity in the absence of the candidate modulator identifies the candidate modulator as a modulator of the downstream activity of the protein complex.

In another aspect, the invention provides a method of identifying a modulator of a downstream activity of a Cebp family transcription factor, the method comprising (a) providing a candidate modulator; (b) contacting the Cebp family transcription factor with a protein complex comprising Ptpn11 and Rfwd2 in the presence or absence of the candidate modulator under conditions permitting the binding of the Cebp family transcription factor to the protein complex; and (c) measuring a downstream activity of the query protein, wherein a change in the downstream activity in the presence of the candidate modulator relative to the downstream activity in the absence of the candidate modulator identifies the candidate modulator as a modulator of the downstream activity of the query protein.

In some aspects, the increase or decrease in binding is at least 50%, as measured by surface plasmon resonance, biolayer interferometry, or an enzyme-linked immunosorbent assay (ELISA).

In some aspects, the modulator is an inhibitor of the downstream activity of the protein complex or the Cebp family transcription factor.

In some aspects, the change in the downstream activity is an increase in the amount, strength, or duration of the downstream activity.

In some aspects, the change in the downstream activity is a decrease in the amount, strength, or duration of the downstream activity.

In some aspects, the modulator is a proteolysis targeting chimera (PROTAC), a small molecule, an antibody or antigen-binding fragment thereof, a peptide, a mimic, or an inhibitory nucleic acid. In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody or antigen-binding fragment thereof binds the protein complex.

In some aspects, the antibody or antigen-binding fragment thereof binds the Cebp family transcription factor.

In another aspect, the invention provides a method for preventing or treating a disease or disorder related to antigen-presenting cells (APCs) and/or inflammation in an individual, the method comprising administering to the individual an effective amount of a modulator of a gene of Table 1 or Table 2, thereby treating the individual.

In some aspects, the modulator modulates expression of the gene. In some aspects, the modulator modulates expression or activity of a protein encoded by the gene.

In some aspects, the modulator causes a change in a downstream activity of a protein encoded by the gene of Table 1 or Table 2 in the presence of the modulator relative to the downstream activity in the absence of the modulator.

In some aspects, the modulator is an inhibitor of the downstream activity of the gene of Table 1 or Table 2.

In some aspects, the change in the downstream activity is a decrease in the amount, strength, or duration of the downstream activity.

In some aspects, the modulator is an activator of the downstream activity of the gene of Table 1 or Table 2.

In some aspects, the change in the downstream activity is an increase in the amount, strength, or duration of the downstream activity.

In some aspects, the modulator is a proteolysis targeting chimera (PROTAC), a small molecule, an antibody or antigen-binding fragment thereof, a peptide, a mimic, or an inhibitory nucleic acid. In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody or antigen-binding fragment thereof binds a protein encoded by the gene of Table 1 or Table 2.

In some aspects, the disease or disorder relating to APCs and/or inflammation is a neurodegenerative disease, arthritis, allergy, eczema, fibrosis, asthma, lupus erythematosus, an inflammatory bowel disease, ulcerative colitis, Crohn's disease, or a blastic plasmacytoid dendritic cell neoplasm. In some aspects, the neurodegenerative disease is MS, AD, ALS, or PD.

In some aspects, the APC is a DC, a macrophage, or a glial cell. In some aspects, the glial cell is a microglial cell, an astrocyte, or an oligodendrocyte. In some aspects, the APC is a DC.

In another aspect, the invention provides a kit comprising a modulator of a gene of Table 1 or Table 2 for treating an individual having a disease or disorder related to APCs and/or inflammation according to a method provided herein. In some aspects, the kit comprises a package insert comprising instructions to administer the modulator to an individual having a disease or disorder related to APCs and/or inflammation.

In another aspect, the invention provides a method of monitoring the response of an individual having a disease or disorder related to APCs and/or inflammation to treatment with a modulator of a gene of Table 1 or Table 2, the method comprising (a) determining, in a biological sample obtained from the individual at a time point following administration of the modulator, the expression level of the gene of Table 1 or Table 2; and (b) comparing the expression level of the gene of Table 1 or Table 2 in the biological sample with a reference level, thereby monitoring the response in the individual to treatment with the modulator. In some aspects, the reference level is selected from the group consisting of (i) the expression level of the gene in a biological sample from the individual obtained prior to administration of the modulator; (ii) the expression level of the gene in a reference population; (iii) a pre-assigned expression level for the gene; or (iv) the expression level of the gene in a biological sample obtained from the individual at a previous time point, wherein the previous time point is following administration of the modulator.

In some aspects, the expression level of the expression level of the gene of Table 1 or Table 2 is decreased in the biological sample obtained from the individual relative to the reference level, and the method further comprises administering to the individual one or more additional doses of the modulator, wherein the modulator is an agent that increases the expression and/or activity of the gene of Table 1 or Table 2.

In some aspects, the expression level of the expression level of the gene of Table 1 or Table 2 is increased in the biological sample obtained from the individual relative to the reference level, and the method further comprises administering to the individual one or more additional doses of the modulator, wherein the modulator is an agent that decreases the expression and/or activity of the gene of Table 1 or Table 2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram showing the design of a large-scale Perturb-seq screen for assessing the function of E3 ligases in the lipopolysaccharide (LPS) response in bone marrow-derived dendritic cells (BMDCs). Top row: experimental flow. Middle row: Perturb-Seq vector design. Bottom row: Exemplary E3 ligases and complex members known to regulate different processes.

FIG. 1B is a schematic diagram showing the key features of the PerturbDecode workflow. The full workflow diagram is shown in FIG. 8E. KO: knockout; PPI: protein-protein interaction; TF: transcription factor.

FIG. 1C is a Uniform Manifold Approximation and Projection (UMAP) showing 519,535 perturbed single cell profiles colored by cluster membership.

FIG. 1D is a UMAP showing 519,535 cell profiles colored by type 2 dendritic cell (DC2)-like cell type signature score.

FIG. 1E is a UMAP showing 519,535 cell profiles colored by regulatory macrophage (MReg)-like cell type signature score.

FIG. 1F is a UMAP showing 519,535 cell profiles colored by type 1 dendritic cell (DC1)-like cell type signature score.

FIG. 1G is a UMAP showing 519,535 cell profiles colored by cell cycle phase.

FIG. 1H is a UMAP showing 519,535 cell profiles colored by difference of macrophage vs. dendritic cell (DC) signature scores (DC maturation).

FIG. 1I is a heat map showing the odds ratio (color bar) of the significant (false discovery rate (FDR)<0.15, one-sided Fisher's exact test) enrichment (pink) or depletion (blue) of guides targeting perturbed genes (rows) in each major cell subset (columns) in the Perturb-seq screen. Mac: macrophage.

FIG. 1J is a pair of tables showing a summary of enrichment (pink) and depletion (blue) of guides targeting key proteins in major subsets (left table) and in DC2 subtypes (right table), colored by E3 family type and grouped by complex. ULD: ubiquitin-like domain; DUB: de-ubiquitylating enzyme; NS: non-significant.

FIG. 2A is a pie chart showing the proportion of the 329 significant regulators (impactful perturbed genes) in each of the indicated E3 family member types (left) and a scatter plot showing UMAP embeddings of the regulatory profiles of the 329 regulators (KO genes), colored by their module membership. DUB: deubiquitinase; ULD: ubiquitin-like domain.

FIG. 2B is a scatter plot showing UMAP embeddings of the regulated profiles of 1,041 affected genes, colored by their membership in gene programs (GPs) 1-11.

FIG. 2C is a set of regulatory matrices. Top left: regulatory matrix (beta) showing regulatory effect size (red/blue) of perturbing (KO) each of 329 genes (rows) on the expression of each of 1,041 affected genes (columns). Red/blue: Induction/repression in response to perturbation (KO) compared to control cells. Black horizonal and vertical line delineate co-functional modules and co-regulated programs, respectively. Top right: regulatory matrix showing co-functional modules. Covariance (green/purple) between the regulatory profiles in beta of perturbing each of 329 genes. Genes are clustered by module (as in FIG. 2A; color code on top and right). Bottom: regulatory matrix showing co-regulated programs. Covariance (green/purple) between the regulatory profiles in beta of the effect on expression of each of 1,041 genes. Genes are clustered by program (as in FIG. 2B; color code on bottom and right).

FIG. 2D is a bipartite graph showing the relationship between the six co-functional modules (left) to the eleven co-regulated programs (right). Red point arrow: module genes activate program (i.e., KO inhibits program); blue blunt arrow: module genes inhibit program (i.e., activates program) (arrow color determined by significant mean difference). Key gene names are noted.

FIG. 3A is a diagram showing an E3 genetic regulatory network. Regulatory relations from the model shown in FIG. 2C based on a perturbation (KO) in an E3 ligase to another E3 ligase whose expression is affected are shown. Red arrows: perturbed E3 activates target's expression (i.e., KO inhibits expression). Blue arrows: perturbed E3 inhibits target's expression (i.e., KO activates expression). Three E3 ligases are highly regulated “authorities” in the E3 network.

FIG. 3B is a UMAP embedding of single cell profiles colored by Gaussian kernel density estimations of cells using control (top left), M1 (top right) or M5 (bottom) guides.

FIG. 3C is a supervised UMAP embedding of DC2.1, DC2.2, and DC2.3 cell profiles using a cells' co-functional module assignment as the response label, colored by the module assignment of their guides (color code).

FIG. 3D is a chart showing the average Wasserstein distances (color bar) between cells with guides from different co-functional modules (rows, columns).

FIG. 3E is a chart showing the odds ratio (color bar) of significant enrichment (pink) or depletion (green) of DC subsets (rows) in cells with guides from each co-functional module (columns) (FDR <0.15, one-sided Fisher's exact test).

FIG. 3F is a chart showing physical interactions (blue, red, grey; experimental score >0, STRING database (DB)) or lack thereof (white) between each pair of 78 E3 ligases and adaptors with at least 24 interactions (with any of the 165 E3 ligases among the 329 regulators). The full matrix is shown in FIG. 11G. Red: the regulatory profiles of the physically interacting genes have significant (P<0.05) positive correlation. Blue: the regulatory profiles of the physically interacting genes have significant (P<0.05) negative correlation. Grey: the regulatory profiles of the physically interacting genes are not significantly correlated. Bars: co-functional modules. Rows and columns are hierarchically clustered.

FIG. 3G is a chart showing the inferred activity scores (color bar) of 32 TFs (columns) whose target genes are significantly (FDR <0.1) induced (yellow) or repressed (blue) when perturbing each of 41 E3 and related genes (rows). The full matrix is shown in FIG. 11J.

FIG. 3H is a set of Venn diagrams showing the intersection between TF targets (Discriminant Regulon Expression Analysis (DoRothEA)), E3 expression targets, and gene programs.

FIG. 3I is a set of Venn diagrams showing the intersection between TF targets (DoRothEA), E3 expression targets, and gene programs.

FIG. 4A is a pair of heat maps showing E3 regulators' association with independent factors from an independent component analysis (ICA). Explained variance of effects on 1,041 genes of each of 203 perturbed genes (left matrix columns, sum of explained variance >25%) or components of the CUL4-RBX1-DET1-RFWD2 complex (right matrix columns) is shown by each of 15 latent factors (rows, main panel) and across all 15 factors (bottom).

FIG. 4B is a chart showing effect sizes (yellow: positive; blue: negative; color bar) on significantly affected genes (columns; outlier loadings, separated by direction of effect) upon perturbation of each regulator gene associated with the indicated factor (rows; outliers based on weights in the mixing matrix). Left bar: co-functional modules; top bar: gene programs from the regulatory model.

FIG. 4C is a UMAP embedding of cell profiles (as in FIG. 1C) colored by expression scores for Factor 5.1.

FIG. 4D is a UMAP embedding of cell profiles (as in FIG. 1C) colored by expression scores for Factor 5.2.

FIG. 4E is a chart showing effect sizes (yellow: positive; blue: negative; color bar) on significantly affected genes (columns; outlier loadings, separated by direction of effect) upon perturbation of each regulator gene associated with the indicated factor (rows; outliers based on weights in the mixing matrix). Left bar: co-functional modules; top bar: gene programs from the regulatory model.

FIG. 4F is a UMAP embedding of cell profiles (as in FIG. 1C) colored by expression scores for Factor 6.1.

FIG. 4G is a UMAP embedding of cell profiles (as in FIG. 1C) colored by expression scores for Factor 6.2.

FIG. 4H is a chart showing effect sizes (yellow: positive; blue: negative; color bar) on significantly affected genes (columns; outlier loadings, separated by direction of effect) upon perturbation of each regulator gene associated with the indicated factor (rows; outliers based on weights in the mixing matrix). Left bar: co-functional modules; top bar: gene programs from the regulatory model.

FIG. 4I is a UMAP embedding of cell profiles (as in FIG. 1C) colored by expression scores for Factor 2.1.

FIG. 4J is a UMAP embedding of cell profiles (as in FIG. 1C) colored by expression scores for Factor 2.2.

FIG. 5A is a bar graph showing the number of genes (y-axis) significantly affected by intra-module (left bar) or inter-module (right bars) combinatorial perturbation (x-axis), additively (grey) or non-additively (blue).

FIG. 5B is a set of scatter plots showing intra-module interactions in the indicated modules. The y-axis shows observed fold changes in expression (vs. control) in cells with perturbations in two genes from the same module and the x-axis shows the expected fold change from an additive model based on the two individual perturbations for each of 1,041 genes (dots). Slope of the first principal component (PC) (red line) and variance in observed double knockouts explained by the single knockouts (R²) are labeled. Module M4 is not shown due to an insufficient number of double-knockout cells.

FIG. 5C is a heat map showing significant effect sizes (red/blue color bar; FDR<0.1) of perturbations at the level of individual modules (Mᵢ), inter-module pairs (Mᵢ:Mⱼ; i≠j), and intra-module pairs (Mᵢ:Mᵢ) (rows) on each of 1,041 genes (columns; labeled by gene program). Bottom row: Row centered mean expression in control cells.

FIG. 5D is a chart showing binarized significant effect size (FDR <0.1) (red: positive; blue: negative) on gene expression (rows; only genes with significant interaction terms) by single perturbations in regulators from M3 or M5, their additive effect (M3+M5), their interaction term (M3:M5), and observed combined effect (columns).

FIG. 5E is a schematic diagram showing an overview of the comβVAE method for predicting combinatorial perturbations.

FIG. 5F is a set of box plots showing the distribution of explained variance in fold changes of the 1,041 genes (y-axis; R² is 7 runs with the same hyperparameters) at different Kullback-Leibler (KL) loss weights (x-axis) for individual modules (Mᵢ) and inter-module combinations (Mᵢ:Mⱼ; i≠j).

FIG. 5G is a set of bar graphs showing distribution of the explained variance (R², y-axis) in fold changes of the 1,041 genes from 7 runs with the same hyperparameters in the indicated inter-module combinations (labels on top) when the model (Beta=6.0) is trained only with data from single KOs from all modules (M) or single KOs from all modules and double KOs from one or two pairs of modules (Mᵢ:Mⱼ; i≠j) (x-axis). Boxes display the first (Q₁), second (Q₂, median) and third (Q₃) quartiles while the bottom and top whiskers show the intervals [Q₁−1.5 IQR, Q₁] and [Q₃, Q_{3+1.5} IQR], respectively.

FIG. 6A is a heat map showing significant MAGMA Z-scores (color bar; per trait; Bonferroni α<0.1) for immunological disease traits (columns) of module M6 E3 family genes (rows) with at least one significant score.

FIG. 6B is a set of heat maps showing significance (−log₁₀(p-value), dot color) and effect size (dot size) of heritability enrichment in each gene program (rows) for different immune traits (columns) associated with genetic disease risk for human immunological disease by sc-linker analysis with single-nucleotide polymorphism (SNP) annotations combined with intersection of Roadmap and ABC gene-enhancer linking strategy (left) or by MAGMA (right).

FIG. 6C is a heat map showing gene programs expressed during immunological disease progression in immune and non-immune cells. Enrichment (color bar) of gene programs (columns) for cell type specific disease progression programs in humans (rows), across diverse cell types and diseases are shown.

FIG. 6D is a heat map showing gene programs expressed during immunological disease progression in immune and non-immune cells. Enrichment (color bar) of gene programs (columns) for cell type specific disease progression programs in in DCs and macrophages in ulcerative colitis (UC), fibrosis, asthma, and COVID-19 are shown.

FIG. 6E is a heat map showing the regulatory coefficient (color bar, from the model of FIG. 2C) of the impact of perturbing regulators (rows) on the expression of genes (columns) with rare variants associated with inflammatory bowel disease (IBD) that also have at least one significantly regulating E3 family member.

FIG. 6F is a phot showing the significance (−log₁₀(p-value), dot color) and effect size (dot size) of heritability enrichment of gene programs (rows) for Crohn's disease (CD) or IBD based on rare (CD:SAIGE-GENE) or common (IBD:MAGMA and CD: MAGMA) variants.

FIG. 7 is a diagram showing the DC life cycle regulated by E3 ligases. ICA factors and their key regulators grouped in each DC lifecycle stage are shown in boxes. Bottom: UMAP embedding of cell profiles (as in FIG. 1C) colored by expression scores (color bar) for migration-related factors.

FIG. 8A is a plot showing forward scatter (x-axis) versus side scatter (y-axis) for selected live perturbed BMDCs.

FIG. 8B is a plot showing forward scatter (x-axis) versus side scatter (y-axis) for selected single perturbed BMDCs.

FIG. 8C GFP fluorescence (x-axis; Cas9 mice cells) versus mKate2 fluorescence (y-axis; Perturb-Seq vector) in select mKate2⁺GFP⁺ cells.

FIG. 8D is a plot showing the distribution of mKate2 expression (x-axis) in sorted live, single cells.

FIG. 8E is a schematic diagram showing the detailed PerturbDecode workflow.

FIG. 8F is a graph showing the cumulative distribution function (CDF) (y-axis) of Pearson's r (x-axis) between the effect sizes of guides targeting the same gene (purple), different genes (red), one gene and one no-target control (green), or one gene and one intergenic control (blue).

FIG. 8G is a graph showing the distribution of number of genes (y-axis) (of 6,685 tested genes) significantly affected (FDR <0.1) by non-targeting controls, intergenic controls or targeting guides (with guides targeting the same gene combined) (x-axis). **P<2.2*10⁻¹⁶, one-sided Wilcoxon rank-sum test.

FIG. 8H is a plot showing the significant effect sizes (color bar; blue/red negative/positive fold-change; FDR<0.1) of perturbing each of 544 targets (rows) that were also among the 6,685 genes with tested expression on itself and the other 544 targets (columns). Rows and columns are ordered alphabetically. 137 of 539 genes significantly negatively affected their own expression (diagonal).

FIG. 8I is a scatter plot showing the number of genes (y-axis) whose expression is significantly (FDR<0.1) affected by perturbation of each of 849 perturbed genes (from 13,811 detected genes) and the mean expression of these perturbed genes (x axis, normalized log 1p). Pearson's rand significance in the upper left corner.

FIG. 8J is a set of violin plots showing the distribution of number of genes (y-axis) significantly affected (FDR <0.1) by the perturbation of genes that are (“expressed”) or are not (“not expressed”) in the 13,811 detected genes. **** P-value <104, one-sided Wilcoxon rank-sum test.

FIG. 9A is a chart showing mean expression (dot color, mean normalized log 1 p expression) and fraction of expressing cells (dot size) for genes differentially expressed (columns) in each of the 10 cell clusters (rows).

FIG. 9B is a chart showing mean expression (dot color, mean normalized log 1 p expression) and fraction of expressing cells (dot size) for genes differentially expressed (columns) in DC2 marker genes (rows).

FIG. 9C is a chart showing mean expression (dot color, mean normalized log 1 p expression) and fraction of expressing cells (dot size) for genes differentially expressed (columns) in mDC marker genes (rows).

FIG. 9D is a chart showing mean expression (dot color, mean normalized log 1 p expression) and fraction of expressing cells (dot size) for genes differentially expressed (columns) in DC1 marker genes (rows).

FIG. 9E is a chart showing mean expression (dot color, mean normalized log 1 p expression) and fraction of expressing cells (dot size) for genes differentially expressed (columns) in pDC marker genes (rows).

FIG. 9F is a chart showing mean expression (dot color, mean normalized log 1 p expression) and fraction of expressing cells (dot size) for genes differentially expressed (columns) in M1 marker genes (rows).

FIG. 9G is a chart showing mean expression (dot color, mean normalized log 1 p expression) and fraction of expressing cells (dot size) for genes differentially expressed (columns) in M2 marker genes (rows).

FIG. 9H is a UMAP embedding of 519,535 cell profiles (as in FIG. 1C) colored by DC (DC1+DC2+mDC) gene signature score.

FIG. 9I is a UMAP embedding of 519,535 cell profiles (as in FIG. 1C) colored by macrophage signature score.

FIG. 9J is a UMAP embedding of 3,655 unperturbed unstimulated and 4,027 unperturbed and LPS stimulated (3 hours) BMDC profiles colored by treatment.

FIG. 9K is a UMAP embedding of 3,655 unperturbed unstimulated and 4,027 unperturbed and LPS stimulated (3 hours) BMDC profiles colored by inferred cell cycle phase.

FIG. 9L is a UMAP embedding of 3,655 unperturbed unstimulated and 4,027 unperturbed and LPS stimulated (3 hours) BMDC profiles colored by signature scores for the top 100 upregulated genes of cluster 1 of FIG. 1C.

FIG. 9M is a UMAP embedding of 3,655 unperturbed unstimulated and 4,027 unperturbed and LPS stimulated (3 hours) BMDC profiles colored by signature scores for the top 100 upregulated genes of cluster 2 of FIG. 1C.

FIG. 9N is a UMAP embedding of 3,655 unperturbed unstimulated and 4,027 unperturbed and LPS stimulated (3 hours) BMDC profiles colored by signature scores for the top 100 upregulated genes of cluster 3 of FIG. 1C.

FIG. 9O is a UMAP embedding of 3,655 unperturbed unstimulated and 4,027 unperturbed and LPS stimulated (3 hours) BMDC profiles colored by signature scores for the top 100 upregulated genes of cluster 4 of FIG. 1C.

FIG. 9P is a UMAP embedding of 3,655 unperturbed unstimulated and 4,027 unperturbed and LPS stimulated (3 hours) BMDC profiles colored by signature scores for the top 100 upregulated genes of cluster 5 of FIG. 1C.

FIG. 9Q is a UMAP embedding of 3,655 unperturbed unstimulated and 4,027 unperturbed and LPS stimulated (3 hours) BMDC profiles colored by signature scores for the top 100 upregulated genes of cluster 6 of FIG. 1C.

FIG. 9R is a UMAP embedding of 3,655 unperturbed unstimulated and 4,027 unperturbed and LPS stimulated (3 hours) BMDC profiles colored by signature scores for the top 100 upregulated genes of cluster 7 of FIG. 1C.

FIG. 9S is a UMAP embedding of 3,655 unperturbed unstimulated and 4,027 unperturbed and LPS stimulated (3 hours) BMDC profiles colored by signature scores for the top 100 upregulated genes of cluster 8 of FIG. 1C.

FIG. 9T is a UMAP embedding of 3,655 unperturbed unstimulated and 4,027 unperturbed and LPS stimulated (3 hours) BMDC profiles colored by signature scores for the top 100 upregulated genes of cluster 9 of FIG. 1C.

FIG. 9U is a UMAP embedding of 3,655 unperturbed unstimulated and 4,027 unperturbed and LPS stimulated (3 hours) BMDC profiles colored by signature scores for the top 100 upregulated genes of cluster 10 of FIG. 1C.

FIG. 9V is a UMAP embedding of 3,655 unperturbed unstimulated and 4,027 unperturbed and LPS stimulated (3 hours) BMDC profiles colored by their predicted major cell subtype.

FIG. 9W is a bar graph showing the percentage of cells (y-axis) of each of four major subtypes in the screen (color legend) in unperturbed unstimulated, unperturbed LPS stimulated and perturbed, LPS stimulated data (x-axis). *P<2.2*10⁻¹⁶, one-sided Fisher's exact test.

FIG. 9X is a heat map showing the odds ratio (color bar) of enrichment (pink) or depletion (blue) (FDR <0.15, one-sided Fisher's exact test) of cells with a perturbed gene (rows) in cell cycle phases in major subtypes (columns) as in FIG. 1C.

FIG. 9Y is a heat map showing the odds ratio (color bar) of enrichment (pink) or depletion (blue) (FDR <0.15, one-sided Fisher's exact test) of cells with a perturbed gene (rows) in cell cycle phases in the 10 cell clusters (columns) as in FIG. 1C.

FIG. 10A is a UMAP embedding of cell profiles (as in FIG. 1C) colored by expression scores of the GP1 program genes.

FIG. 10B is a UMAP embedding of cell profiles (as in FIG. 1C) colored by expression scores of the GP2 program genes.

FIG. 10C is a UMAP embedding of cell profiles (as in FIG. 1C) colored by expression scores of the GP3 program genes.

FIG. 10D is a UMAP embedding of cell profiles (as in FIG. 1C) colored by expression scores of the GP4 program genes.

FIG. 10E is a UMAP embedding of cell profiles (as in FIG. 1C) colored by expression scores of the GP5 program genes.

FIG. 10F is a UMAP embedding of cell profiles (as in FIG. 1C) colored by expression scores of the GP6 program genes.

FIG. 10G is a UMAP embedding of cell profiles (as in FIG. 1C) colored by expression scores of the GP7 program genes.

FIG. 10H is a UMAP embedding of cell profiles (as in FIG. 1C) colored by expression scores of the GP8 program genes.

FIG. 10I is a UMAP embedding of cell profiles (as in FIG. 1C) colored by expression scores of the GP9 program genes.

FIG. 10J is a UMAP embedding of cell profiles (as in FIG. 1C) colored by expression scores of the GP10 program genes.

FIG. 10K is a UMAP embedding of cell profiles (as in FIG. 1C) colored by expression scores of the GP11 program genes.

FIG. 10L is a pair of heat maps showing the Jaccard index (left) and fractional overlap (right) between each gene program (rows, “A”) and programs in an earlier small Perturb-Seq screen of 24 TFs in the LPS-stimulated BMDCs (Dixit et al., Cell, 167: 1853-1866.e17, 2016) (left, columns, “B”) or DC subset signatures (Maier et al., Nature, 580: 257-262, 2020) (right, columns, “B”).

FIG. 10M is a set of violin plots showing the distribution of program scores (GP1-11; y-axis) for DC1-, DC2-, mDC-, and macrophage-like cell subsets (x-axis). *P<0.05 one-vs.-rest one-sided Wilcoxon rank sum test.

FIG. 11A is a chart showing the binarized regulatory effects (blue: negative; red: positive) of perturbing each of 60 E3 ligases in the model that significantly affected the expression of at least one other of the 60 E3 ligases for all 60 E3 ligases. Module membership is labeled by color on left and top. Negative effects of the KOs on its own RNA level are not shown.

FIG. 11B is a chart showing the binarized regulatory effects (blue: negative; red: positive) of perturbing each of 60 E3 ligases in the model that significantly affected the expression of at least one other of the 60 E3 ligases for the 15 E3 ligases that both impact and are impacted by another E3. Module membership is labeled by color on left and top. Negative effects of the KOs on its own RNA level are shown.

FIG. 11C is a set of UMAP embeddings of cell profiles (as in FIG. 1C) colored by Gaussian kernel density estimations of cells with control guides (top left) or guides targeting genes with each module (label on top).

FIG. 11D is a supervised UMAP embedding of DC2.1, DC2.2, and DC2.3 cell profiles using a cell's co-functional module assignment as the response label (as in FIG. 3C), colored by the difference of macrophage and DC signature Z scores.

FIG. 11E is a stacked bar graph showing the percentage of cells (y axis) with guides targeting genes in each module (x-axis) belonging to each of the cell clusters of FIG. 1C.

FIG. 11F is a stacked bar graph showing the percentage of cells (y axis) in each of the cell clusters of FIG. 1C (x-axis) with guides targeting genes in each module.

FIG. 11G is a chart showing physical interactions (blue, red, grey; experimental score >0, STRING DB) or lack thereof (white) between each pair of 165 E3 ligases among the 329 regulators (rows, columns). Red: the regulatory profiles of the physically interacting genes have significant (P<0.05) positive correlation. Blue: the regulatory profiles of the physically interacting genes have significant (P<0.05) negative correlation. Grey: the regulatory profiles of the physically interacting genes are not significantly correlated. Genes are sorted by module membership (colors on top and left).

FIG. 11H is a network diagram showing physical interactions (edges: STRING DB experimental score >0) between NFkB signaling pathway components included in the regulatory model (nodes), colored by significant (P<0.05) positive (red) or negative (blue) correlation of their perturbation effects.

FIG. 11I is a pair of charts showing: Top: physical interactions (blue, red, grey; experimental score >0, STRING DB; color code as in G) or lack thereof (white) between each perturbed CLR E3 ligases (rows) and their CLR complex members, including adaptor domain proteins (columns). Columns are ordered by CLR physical complexes/interactions (boxes and dashed lines). Bottom: As on top, except all significant covariances are displayed regardless of interaction evidence.

FIG. 11J is a heat map showing the inferred activity scores (colorbar) of 109 TFs (columns) whose target genes are significantly (FDR <0.1) induced (yellow) or repressed (blue) (by at least 10 perturbations) when perturbing each of 156 E3 and related genes (rows) (that affected at least 10 of the 109 TFs).

FIG. 11K is a set of Venn diagrams showing the intersection between TF targets (DoRothEA), E3 expression targets, and gene programs.

FIG. 12A is a chart showing Regulators (rows) associated (green) with each factor (columns) by their outlier weights in the mixing matrix.

FIG. 12B is a chart showing member genes (columns) associated (red) with each factor (columns) by their outlier loadings in the matrix of source signal estimates.

FIG. 12C is a graph showing the ‘gn’-main criterion for the ladle estimate (y-axis) in randomly sampled unseen perturbation responses for ICA decomposition with different numbers of components (x-axis).

FIG. 12D is a graph showing the explained variance (R², y-axis) after matrix reconstruction with estimated components in randomly sampled unseen perturbation responses for ICA decomposition with different numbers of components (x-axis).

FIG. 12E is a set of box plots showing the distribution of explained variance (y axis) in randomly sampled unseen perturbation responses for ICA decomposition with different numbers of components (x-axis).

FIG. 12F is a set of UMAP embeddings of cell profiles (as in FIG. 1C) colored by expression scores for each sub-factor (label on top).

FIG. 12G is a heat map showing the Jaccard index (color bar) for each pair of factors (columns and rows) based on genes with outlier loadings in the matrix of source signal estimates.

FIG. 12H is a heat map showing the Jaccard index (color bar) for each pair of factors (columns and rows) based on regulators with outlier weights in the mixing matrix per factor.

FIG. 13A is a heat map showing the number of cells (color bar, number) in the E3 screen with pairs of guides targeting genes in the same or pair of modules (rows, columns).

FIG. 13B is a chart showing the number of significant interaction terms (FDR <0.1, y-axis) on the expression of each gene (x-axis) from combinatorial perturbations across all intra-module (red) and inter-(orange) module interactions.

FIG. 13C is a stacked bar graph showing the number of target genes (y-axis, of 1,041 in the regulatory model) with a significant effect on expression (blue) due to single perturbations in genes in one module (Mᵢ, x-axis) or with a significant interaction term due to perturbation in two genes from the same (Mᵢ:Mᵢ, x-axis) or different (Mᵢ:Mⱼ, i≠j, x-axis) module.

FIG. 13D is a chart showing the binarized significant effect (FDR <0.1) (red: positive; blue: negative) on the expression of genes (row, only genes with significant interaction terms) by single perturbations in regulators from two different modules (Mᵢ, Mⱼ, i≠j), their additive effect (Mᵢ+Mⱼ), their interaction term (Mᵢ:Mⱼ), and the observed effect (columns). Gene program membership is labeled on left.

FIG. 13E is a chart showing the binarized significant effect (FDR <0.1) (red: positive; blue: negative) on the expression of genes (row, only genes with significant interaction terms) by single perturbations in regulators from two different modules (Mᵢ, Mⱼ, i≠j), their additive effect (Mᵢ+Mⱼ), their interaction term (Mᵢ:Mⱼ), and the observed effect (columns). Gene program membership is labeled on left.

FIG. 13F is a set of scatter plots showing fold changes in gene expression observed (y-axis) following inter-module combinatorial perturbation or predicted by an additive model (x-axis) for each of the 1,041 genes (dots). R²: explained variance in observed fold changes; MAE: mean absolute error of the predictions.

FIG. 13G is a set of scatter plots showing fold changes in gene expression observed (y-axis) following inter-module combinatorial perturbation or predicted by an additive model (x-axis) for each of the 1,041 genes with significant (FDR <0.1) inter-module interaction terms. R²: explained variance in observed fold changes.

FIG. 13H is a set of scatter plots showing fold changes in gene expression observed (y-axis) following inter-module combinatorial perturbation or predicted by an additive model (x-axis) for each of the 1,041 genes with non-significant (FDR >=0.1) inter-module interaction terms. R²: explained variance in observed fold changes.

FIG. 14A is a set of scatter plots showing fold changes in gene expression observed (y-axis) following inter-module combinatorial perturbation (y-axis) or predicted by comβVAE (x-axis) for each of the 1,041 genes (dots) or only genes with significant (B, FDR <0.1) inter-module interaction terms. The diagonal entries reflect the prediction in single knockouts.

FIG. 14B is a set of box plots showing fold changes in gene expression observed (y-axis) following inter-module combinatorial perturbation (y-axis) or predicted by comβVAE (x-axis) for each of the 1,041 genes with significant (FDR <0.1) inter-module interaction terms. All boxes in Box plots display the first (Q₁), second (Q₂,median) and third (Q₃) quartiles, and bottom and top whiskers show the intervals [Q₁-1.5 IQR, Q₁] and [Q₃, Q_{3+1.5} IQR], respectively.

FIG. 14C is a set of scatter plots showing the distribution of explained variance (top, y-axis, R²) and mean absolute error (bottom, y-axis, MAE) of the predictions of the comβVAE model for each module (Mᵢ) or inter-module combination (Mᵢ:Mⱼ, i≠j) (x-axis) across 7 runs with the same hyperparameters.

FIG. 14D is a set of box plots showing the distribution of explained variance in fold changes (D, y-axis) of the comβVAE model at different KL loss weight values (x-axis) for each module (Mᵢ) or inter-module combination (Mᵢ:Mⱼ, i≠j) across 7 runs with the same hyperparameters.

FIG. 14E is a set of box plots showing the distribution of the explained variance in fold changes (y-axis, R²) in the indicated inter-module combinations (labels on top of panel) when the model (Beta=6.0) is trained only with data from singly perturbed cells from all modules (M) or singly perturbed cells from all modules and doubly perturbed cells from one or two pairs of modules (Mᵢ:Mⱼ, i≠j) (x-axis) across 7 runs with the same hyperparameters.

FIG. 14F is a set of box plots showing the explained variance in fold changes (y-axis) in each module pair (panels) when the comβVAE model is learned with different KL loss weight values (x-axis) and trained either only with singly perturbed cells (red) or with both singly perturbed and doubly perturbed cells of specific module pairs (green: M3M5, blue: M5M6, purple: M3M5 and M5M6).

FIG. 14G is a set of box plots showing the explained variance in fold changes (y-axis) in select module pairs with relatively high number of genes with significant inter-module interaction terms (column headers) when the comβVAE is learned at different KL loss weight values (x-axis) and trained either only with singly perturbed cells (red) or with both singly perturbed and doubly perturbed cells of specific module pairs (row labels).

### DETAILED DESCRIPTION OF THE INVENTION

### I. Definitions

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) aspects that are directed to that value or parameter per se.

As used herein, the singular forms “a,” “an,” and “the” include plural referents unless the context clearly dictates otherwise. For example, reference to “an isolated peptide” means one or more isolated peptides.

Throughout this specification and claims, the word “comprise,” or variations such as “comprises” or “comprising” will be understood to imply the inclusion of a stated integer or group of integers but not the exclusion of any other integer or group of integers.

The terms “patient,” “subject,” or “individual,” as used interchangeably herein, refer to a human patient.

An “effective amount” refers to an amount of an agent (e.g., a therapeutic agent) that is effective to bring about a therapeutic/prophylactic benefit (e.g., as described herein) that is not outweighed by unwanted/undesirable side effects.

The term “pharmaceutical formulation” refers to a preparation which is in such form as to permit the biological activity of the active ingredient or ingredients to be effective, and which contains no additional components which are unacceptably toxic to a subject to which the formulation would be administered. Such formulations are sterile. In one embodiment, the formulation is for intravenous (iv) administration. In another embodiment, the formulation is for subcutaneous (sc) administration.

A “native sequence” protein herein refers to a protein comprising the amino acid sequence of a protein found in nature, including naturally occurring variants of the protein. The term as used herein includes the protein as isolated from a natural source thereof or as recombinantly produced.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein any form of the protein that results from processing in the cell. The term also encompasses naturally occurring variants of the protein, e.g., splice variants or allelic variants, e.g., amino acid substitution mutations or amino acid deletion mutations.

The term also includes isolated regions or domains of the protein, e.g., the extracellular domain (ECD).

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, a “modulator” is an agent that modulates (e.g., increases, decreases, activates, or inhibits) a given biological activity, e.g., an interaction or a downstream activity resulting from an interaction between two proteins (e.g., a direct interaction or an indirect interaction). A modulator or candidate modulator may be, e.g., a small molecule, an antibody (e.g., a bispecific or multispecific antibody), an antigen-binding fragment (e.g., a bis-Fab, an Fv, a Fab, a Fab′-SH, a F(ab′)₂, a diabody, a linear antibody, an scFv, an ScFab, a VH domain, or a VHH domain), a peptide, a mimic, an antisense oligonucleotide, or an inhibitory nucleic acid (e.g., an antisense oligonucleotide (ASO) or a small interfering RNA (siRNA)).

By “increase” or “activate” is meant the ability to cause an overall increase, for example, of 20% or greater, of 50% or greater, or of 75%, 85%, 90%, or 95% or greater. In certain aspects, increase or activate can refer to a downstream activity of a protein-protein interaction.

By “reduce” or “inhibit” is meant the ability to cause an overall decrease, for example, of 20% or greater, of 50% or greater, or of 75%, 85%, 90%, or 95% or greater. In certain aspects, reduce or inhibit can refer to a downstream activity of a protein-protein interaction.

“Affinity” refers to the strength of the sum total of noncovalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and ligand). The affinity of a molecule X for its partner Y can generally be represented by the dissociation constant (K_{D}). Affinity can be measured by common methods known in the art, including those described herein.

“Complex” or “complexed” as used herein refers to the association of two or more molecules that interact with each other through bonds and/or forces (e.g., Van der Waals, hydrophobic, hydrophilic forces) that are not peptide bonds. In one aspect, a complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

The terms “host cell,” “host cell line,” and “host cell culture” are used interchangeably and refer to cells into which exogenous nucleic acid has been introduced, including the progeny of such cells. Host cells include “transfected cells,” “transformed cells,” and “transformants,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In some aspects, the host cell is stably transformed with the exogenous nucleic acid. In other aspects, the host cell is transiently transformed with the exogenous nucleic acid.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

An “antigen-binding fragment” or “antibody fragment” refers to a molecule other than an intact antibody that comprises a portion of an intact antibody that binds the antigen to which the intact antibody binds. Examples of antigen-binding fragments include but are not limited to bis-Fabs; Fv; Fab; Fab, Fab′-SH; F(ab′)₂; diabodies; linear antibodies; single-chain antibody molecules (e.g., scFv, scFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The terms “full-length antibody,” “intact antibody,” and “whole antibody” are used herein interchangeably to refer to an antibody having a structure substantially similar to a native antibody structure or having heavy chains that contain an Fc region as defined herein.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

The term “small molecule” refers to any molecule with a molecular weight of about 2000 daltons or less, e.g., about 1000 daltons or less. In some aspects, the small molecule is a small organic molecule.

The term “mimic” or “molecular mimic,” as used herein, refers to a polypeptide having sufficient similarity in conformation and/or binding ability (e.g., secondary structure, tertiary structure) to a given polypeptide or to a portion of said polypeptide to bind to a binding partner of said polypeptide. The mimic may bind the binding partner with equal, less, or greater affinity than the polypeptide it mimics. A molecular mimic may or may not have obvious amino acid sequence similarity to the polypeptide it mimics. A mimic may be naturally occurring or may be engineered. In some aspects, the mimic is a mimic of a member of a binding pair. In yet other aspects, the mimic is a mimic of another protein that binds to a member of the binding pair. In some aspects, the mimic may perform all functions of the mimicked polypeptide. In other aspects, the mimic does not perform all functions of the mimicked polypeptide.

As used herein, the term “conditions permitting the binding” of two or more proteins to each other refers to conditions (e.g., protein concentration, temperature, pH, salt concentration) under which the two or more proteins would interact in the absence of a modulator or a candidate modulator. Conditions permitting binding may differ for individual proteins and may differ between protein-protein interaction assays (e.g., surface plasmon resonance assays, biolayer interferometry assays, enzyme-linked immunosorbent assays (ELISA), extracellular interaction assays, and cell surface interaction assays.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease (e.g., preventing a disease or disorder related to dendritic cells and/or inflammation or symptoms thereof), reducing or preventing secondary infection in a patient having an infection (e.g., reducing or preventing secondary infection of nervous tissue, immune cells, lymphoid tissue, and/or lung tissue), alleviation of symptoms, diminishment of any direct or indirect pathological consequences of the disease, decreasing the rate of disease progression, amelioration or palliation of the disease state, and remission or improved prognosis.

The “pathology” of a disease or condition includes all phenomena that compromise the well-being of the patient.

“Amelioration,” “ameliorating,” “alleviation,” “alleviating,” or equivalents thereof, refers to both therapeutic treatment and prophylactic or preventative measures, wherein the object is to ameliorate, prevent, slow down (lessen), decrease or inhibit a disease or condition, e.g., a disease or disorder related to dendritic cells and/or inflammation. Those in need of treatment include those already with the disease or condition as well as those prone to having the disease or condition or those in whom the disease or condition is to be prevented.

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with a cancer, an inflammatory disease, or an autoimmune disease are mitigated or eliminated. Indicators of successful treatment of a cancer include, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals. Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment.

As used herein, “in combination with” or “in conjunction with” refers to administration of one treatment modality in addition to another treatment modality, for example, a treatment regimen that includes administration of a modulator or a modified cell as provided herein and one or more additional agents. As such, “in combination with” refers to administration of one treatment modality before, during, or after administration of the other treatment modality to the patient.

The terms “cancer” and “cancerous” refer to or describe the physiological condition in mammals that is typically characterized by unregulated cell growth. Cancers include solid tumor cancers and non-solid tumor cancers and locally advanced or metastatic cancers (e.g., locally advanced or metastatic tumors). Examples of cancer include but are not limited to, carcinoma, lymphoma, blastoma, sarcoma, and leukemia or lymphoid malignancies. More particular examples of such cancers include, but are not limited to urothelial carcinoma (UC), including locally advanced and metastatic UC (mUC), bladder cancer (e.g., muscle invasive bladder cancer (MIBC) and non-muscle invasive bladder cancer (NMIBC), e.g., BCG-refractory NMIBC), MIBC urothelial bladder cancer (UBC); kidney or renal cancer (e.g., renal cell carcinoma (RCC)); cancer of the urinary tract; lung cancer, such as small cell lung cancer (SCLC), which includes extensive stage SCLC (ES-SCLC); non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer (e.g., epithelial squamous cell cancer (e.g., squamous carcinoma of the lung); pancreatic cancer (e.g., pancreatic ductal adenocarcinoma (PDAC), e.g., metastatic PDAC)); head and neck cancer (e.g., SCCHN, e.g., recurrent/metastatic PD-L1-positive SCCHN, and head and neck squamous cell cancer (HNSCC); ovarian cancer (OC); esophageal cancer; cancer of the peritoneum; hepatocellular cancer; gastric cancer (GC) (e.g., gastroesophageal junction (GEJ) cancer) or stomach cancer, including gastrointestinal cancer and gastrointestinal stromal cancer; glioblastoma; cancer of the urinary tract; hepatoma; breast cancer (e.g., HER2+breast cancer and triple-negative breast cancer (TNBC (e.g., early TNBC (eTNBC)), which are estrogen receptors (ER−), progesterone receptors (PgR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer (e.g., a Stage IVB, metastatic, recurrent, or persistent cervical cancer, e.g., a metastatic and/or recurrent PD-L1-positive cervical carcinoma); ovarian cancer; liver cancer (e.g., hepatocellular carcinoma (HCC), e.g., locally advanced or metastatic HCC and/or unresectable HCC); hepatoma; colon cancer; rectal cancer; colorectal cancer (CRC; e.g., CRC with microsatellite-stable (MSS) and microsatellite instability (MSI) low (MSI-Low)); endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

A “disorder” or “disease” is any condition that would benefit from treatment including, but not limited to, disorders that are associated with some degree of abnormal cell proliferation, e.g., cancer, and disorders that are associated with dysregulation of inflammation and/or immune response, e.g., inflammatory disease and autoimmune disease. Inflammatory and/or autoimmune diseases include, but are not limited to, neurodegenerative diseases (e.g., multiple sclerosis (MS), Alzheimer's disease (AD), amyotrophic lateral sclerosis (ALS), and Parkinson's disease (PD)), arthritis, allergy, eczema, fibrosis, asthma, lupus erythematosus, inflammatory bowel disease, ulcerative colitis, and Crohn's disease.

### II. Modulators of Protein-Protein Interactions

In some aspects, the disclosure features modulators of protein-protein interactions; methods of identifying such modulators; and methods of treating a disease (e.g., a cancer, an inflammatory disease, or an autoimmune disease) comprising administering a modulator of a protein-protein interaction. In any of these aspects, the protein-protein interaction may be a direct interaction, e.g., an interaction in which the members of the interaction physically contact one another (e.g., bind to one another). Alternatively, in some aspects, the protein-protein interaction is an indirect interaction, e.g., an interaction in which the members of the interaction do not physically contact one another. Indirect protein-protein interactions may be identified by determination of a causal relationship between expression, activity, and/or abundance of a first member of the protein-protein interaction and expression, activity, and/or abundance of a second member of the protein-protein interaction (e.g., perturbation of a first member of the protein-protein interaction in a biological system (e.g., organism, tissue, or cell) has a measurable effect on (e.g., affects expression, activity, and/or abundance of) the second member of the protein-protein interaction). In some aspects, proteins having an indirect interaction are associated in a pathway or network.

In some aspects, a modulator of a protein-protein interaction directly interacts with (e.g., binds to) one or both members of the protein-protein interaction. In other aspects, a modulator of a protein-protein interaction does not directly interact with either member of the protein-protein interaction.

In some aspects, the disclosure features an isolated modulator of the interaction between a first protein and a second protein, wherein the protein-protein interaction is a direct interaction and the modulator causes a decrease in the binding of the first protein to the second protein relative to binding in the absence of the modulator.

In some aspects, the disclosure features an isolated modulator of the interaction between a first protein and a second protein, wherein the protein-protein interaction is a direct interaction and the modulator causes an increase in the binding of the first protein to the second protein relative to binding in the absence of the modulator.

In some aspects, the disclosure features an isolated modulator of the interaction between a first protein and a second protein, wherein the protein-protein interaction is an indirect interaction and the modulator causes a decrease in a downstream activity of one or both members of a protein-protein interaction relative to the downstream activity in the absence of the modulator.

In some aspects, the disclosure features an isolated modulator of the interaction between a first protein and a second protein, wherein the protein-protein interaction is an indirect interaction and the modulator causes an increase in a downstream activity of one or both members of a protein-protein interaction relative to the downstream activity in the absence of the modulator.

In some aspects, the modulator comprises a pharmaceutically acceptable carrier.

### A. Modulators of the Interaction Between a First Protein and a Second Protein Direct Interactions

In some aspects, the disclosure features a modulator of the interaction between a first protein and a second protein, wherein the protein-protein interaction is a direct interaction and the modulator causes a decrease in the binding of the first protein to the second protein and/or binding of the second protein to the first protein.

In some aspects, the modulator decreases binding of the first protein to the second protein and/or binding of the second protein to the first protein by at least 50%. In some aspects, the decrease in binding is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., binding is abolished), e.g., the decrease is 5%-15%, 15%-25%, 25%-35%, 35%-45%, 45%-55%, 55%-65%, 65%-75%, 75%-85%, 85%-95%, or 95%-100%, relative to binding in the absence of the modulator. In some aspects, the modulator decreases binding of the first protein to the second protein and/or binding of the second protein to the first protein by at least 90% (e.g., 90%-100%). In some aspects, the decrease in binding is at least 50% (e.g., is 50%-55%, 55%-65%, 65%-75%, 75%-85%, 85%-95%, or 95%-100%), e.g., as measured by surface plasmon resonance, biolayer interferometry, or an enzyme-linked immunosorbent assay (ELISA).

In some aspects, the disclosure features a modulator of the interaction between a first protein and a second protein, wherein the protein-protein interaction is a direct interaction and the modulator causes an increase in the binding of the first protein to the second protein and/or binding of the second protein to the first protein.

In some aspects, the modulator increases binding of the first protein to the second protein and/or binding of the second protein to the first protein by at least 50%. In some aspects, the increase in binding is at least 5%, 10%, 15%, 20%, 25%, 30%, 35%, 40%, 45%, 50%, 55%, 60%, 65%, 70%, 75%, 80%, 85%, 90%, 95%, 98%, 99% 100%, or more than 100%, e.g., the increase is 5%-15%, 15%-25%, 25%-35%, 35%-45%, 45%-55%, 55%-65%, 65%-75%, 75%-85%, 85%-95%, or 95%-100%, relative to binding in the absence of the modulator. In some aspects, the modulator increases binding of the first protein to the second protein and/or binding of the second protein to the first protein by at least 90% (e.g., 90%-100%). In some aspects, the increase in binding is at least 50% (e.g., is 50%-55%, 55%-65%, 65%-75%, 75%-85%, 85%-95%, or 95%-100%), e.g., as measured by surface plasmon resonance, biolayer interferometry, or an enzyme-linked immunosorbent assay (ELISA).

### Indirect Interactions

In some aspects, the disclosure features a modulator of the interaction between a first protein and a second protein, wherein the protein-protein interaction is an indirect interaction and the modulator disrupts a causal relationship between expression, activity, and/or abundance of a first member of the protein-protein interaction and expression, activity, and/or abundance of a second member of the protein-protein interaction. For example, in some aspects, the first protein regulates the expression of the second protein (e.g., by regulating transcription or translation) and the modulator disrupts regulation of expression; the first protein regulates the activity of the second protein (e.g., as a component of an upstream signaling pathway) and the modulator disrupts regulation of activity; and/or the first protein regulates abundance of the second protein (e.g., by targeting the second protein for degradation, e.g., by acting as a ubiquitin ligase) and the modulator disrupts regulation of abundance.

In some aspects, disruption of the causal relationship results in a change in a downstream activity (e.g., an increase or decrease in the amount, strength, or duration of the downstream activity) of one or both members of the protein-protein interaction relative to the downstream activity in the absence of the modulator.

### Direct and Indirect Interactions

In some aspects, the modulator causes an increase in a downstream activity (e.g., an increase in the amount, strength, or duration of the downstream activity) of one or both members of a protein-protein interaction relative to the downstream activity in the absence of the modulator. Downstream activities may include any biological activity that occurs as a direct or indirect result of expression and/or activity of the member of the protein-protein interaction, e.g., transcriptional regulation, signaling, and catalysis. In some aspects, the downstream activity is increased by at least 40%. In some aspects, the increase is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100%, e.g., the increase is 5%-15%, 15%-25%, 25%-35%, 35%-45%, 45%-55%, 55%-65%, 65%-75%, 75%-85%, 85%-95%, or 95%-100%).

In some aspects, the modulator causes a decrease in a downstream activity relative to the downstream activity in the absence of the modulator. In some aspects, the downstream activity is decreased by at least 40%. In some aspects, the decrease is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., the downstream activity does not occur at a detectable level), e.g., the decrease is 5%-15%, 15%-25%, 25%-35%, 35%-45%, 45%-55%, 55%-65%, 65%-75%, 75%-85%, 85%-95%, or 95%-100%).

### B. Small Molecules

In some aspects, the modulator or candidate modulator is a small molecule. Small molecules are molecules other than binding polypeptides or antibodies as defined herein. Binding small molecules may be identified and chemically synthesized using known methodology (see, e.g., PCT Publication Nos. WO00/00823 and WO00/39585). Binding small molecules are usually less than about 2000 daltons in size (e.g., less than about 2000, 1500, 750, 500, 250 or 200 daltons in size), wherein such organic small molecules that are capable of binding, preferably specifically, to a polypeptide as described herein may be identified without undue experimentation using well known techniques. In this regard, it is noted that techniques for screening small molecule libraries for molecules that are capable of binding to a polypeptide target are well known in the art (see, e.g., PCT Publication Nos. WO00/00823 and WO00/39585). Binding small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some aspects, the binding of the first protein to the second protein is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the small molecule.

In some aspects, the binding of the first protein to the second protein is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the small molecule.

In some aspects, a downstream activity of the first protein and/or the second protein is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the small molecule.

### C. Antibodies and Antigen-Binding Fragments

In some aspects, the modulator or candidate modulator is an antibody or an antigen-binding fragment thereof binding one or both members of the protein-protein interaction. In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the modulator is a multispecific antibody, e.g., a bispecific antibody. In some aspects, the modulator is a bispecific or multispecific antibody that binds multiple epitopes of one or both members of the protein-protein interaction. In some aspects, the modulator is a bispecific or multispecific antibody that binds both members of the protein-protein interaction.

In some aspects, the binding of a first member of the protein-protein interaction to a second member of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the antibody or antigen-binding fragment.

In some aspects, the binding of the first member to the second member is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the antibody or antigen-binding fragment.

In some aspects, a downstream activity of one or both members of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the antibody or antigen-binding fragment.

In some aspects, a downstream activity of one or both members of the protein-protein interaction is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the antibody or antigen-binding fragment.

### D. Peptides

In some aspects, the modulator or candidate modulator is a peptide that binds to one or both members of the protein-protein interaction. The peptide may be the peptide may be naturally occurring or may be engineered. The peptide may bind the binding partner with equal, less, or greater affinity than the full-length protein. In some aspects, the peptide performs all functions of the full-length protein. In other aspects, the peptide does not perform all functions of the full-length protein.

In some aspects, the binding of a first member of the protein-protein interaction to a second member of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the peptide.

In some aspects, the binding of the first member to the second member is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the peptide.

In some aspects, a downstream activity of one or both members of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the peptide.

In some aspects, a downstream activity of one or both members of the protein-protein interaction is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the peptide.

### E. Mimics

In some aspects, the modulator or candidate modulator is a mimic, e.g., a molecular mimic, that binds to one or both members of the protein-protein interaction. In some aspects, the mimic may perform all functions of the mimicked polypeptide. In other aspects, the mimic does not perform all functions of the mimicked polypeptide.

In some aspects, the binding of a first member of the protein-protein interaction to a second member of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the mimic.

In some aspects, the binding of the first member to the second member is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the mimic.

In some aspects, a downstream activity of one or both members of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the mimic.

In some aspects, a downstream activity of one or both members of the protein-protein interaction is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the mimic.

### F. PROTACs

In some aspects, the modulator or candidate modulator is a proteolysis targeting chimera (PROTAC) that binds to one or both members of the protein-protein interaction. PROTACs are described, e.g., in Sakamoto et al., Proc Natl Acad Sci USA, 98(15): 8554-8559, 2001.

In some aspects, the binding of a first member of the protein-protein interaction to a second member of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the PROTAC.

In some aspects, the binding of the first member to the second member is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the PROTAC.

In some aspects, a downstream activity of one or both members of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the PROTAC.

In some aspects, a downstream activity of one or both members of the protein-protein interaction is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the PROTAC.

In some aspects, the abundance of one or both members of the protein-protein interaction is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the PROTAC.

### G. Assays for Modulation of Protein-Protein Interactions

In some aspects, the binding of a first member of the protein-protein interaction to a second member of the protein-protein interaction in the presence or absence of the candidate modulator is assessed in an assay for protein-protein interaction. Modulation of the interaction may be identified as an increase in protein-protein interaction in the presence of the modulator compared to protein-protein interaction in the absence of the modulator, e.g., an increase of 5%, 10%, 15%, 20%, 25%, 30%, 35%, 40%, 45%, 50%, 55%, 60%, 65%, 70%, 80%, 90%, 95%, 100%, or more than 100% (e.g., 5%-15%, 15%-25%, 25%-35%, 35%-45%, 45%-55%, 55%-65%, 65%-75%, 75%-85%, 85%-95%, 95%-100%, or more than 100%) in protein-protein interaction. Alternatively, modulation may be identified as a decrease in protein-protein interaction in the presence of the modulator compared to protein-protein interaction in the absence of the modulator, e.g., a decrease of 5%, 10%, 15%, 20%, 25%, 30%, 35%, 40%, 45%, 50%, 55%, 60%, 65%, 70%, 80%, 90%, 95%, or 100% (e.g., 5%-15%, 15%-25%, 25%-35%, 35%-45%, 45%-55%, 55%-65%, 65%-75%, 75%-85%, 85%-95%, or 95%-100%) in protein-protein interaction. The assay for protein-protein interaction may be, e.g., an SPR assay, a biolayer interferometry (BLI) assay, an enzyme-linked immunosorbent assay (ELISA), an extracellular interaction assay, or a cell surface interaction assay.

Exemplary methods for identifying modulators of protein-protein interactions, as well as agents that may modulate such interactions, are described in WO 2020/205626, which is hereby incorporated by reference in its entirety.

### H. Methods of Delivery

The compositions utilized in the methods described herein (e.g., a PROTAC, a small molecule, an antibody, an antigen-binding fragment, a peptide, a mimic, an antisense oligonucleotide, or an siRNA) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, a modulator of a protein-protein interaction is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A modulator of a protein-protein interaction described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The modulator need not be, but is optionally formulated with and/or administered concurrently with one or more agents currently used to prevent or treat the disorder in question. The effective amount of such other agents depends on the amount of the modulator present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### III. Methods of Identifying a Modulator of a Protein-Protein Interaction

In some aspects, the disclosure features methods of identifying a modulator of the interaction between a first and a second member of a protein-protein interaction and methods of identifying a modulator of a downstream activity of one or both members of a protein-protein interaction, wherein the methods comprise: (a) providing a candidate modulator (e.g., a candidate modulator described in Section II herein); (b) contacting a first member of the protein-protein interaction with a second member of a protein-protein interaction in the presence or absence of the candidate modulator under conditions permitting the binding of the members of the protein-protein interaction; and (c) measuring the binding of the members of the protein-protein interaction.

In some aspects, the candidate modulator is provided to a cell (e.g., a mammalian cell), to cell culture media, to conditioned media, and/or to a purified form of the first and second members of the protein-protein interaction. In some aspects, the candidate modulator is provided at a concentration of at least 0.1 nM, 0.5 nM, 1 nM, 10 nM, 50 nM, 100 nM, 250 nM, 500 nM, 750 nM, 1 μM, 2 μM, 3 μM, 5 μM, or 10 μM. In some aspects, the candidate modulator is provided at a concentration of between 0.1 nM and 10 μM. In some aspects, the candidate modulator is provided in a solution, e.g., in a soluble form.

In some aspects, the candidate modulator is identified as a modulator if the increase in binding is at least 70%. In some aspects, the increase in binding is at least 5%, at least 10%, at least 20%, at least 30%, at least 40%, at least 50%, at least 60%, at least 70%, at least 80%, at least 90%, at least 100%, or more than 100% (e.g., the increase is 5%-15%, 15%-25%, 25%-35%, 35%-45%, 45%-55%, 55%-65%, 65%-75%, 75%-85%, 85%-95%, 95%-100%, or more than 100%). In some aspects, the increase in binding is at least 70%.

In some aspects, the candidate modulator is identified as a modulator if the decrease in binding is at least 70%. In some aspects, the decrease in binding is at least 5%, at least 10%, at least 20%, at least 30%, at least 40%, at least 50%, at least 60%, at least 70%, at least 80%, at least 90%, or 100% (e.g., the decrease in binding is 5%-15%, 15%-25%, 25%-35%, 35%-45%, 45%-55%, 55%-65%, 65%-75%, 75%-85%, 85%-95%, or 95%-100%). In some aspects, the decrease in binding is at least 70%.

The assay for protein-protein interaction may be, e.g., an SPR assay, a biolayer interferometry (BLI) assay, an enzyme-linked immunosorbent assay (ELISA), an extracellular interaction assay as described in WO 2020/205626, or a cell surface interaction assay as described in WO 2020/205626.

### A. Assays for Modulation of the Interaction Between Fbxw11 and Nfkb1 and/or Nfkb2

In some aspects, the disclosure features a method of identifying a modulator of the interaction between F-box and WD repeat domain containing 11 (Fbxw11) and nuclear factor kappa B subunit 1 (Nfkb1) or nuclear factor kappa B subunit 2 (Nfkb2), the method comprising: (a) providing a candidate modulator (e.g., a candidate modulator described in Section II herein); (b) contacting Fbxw11 with Nfkb1 or Nfkb2 in the presence or absence of the candidate modulator under conditions permitting the binding of Fbxw11 to Nfkb1 or Nfkb2; and (c) measuring the binding of Fbxw11 to Nfkb1 or Nfkb2, wherein an increase or decrease in binding in the presence of the candidate modulator relative to binding in the absence of the candidate modulator identifies the candidate modulator as a modulator of the interaction between Fbxw11 and Nfkb1 or Nfkb2.

In some aspects, the disclosure features a method of identifying a modulator of a downstream activity of Fbxw11, the method comprising (a) providing a candidate modulator; (b) contacting Fbxw11 with Nfkb1 or Nfkb2 in the presence or absence of the candidate modulator under conditions permitting the binding of Fbxw11 to Nfkb1 or Nfkb2; and (c) measuring a downstream activity of Fbxw11, wherein a change in the downstream activity in the presence of the candidate modulator relative to the downstream activity in the absence of the candidate modulator identifies the candidate modulator as a modulator of the downstream activity of Fbxw11.

In some aspects, the disclosure features a method of identifying a modulator of a downstream activity of Nfkb1 or Nfkb2, the method comprising (a) providing a candidate modulator; (b) contacting Nfkb1 or Nfkb2 with Fbxw11 in the presence or absence of the candidate modulator under conditions permitting the binding of Nfkb1 or Nfkb2 to Fbxw11; and (c) measuring a downstream activity of Nfkb1 or Nfkb2, wherein a change in the downstream activity in the presence of the candidate modulator relative to the downstream activity in the absence of the candidate modulator identifies the candidate modulator as a modulator of the downstream activity of Nfkb1 or Nfkb2.

In some aspects, the increase or decrease in binding is at least 50% (e.g., 50%-55%, 55%-65%, 65%-75%, 75%-85%, 85%-95%, or 95%-100%), as measured by surface plasmon resonance, biolayer interferometry, or an enzyme-linked immunosorbent assay (ELISA).

In some aspects, the modulator is an inhibitor of the downstream activity of Fbxw11 or Nfkb1 or Nfkb2.

In some aspects, the modulator is a modulator as described in Section II herein, e.g., is a proteolysis targeting chimera (PROTAC), a small molecule, an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain), a peptide, a mimic, or an inhibitory nucleic acid (e.g., an ASO or a siRNA).

In some aspects in which the modulator is an antibody or antigen-binding fragment thereof, the antibody or antigen-binding fragment thereof binds Fbxw11. In some aspects, the antibody or antigen-binding fragment thereof binds Nfkb1 and/or Nfkb2. For example, in some aspects, the modulator is an antibody or antigen-binding fragment thereof that binds Fbxw11, an antibody or antigen-binding fragment thereof that binds Nfkb1 or Nfkb2, or an antibody or antigen-binding fragment thereof that binds Fbxw11 and Nfkb1 and/or Nfkb2.

In some aspects, the change in the downstream activity is a decrease in the amount, strength, or duration of the downstream activity.

In some aspects, the downstream activity of Fbxw11 is processing of Nfkb1 to generate active p50 and/or processing of Nfkb2 to generate active p52. In some aspects, processing of Nfkb1 to generate active p50 and/or processing of Nfkb2 to generate active p52 is decreased in the presence of the modulator. The decrease in processing may be a decrease of at least 5%, at least 10%, at least 20%, at least 30%, at least 40%, at least 50%, at least 60%, at least 70%, at least 80%, at least 90%, or 100% (e.g., may be a decrease of 5%-15%, 15%-25%, 25%-35%, 35%-45%, 45%-55%, 55%-65%, 65%-75%, 75%-85%, 85%-95%, or 95%-100%). In other aspects, processing of Nfkb1 to generate active p50 and/or processing of Nfkb2 to generate active p52 is increased in the presence of the modulator. The increase in processing may be an increase of at least 5%, at least 10%, at least 20%, at least 30%, at least 40%, at least 50%, at least 60%, at least 70%, at least 80%, at least 90%, at least 100%, or more than 100% (e.g., may be an increase of 5%-15%, 15%-25%, 25%-35%, 35%-45%, 45%-55%, 55%-65%, 65%-75%, 75%-85%, 85%-95%, 95%-100%, or more than 100%).

In some aspects, the downstream activity of Fbxw11, Nfkb1, and/or Nfkb2 is immune response activation. In some aspects, immune response activation is decreased in the presence of the modulator. The decrease in activation may be a decrease of at least 5%, at least 10%, at least 20%, at least 30%, at least 40%, at least 50%, at least 60%, at least 70%, at least 80%, at least 90%, or 100% (e.g., may be a decrease of 5%-15%, 15%-25%, 25%-35%, 35%-45%, 45%-55%, 55%-65%, 65%-75%, 75%-85%, 85%-95%, or 95%-100%). In other aspects, immune response activation is increased in the presence of the modulator. The increase in activation may be an increase of at least 5%, at least 10%, at least 20%, at least 30%, at least 40%, at least 50%, at least 60%, at least 70%, at least 80%, at least 90%, at least 100%, or more than 100% (e.g., may be an increase of 5%-15%, 15%-25%, 25%-35%, 35%-45%, 45%-55%, 55%-65%, 65%-75%, 75%-85%, 85%-95%, 95%-100%, or more than 100%).

### B. Methods of Treatment Using a Modulator of the Interaction Between Fbxw11 and Nfkb1 and/or Nfkb2

In some aspects, the disclosure features a method for preventing or treating a cancer, an inflammatory disease, or an autoimmune disease in an individual, the method comprising administering to the individual an effective amount of a modulator identified by a method presented in Section III(A), above, thereby treating the individual.

In some aspects, the disclosure features a method for treating a cancer in an individual, the method comprising administering to the individual an effective amount of a modulator of the interaction between Fbxw11 and one or both of Nfkb1 and Nfkb2, wherein immune response activation is increased in the presence of the modulator.

In some aspects, the disclosure features a method for treating an inflammatory disease or an autoimmune disease in an individual, the method comprising administering to the individual an effective amount of a modulator of the interaction between Fbxw11 and one or both of Nfkb1 and Nfkb2, wherein immune response activation is decreased in the presence of the modulator.

### C. Assays for Modulation of the Interaction Between Rfwd2 and Wdr82, Ep300, Anapc13, Cul2, Cul5, Huwe1, Crebbp, Skp1a, Nedd8, Cul1, or Wdr5

In some aspects, the disclosure features a method of identifying a modulator of the interaction between Ring finger and WD repeat domain 2 (Rfwd2) and a query protein selected from Forkhead box L2 (Foxl2), JunD, WD repeat domain 82 (Wdr82); E1A binding protein p300 (Ep300); Anaphase promoting complex subunit 13 (Anapc13); Cullin 2 (Cul2); Cullin 5 (Cul5); HECT, UBA and WWE domain containing E3 ubiquitin protein ligase 1 (Huwe1); CREB binding protein (Crebbp); S-phase kinase associated protein 1 (Skp1 a); Neural precursor cell expressed, developmentally down-regulated gene 8 (Nedd8); Cullin 1 (Cul1); and WD repeat domain 5 (Wdr5), the method comprising: (a) providing a candidate modulator (e.g., a candidate modulator described in Section II herein); (b) contacting Rfwd2 with the query protein in the presence or absence of the candidate modulator under conditions permitting the binding of Rfwd2 to the query protein; and (c) measuring the binding of Rfwd2 to the query protein, wherein an increase or decrease in binding in the presence of the candidate modulator relative to binding in the absence of the candidate modulator identifies the candidate modulator as a modulator of the interaction between Rfwd2 and the query protein.

In some aspects, the disclosure features a method of identifying a modulator of a downstream activity of Rfwd2, the method comprising (a) providing a candidate modulator; (b) contacting Rfwd2 with a query protein selected Wdr82, Ep300, Anapc13, Cul2, Cul5, Huwe1, Crebbp, Skp1 a, Nedd8, Cul1, and Wdr5 in the presence or absence of the candidate modulator under conditions permitting the binding of a query protein selected Wdr82, Ep300, Anapc13, Cul2, Cul5, Huwe1, Crebbp, Skp1 a, Nedd8, Cul1, and Wdr5 to the query protein; and (c) measuring a downstream activity of Rfwd2, wherein a change in the downstream activity in the presence of the candidate modulator relative to the downstream activity in the absence of the candidate modulator identifies the candidate modulator as a modulator of the downstream activity of Rfwd2.

In some aspects, the disclosure features a method of identifying a modulator of a downstream activity of a query protein selected from Wdr82, Ep300, Anapc13, Cul2, Cul5, Huwe1, Crebbp, Skp1 a, Nedd8, Cul1, and Wdr5, the method comprising (a) providing a candidate modulator; (b) contacting the query protein with Rfwd2 in the presence or absence of the candidate modulator under conditions permitting the binding of the query protein to Rfwd2; and (c) measuring a downstream activity of the query protein, wherein a change in the downstream activity in the presence of the candidate modulator relative to the downstream activity in the absence of the candidate modulator identifies the candidate modulator as a modulator of the downstream activity of the query protein.

In some aspects, the increase or decrease in binding is at least 50% (e.g., 50%-55%, 55%-65%, 65%-75%, 75%-85%, 85%-95%, or 95%-100%), as measured by surface plasmon resonance, biolayer interferometry, or an enzyme-linked immunosorbent assay (ELISA).

In some aspects, the modulator is an inhibitor of the downstream activity of Rfwd2 or the query protein.

In some aspects, the modulator is a modulator as described in Section II herein, e.g., is a proteolysis targeting chimera (PROTAC), a small molecule, an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain), a peptide, a mimic, or an inhibitory nucleic acid (e.g., an ASO or a siRNA).

In some aspects in which the modulator is an antibody or antigen-binding fragment thereof, the antibody or antigen-binding fragment thereof binds Rfwd2. In some aspects, the antibody or antigen-binding fragment thereof binds the query protein. For example, in some aspects, the modulator is an antibody or antigen-binding fragment thereof that binds Rfwd2, an antibody or antigen-binding fragment thereof that binds the query protein, or an antibody or antigen-binding fragment thereof that binds Rfwd2 and the query protein.

In some aspects, the change in the downstream activity is a decrease in the amount, strength, or duration of the downstream activity.

In some aspects, the downstream activity of Fbxw11, Wdr82, Ep300, Anapc13, Cul2, Cul5, Huwe1, Crebbp, Skp1 a, Nedd8, Cul1, and/or Wdr5 is dendritic cell and/or macrophage migration. In some aspects, dendritic cell and/or macrophage migration is decreased in the presence of the modulator.

The decrease in dendritic cell and/or macrophage migration may be a decrease of at least 5%, at least 10%, at least 20%, at least 30%, at least 40%, at least 50%, at least 60%, at least 70%, at least 80%, at least 90%, or 100% (e.g., may be a decrease of 5%-15%, 15%-25%, 25%-35%, 35%-45%, 45%-55%, 55%-65%, 65%-75%, 75%-85%, 85%-95%, or 95%-100%). In other aspects, dendritic cell and/or macrophage migration is increased in the presence of the modulator. The increase in dendritic cell and/or macrophage migration may be an increase of at least 5%, at least 10%, at least 20%, at least 30%, at least 40%, at least 50%, at least 60%, at least 70%, at least 80%, at least 90%, at least 100%, or more than 100% (e.g., may be an increase of 5%-15%, 15%-25%, 25%-35%, 35%-45%, 45%-55%, 55%-65%, 65%-75%, 75%-85%, 85%-95%, 95%-100%, or more than 100%).

### D. Methods of Treatment Using a Modulator of the Interaction Between Rfwd2 and Wdr82, Ep300, Anapc13, Cul2, Cul5, Huwe1, Crebbp, Skp1a, Nedd8, Cul1, or Wdr5

In some aspects, the disclosure features a method for preventing or treating a cancer, an inflammatory disease, or an autoimmune disease in an individual, the method comprising administering to the individual an effective amount of a modulator identified by a method presented in Section III(C), above, thereby treating the individual.

In some aspects, the disclosure features a method for treating an inflammatory disease or an autoimmune disease in an individual, the method comprising administering to the individual an effective amount of a modulator of the interaction between Rfwd2 and one or more of Wdr82, Ep300, Anapc13, Cul2, Cul5, Huwe1, Crebbp, Skp1 a, Nedd8, Cul1, and Wdr5, wherein dendritic cell or macrophage migration is decreased in the presence of the modulator.

In some aspects, the disclosure features a method for treating a cancer in an individual, the method comprising administering to the individual an effective amount of a modulator of the interaction between Rfwd2 and one or more of Wdr82, Ep300, Anapc13, Cul2, Cul5, Huwe1, Crebbp, Skp1 a, Nedd8, Cull, and Wdr5, wherein dendritic cell or macrophage migration is increased in the presence of the modulator.

### E. Assays for Modulation of the Interaction Between a Protein Complex Comprising Ptpn11 and Rfwd2 and a Cebp Family Transcription Factor

In some aspects, the disclosure features a method of identifying a modulator of the interaction between a protein complex comprising Tyrosine-protein phosphatase non-receptor type 11 (Ptpn11) and Ring finger and WD repeat domain 2 (Rfwd2) and a CCAAT enhancer-binding protein (Cebp) family transcription factor, the method comprising: (a) providing a candidate modulator (e.g., a candidate modulator described in Section II herein); (b) contacting the protein complex with the Cebp family transcription factor in the presence or absence of the candidate modulator under conditions permitting the binding of the protein complex to the Cebp family transcription factor; and (c) measuring the binding of the protein complex to the Cebp family transcription factor, wherein an increase or decrease in binding in the presence of the candidate modulator relative to binding in the absence of the candidate modulator identifies the candidate modulator as a modulator of the interaction between the protein complex and the Cebp family transcription factor.

In some aspects, the disclosure features a method of identifying a modulator of a downstream activity of a protein complex comprising Ptpn11 and Rfwd2, the method comprising (a) providing a candidate modulator; (b) contacting the protein complex with a Cebp family transcription factor in the presence or absence of the candidate modulator under conditions permitting the binding of the protein complex to the Cebp family transcription factor; and (c) measuring a downstream activity of the protein complex, wherein a change in the downstream activity in the presence of the candidate modulator relative to the downstream activity in the absence of the candidate modulator identifies the candidate modulator as a modulator of the downstream activity of the protein complex.

In some aspects, the disclosure features a method of identifying a modulator of a Cebp family transcription factor, the method comprising (a) providing a candidate modulator; (b) contacting the Cebp family transcription factor with a protein complex comprising Ptpn11 and Rfwd2 in the presence or absence of the candidate modulator under conditions permitting the binding of the Cebp family transcription factor to the protein complex; and (c) measuring a downstream activity of the query protein, wherein a change in the downstream activity in the presence of the candidate modulator relative to the downstream activity in the absence of the candidate modulator identifies the candidate modulator as a modulator of the downstream activity of the query protein.

In some aspects, the increase or decrease in binding is at least 50% (e.g., 50%-55%, 55%-65%, 65%-75%, 75%-85%, 85%-95%, or 95%-100%), as measured by surface plasmon resonance, biolayer interferometry, or an enzyme-linked immunosorbent assay (ELISA).

In some aspects, the modulator is an inhibitor of the downstream activity of a Cebp family transcription factor and/or a protein complex comprising Ptpn11 and Rfwd2.

In some aspects, the modulator is a modulator as described in Section II herein, e.g., is a proteolysis targeting chimera (PROTAC), a small molecule, an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain), a peptide, a mimic, or an inhibitory nucleic acid (e.g., an ASO or a siRNA).

In some aspects in which the modulator is an antibody or antigen-binding fragment thereof, the antibody or antigen-binding fragment thereof binds one or both of Ptpn11 and Rfwd2. In some aspects, the antibody or antigen-binding fragment thereof binds the Cebp family transcription factor. For example, in some aspects, the modulator is an antibody or antigen-binding fragment thereof that binds Fbxw11, an antibody or antigen-binding fragment thereof that binds one, two, or all three of Ptpn11, Rfwd2, and the Cebp family transcription factor or an antibody or antigen-binding fragment thereof that binds the Cebp family transcription factor and one or both of Ptpn11 and Rfwd2.

### IV. Methods of Preventing or Treating a Disease or Disorder Related to APCs

In some aspects, the disclosure features a method for preventing or treating a disease or disorder related to antigen-presenting cells (APCs) and/or inflammation in an individual, the method comprising administering to the individual an effective amount of a modulator of a gene of Table 1 or Table 2, thereby treating the individual. Accordingly, in some aspects, the disclosure features a method for preventing or treating a disease or disorder related to APCs and/or inflammation in an individual, the method comprising administering to the individual an effective amount of a modulator of a gene of Table 1, thereby treating the individual. In some aspects, the disclosure features a method for preventing or treating a disease or disorder related to APCs and/or inflammation in an individual, the method comprising administering to the individual an effective amount of a modulator of a gene of Table 2, thereby treating the individual.

TABLE 1
Co-functional gene module members with no known
role in dendritic cells or inflammation

| | Co-functional | | |
|---|---|---|---|
| Gene | gene module | GeneID | Database |
| Dcaf13 | M1 | 223499 | iUUCD 2.0 |
| Grb2 | M1 | 14784 | NCBI |
| Tbl3 | M1 | 213773 | iUUCD 2.0 |
| Wdr3 | M1 | 269470 | iUUCD 2.0 |
| Anapc13 | M2 | 69010 | iUUCD 2.0 |
| Brca1 | M2 | 12189 | iUUCD 2.0 |
| Brwd3 | M2 | 382236 | iUUCD 2.0 |
| Btbd1 | M2 | 83962 | iUUCD 2.0 |
| Ccnf | M2 | 12449 | iUUCD 2.0 |
| Cdc27 | M2 | 217232 | iUUCD 2.0 |
| E4f1 | M2 | 13560 | iUUCD 2.0 |
| Fbxl14 | M2 | 101358 | iUUCD 2.0 |
| Fbxl5 | M2 | 242960 | iUUCD 2.0 |
| Fbxo42 | M2 | 213499 | iUUCD 2.0 |
| Fzr1 | M2 | 56371 | iUUCD 2.0 |
| Hectd1 | M2 | 207304 | iUUCD 2.0 |
| Katnb1 | M2 | 74187 | iUUCD 2.0 |
| Kbtbd13 | M2 | 74492 | iUUCD 2.0 |
| Klhl3 | M2 | 100503085 | iUUCD 2.0 |
| Kmt2b | M2 | 75410 | iUUCD 2.0 |
| Lrr1 | M2 | 69706 | iUUCD 2.0 |
| Lrrc41 | M2 | 230654 | iUUCD 2.0 |
| Mdm4 | M2 | 17248 | iUUCD 2.0 |
| Mkrn1 | M2 | 54484 | iUUCD 2.0 |
| Pa2g4 | M2 | 18813 | NCBI |
| Pcif1 | M2 | 228866 | iUUCD 2.0 |
| Ring1 | M2 | 19763 | iUUCD 2.0 |
| Taf3 | M2 | 209361 | iUUCD 2.0 |
| Ttc3 | M2 | 22129 | iUUCD 2.0 |
| Wdhd1 | M2 | 218973 | iUUCD 2.0 |
| Zmiz1 | M2 | 328365 | iUUCD 2.0 |
| Ankfy1 | M3 | 11736 | iUUCD 2.0 |
| Cul2 | M3 | 71745 | iUUCD 2.0 |
| Dcaf4 | M3 | 73828 | iUUCD 2.0 |
| Fbxl13 | M3 | 320118 | iUUCD 2.0 |
| Fbxo28 | M3 | 67948 | iUUCD 2.0 |
| Gnb2 | M3 | 14693 | iUUCD 2.0 |
| Klhl24 | M3 | 75785 | iUUCD 2.0 |
| Klhl7 | M3 | 52323 | iUUCD 2.0 |
| Med8 | M3 | 80509 | iUUCD 2.0 |
| Nosip | M3 | 66394 | iUUCD 2.0 |
| Rnf113a1 | M3 | 69942 | iUUCD 2.0 |
| Traf7 | M3 | 224619 | iUUCD 2.0 |
| Ube3c | M3 | 100763 | iUUCD 2.0 |
| Wdr1 | M3 | 22388 | iUUCD 2.0 |
| Ppil2 | M4 | 66053 | iUUCD 2.0 |
| Sart1 | M4 | 20227 | iUUCD 2.0 |
| Smu1 | M4 | 74255 | iUUCD 2.0 |
| Wdr70 | M4 | 545085 | iUUCD 2.0 |
| Ambra1 | M5 | 228361 | iUUCD 2.0 |
| Arih1 | M5 | 23806 | iUUCD 2.0 |
| Cnot4 | M5 | 53621 | iUUCD 2.0 |
| Dcaf7 | M5 | 71833 | iUUCD 2.0 |
| Det1 | M5 | 76375 | NCBI |
| March6 | M5 | 223455 | iUUCD 2.0 |
| Msl2 | M5 | 77853 | iUUCD 2.0 |
| Rnf139 | M5 | 75841 | iUUCD 2.0 |
| Strap | M5 | 20901 | iUUCD 2.0 |
| Trim45 | M5 | 229644 | iUUCD 2.0 |
| Zmiz2 | M5 | 52915 | iUUCD 2.0 |
| Anapc11 | M6 | 66156 | iUUCD 2.0 |
| Cul3 | M6 | 26554 | iUUCD 2.0 |
| Dda1 | M6 | 66498 | NCBI |
| Fbxo33 | M6 | 70611 | iUUCD 2.0 |
| Huwe1 | M6 | 59026 | iUUCD 2.0 |
| Kcmf1 | M6 | 74287 | iUUCD 2.0 |
| Mycbp2 | M6 | 105689 | iUUCD 2.0 |
| Rbbp6 | M6 | 19647 | iUUCD 2.0 |
| Rlim | M6 | 19820 | iUUCD 2.0 |
| Skp1a | M6 | 21402 | iUUCD 2.0 |
| Tbl1xr1 | M6 | 81004 | iUUCD 2.0 |
| Tceb1 | M6 | 67923 | iUUCD 2.0 |
| Tceb2 | M6 | 67673 | NCBI |
| Tceb3 | M6 | 27224 | iUUCD 2.0 |
| Ubr4 | M6 | 69116 | iUUCD 2.0 |
| Vhl | M6 | 22346 | iUUCD 2.0 |
| Wdr20 | M6 | 69641 | iUUCD 2.0 |
| Kctd13 | M5 | 233877 | iUUCD 2.0 |
| Kctd21 | M5 | 622320 | iUUCD 2.0 |
| Kctd5 | M5 | 69259 | iUUCD 2.0 |
| Lztr1 | M5 | 66863 | iUUCD 2.0 |
| Wdr26 | M6 | 226757 | iUUCD 2.0 |

TABLE 2
Co-functional gene module members with no known
role in dendritic cells or inflammation

| | Co-functional | | |
|---|---|---|---|
| Gene | gene module | GeneID | Database |
| Dcaf13 | M1 | 223499 | iUUCD 2.0 |
| Grb2 | M1 | 14784 | NCBI |
| Tbl3 | M1 | 213773 | iUUCD 2.0 |
| Wdr3 | M1 | 269470 | iUUCD 2.0 |
| Anapc13 | M2 | 69010 | iUUCD 2.0 |
| Brca1 | M2 | 12189 | iUUCD 2.0 |
| Brwd3 | M2 | 382236 | iUUCD 2.0 |
| Btbd1 | M2 | 83962 | iUUCD 2.0 |
| Ccnf | M2 | 12449 | iUUCD 2.0 |
| E4f1 | M2 | 13560 | iUUCD 2.0 |
| Fbxl14 | M2 | 101358 | iUUCD 2.0 |
| Fbxl5 | M2 | 242960 | iUUCD 2.0 |
| Fbxo42 | M2 | 213499 | iUUCD 2.0 |
| Fzr1 | M2 | 56371 | iUUCD 2.0 |
| Hectd1 | M2 | 207304 | iUUCD 2.0 |
| Katnb1 | M2 | 74187 | iUUCD 2.0 |
| Kbtbd13 | M2 | 74492 | iUUCD 2.0 |
| Klhl3 | M2 | 100503085 | iUUCD 2.0 |
| Kmt2b | M2 | 75410 | iUUCD 2.0 |
| Lrr1 | M2 | 69706 | iUUCD 2.0 |
| Lrrc41 | M2 | 230654 | iUUCD 2.0 |
| Mdm4 | M2 | 17248 | iUUCD 2.0 |
| Mkrn1 | M2 | 54484 | iUUCD 2.0 |
| Pa2g4 | M2 | 18813 | NCBI |
| Pcif1 | M2 | 228866 | iUUCD 2.0 |
| Ring1 | M2 | 19763 | iUUCD 2.0 |
| Taf3 | M2 | 209361 | iUUCD 2.0 |
| Ttc3 | M2 | 22129 | iUUCD 2.0 |
| Wdhd1 | M2 | 218973 | iUUCD 2.0 |
| Ankfy1 | M3 | 11736 | iUUCD 2.0 |
| Dcaf4 | M3 | 73828 | iUUCD 2.0 |
| Fbxl13 | M3 | 320118 | iUUCD 2.0 |
| Fbxo28 | M3 | 67948 | iUUCD 2.0 |
| Gnb2 | M3 | 14693 | iUUCD 2.0 |
| Klhl24 | M3 | 75785 | iUUCD 2.0 |
| Klhl7 | M3 | 52323 | iUUCD 2.0 |
| Med8 | M3 | 80509 | iUUCD 2.0 |
| Nosip | M3 | 66394 | iUUCD 2.0 |
| Rnf113a1 | M3 | 69942 | iUUCD 2.0 |
| Traf7 | M3 | 224619 | iUUCD 2.0 |
| Ube3c | M3 | 100763 | iUUCD 2.0 |
| Wdr1 | M3 | 22388 | iUUCD 2.0 |
| Ppil2 | M4 | 66053 | iUUCD 2.0 |
| Sart1 | M4 | 20227 | iUUCD 2.0 |
| Smu1 | M4 | 74255 | iUUCD 2.0 |
| Wdr70 | M4 | 545085 | iUUCD 2.0 |
| Ambra1 | M5 | 228361 | iUUCD 2.0 |
| Arih1 | M5 | 23806 | iUUCD 2.0 |
| Cnot4 | M5 | 53621 | iUUCD 2.0 |
| Dcaf7 | M5 | 71833 | iUUCD 2.0 |
| Det1 | M5 | 76375 | NCBI |
| March6 | M5 | 223455 | iUUCD 2.0 |
| Msl2 | M5 | 77853 | iUUCD 2.0 |
| Rnf139 | M5 | 75841 | iUUCD 2.0 |
| Strap | M5 | 20901 | iUUCD 2.0 |
| Trim45 | M5 | 229644 | iUUCD 2.0 |
| Zmiz2 | M5 | 52915 | iUUCD 2.0 |
| Anapc11 | M6 | 66156 | iUUCD 2.0 |
| Cul3 | M6 | 26554 | iUUCD 2.0 |
| Fbxo33 | M6 | 70611 | iUUCD 2.0 |
| Huwe1 | M6 | 59026 | iUUCD 2.0 |
| Kcmf1 | M6 | 74287 | iUUCD 2.0 |
| Mycbp2 | M6 | 105689 | iUUCD 2.0 |
| Rbbp6 | M6 | 19647 | iUUCD 2.0 |
| Rlim | M6 | 19820 | iUUCD 2.0 |
| Skp1a | M6 | 21402 | iUUCD 2.0 |
| Tbl1xr1 | M6 | 81004 | iUUCD 2.0 |
| Tceb1 | M6 | 67923 | iUUCD 2.0 |
| Tceb2 | M6 | 67673 | NCBI |
| Tceb3 | M6 | 27224 | iUUCD 2.0 |
| Ubr4 | M6 | 69116 | iUUCD 2.0 |
| Vhl | M6 | 22346 | iUUCD 2.0 |
| Wdr20 | M6 | 69641 | iUUCD 2.0 |
| Kctd13 | M5 | 233877 | iUUCD 2.0 |
| Kctd21 | M5 | 622320 | iUUCD 2.0 |
| Kctd5 | M5 | 69259 | iUUCD 2.0 |
| Lztr1 | M5 | 66863 | iUUCD 2.0 |
| Wdr26 | M6 | 226757 | iUUCD 2.0 |

In some aspects, the modulator modulates expression of the gene of Table 1 or Table 2. In some aspects, the modulator modulates expression or activity of a protein encoded by the gene of Table 1 or Table 2.

In some aspects, the modulator modulates abundance of a protein encoded by the gene of Table 1 or Table 2, e.g., modulates degradation of the protein.

In some aspects, the modulator causes a change in a downstream activity of a protein encoded by the gene of Table 1 or Table 2 in the presence of the modulator relative to the downstream activity in the absence of the modulator.

In some aspects, the modulator is an activator of the downstream activity of the gene of Table 1 or Table 2. In some aspects, the modulator causes an increase in a downstream activity (e.g., an increase in the amount, strength, or duration of the downstream activity) of the protein encoded by the gene of Table 1 or Table 2 relative to the downstream activity in the absence of the modulator.

Downstream activities may include any biological activity that occurs as a direct or indirect result of expression and/or activity of the protein encoded by the gene of Table 1 or Table 2 in, e.g., transcriptional regulation, signaling, and catalysis. In some aspects, the downstream activity is increased by at least 40%. In some aspects, the increase is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100%, e.g., the increase is 5%-15%, 15%-25%, 25%-35%, 35%-45%, 45%-55%, 55%-65%, 65%-75%, 75%-85%, 85%-95%, or 95%-100%).

In some aspects, the modulator is an inhibitor of the downstream activity of the gene of Table 1 or Table 2. In some aspects, the modulator causes a decrease in a downstream activity of the protein encoded by the gene of Table 1 or Table 2 relative to the downstream activity in the absence of the modulator. In some aspects, the downstream activity is decreased by at least 40%. In some aspects, the decrease is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., the downstream activity does not occur at a detectable level), e.g., the decrease is 5%-15%, 15%-25%, 25%-35%, 35%-45%, 45%-55%, 55%-65%, 65%-75%, 75%-85%, 85%-95%, or 95%-100%).

In some aspects, the modulator is a modulator as described in Section II herein, e.g., is a proteolysis targeting chimera (PROTAC), a small molecule, an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain), a peptide, a mimic, or an inhibitory nucleic acid (e.g., an ASO or a siRNA).

In some aspects in which the modulator is an antibody or antigen-binding fragment thereof, the antibody or antigen-binding fragment thereof binds a protein encoded by the gene of Table 1 or Table 2.

In some aspects, the disease or disorder related to APCs and/or inflammation in an individual is a disease or disorder relating to dendritic cells (DCs), macrophages, glial cells, or B cells, e.g., the APC is a DC, a macrophage, a glial cell (e.g., a microglial cell, an astrocyte, or an oligodendrocyte), or a B cell. In some aspects, the APC is a DC.

In some aspects, the disease or disorder relating to APCs and/or inflammation is a neurodegenerative disease (e.g., multiple sclerosis (MS), Alzheimer's disease (AD), amyotrophic lateral sclerosis (ALS), or Parkinson's disease (PD)), arthritis, allergy, eczema, fibrosis, asthma, lupus erythematosus, an inflammatory bowel disease, ulcerative colitis, Crohn's disease, or a blastic plasmacytoid dendritic cell neoplasm. In some aspects, the disease or disorder relating to APCs and/or inflammation is encephalitis, myelitis, meningitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, or lymphadenitis.

In some aspects, the gene of Table 1 or Table 2 is Vhl or Huwe1 and the modulator is a PROTAC that acts with Vhl as the E3 ligase, e.g., a PROTAC provided in Wang et al., Eur. J. Med. Chem., Jan 5; 227:113906, 2022. In some aspects, the gene of Table 1 or Table 2 is Huwe1 and the modulator is an agent provided in Crawford et al., Oncogene, 39(27): 5001-5014, 2020.

In some aspects, the disclosure features a method of monitoring the response of an individual having a disease or disorder related to APCs and/or inflammation to treatment with a modulator of a gene of Table 1 or Table 2, the method comprising: (a) determining, in a biological sample obtained from the individual at a time point following administration of the modulator, the expression level of the gene of Table 1 or Table 2; and (b) comparing the expression level of the gene of Table 1 or Table 2 in the biological sample with a reference level, thereby monitoring the response in the individual to treatment with the modulator.

In some aspects, the disclosure features a method of monitoring the response of an individual having a disease or disorder related to APCs and/or inflammation to treatment with a modulator of a gene of Table 1, the method comprising: (a) determining, in a biological sample obtained from the individual at a time point following administration of the modulator, the expression level of the gene of Table 1; and (b) comparing the expression level of the gene of Table 1 in the biological sample with a reference level, thereby monitoring the response in the individual to treatment with the modulator.

In some aspects, the disclosure features a method of monitoring the response of an individual having a disease or disorder related to APCs and/or inflammation to treatment with a modulator of a gene of Table 2, the method comprising: (a) determining, in a biological sample obtained from the individual at a time point following administration of the modulator, the expression level of the gene of Table 2; and (b) comparing the expression level of the gene of Table 2 in the biological sample with a reference level, thereby monitoring the response in the individual to treatment with the modulator.

In some aspects, the reference level is selected from the group consisting of (i) the expression level of the gene in a biological sample from the individual obtained prior to administration of the modulator; (ii) the expression level of the gene in a reference population; (iii) a pre-assigned expression level for the gene; or (iv) the expression level of the gene in a biological sample obtained from the individual at a previous time point, wherein the previous time point is following administration of the modulator.

In some aspects, the expression level of the expression level of the gene of Table 1 or Table 2 is decreased in the biological sample obtained from the individual relative to the reference level, and the method further comprises administering to the individual one or more additional doses of the modulator, wherein the modulator is an agent that increases the expression and/or activity of the gene of Table 1 or Table 2.

In some aspects, the expression level of the expression level of the gene of Table 1 or Table 2 is increased in the biological sample obtained from the individual relative to the reference level, and the method further comprises administering to the individual one or more additional doses of the modulator, wherein the modulator is an agent that decreases the expression and/or activity of the gene of Table 1 or Table 2.

### V. Methods Targeting CCR7 and its Interacting Partners

### A. Methods of Treatment

In some aspects, the disclosure features a method for treating a cancer, an inflammatory disease, or an autoimmune disease in an individual, the method comprising administering to the individual an effective amount of a modulator of the interaction between (a) one, two, or all three of LIM domain-binding protein 2 (Ldb2), Ring finger protein 165 (Rnf165), and TNF receptor-associated factor 2 (Traf2) and (b) chemokine receptor type 7 (CCR7).

In some aspects, the modulator is a modulator as described in Section II herein, e.g., is a proteolysis targeting chimera (PROTAC), a small molecule, an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to one, two, or all three of Ldb2, Rnf165, and Traf2 and/or binds to CCR7), a peptide, a mimic, or an inhibitory nucleic acid (e.g., an ASO or a siRNA). In some aspects, the modulator is a bispecific antibody comprising an antigen-binding domain that targets the tumor microenvironment (e.g., a bispecific antibody comprising a first binding domain that binds to one, two, or all three of Ldb2, Rnf165, and Traf2 and/or binds to CCR7 and a second binding domain that targets the tumor microenvironment).

In some aspects, the individual has a cancer and the modulator is an agent that decreases the expression and/or activity of one, two, or all three of Ldb2, Rnf165, and Traf2.

In some aspects, the individual has an inflammatory disease or an autoimmune disease and the modulator is an agent that increases the expression and/or activity of one, two, or all three of Ldb2, Rnf165, and Traf2. In other aspects, the individual has a cancer and the modulator is an agent that increases the expression and/or activity of one, two, or all three of Ldb2, Rnf165, and Traf2.

In some aspects, the inflammatory disease or autoimmune disease is a neurodegenerative disease (e.g., multiple sclerosis (MS), Alzheimer's disease (AD), amyotrophic lateral sclerosis (ALS), or Parkinson's disease (PD)), arthritis, allergy, eczema, fibrosis, asthma, lupus erythematosus, an inflammatory bowel disease, ulcerative colitis, or Crohn's disease. In some aspects, the inflammatory disease or autoimmune disease is Crohn's disease. In some aspects, the inflammatory disease or autoimmune disease is encephalitis, myelitis, meningitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, or lymphadenitis.

### B. Methods of Increasing CCR7 Expression

In some aspects, the disclosure features a method for increasing expression of CCR7 in an antigen-presenting cell (APC), the method comprising contacting the APC with an effective amount of an agent that decreases the expression and/or activity of one, two, or all three of Ldb2, Rnf165, and Traf2.

The agent that decreases the expression and/or activity of one, two, or all three of Ldb2, Rnf165, and Traf2 may be, e.g., a proteolysis targeting chimera (PROTAC) (e.g., a PROTAC that directs proteolysis of one, two, or all three of Ldb2, Rnf165, and Traf2); a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to one, two, or all three of Ldb2, Rnf165, and Traf2 and/or binds to CCR7); a peptide; a mimic; or an inhibitory nucleic acid (e.g., an ASO or a siRNA). In some aspects, the agent is a bispecific antibody comprising an antigen-binding domain that targets the tumor microenvironment (e.g., a bispecific antibody comprising a first binding domain that binds to one, two, or all three of Ldb2, Rnf165, and Traf2 and/or binds to CCR7 and a second binding domain that targets the tumor microenvironment).

In some aspects, CCR7 expression in the APC is increased by at least 5%, at least 10%, at least 20%, at least 30%, at least 40%, at least 50%, at least 60%, at least 70%, at least 80%, at least 90%, at least 100%, or more than 100% relative to expression in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to expression in the absence of the agent. In some aspects, CCR7 expression in the APC is increased by at least 10% relative to expression in the absence of the agent.

In some aspects, the APC is a dendritic cell (DC), a macrophage, a glial cell (e.g., a microglial cell, an astrocyte, or an oligodendrocyte), or a B cell. In some aspects, the APC is a DC.

In some aspects, the APC is in an individual. In some aspects, the individual has a cancer.

### C. Methods of Increasing APC Migration to Tumors and/or Lymph Nodes

In some aspects, the disclosure features a method for increasing APC migration to a tumor and/or one or more lymph nodes in an individual (e.g., an individual having a cancer), the method comprising administering to the individual an effective amount of an agent that decreases the expression and/or activity of one, two, or all three of Ldb2, Rnf165, and Traf2.

The agent that decreases the expression and/or activity of one, two, or all three of Ldb2, Rnf165, and Traf2 may be, e.g., a proteolysis targeting chimera (PROTAC) (e.g., a PROTAC that directs proteolysis of one, two, or all three of Ldb2, Rnf165, and Traf2); a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to one, two, or all three of Ldb2, Rnf165, and Traf2 and/or binds to CCR7); a peptide; a mimic; or an inhibitory nucleic acid (e.g., an ASO or a siRNA). In some aspects, the agent is a bispecific antibody comprising an antigen-binding domain that targets the tumor microenvironment (e.g., a bispecific antibody comprising a first binding domain that binds to one, two, or all three of Ldb2, Rnf165, and Traf2 and/or binds to CCR7 and a second binding domain that targets the tumor microenvironment).

In some aspects, the individual has a cancer and APC migration to a tumor site in the individual is increased by at least 5%, at least 10%, at least 20%, at least 30%, at least 40%, at least 50%, at least 60%, at least 70%, at least 80%, at least 90%, at least 100%, or more than 100% relative to migration in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to migration in the absence of the agent. In some aspects, APC migration to a tumor site in the individual is increased by at least 10% relative to migration in the absence of the agent.

In some aspects, APC migration to one or more lymph nodes in the individual is increased by at least 5%, at least 10%, at least 20%, at least 30%, at least 40%, at least 50%, at least 60%, at least 70%, at least 80%, at least 90%, at least 100%, or more than 100% relative to migration in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to migration in the absence of the agent.

In some aspects, APC migration to one or more lymph nodes in the individual is increased by at least 10% relative to migration in the absence of the agent.

In some aspects, the APC is a DC, a macrophage, a glial cell (e.g., a microglial cell, an astrocyte, or an oligodendrocyte), or a B cell. In some aspects, the APC is a DC.

In some aspects, the individual has a cancer.

### D. Methods of Increasing T Cell Homing to Tumors

In some aspects, the disclosure features a method for increasing T cell homing to a tumor in an individual (e.g., an individual having a cancer), the method comprising administering to the individual an effective amount of an agent that decreases the expression and/or activity of one, two, or all three of Ldb2, Rnf165, and Traf2, wherein the agent increases dendritic cell migration to the tumor in the individual.

The agent that decreases the expression and/or activity of one, two, or all three of Ldb2, Rnf165, and Traf2 may be, e.g., a proteolysis targeting chimera (PROTAC) (e.g., a PROTAC that directs proteolysis of one, two, or all three of Ldb2, Rnf165, and Traf2); a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to one, two, or all three of Ldb2, Rnf165, and Traf2 and/or binds to CCR7); a peptide; a mimic; or an inhibitory nucleic acid (e.g., an ASO or a siRNA). In some aspects, the agent is a bispecific antibody comprising an antigen-binding domain that targets the tumor microenvironment (e.g., a bispecific antibody comprising a first binding domain that binds to one, two, or all three of Ldb2, Rnf165, and Traf2 and/or binds to CCR7 and a second binding domain that targets the tumor microenvironment).

In some aspects, T cell homing to the tumor in the individual is increased by at least 5%, at least 10%, at least 20%, at least 30%, at least 40%, at least 50%, at least 60%, at least 70%, at least 80%, at least 90%, at least 100%, or more than 100% relative to T cell homing in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to T cell homing in the absence of the agent. In some aspects, T cell homing to the tumor in the individual is increased by at least 10% relative to T cell homing in the absence of the agent.

### E. Combination Therapies

Any of the methods of Sections V(A)-V(D) may further comprise administering to the individual or contacting the APC with one or more additional agents (e.g., administering one or more additional agents before, during, or after treatment with the modulator of the interaction between (a) one, two, or all three of Ldb2, Rnf165, and Traf2 and (b) CCR7 or the agent that decreases the expression and/or activity of one, two, or all three of Ldb2, Rnf165, and Traf2).

In some aspects, the additional agent is an agent that modulates the expression of one or more members of Module M3 as presented in Example 3, e.g., modulates the expression of one or more of Akt1, Ankfy1, Apc, Arpc1 b, Birc2, Bmi1, Bub3, Cacybp, Cebpb, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31a, Smad2, Syvn1, Taf5l, Traf2, Traf3, Traf7, Trim24, Trp53, Ube2e1, Ube2e3, Ube3c, Ufm1, Wdfy3, Wdr1, Wdr82, Whsc1, and Zbtb11.

### F. Cell Therapies with Alterations in CCR7 or Interactors Thereof

In some aspects, the disclosure features a method for treating a cancer, an inflammatory disease, or an autoimmune disease in an individual, the method comprising administering to the individual an effective amount of a cell therapy (e.g., a cell therapy as described in Section VIII herein) comprising loss-of-function alterations in one, two, or all three of Ldb2, Rnf165, and Traf2.

In some aspects, the disclosure provides a genetically modified isolated cell comprising loss-of-function alterations in in one, two, or all three of Ldb2, Rnf165, and Traf2.

In some aspects, the loss-of-function alterations are loss-of-function mutations (e.g., mutations that result in reduced or abolished protein function, including deletions). In some aspects, the loss-of-function alterations are knockout (KO) mutations.

In some aspects, the disclosure features a method for treating a cancer, an inflammatory disease, or an autoimmune disease in an individual, the method comprising administering to the individual an effective amount of a cell therapy comprising a gain-of-function alteration in CCR7.

In some aspects, the disclosure provides a genetically modified isolated cell comprising a gain-of-function alteration in CCR7.

In some aspects, the gain-of-function alteration is a gain-of-function mutation (e.g., a mutation that results in increased gene function, including overexpression and gene duplications). In some aspects, the gain-of-function alteration is overexpression.

In some aspects, the genetically modified isolated cell is a dendritic cell, a macrophage, a T cell, a TIL, or a NK cell.

In some aspects, the cell therapy is a dendritic cell therapy, a macrophage cell therapy, an ACT, a TIL therapy, an engineered TCR therapy, a CAR-T therapy, a CAR-Treg therapy, or a NK cell therapy.

### G. Methods of Monitoring Response to Treatment

In another aspect, the disclosure features a method of monitoring the response of an individual having a cancer, an inflammatory disease, or an autoimmune disease to treatment with a modulator of the interaction between (a) one, two, or all three of Ldb2, Rnf165, and Traf2 and (b) CCR7, the method comprising (i) determining, in a biological sample obtained from the individual at a time point following administration of the modulator, the expression level of one or more of Ldb2, Rnf165, and Traf2; and (ii) comparing the expression level of the one or more genes in the biological sample with a reference level, thereby monitoring the response in the individual to treatment with the modulator.

In some aspects, the reference level is selected from the group consisting of (i) the expression level of the one or more genes in a biological sample from the individual obtained prior to administration of the modulator; (ii) the expression level of the one or more genes in a reference population; (iii) a pre-assigned expression level for the one or more genes; or (iv) the expression level of the one or more genes in a biological sample obtained from the individual at a previous time point, wherein the previous time point is following administration of the modulator.

In some aspects, the individual has a cancer, the expression level of the one or more genes is increased in the biological sample obtained from the individual relative to the reference level, and the method further comprises administering to the individual one or more additional doses of the modulator, wherein the modulator is an agent that decreases the expression and/or activity of one, two, or all three of Ldb2, Rnf165, and Traf2.

In some aspects, the individual has an inflammatory disease or an autoimmune disease, the expression level of the one or more genes is decreased in the biological sample obtained from the individual relative to the reference level, and the method further comprises administering to the individual one or more additional doses of the modulator; wherein the modulator is an agent that increases the expression and/or activity of one, two, or all three of Ldb2, Rnf165, and Traf2.

### VI. Methods of Regulating Migratory Dendritic Cells

### A. Methods of Treating a Cancer, Inflammatory Disease, or Autoimmune Disease

In some aspects, the disclosure features a method for treating a cancer, an inflammatory disease, an autoimmune disease, or an infectious disease (e.g., an infectious disease that would benefit from an enhanced immune response) in an individual, the method comprising administering to the individual an effective amount of (a) an agent that decreases the expression and/or activity of CCAAT/enhancer-binding protein beta (Cebpb); (b) an agent that decreases the expression and/or activity of TNF receptor-associated factor 2 (Traf2); and/or (c) an agent that increases the expression and/or activity of Death-inducer obliterator 1 (Dido1).

The agent that decreases the expression and/or activity of Cebpb, decreases the expression and/or activity of Traf2, or increases the expression and/or activity of Dido1 may be, e.g., a proteolysis targeting chimera (PROTAC); a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Cebpb, Traf2; and/or Dido1); a peptide; a mimic; or an inhibitory nucleic acid (e.g., an ASO or a siRNA).

In another aspect, the disclosure features a method for treating an inflammatory disease, an autoimmune disease, or an infectious disease (e.g., an infectious disease occurring with an excessive immune response) in an individual, the method comprising administering to the individual an effective amount of (a) an agent that increases the expression and/or activity of Cebpb; (b) an agent that increases the expression and/or activity of Traf2; and/or (c) an agent that decreases the expression and/or activity of Dido1.

The agent that increases the expression and/or activity of Cebpb, increases the expression and/or activity of Traf2, or decreases the expression and/or activity of Dido1 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Cebpb, Traf2; and/or Dido1); a peptide; a mimic; or an inhibitory nucleic acid (e.g., an ASO or a siRNA).

In some aspects, the inflammatory disease or autoimmune disease is a neurodegenerative disease (e.g., multiple sclerosis (MS), Alzheimer's disease (AD), amyotrophic lateral sclerosis (ALS), or Parkinson's disease (PD)), arthritis, allergy, eczema, fibrosis, asthma, lupus erythematosus, an inflammatory bowel disease, ulcerative colitis, or Crohn's disease. In some aspects, the inflammatory disease or autoimmune disease is Crohn's disease. In some aspects, the inflammatory disease or autoimmune disease is encephalitis, myelitis, meningitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, or lymphadenitis.

In some aspects, the autoimmune disease is associated with a reduced proportion of migratory dendritic cells (mDCs). In some aspects, the individual has a loss-of-function mutation in Dido1.

### B. Methods of Increasing Proportion of MDCs

In another aspect, the disclosure features a method for increasing the proportion of migratory dendritic cells (mDCs) in an individual (e.g., an individual having a cancer, an inflammatory disease, or an autoimmune disease), the method comprising administering to the individual an effective amount of (a) an agent that decreases the expression and/or activity of Cebpb; (b) an agent that decreases the expression and/or activity of Traf2; and/or (c) an agent that increases the expression and/or activity of Death-inducer obliterator 1 (Dido1).

The agent that decreases the expression and/or activity of Cebpb, decreases the expression and/or activity of Traf2, or increases the expression and/or activity of Dido1 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Cebpb, Traf2; and/or Dido1); a peptide; a mimic; or an inhibitory nucleic acid (e.g., an ASO or a siRNA).

In some aspects, the proportion of mDCs in the individual is a proportion in a tumor or a tissue of the individual.

In some aspects, the proportion of mDCs in the individual (e.g., in a tumor or tissue of the individual) is increased by at least 5%, at least 10%, at least 20%, at least 30%, at least 40%, at least 50%, at least 60%, at least 70%, at least 80%, at least 90%, at least 100%, or more than 100% relative to the proportion of mDCs in the individual in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to the proportion of mDCs in the individual in the absence of the agent. In some aspects, the proportion of mDCs in the individual (e.g., in a tumor or tissue of the individual) is increased by at least 10% relative to the proportion in the absence of the agent.

### C. Methods of Increasing Anti-Tumor Immunity

In another aspect, the disclosure features a method for increasing anti-tumor immunity in an individual (e.g., an individual having a cancer), the method comprising administering to the individual an effective amount of (a) an agent that decreases the expression and/or activity of Cebpb; (b) an agent that decreases the expression and/or activity of Traf2; and/or (c) an agent that increases the expression and/or activity of Dido1.

The agent that decreases the expression and/or activity of Cebpb, decreases the expression and/or activity of Traf2, or increases the expression and/or activity of Dido1 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Cebpb, Traf2; and/or Dido1); a peptide; a mimic; or an inhibitory nucleic acid (e.g., an ASO or a siRNA).

In some aspects, anti-tumor immunity in the individual (e.g., in a tumor or tissue of the individual) is increased by at least 5%, at least 10%, at least 20%, at least 30%, at least 40%, at least 50%, at least 60%, at least 70%, at least 80%, at least 90%, at least 100%, or more than 100% relative to anti-tumor immunity in the individual in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to anti-tumor immunity in the individual in the absence of the agent. In some aspects, anti-tumor immunity in the individual (e.g., in a tumor or tissue of the individual) is increased by at least 10% relative to anti-tumor immunity in the absence of the agent.

### D. Methods of Decreasing Proportion of MDCs

In another aspect, the disclosure features a method for decreasing the proportion of mDCs in an individual (e.g., an individual having an inflammatory disease or an autoimmune disease), the method comprising administering to the individual an effective amount of (a) an agent that increases the expression and/or activity of Cebpb; (b) an agent that increases the expression and/or activity of Traf2; and/or (c) an agent that decreases the expression and/or activity of Dido1.

The agent that increases the expression and/or activity of Cebpb, increases the expression and/or activity of Traf2, or decreases the expression and/or activity of Dido1 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Cebpb, Traf2; and/or Dido1); a peptide; a mimic; or an inhibitory nucleic acid (e.g., an ASO or a siRNA).

In some aspects, the proportion of mDCs in the individual is a proportion in a tumor or a tissue of the individual.

In some aspects, the proportion of mDCs in the individual (e.g., in a tissue of the individual that is affected by an inflammatory disease or an autoimmune disease) is decreased by at least 5%, at least 10%, at least 20%, at least 30%, at least 40%, at least 50%, at least 60%, at least 70%, at least 80%, at least 90%, or 100%, relative to the proportion of mDCs in the individual in the absence of the agent (e.g., decreased by 5%-15%, 15%-25%, 25%-35%, 35%-45%, 45%-55%, 55%-65%, 65%-75%, 75%-85%, 85%-95%, or 95%-100%) relative to the proportion of mDCs in the individual in the absence of the agent. In some aspects, the proportion of mDCs in the individual (e.g., in a tumor or tissue of the individual) is decreased by at least 10% relative to the proportion in the absence of the agent.

### E. Method for Decreasing Autoimmune Activity (by Decreasing Fraction of mDCs)

In another aspect, the disclosure features a method for decreasing autoimmune activity in an individual (e.g. an individual having an autoimmune disease), the method comprising administering to the individual an effective amount of (a) an agent that increases the expression and/or activity of Cebpb; (b) an agent that increases the expression and/or activity of Traf2; and/or (c) an agent that decreases the expression and/or activity of Dido1.

The agent that increases the expression and/or activity of Cebpb, increases the expression and/or activity of Traf2, or decreases the expression and/or activity of Dido1 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Cebpb, Traf2; and/or Dido1); a peptide; a mimic; or an inhibitory nucleic acid (e.g., an ASO or a siRNA).

In some aspects, autoimmune activity in the individual (e.g., in a tissue of the individual that is affected by an autoimmune disease) is decreased by at least 5%, at least 10%, at least 20%, at least 30%, at least 40%, at least 50%, at least 60%, at least 70%, at least 80%, at least 90%, or 100% relative to autoimmune activity in the individual in the absence of the agent (e.g., decreased by 5%-15%, 15%-25%, 25%-35%, 35%-45%, 45%-55%, 55%-65%, 65%-75%, 75%-85%, 85%-95%, 95%-100%, or 100%) relative to autoimmune activity in the individual in the absence of the agent. In some aspects, autoimmune activity in the individual (e.g., in a tumor or tissue of the individual) is decreased by at least 10% relative to autoimmune activity in the absence of the agent.

### F. Combination Therapies

Any of the methods of Sections VI(A)-VI(E) may further comprise administering to the individual one or more additional agents (e.g., administering one or more additional agents before, during, or after treatment with the agent that decreases the expression and/or activity of Cebpb; agent that decreases the expression and/or activity of Traf2; agent that increases the expression and/or activity of Dido1; agent that increases the expression and/or activity of Cebpb; agent that increases the expression and/or activity of Traf2; or agent that decreases the expression and/or activity of Dido1).

In some aspects, the additional agent is an agent that modulates the expression of one or more members of Module M2 as presented in Example 3, e.g., modulates the expression of one or more of Ago2, Ahr, Anapc13, Bach1, Baz1a, Bid, Bptf, Brca1, Brwd3, Btbd1, Cblc, Ccnf, Cdc27, Cntn4, Copa, Copb2, Coro1a, Cpne9, Cul4b, Ddb1, E4f1, Ecel1, Fbxl14, Fbxl5, Fbxo11, Fbxo42, Fzr1, Gemin5, Gm10697, Gm9117, Gtf2 h2, Gtf3c1, Hdac4, Hectd1, Ift122, Ikbkg, Ing2, Jun, Katnb1, Kbtbdi3, Kdm2a, Klhl23, Klhl3, Kmt2b, LOC100861784, Lrr1, Lrrc41, Map3k7, Mdm4, Mib1, Mkrn1, Mnat1, Naca, Nsmaf, Ogt, Pa2g4, Pcif1, Ppp1r11, Prc1, Ring1, Rnf128, Rnf20, Rnf225, Rnf40, Siah1a, Siah2, Taf3, Tdpoz2, Tmem183a, Tnfsf 1i, Tradd, Traf3ip2, Trim35, Trim7, Tssc1, Ttc3, Ube2n, Ufl1, Unk1, Upf1, Vdr, Wdhd1, Wdr48, Wdr95, Wwp1, Ybx1, Zbtb14, Zbtb49, Zbtb7a, and Zmiz1.

In some aspects, the additional agent is an agent that modulates the expression of one or more members of Module M3 as presented in Example 3, e.g., modulates the expression of one or more of Akt1, Ankfy1, Apc, Arpc1 b, Birc2, Bmi1, Bub3, Cacybp, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31 a, Smad2, Syvn1, Taf5l, Traf3, Traf7, Trim24, Trp53, Ube2e1, Ube2e3, Ube3c, Ufm1, Wdfy3, Wdr1, Wdr82, Whsc1, and Zbtb11.

### G. Methods of Monitoring Response to Treatment

In another aspect, the disclosure features a method of monitoring the response of an individual having a cancer, an inflammatory disease, an autoimmune disease, or an infectious disease to treatment with (a) an agent that decreases the expression and/or activity of Cebpb; (b) an agent that decreases the expression and/or activity of Traf2; and/or (c) an agent that increases the expression and/or activity of Dido1, the method comprising (i) determining, in a biological sample obtained from the individual at a time point following administration of the agent, the expression level of one or more of Cebpb, Traf2, and Dido1; and (ii) comparing the expression level of the one or more genes in the biological sample with a reference level, thereby monitoring the response in the individual to treatment with the agent.

In another aspect, the disclosure features a method of monitoring the response of an individual having an inflammatory disease, an autoimmune disease, or an infectious disease to treatment with (a) an agent that increases the expression and/or activity of Cebpb; (b) an agent that increases the expression and/or activity of Traf2; and/or (c) an agent that decreases the expression and/or activity of Dido1, the method comprising (i) determining, in a biological sample obtained from the individual at a time point following administration of the agent, the expression level of one or more of Cebpb, Traf2, and Dido1; and (ii) comparing the expression level of the one or more genes in the biological sample with a reference level, thereby monitoring the response in the individual to treatment with the agent.

In some aspects, the reference level is selected from the group consisting of (i) the expression level of the one or more genes in a biological sample from the individual obtained prior to administration of the agent; (ii) the expression level of the one or more genes in a reference population; (iii) a pre-assigned expression level for the one or more genes; or (iv) the expression level of the one or more genes in a biological sample obtained from the individual at a previous time point, wherein the previous time point is following administration of the agent.

In some aspects, (a) the expression and/or activity of Cebpb is increased in the biological sample obtained from the individual relative to the reference level; (b) the expression and/or activity of Traf2 is increased in the biological sample obtained from the individual relative to the reference level; and/or (c) the expression and/or activity of Dido1 is decreased in the biological sample obtained from the individual relative to the reference level, and the method further comprises administering to the individual one or more additional doses of the agent, wherein the agent decreases the expression and/or activity of Cebpb; decreases the expression and/or activity of Traf2; and/or increases the expression and/or activity of Dido1.

In some aspects, (a) the expression and/or activity of Cebpb is decreased in the biological sample obtained from the individual relative to the reference level; (b) the expression and/or activity of Traf2 is decreased in the biological sample obtained from the individual relative to the reference level; and/or (c) the expression and/or activity of Dido1 is increased in the biological sample obtained from the individual relative to the reference level, and the method further comprises administering to the individual one or more additional doses of the agent, wherein the agent increases the expression and/or activity of Cebpb; increases the expression and/or activity of Traf2; and/or decreases the expression and/or activity of Dido1.

### VII. Methods of Regulating Processing of Nfkb1 and/or Nfkb2

### A. Methods of Treating a Cancer, Inflammatory Disease, or Autoimmune Disease

In some aspects, the disclosure features a method for treating a cancer, an inflammatory disease, or an autoimmune disease in an individual, the method comprising administering to the individual an effective amount of a modulator of the interaction between (a) F-box and WD repeat domain containing 11 (Fbxw11) and (b) nuclear factor kappa B subunit 1 (Nfkb1) or nuclear factor kappa B subunit 2 (Nfkb2).

In some aspects, the modulator is a modulator as described in Section II herein, e.g., is a proteolysis targeting chimera (PROTAC), a small molecule, an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to one, two, or all three of Fbxw11, Nfkb1, and Nfkb2), a peptide, a mimic, or an inhibitory nucleic acid (e.g., an ASO or a siRNA).

In some aspects, the individual has a cancer and the modulator is an agent that increases the expression and/or activity of Fbxw11.

In some aspects, the individual has an inflammatory disease or an autoimmune disease and the modulator is an agent that decreases the expression and/or activity of Fbxw11.

In some aspects, the inflammatory disease or autoimmune disease is a neurodegenerative disease (e.g., multiple sclerosis (MS), Alzheimer's disease (AD), amyotrophic lateral sclerosis (ALS), or Parkinson's disease (PD)), arthritis, allergy, eczema, fibrosis, asthma, lupus erythematosus, an inflammatory bowel disease, ulcerative colitis, or Crohn's disease. In some aspects, the inflammatory disease or autoimmune disease is Crohn's disease. In some aspects, the inflammatory disease or autoimmune disease is encephalitis, myelitis, meningitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, or lymphadenitis.

### B. Methods of Increasing Processing of Nfkb1 and/or Nfkb2

In some aspects, the disclosure features a method for increasing processing of Nfkb1 and/or Nfkb2 into an active form, the method comprising contacting a cell capable of expressing Fbxw11 with an agent that increases expression and/or activity of Fbxw11.

The agent that increases the expression and/or activity of Fbxw11 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Fbxw11); a peptide; a mimic; or an inhibitory nucleic acid (e.g., an ASO or a siRNA).

In some aspects, the cell capable of expressing Fbxw11 is in an individual. In some aspects, the individual has a cancer.

In some aspects, processing of Nfkb1 into an active form is increased by at least 5%, at least 10%, at least 20%, at least 30%, at least 40%, at least 50%, at least 60%, at least 70%, at least 80%, at least 90%, at least 100%, or more than 100% relative to processing of Nfkb1 into an active form in the individual in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to processing of Nfkb1 into an active form in the individual in the absence of the agent. In some aspects, processing of Nfkb1 into an active form in the individual is increased by at least 10% relative to processing of Nfkb1 into an active form in the absence of the agent.

In some aspects, processing of Nfkb2 into an active form is increased by at least 5%, at least 10%, at least 20%, at least 30%, at least 40%, at least 50%, at least 60%, at least 70%, at least 80%, at least 90%, at least 100%, or more than 100% relative to processing of Nfkb2 into an active form in the individual in the absence of the agent (e.g., increased by 5%-15%, 15%-25%, 25%-35%, 35%-45%, 45%-55%, 55%-65%, 65%-75%, 75%-85%, 85%-95%, 95%-100%, or more than 100%) relative to processing of Nfkb2 into an active form in the individual in the absence of the agent. In some aspects, processing of Nfkb2 into an active form in the individual is increased by at least 10% relative to processing of Nfkb2 into an active form in the absence of the agent.

### C. Methods of Decreasing Processing of Nfkb1 and/or Nfkb2

In some aspects, the disclosure features a method for decreasing processing of Nfkb1 and/or Nfkb2 into an active form, the method comprising contacting a cell capable of expressing Fbxw11 with an agent that decreases expression and/or activity of Fbxw11.

The agent that decreases the expression and/or activity of Fbxw11 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Fbxw11); a peptide; a mimic; or an inhibitory nucleic acid (e.g., an ASO or a siRNA).

In some aspects, the cell capable of expressing Fbxw11 is in an individual. In some aspects, the individual has an inflammatory disease or an autoimmune disease. In some aspects, the inflammatory disease or autoimmune disease is a neurodegenerative disease (e.g., MS, AD, ALS, or PD), arthritis, allergy, eczema, fibrosis, asthma, lupus erythematosus, an inflammatory bowel disease, ulcerative colitis, or Crohn's disease. In some aspects, the inflammatory disease or autoimmune disease is Crohn's disease. In some aspects, the inflammatory disease or autoimmune disease is encephalitis, myelitis, meningitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, or lymphadenitis.

In some aspects, processing of Nfkb1 into an active form is decreased by at least 5%, at least 10%, at least 20%, at least 30%, at least 40%, at least 50%, at least 60%, at least 70%, at least 80%, at least 90%, or 100% relative to processing of Nfkb1 into an active form in the individual in the absence of the agent (e.g., decreased by 5%-15%, 15%-25%, 25%-35%, 35%-45%, 45%-55%, 55%-65%, 65%-75%, 75%-85%, 85%-95%, or 95%-100%) relative to processing of Nfkb1 into an active form in the individual in the absence of the agent. In some aspects, processing of Nfkb1 into an active form in the individual is decreased by at least 10% relative to processing of Nfkb1 into an active form in the absence of the agent.

In some aspects, processing of Nfkb2 into an active form is decreased by at least 5%, at least 10%, at least 20%, at least 30%, at least 40%, at least 50%, at least 60%, at least 70%, at least 80%, at least 90%, or 100% relative to processing of Nfkb2 into an active form in the individual in the absence of the agent (e.g., decreased by 5%-15%, 15%-25%, 25%-35%, 35%-45%, 45%-55%, 55%-65%, 65%-75%, 75%-85%, 85%-95%, or 95%-100%) relative to processing of Nfkb2 into an active form in the individual in the absence of the agent. In some aspects, processing of Nfkb2 into an active form in the individual is decreased by at least 10% relative to processing of Nfkb2 into an active form in the absence of the agent.

### D. Methods of Increasing Immune Response Directed by Nfkb1 and/or Nfkb2

In another aspect, the disclosure features a method for increasing an immune response directed by Nfkb1 and/or Nfkb2 in an individual (e.g., an individual having a cancer), the method comprising administering to the individual an effective amount of an agent that increases expression and/or activity of Fbxw11.

The agent that increases the expression and/or activity of Fbxw11 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Fbxw11); a peptide; a mimic; or an inhibitory nucleic acid (e.g., an ASO or a siRNA).

### E. Methods of Decreasing Immune Response Directed by Nfkb1 and/or Nfkb2

In another aspect, the disclosure features a method for decreasing an immune response directed by Nfkb1 and/or Nfkb2 in an individual (e.g., an individual having an inflammatory disease or an autoimmune disease), the method comprising administering to the individual an effective amount of an agent that decreases expression and/or activity of Fbxw11.

The agent that decreases the expression and/or activity of Fbxw11 may be, e.g., a PROTAC; a small molecule; an antibody or antigen-binding fragment thereof (e.g., a bis-Fab, an Fv, a Fab, a Fab′-SH, a F(ab′)₂, a diabody, a linear antibody, an scFv, an scFab, a VH domain, or a VHH domain) (e.g., an antibody or antigen-binding fragment thereof that binds to Fbxw11); a peptide; a mimic; or an inhibitory nucleic acid (e.g., an ASO or a siRNA).

In some aspects, the inflammatory disease or autoimmune disease is a neurodegenerative disease (e.g., MS, AD, ALS, or PD), arthritis, allergy, eczema, fibrosis, asthma, lupus erythematosus, an inflammatory bowel disease, ulcerative colitis, or Crohn's disease. In some aspects, the inflammatory disease or autoimmune disease is Crohn's disease. In some aspects, the inflammatory disease or autoimmune disease is encephalitis, myelitis, meningitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, or lymphadenitis.

### F. Combination Therapies

Any of the methods of Sections VII(A)-VII(E) may further comprise administering to the individual or the cell capable of expressing Fbxw11 one or more additional agents (e.g., administering one or more additional agents before, during, or after treatment with the modulator of the interaction between (a) Fbxw11 and (b) Nfkb1 or Nfkb2, the agent that increases expression and/or activity of Fbxw11, or the agent that decreases expression and/or activity of Fbxw11).

In some aspects, the additional agent is an agent that modulates the expression of one or more members of Module M5 as presented in Example 3, e.g., modulates the expression of one or more of Acaca, Ambra1, Amfr, Arih1, Cbll1, Cfap57, Cnot4, Cyld, Dcaf7, Det1, Dpf2, Eed, Efcab8, Egr2, Fasn, Fbxw7, Foxo3, Gsk3b, Hectd3, Hira, Icos, Ifnarl, Ikbke, Ints12, Junb, Kat6a, Kctd10, Kctd13, Kctd21, Kctd5, Klhl30, Klhl6, Lztr1, March6, Msl2, Nf1, Nsd1, Patz1, Pias1, Prdm1, Pten, Rfwd2, Rnf139, Socs3, Spag16, Strap, Stub1, Syk, Tab1, Tank, Tbk1, Tnf, Trim45, Trip12, Ube2j2, Wdfy2, Wdr61, Wdr81, Wdr91, Zbtb25, Zfp106, Zfp91, and Zmiz2.

In some aspects, the additional agent is an agent that modulates the expression of one or more members of Module M6 as presented in Example 3, e.g., modulates the expression of one or more of Ahctfl, Anapcl 1, Arih2, Arnt, Bcl6, Brap, Cbl, Cd28, Cstf1, Cul1, Cul3, Cul5, Dda1, Fbxo33, Fus, Gm9840, Hif1 a, Huwe1, Ing3, Kcmf1, Kdm5c, Keap1, Maea, Mycbp2, Nbeall, Nedd8, Nup43, Nup62, Phf8, Ptpn1, Rae1, Ranbp2, Rbbp6, Rbck1, Rbx1, Rc3 h1, Rela, Rlim, Rnf144a, Rnf31, Rnf7, Seh1I, Skp1a, Spop, Ssr3, Tbl1xr1, Tceb1, Tceb2, Tceb3, Tdpoz5, Thoc3, Tlr4, Traf6, Trim28, Trim33, Ube2d3, Ube2f, Ube2h, Ube2i, Ubr4, Ubr5, Vhl, Wdr20, Wdr26, Wdr33, Zbtb17, and Zbtb7b.

### G. Methods of Monitoring Response to Treatment

In another aspect, the disclosure features a method of monitoring the response of an individual having a cancer, an inflammatory disease, or an autoimmune disease to treatment with a modulator of the interaction between (a) Fbxw11 and (b) Nfkb1 or Nfkb2, the method comprising (i) determining, in a biological sample obtained from the individual at a time point following administration of the modulator, the expression level of an active form of one or both of Nfkb1 and Nfkb2; and (ii) comparing the expression level of the active form of one or both of Nfkb1 and Nfkb2 in the biological sample with a reference level, thereby monitoring the response in the individual to treatment with the modulator.

In some aspects, the reference level is selected from the group consisting of (i) the expression level of the one or both genes in a biological sample from the individual obtained prior to administration of the modulator; (ii) the expression level of the one or both genes in a reference population; (iii) a pre-assigned expression level for the one or both genes; or (iv) the expression level of the one or both genes in a biological sample obtained from the individual at a previous time point, wherein the previous time point is following administration of the modulator.

In some aspects, the individual has a cancer, the expression level of the active form of one or both of Nfkb1 and Nfkb2 in is decreased in the biological sample obtained from the individual relative to the reference level, and the method further comprises administering to the individual one or more additional doses of the modulator, wherein the modulator is an agent that increases the expression and/or activity of Fbxw11.

In some aspects, the individual has an inflammatory disease or an autoimmune disease, the expression level of the active form of one or both of Nfkb1 and Nfkb2 is increased in the biological sample obtained from the individual relative to the reference level, and the method further comprises administering to the individual one or more additional doses of the modulator, wherein the modulator is an agent that decreases the expression and/or activity of Fbxw11.

### VIII. Cell Therapies

### A. Methods of Treating a Cancer, an Inflammatory Disease, or an Autoimmune Disease Using a Cell Therapy

In some aspects, the disclosure features a method for treating a cancer, an inflammatory disease, or an autoimmune disease in an individual, the method comprising administering to the individual an effective amount of a cell therapy comprising a cell comprising alterations in at least two of the genes in one or more of the following co-functional gene modules:
(a) Module M1 comprising Aamp, Bop1, Cirh1a, Dcaf13, Grb2, Myc, Nle1, Noll0, Pak1ip1, Ptpn11, Rack1, Raf1, Rrp9, Taf5, Tbl3, Uhrf1, Utp15, Utp18, Vprbp, Wdr3, Wdr36, Wdr43, Wdr5, Wdr74, and Wdr75;
(b) Module M2 comprising Ago2, Ahr, Anapc13, Bach1, Baz1 a, Bid, Bptf, Brca1, Brwd3, Btbd1, Cblc, Ccnf, Cdc27, Cntn4, Copa, Copb2, Coro1a, Cpne9, Cul4b, Ddb1, Dido1, E4f1, Ecel1, Fbxl14, Fbxl5, Fbxo11, Fbxo42, Fzr1, Gemin5, Gm10697, Gm9117, Gtf2 h2, Gtf3c1, Hdac4, Hectdl, Ift122, Ikbkg, Ing2, Jun, Katnbl, Kbtbdl3, Kdm2a, Klhl23, Klhl3, Kmt2b, LOC100861784, Lrr1, Lrrc41, Map3k7, Mdm4, Mib1, Mkrn1, Mnat1, Naca, Nsmaf, Ogt, Pa2g4, Pcif1, Ppp1r11, Prc1, Ring1, Rnf128, Rnf20, Rnf225, Rnf40, Siah1 a, Siah2, Taf3, Tdpoz2, Tmem183a, Tnfsf1 1, Tradd, Traf3ip2, Trim35, Trim7, Tssc1, Ttc3, Ube2n, Ufl1, Unk1, Upf1, Vdr, Wdhd1, Wdr48, Wdr95, Wwp1, Ybx1, Zbtb14, Zbtb49, Zbtb7a, and Zmiz1;
(c) Module M3 comprising Akt1, Ankfy1, Apc, Arpc1 b, Birc2, Bmi1, Bub3, Cacybp, Cebpb, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31a, Smad2, Syvn1, Taf51, Traf2, Traf3, Traf7, Trim24, Trp53, Ube2e1, Ube2e3, Ube3c, Ufm1, Wdfy3, Wdr1, Wdr82, Whsc1, and Zbtb11;
(d) Module M4 comprising Cdc40, Ddx41, Plrg1, Ppil2, Ppwd1, Prpf19, Prpf4, Sart1, Smu1, Snrnp40, and Wdr70;
(e) Module M5 comprising Acaca, Ambra1, Amfr, Arih1, Cbll1, Cfap57, Cnot4, Cyld, Dcaf7, Det1, Dpf2, Eed, Efcab8, Egr2, Fasn, Fbxw7, Foxo3, Gsk3b, Hectd3, Hira, Icos, Ifnarl, Ikbke, Ints12, Junb, Kat6a, Kctd10, Kctd13, Kctd21, Kctd5, Klhl30, Klhl6, Lztr1, March6, Msl2, Nf1, Nfkb1, Nsd1, Patz1, Pias1, Prdm1, Pten, Rfwd2, Rnf139, Socs3, Spag16, Strap, Stub1, Syk, Tab1, Tank, Tbk1, Tnf, Trim45, Trip12, Ube2j2, Wdfy2, Wdr61, Wdr81, Wdr91, Zbtb25, Zfp106, Zfp91, and Zmiz2; and
(f) Module M6 comprising Ahctfl, Anapcl 1, Arih2, Arnt, Bcl6, Brap, Cbl, Cd28, Cstf1, Cul1, Cul3, Cul5, Dda1, Fbxo33, Fbxw11, Fus, Gm9840, Hif1a, Huwe1, Ing3, Kcmf1, Kdm5c, Keap1, Maea, Mycbp2, Nbeall, Nedd8, Nup43, Nup62, Phf8, Ptpn1, Rae1, Ranbp2, Rbbp6, Rbck1, Rbx1, Rc3 h1, Rela, Rlim, Rnf144a, Rnf31, Rnf7, Seh11, Skp1a, Spop, Ssr3, Tbllxr1, Tceb1, Tceb2, Tceb3, Tdpoz5, Thoc3, Tlr4, Traf6, Trim28, Trim33, Ube2d3, Ube2f, Ube2h, Ube2i, Ubr4, Ubr5, Vhl, Wdr20, Wdr26, Wdr33, Zbtb17, and Zbtb7b.

For example, the cell may comprise (1) alterations in at least two of the genes of Module M1 (e.g., alterations in two, three, four, five, six, seven, eight, nine, ten, or more than ten of the genes of Module M1); (2) alterations in at least two of the genes of Module M2 (e.g., alterations in two, three, four, five, six, seven, eight, nine, ten, or more than ten of the genes of Module M2); (3) alterations in at least two of the genes of Module M3 (e.g., alterations in two, three, four, five, six, seven, eight, nine, ten, or more than ten of the genes of Module M3); (4) alterations in at least two of the genes of Module M4 (e.g., alterations in two, three, four, five, six, seven, eight, nine, ten, or more than ten of the genes of Module M4); (5) alterations in at least two of the genes of Module M5 (e.g., alterations in two, three, four, five, six, seven, eight, nine, ten, or more than ten of the genes of Module M5); or (6) alterations in at least two of the genes of Module M6 (e.g., alterations in two, three, four, five, six, seven, eight, nine, ten, or more than ten of the genes of Module M6).

In some aspects, the cell comprises alterations in at least two genes in a first module and one or more genes in a second module, e.g., comprises alterations in at least two of the genes of Module M1 and at least one of the genes of any one of Modules M2-M6.

In some aspects, the cell comprises alterations in at least two genes in a first module and at least two genes in a second module, e.g., comprises alterations in at least two of the genes of Module M1 and at least two of the genes of any one of Modules M2-M6.

In some aspects, the disclosure features a method for treating a cancer, an inflammatory disease, or an autoimmune disease in an individual, the method comprising administering to the individual an effective amount of a cell therapy comprising a cell comprising alterations in at least two of the genes in one or more of the following gene sets:
(a) Gene Set 1 comprising Aamp, Actb, Alcam, Ambra1, Anxa2, Aprt, Atp5e, B2m, Btf3, Ccdc88a, Cdh1, Chd4, Cirh1a, Cox4i1, Cox7a21, Crebbp, Ctsb, Dcaf13, Ddx41, Eef1a1, Eef1b2, Eef1g, Eef2, Eif1, Eif3e, Eif3f, Eif3i, Eif3k, Fau, Gapdh, H2-D1, H2-K1, H2-M2, Hsp90ab1, Hspa5, Hspa8, Il1rn, Laptm5, Lhfpl2, March6, Ms4a7, Mtor, Myc, Naca, Ncl, Nf1, Noll0, Npm1, Ogt, Pabpc1, Paf1, Plrg1, Pparg, Psap, Rack1, Raf1, Rheb, Rpl10, Rpl10a, Rpl11, Rpl12, Rpl13, Rpl13a, Rpl14, Rpl15, Rpl17, Rpl18, Rpl18a, Rpl19, Rpl21, Rpl22, Rpl2211, Rpl23, Rpl23a, Rpl24, Rpl26, Rpl27a, Rpl28, Rpl29, Rpl3, Rpl30, Rpl31, Rpl32, Rpl34, Rpl35, Rpl35a, Rpl36, Rpl36a, Rpl37, Rpl37a, Rpl38, Rpl39, Rpl4, Rpl41, Rpl5, Rpl6, Rpl7, Rpl7a, Rpl8, Rpl9, Rplp0, Rplp1, Rplp2, Rps10, Rps11, Rps12, Rps13, Rps14, Rps15, Rps15a, Rps16, Rps17, Rps18, Rps19, Rps2, Rps20, Rps21, Rps23, Rps24, Rps25, Rps26, Rps27, Rps27a, Rps28, Rps29, Rps3, Rps3a1, Rps4x, Rps5, Rps6, Rps7, Rps8, Rps9, Rpsa, Rptor, Sgk1, Ssr4, Tab1, Taf5, Tpt1, Uhrf1, Uqcrh, Utp15, Wdr3, Wdr36, Wdr43, Wdr5, and Zbtb25;
(b) Gene Set 2 comprising A1314180, Abcc1, Acod1, Akr1 a1, Alas1, Alox5ap, Ampd3, Arih1, Ass1, B430306N03Rik, Bach1, Blvrb, Bmi1, Brca1, Btbd1, Btg1, Cat, Ccr5, Cd36, Cd52, Cd53, Cd81, Chd4, Chpf2, Clec4n, Crebbp, Creg1, Cul3, Cxcl3, Cyb5a, Dap, Dars, Dck, Ddb1, Ddit3, Egr2, Eif3f, Eif3i, Ep300, Esd, Fbxl5, Fbxw11, Gbe1, Gclm, Gdap10, Gm9840, Gss, Gstm1, H3f3b, Hmox1, Hvcn1, Il1f9, Inhba, Keap1, Lipa, Lmo4, Map3k7, Mcli, Mcoln2, Met, Mgst2, Mmp12, Mmp19, Mmp8, Mylip, Nampt, Nedd8, Nf1, Npy, Nrp1, Nup43, Nupr1, Paf1, Pf4, Pgd, Phldal, Pla2g7, Plet1, Ppfibp2, Prdx1, Prdx6, Preb, Prkcb, Procr, Ptgr1, Ptpn1, Raf1, Rbx1, Rhob, Rnasel, Rnf128, Runx2, Sdc4, Sec13, Seh11, Skp1a, Slc43a2, Slc48a1, Slc7a11, Slpi, Smad2, Srxn1, Taldo1, Tarm1, Thbs1, Tlr2, Tlr4, Tma16, Tpm4, Traf2, Traf5, Traf6, Trip12, Tubb2a, Txnrd1, Ube2d3, Ube2n, Ubr5, Uchl1, Upf1, Wdr43, Wdr61, Zbtb17, and Zyx;
(c) Gene Set 3 comprising Acp5, Ankfy1, Arpc1 b, Atp6v0d2, Bptf, Brap, C5ar1, Ccdc88a, Cd14, Cd36, Cd63, Cebpb, Chd4, Clec4d, Clec5a, Cpd, Creg1, Ctsb, Ctsz, Cul3, Ddhd1, Dnmt3a, Egr2, Emb, F630028010Rik, Fabp5, Fam46c, Fbxo42, Fcgr2b, Fn1, Foxo3, Fpr1, Ftl1, Gadd45a, Glrx, Gpnmb, Gpr84, Huwe1, Icam1, Id1, Il1f9, Kctd10, Keap1, Klhl6, Lcn2, Lgals1, Lgals3, Lgmn, Lipa, Lpcat2, Ly6c2, March6, Metrnl, Mgll, Mt1, Mtor, Myof, Naaa, Naca, Nf1, Paf1, Phldal, Pid1, Pik3r4, Pld3, Plet1, Plk2, Pou2f2, Pparg, Prdx5, Psap, Ptpn11, Rab3il1, Rela, Rfwd2, Rnase2a, S100a1, S100a11, S100a8, Saa3, Sdc3, Serpinb2, Slamf7, Snx18, Sod2, Spatal3, Stap1, Strap, Tab1, Tceb2, Tgfbi, Thbs1, Trem1, Upf1, Upp1, Vat1, Wdfy3, Wfdc21, 2010005H115Rik, and Zbtb25;
(d) Gene Set 4 comprising AC160336.1, Actb, Actg1, Ankfy1, Arhgdib, Bptf, Brap, Bri3, Ccr2, Ccr5, Cd274, Cdkn1a, Cfl1, Chd4, Clec4a2, Copa, Coro1a, Cotl1, Crip1, Cul1, Cul3, Dars, Ddhd1, Ddit3, Ear2, Eif3f, Eif3i, Ep300, Fbxw11, FIna, Gbp2, Gbp5, Grb2, Gtf3c1, H2-D1, H2-K1, Huwe1, Ifi2712a, Ill rn, Keap1, Klk1 b1, Lcp1, Lgals1, Lpl, Lrr1, Lsp1, Malat1, Marcksl1, Med8, Mgll, Mndal, Mtor, Naca, Nedd8, Nf1, Paf1, Pfn1, Pik3r4, Pten, Ptma, Ptpn11, Rack1, Rela, Rnf20, Sdc4, Skp1 a, Taf3, Taf5, Tir4, Tmsb4x, Ubb, Ube2i, Upf1, Vhl, Wdfy3, Wdr43, Wdr82, and Wfdc17;
(e) Gene Set 5 comprising AA467197, AW112010, Abcg1, Acod1, Bcl2a1 b, Bcl2a1d, Cav1, Ccll 7, Ccl3, Ccl4, Cd14, Cd200r1, Cd300lf, Cdkn1a, Cebpb, Cflar, Chd4, Clec4e, Ciic4, Copa, Cpd, Cpeb4, Cull, Cul3, Cxcl1, Cxcl2, Cxcl3, Ehd1, Ep300, Fam102b, Fam20c, Fbxw11, Gda, Gpr84, Hist1h1c, Hivep3, Ikbke, Ikbkg, 1112b, Il1a, Il1b, 116, Ing3, Inhba, Kctd21, Klf4, Laptm5, Mafb, Malat1, Malt1, Marcks, Marcksl1, Marco, Met, Mtpn, Nabp1, Nedd8, Nfkb1, Nfkbiz, Nlrp3, Nrp2, Nup62, Ogt, Paf1, Plek, Plrg1, Ppfia3, Prpf19, Ptgs2, Ptx3, Rassf4, Rbx1, Rela, Rfwd2, Rnf31, Serpinb2, Sh3bp5, Skp1 a, Slc7a11, Slc7a2, Slco3a1, Slfn2, Smad2, Smu1, Socs6, Sod2, Spop, Stub1, Tank, Tbk1, Tceb3, Tir4, Tnf, Tnfaip3, Tnfsf15, Tradd, Traf6, Trip12, Txnip, Ube2d3, Ube2i, Ube2n, Wdr82, Zbtb17, and Zc3h12c; (f) Gene Set 6 comprising AA467197, Ahr, Akt1, Ankfy1, Axl, Bhlhe40, Bhlhe41, Btg1, Ccii 7, Cc122, Ccr2, Cd40, Cd52, Cd74, Cebpb, Chd4, Clec4e, Clec4n, CIic4, Cst3, Cstf1, Ctsb, Ctsd, Cxcl16, Dcstamp, Egr2, Etv3, Fabp4, Fabp5, Fam20c, Fbxw7, Fbxw9, Foxo3, Fpr1, Fth1, Ft|l, Gbp2, Gbp5, Gm2a, Gnb1, Gnb2, Grb2, Grk3, Gsk3b, H2-Aa, H2-Ab1, Hmox1, Igf1, 114i1, Irf4, Itgax, Jak2, Jund, Kcmf1, Klhl6, Kmt2d, Lgals1, Lyz2, March6, Mg12, Mmp12, Mtor, Myc, Ndufa4, Nectin2, Nf1, Nfkb1, Pfkp, Pid1, Pik3r4, Plet1, Pmp22, Pten, Ptpn1, Ptpn11, Rheb, Rilpl2, Rptor, S100a8, Sart1, Scimp, Sdcbp, Sema4a, Sgk1, Slamf9, Smad2, Srgn, Stat5a, Tab1, Taf51, Tank, Tceb1, Tceb2, Tlr2, TIr4, Traf2, Traf3, Ube2n, Vcan, Wdfy3, Wdr26, Wdr61, and Zfp3611;
(g) Gene Set 7 comprising Abca1, Actb, Ambra1, Atf4, Atp5g1, Atp5j, Atp5j2, Bcl2a1 b, Calm1, Cfl1, Chd4, Copa, Copb2, Cot|l, Cox8a, Cul3, Cybb, Dbi, Ddit3, Eef1a1, Eif3f, Eif3i, Fbxo28, Fcer1g, Gpx1, Grb2, H2-M2, H2afz, H3f3a, Ilrn, Inhba, Keap1, Kmt2d, Lhfpl2, Ly6e, March6, Med8, Mtor, Nedd8, Nf1, Nme1, Ogt, Paf1, Plrg1, Pnp, Pparg, Rack1, S100a10, S100a4, S100a6, Sdc4, Sec13, Serf2, Sgk1, Smad2, Smu1, Sqstm1, Tab1, Taf3, Trp53, Uhrf1, Wdr43, Wdr61, and Zbtb25;
(h) Gene Set 8 comprising Aamp, Acsl1, Ambra1, Arf4, Arih2, Atf4, Bop1, C1 qb, Calr, Canx, Ccng1, Cdkn1a, Chd4, Cirh1a, Clec2d, Copa, Copb2, Cope, Cpd, Ctss, Cul3, Dad1, Dap, Dcaf13, Ddit3, Ddx41, Dstn, Eif3f, Eif3i, Erp29, Fbxw7, Fth1, Ft|l, Gm9840, Grb2, Gtf3c1, Herpud1, Hif1a, Hnrnpa3, Hsp90b1, Hspa5, Ift20, Keap1, Kmt2d, Krtcap2, Lgals3, Lrr1, Lyz2, Manf, Map3k7, Mthfd2, Mtor, Myc, Naca, Nedd8, Nf1, Nol10, Ostc, P4 hb, Pdia3, Pdia4, Pdia6, Phgdh, Plrg1, Preb, Prpf19, Pten, Ptpn1, Rack1, Rbx1, Rela, Rp12211, Rpn1, Rps19, Rrp9, Sdf2l1, Sec11c, Sec13, Sec22b, Sec31a, Sec61b, Sec61g, Selenos, Serf2, Serp1, Sf3b5, Spcs2, Ssr3, Surf4, Syvn1, Tceal9, Tceb1, Tceb2, Timm13, Tpt1, Tram1, Trp53, Ube2f, Ufm1, Uqcrq, Utp15, Vcp, Vhl, Vprbp, Wdr36, Wdr43, Wdr5, Wdr74, Wdr75, and Xbp1; (i) Gene Set 9 comprising Acod1, Adam8, Atp5g3, Brap, C3ar1, Cc12, Ccl3, Cc14, Ccl7, Ccnd1, Cd300ld, Cd63, Ch25h, Chd4, Chil3, Crip1, Ctsb, Ctsl, Cul1, Cul3, Cxcl1, Cyp51, Det1, Ear2, Egr2, F10, Fbxo42, Fbxw11, Ffar2, Fpr2, Fyb, Gas7, Gm9840, Gnb2, Gpnmb, Grb2, Gsk3b, Hmgcs1, Huwe1, Ifitm3, Il1f9, Itgam, Jun, Kctd12, Kctd5, Keap1, Klhdc4, Kmt2c, Kmt2d, Lgals1, Lgals3, Lmna, Lmo4, Lrpap1, Ly6c2, Lztr1, Maf, March6, Mcemp1, Mmp12, Mmp13, Mmp8, Msr1, Mtor, Naaa, Naca, Nf1, Nfkbiz, Npc2, Npy, Paf1, Pdpn, Pf4, Plet1, Pparg, Prkcd, Pten, Ptgs2, Ptpn1, Ptpn11, Ptprc, Ptx3, Rbbp5, Rela, Rfwd2, Rheb, Rptor, S100a6, Saa3, Scap, Scd2, Serpinb2, Serpinb6a, Sgk1, Slc7a11, Smad2, Srgn, Syk, Syngr1, Timp2, Trem2, Ube2h, Ube2i, Ucp2, Vasp, Vhl, Wdr26, Wfdc21, Ybx1, Zbtb7a, and Zfp3612;
(j) Gene Set 10 comprising Acaca, Ak4, Aldoa, Aldoc, Anapc13, Anxa2, Arih2, Arnt, Basp1, Bnip31, Bsg, C3ar1, Cc19, Cd52, Chil3, Copa, Cul2, Cul3, Cul5, Egr2, Eif3i, Eif4ebpl, Emilin2, Eno1, Ep300, Fam162a, Gapdh, Gbe1, Gpi1, Gsn, Herpud1, Hif1 a, Higd1 a, Hilpda, Hk1, Hk2, Hmox1, Huwe1, ler3, Kctd10, Klk1 b1, Ldha, Lgals3, Lipa, Lmo4, Lpcat2, Lyz2, March6, Mif, Mt1, Mt2, Mtor, Myc, Ndufv3, Nf1, Pdk1, Pfkl, Pgam1, Pgk1, Pgm2, Pkm, Prdx1, Prelid1, Ptpn1, Ptpn11, Rbpj, Rfwd2, Rilpl2, Rnase2a, Sacs, Scd2, Sdc3, Sdc4, Sec13, Slamf9, Slc16a3, Slc2a1, Slc7a2, Smu1, Socs3, Strap, Tarm1, Tceb1, Tceb2, Tgm2, Tlr4, Tpi1, Trf, Ube2f, Vhl, Vim, Wdr43, Wdr82, Wfdc17, and 2010005H15Rik;
(k) Gene Set 11 comprising AA467197, Apobec1, Apoe, Clqa, Clqb, Clqc, C3, Car4, Cc122, Ccl3, Ccl4, Ccl6, Cc19, Cd83, Cdc40, Cebpb, Ch25h, Chd4, Copa, Crebbp, Cul1, Ddhd1, Ddx41, Egr2, Eif3f, Eif3i, Ep300, Fam49a, Fbxw11, Fn1, Fnbp11, Gadd45b, Hdac4, Icam1, Icosl, Id2, Ikbkg, Il1a, 114i1, Inhba, Itgax, Itgb2, Kctd10, Klk1b11, Lpl, Maf, Marcks, Marcksll, Med8, Met, Mmp12, Ms4a6c, Ms4a7, Mt2, Mycbp2, Naca, Nedd8, Nfkbia, Phldal, Plaur, Plrg1, Pparg, Ppfibp2, Prpf19, Ptpn1, Rassf4, Rfwd2, Ring1, Rnase2a, Rpl12, Scimp, Sec13, Skp1 a, Slc43a2, Smu1, Sqstml, Syk, Syvn1, Taf51, Tceb2, Tmem176a, Tmem176b, Tnfaip2, Traf3, Ufm1, Upp1, Wdr5, Wdr70, Wdr82, Wfdc17, Wfdc21, 0610012G03Rik, Zbtb7a, and Zyx;
(l) Gene Set 12 comprising Ambra1, Aplp2, Atp5g1, Atpif1, B2m, Ccdc88a, Chd4, Copa, Cyba, Ddit3, Ear2, Egr2, Eif3f, Eif3i, Eif5, Fcgrt, Grn, H2-M2, H2-Q₆, Hint1, Id1, Ifi204, Itgal, Kctd12, Laptm5, Lgals3, Ly6e, Mgst1, Mpeg1, Mtdh, Nf1, Nfe212, Nupr1, Paf1, Pparg, Prpf19, Psmb5, Psmb6, Pycard, Rack1, Rnase4, Rp12211, Rpl37a, RplpO, Sart1, Sdc3, Sec61 b, Smad2, Smdt1, Smu1, Spp1, Syvn1, Tab1, Taf5, Taf51, Tagln2, Tmsb10, Traf2, Traf3, Trf, Trp53, Upf1, and Wdr5;
(m) Gene Set 13 comprising Ankfy1, Anxa1, Anxa5, Aph1c, Brap, C3ar1, Ccnd2, Ccr1, Cd300lf, Cd38, Cd68, Cd9, Cdc27, Cdc40, Cebpb, Chd4, Chst11, Clec4e, Creb5, Cul1, Cul3, Cxcl3, Cyba, Dstn, Eif3f, Eif3i, Emp1, Epha4, Fam102b, Fam46a, Fbxw11, Fn1, Foxo3, Ft|l, Furin, Gas7, Gdf15, Grb2, H2-K1, Huwe1, Icam1, 117r, Inhba, Keap1, Klhdc4, Klk1b11, Lgals3, Lpl, Ly6c2, Lyz2, March6, Mbnl, Mmp14, Mmp8, Ms4a7, Naca, Neat1, Nf1, Nrp2, Plin2, Plk2, Plrg1, Polr21, Prdx1, Pten, Ptpn1, Rack1, Rasgeflb, Rasgrpl, Rela, Rnf20, Rnh1, Rp12211, Rrp9, Saa3, Scd2, Sdc4, Sec13, Selenoh, Serp1, Skp1 a, Slamf7, Slc7a2, Smu1, Spp1, Tab1, Taf5, Ube2d3, Ubr4, Upf1, Vim, Wdr43, Wdr5, Wdr70, Wdr82, and Zbtb25;
(n) Gene Set 14 comprising AC160336.1, Adgrel, Adgre4, Adgrl2, Anxa1, B2m, Clqb, C3, Car4, Ccdc88a, Cc16, Cd52, Cdc40, Chd4, Chil3, Crip1, Ctsk, Ddx41, Dpf2, Egr2, Eif3i, Ep300, F7, Fcer1g, Fn1, Foxo3, Gpx3, H2-D1, H2-K1, H2-Q₆, H2-Q₇, H3f3b, Hira, Hsp90aal, Hvcn1, Id2, Ifi203, 1118, Il1f9, Kdm5c, Klhl6, Lgals1, Lgals3, Ly6e, Malt1, March6, Marcks, Mcub, Med8, Mpc1, Ms4a6d, Msrb1, Mt1, Mt2, Nedd8, Nfe212, Nov, Npc2, Paf1, Pdzk1ip1, Phgdh, Pias1, Pla2g7, Plrg1, Ppic, Ppil2, Ppwd1, Prkcd, Prpf19, Ptges, Rab32, Rbx1, Rela, Rps20, S100a 11, Sart1, Selenow, Smu1, St8sia4, Tab1, Taf51, Tceb2, Tmem176a, Tmem176b, Tnip3, Traf2, Tyrobp, Ube2i, Uchl1, Wdr5, Wdr70, Wdr82, Zbtb25, and Zfp106; and
(o) Gene Set 15 comprising AC160336.1, Adgrel, Ahnak, Alcam, Aprt, Bcl2l 1, Blvrb, Brap, Bub3, Clqb, Clqc, C3ar1, Cd300c2, Cd33, Cd68, Cdc40, Cebpb, Chchd2, Clec12a, Clec4n, Copa, Csf1r, Ctsz, Cul3, Cul5, Cyba, Ddx41, Dstn, Egr2, Ep300, F7, Fbxw7, Fcerlg, Fcgr2b, Gmfg, Gngt2, Gpr84, Hsp90aal, Huwe1, Igf1, Kat6a, Kctdl2b, Kdm5c, Keap1, Kmt2d, Lst1, Mmp14, Mpeg1, Myc, Naca, P2ry14, Paf1, Pirb, Plrg1, Pou2f2, Pparg, Ppil2, Ppwd1, Prkcd, Prpf19, Prpf4, Ptpn1, Ptpn18, Rack1, Rbbp5, Rnf20, Rnf40, Rnf7, Rps271, Sat1, Serpinb2, Smu1, Socs3, Spp1, Taf5, Tank, Tceb1, Tceb2, Tgm2, Tnfsf15, Traf2, Trem2, Tyrobp, Ufm1, Vcan, Wdr1, Wdr33, Wdr43, Wdr5, Wdr61, Wdr70, Wdr82, Wfdc21, and Ybx1.

For example, the cell may comprise (1) alterations in at least two of the genes of Gene Set 1 (e.g., alterations in two, three, four, five, six, seven, eight, nine, ten, or more than ten of the genes of Gene Set 1); (2) alterations in at least two of the genes of Gene Set 2 (e.g., alterations in two, three, four, five, six, seven, eight, nine, ten, or more than ten of the genes of Gene Set 2); (3) alterations in at least two of the genes of Gene Set 3 (e.g., alterations in two, three, four, five, six, seven, eight, nine, ten, or more than ten of the genes of Gene Set 3); (4) alterations in at least two of the genes of Gene Set 4 (e.g., alterations in two, three, four, five, six, seven, eight, nine, ten, or more than ten of the genes of Gene Set 4); (5) alterations in at least two of the genes of Gene Set 5 (e.g., alterations in two, three, four, five, six, seven, eight, nine, ten, or more than ten of the genes of Gene Set 5); (6) alterations in at least two of the genes of Gene Set 6 (e.g., alterations in two, three, four, five, six, seven, eight, nine, ten, or more than ten of the genes of Gene Set 6); (7) alterations in at least two of the genes of Gene Set 7 (e.g., alterations in two, three, four, five, six, seven, eight, nine, ten, or more than ten of the genes of Gene Set 7), (8) alterations in at least two of the genes of Gene Set 8 (e.g., alterations in two, three, four, five, six, seven, eight, nine, ten, or more than ten of the genes of Gene Set 8); (9) alterations in at least two of the genes of Gene Set 9 (e.g., alterations in two, three, four, five, six, seven, eight, nine, ten, or more than ten of the genes of Gene Set 9); (10) alterations in at least two of the genes of Gene Set 10 (e.g., alterations in two, three, four, five, six, seven, eight, nine, ten, or more than ten of the genes of Gene Set 10); (11) alterations in at least two of the genes of Gene Set 11 (e.g., alterations in two, three, four, five, six, seven, eight, nine, ten, or more than ten of the genes of Gene Set 11); (12) alterations in at least two of the genes of Gene Set 12 (e.g., alterations in two, three, four, five, six, seven, eight, nine, ten, or more than ten of the genes of Gene Set 12); (13) alterations in at least two of the genes of Gene Set 13 (e.g., alterations in two, three, four, five, six, seven, eight, nine, ten, or more than ten of the genes of Gene Set 13); (14) alterations in at least two of the genes of Gene Set 14 (e.g., alterations in two, three, four, five, six, seven, eight, nine, ten, or more than ten of the genes of Gene Set 14); or (15) alterations in at least two of the genes of Gene Set 15 (e.g., alterations in two, three, four, five, six, seven, eight, nine, ten, or more than ten of the genes of Gene Set 15).

In some aspects, the cell comprises alterations in at least two genes in a first gene set and one or more genes in a second gene set, e.g., comprises alterations in at least two of the genes of Gene Set 1 and at least one of the genes of any one of Gene Sets 2-15.

In some aspects, the cell comprises alterations in at least two genes in a first module and at least two genes in a second module, e.g., comprises alterations in at least two of the genes of Gene Set 1 and at least two of the genes of any one of Gene Sets 2-15.

The alterations may be loss-of-function mutations (e.g., mutations that result in reduced or abolished protein function, including deletions) or gain-of-function mutations (e.g., mutations that result in increased gene function, including gene duplications). For example, a cell comprising alterations in two of the genes of Module M1 may comprise loss-of-function mutations in both genes; may comprise gain-of-function mutations in both genes; or may comprise a loss-of-function mutation in the first gene and a gain-of-function mutation in the second gene. In some aspects, at least one of the alterations is a loss-of-function alteration. In some aspects, at least one of the alterations is a gain-of-function alteration.

In some aspects, the cell therapy is a dendritic cell therapy, a macrophage cell therapy, an adoptive T cell therapy (ACT), a tumor-infiltrating lymphocyte (TIL) therapy, an engineered T cell receptor (TCR) therapy (TCR-T), a chimeric antigen receptor T cell (CAR-T) therapy, a CAR-Treg therapy, or a natural killer (NK) cell therapy.

### B. Modified Cells

In another aspect, the disclosure provides a genetically modified isolated cell comprising alterations in at least two of the genes in one or more of the following co-functional gene modules:
(a) Module M1 comprising Aamp, Bop1, Cirh1a, Dcaf13, Grb2, Myc, Nle1, Noll0, Pak1ip1, Ptpn11, Rack1, Raf1, Rrp9, Taf5, Tbl3, Uhrf1, Utp15, Utp18, Vprbp, Wdr3, Wdr36, Wdr43, Wdr5, Wdr74, and Wdr75;
(b) Module M2 comprising Ago2, Ahr, Anapc13, Bach1, Baz1 a, Bid, Bptf, Brca1, Brwd3, Btbd1, Cblc, Ccnf, Cdc27, Cntn4, Copa, Copb2, Coro1a, Cpne9, Cul4b, Ddb1, Dido1, E4f1, Ecel1, Fbxl14, Fbxl5, Fbxo11, Fbxo42, Fzr1, Gemin5, Gm10697, Gm9117, Gtf2 h2, Gtf3c1, Hdac4, Hectdl, Ift122, Ikbkg, Ing2, Jun, Katnbl, Kbtbdl3, Kdm2a, Klhl23, Klhl3, Kmt2b, LOC100861784, Lrr1, Lrrc41, Map3k7, Mdm4, Mib1, Mkrn1, Mnat1, Naca, Nsmaf, Ogt, Pa2g4, Pcif1, Ppp1r11, Prc1, Ring1, Rnf128, Rnf20, Rnf225, Rnf40, Siah1 a, Siah2, Taf3, Tdpoz2, Tmem183a, Tnfsf1 1, Tradd, Traf3ip2, Trim35, Trim7, Tssc1, Ttc3, Ube2n, Ufl1, Unk1, Upf1, Vdr, Wdhd1, Wdr48, Wdr95, Wwp1, Ybx1, Zbtb14, Zbtb49, Zbtb7a, and Zmiz1;
(c) Module M3 comprising Akt1, Ankfy1, Apc, Arpc1 b, Birc2, Bmi1, Bub3, Cacybp, Cebpb, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31a, Smad2, Syvn1, Taf51, Traf2, Traf3, Traf7, Trim24, Trp53, Ube2e1, Ube2e3, Ube3c, Ufm1, Wdfy3, Wdr1, Wdr82, Whsc1, and Zbtb11;
(d) Module M4 comprising Cdc40, Ddx41, Plrg1, Ppil2, Ppwd1, Prpf19, Prpf4, Sart1, Smu1, Snrnp40, and Wdr70;
(e) Module M5 comprising Acaca, Ambra1, Amfr, Arih1, Cbl1, Cfap57, Cnot4, Cyld, Dcaf7, Det1, Dpf2, Eed, Efcab8, Egr2, Fasn, Fbxw7, Foxo3, Gsk3b, Hectd3, Hira, Icos, Ifnarl, Ikbke, Ints12, Junb, Kat6a, Kctd10, Kctd13, Kctd21, Kctd5, Klhl30, Klhl6, Lztr1, March6, Msl2, Nf1, Nfkb1, Nsd1, Patz1, Pias1, Prdm1, Pten, Rfwd2, Rnf139, Socs3, Spag16, Strap, Stub1, Syk, Tab1, Tank, Tbk1, Tnf, Trim45, Trip12, Ube2j2, Wdfy2, Wdr61, Wdr81, Wdr91, Zbtb25, Zfp106, Zfp91, and Zmiz2; and
(f) Module M6 comprising Ahctfl, Anapcl 1, Arih2, Arnt, Bcl6, Brap, Cbl, Cd28, Cstf1, Cul1, Cul3, Cul5, Dda1, Fbxo33, Fbxw11, Fus, Gm9840, Hif1a, Huwe1, Ing3, Kcmf1, Kdm5c, Keap1, Maea, Mycbp2, Nbeall, Nedd8, Nup43, Nup62, Phf8, Ptpn1, Rae1, Ranbp2, Rbbp6, Rbck1, Rbx1, Rc3 h1, Rela, Rlim, Rnf144a, Rnf31, Rnf7, Seh11, Skp1a, Spop, Ssr3, Tbllxr1, Tceb1, Tceb2, Tceb3, Tdpoz5, Thoc3, Tlr4, Traf6, Trim28, Trim33, Ube2d3, Ube2f, Ube2h, Ube2i, Ubr4, Ubr5, Vhl, Wdr20, Wdr26, Wdr33, Zbtb17, and Zbtb7b.

For example, the genetically modified isolated cell may comprise (1) alterations in at least two of the genes of Module M1 (e.g., alterations in two, three, four, five, six, seven, eight, nine, ten, or more than ten of the genes of Module M1); (2) alterations in at least two of the genes of Module M2 (e.g., alterations in two, three, four, five, six, seven, eight, nine, ten, or more than ten of the genes of Module M2); (3) alterations in at least two of the genes of Module M3 (e.g., alterations in two, three, four, five, six, seven, eight, nine, ten, or more than ten of the genes of Module M3); (4) alterations in at least two of the genes of Module M4 (e.g., alterations in two, three, four, five, six, seven, eight, nine, ten, or more than ten of the genes of Module M4); (5) alterations in at least two of the genes of Module M5 (e.g., alterations in two, three, four, five, six, seven, eight, nine, ten, or more than ten of the genes of Module M5); or (6) alterations in at least two of the genes of Module M6 (e.g., alterations in two, three, four, five, six, seven, eight, nine, ten, or more than ten of the genes of Module M6).

In some aspects, the genetically modified isolated cell comprises alterations in at least two genes in a first module and one or more genes in a second module, e.g., comprises alterations in at least two of the genes of Module M1 and at least one of the genes of any one of Modules M2-M6.

In some aspects, the genetically modified isolated cell comprises alterations in at least two genes in a first module and at least two genes in a second module, e.g., comprises alterations in at least two of the genes of Module M1 and at least two of the genes of any one of Modules M2-M6.

In another aspect, the disclosure provides a genetically modified isolated cell comprising alterations in at least two of the genes in one or more of the following gene sets:
(a) Gene Set 1 comprising Aamp, Actb, Alcam, Ambra1, Anxa2, Aprt, Atp5e, B2m, Btf3, Ccdc88a, Cdh1, Chd4, Cirh1a, Cox4i1, Cox7a21, Crebbp, Ctsb, Dcaf13, Ddx41, Eef1a1, Eef1b2, Eef1g, Eef2, Eif1, Eif3e, Eif3f, Eif3i, Eif3k, Fau, Gapdh, H2-D1, H2-K1, H2-M2, Hsp90abl, Hspa5, Hspa8, Ill rn, Laptm5, Lhfpl2, March6, Ms4a7, Mtor, Myc, Naca, Ncl, Nf1, Noll0, Npm1, Ogt, Pabpcl, Paf1, Plrg1, Pparg, Psap, Rack1, Raf1, Rheb, Rpl10, Rpl10a, Rpl11, Rpl12, Rpl13, Rpl13a, Rpl14, Rpl15, Rpl17, Rpl18, Rpl18a, Rpl19, Rpl21, Rpl22, Rpl2211, Rpl23, Rpl23a, Rpl24, Rpl26, Rpl27a, Rpl28, Rpl29, Rpl3, Rpl30, Rpl31, Rpl32, Rpl34, Rpl35, Rpl35a, Rpl36, Rpl36a, Rpl37, Rpl37a, Rpl38, Rpl39, Rpl4, Rpl41, Rpl5, Rpl6, Rpl7, Rpl7a, Rpl8, Rpl9, Rplp0, Rplp1, Rplp2, Rps10, Rps11, Rps12, Rps13, Rps14, Rps15, Rps15a, Rps16, Rps17, Rps18, Rps19, Rps2, Rps20, Rps21, Rps23, Rps24, Rps25, Rps26, Rps27, Rps27a, Rps28, Rps29, Rps3, Rps3a1, Rps4x, Rps5, Rps6, Rps7, Rps8, Rps9, Rpsa, Rptor, Sgk1, Ssr4, Tab1, Taf5, Tpt1, Uhrf1, Uqcrh, Utp15, Wdr3, Wdr36, Wdr43, Wdr5, and Zbtb25;
(b) Gene Set 2 comprising A1314180, Abcc1, Acod1, Akr1 a1, Alas1, Alox5ap, Ampd3, Arih1, Ass1, B430306N03Rik, Bach1, Blvrb, Bmi1, Brca1, Btbd1, Btg1, Cat, Ccr5, Cd36, Cd52, Cd53, Cd81, Chd4, Chpf2, Clec4n, Crebbp, Creg1, Cul3, Cxcl3, Cyb5a, Dap, Dars, Dck, Ddb1, Ddit3, Egr2, Eif3f, Eif3i, Ep300, Esd, Fbxl5, Fbxw11, Gbe1, Gclm, Gdap10, Gm9840, Gss, Gstm1, H3f3b, Hmox1, Hvcn1, Il1f9, Inhba, Keap1, Lipa, Lmo4, Map3k7, Mcli, Mcoln2, Met, Mgst2, Mmp12, Mmp19, Mmp8, Mylip, Nampt, Nedd8, Nf1, Npy, Nrp1, Nup43, Nupr1, Paf1, Pf4, Pgd, Phldal, Pla2g7, Plet1, Ppfibp2, Prdx1, Prdx6, Preb, Prkcb, Procr, Ptgr1, Ptpn1, Raf1, Rbx1, Rhob, Rnasel, Rnf128, Runx2, Sdc4, Sec13, Seh11, Skp1a, Slc43a2, Slc48a1, Slc7a11, Slpi, Smad2, Srxn1, Taldo1, Tarm1, Thbs1, Tlr2, Tlr4, Tma16, Tpm4, Traf2, Traf5, Traf6, Trip12, Tubb2a, Txnrd1, Ube2d3, Ube2n, Ubr5, Uchl1, Upf1, Wdr43, Wdr61, Zbtb17, and Zyx;
(c) Gene Set 3 comprising Acp5, Ankfy1, Arpc1 b, Atp6v0d2, Bptf, Brap, C5ar1, Ccdc88a, Cd14, Cd36, Cd63, Cebpb, Chd4, Clec4d, Clec5a, Cpd, Creg1, Ctsb, Ctsz, Cul3, Ddhd1, Dnmt3a, Egr2, Emb, F630028010Rik, Fabp5, Fam46c, Fbxo42, Fcgr2b, Fn1, Foxo3, Fpr1, Ftl1, Gadd45a, Glrx, Gpnmb, Gpr84, Huwe1, Icam1, Id1, Il1f9, KctdlO, Keap1, Klhl6, Lcn2, Lgalsl, Lgals3, Lgmn, Lipa, Lpcat2, Ly6c2, March6, Metrnl, Mgll, Mt1, Mtor, Myof, Naaa, Naca, Nf1, Paf1, Phldal, Pid1, Pik3r4, Pld3, Plet1, Plk2, Pou2f2, Pparg, Prdx5, Psap, Ptpn11, Rab3il1, Rela, Rfwd2, Rnase2a, S100a1, S100a11, S100a8, Saa3, Sdc3, Serpinb2, Slamf7, Snx18, Sod2, Spatal3, Stap1, Strap, Tab1, Tceb2, Tgfbi, Thbs1, Trem1, Upf1, Upp1, Vat1, Wdfy3, Wfdc21, 2010005H115Rik, and Zbtb25;
(d) Gene Set 4 comprising AC160336.1, Actb, Actg1, Ankfy1, Arhgdib, Bptf, Brap, Bri3, Ccr2, Ccr5, Cd274, Cdkn1a, Cfl1, Chd4, Clec4a2, Copa, Coro1a, Cotl1, Crip1, Cul1, Cul3, Dars, Ddhd1, Ddit3, Ear2, Eif3f, Eif3i, Ep300, Fbxw11, FIna, Gbp2, Gbp5, Grb2, Gtf3c1, H2-D1, H2-K1, Huwe1, Ifi2712a, Ill rn, Keap1, Klk1 b1, Lcp1, Lgals1, Lpl, Lrr1, Lsp1, Malat1, Marcksll, Med8, Mgll, Mndal, Mtor, Naca, Nedd8, Nf1, Paf1, Pfn1, Pik3r4, Pten, Ptma, Ptpn11, Rack1, Rela, Rnf20, Sdc4, Skp1 a, Taf3, Taf5, TIr4, Tmsb4x, Ubb, Ube2i, Upf1, Vhl, Wdfy3, Wdr43, Wdr82, and Wfdc17;
(e) Gene Set 5 comprising AA467197, AW112010, Abcg1, Acod1, Bcl2a1 b, Bcl2a1d, Cav1, Cll 7, Ccl3, Ccl4, Cd14, Cd200rl, Cd300lf, Cdkn1a, Cebpb, Cflar, Chd4, Clec4e, CIic4, Copa, Cpd, Cpeb4, Cull, Cul3, Cxcl1, Cxcl2, Cxcl3, Ehd1, Ep300, Fam102b, Fam20c, Fbxw11, Gda, Gpr84, Hist1h1c, Hivep3, Ikbke, Ikbkg, 1112b, Il1a, Il1b, 116, Ing3, Inhba, Kctd21, Klf4, Laptm5, Mafb, Malat1, Malt1, Marcks, Marcksll, Marco, Met, Mtpn, Nabp1, Nedd8, Nfkb1, Nfkbiz, Nlrp3, Nrp2, Nup62, Ogt, Paf1, Plek, Plrg1, Ppfia3, Prpf19, Ptgs2, Ptx3, Rassf4, Rbx1, Rela, Rfwd2, Rnf31, Serpinb2, Sh3bp5, Skp1 a, Slc7a11, Slc7a2, Slco3al, Slfn2, Smad2, Smu1, Socs6, Sod2, Spop, Stub1, Tank, Tbk1, Tceb3, TIr4, Tnf, Tnfaip3, Tnfsf15, Tradd, Traf6, Trip12, Txnip, Ube2d3, Ube2i, Ube2n, Wdr82, Zbtb17, and Zc3h12c;
(f) Gene Set 6 comprising AA467197, Ahr, Akt1, Ankfy1, Axl, Bhlhe40, Bhlhe41, Btg1, Cll 7, Cc122, Ccr2, Cd40, Cd52, Cd74, Cebpb, Chd4, Clec4e, Clec4n, CIic4, Cst3, Cstf1, Ctsb, Ctsd, Cxcl16, Dcstamp, Egr2, Etv3, Fabp4, Fabp5, Fam20c, Fbxw7, Fbxw9, Foxo3, Fpr1, Fth1, Ft|l, Gbp2, Gbp5, Gm2a, Gnb1, Gnb2, Grb2, Grk3, Gsk3b, H2-Aa, H2-Ab1, Hmox1, Igf1, 114i1, Irf4, Itgax, Jak2, Jund, Kcmf1, Klhl6, Kmt2d, Lgals1, Lyz2, March6, Mg12, Mmp12, Mtor, Myc, Ndufa4, Nectin2, Nf1, Nfkb1, Pfkp, Pid1, Pik3r4, Plet1, Pmp22, Pten, Ptpn1, Ptpn11, Rheb, Rilpl2, Rptor, S100a8, Sart1, Scimp, Sdcbp, Sema4a, Sgk1, Slamf9, Smad2, Srgn, Stat5a, Tab1, Taf51, Tank, Tceb1, Tceb2, Tlr2, TIr4, Traf2, Traf3, Ube2n, Vcan, Wdfy3, Wdr26, Wdr61, and Zfp3611;
(g) Gene Set 7 comprising Abca1, Actb, Ambra1, Atf4, Atp5g1, Atp5j, Atp5j2, Bcl2a1 b, Calm1, Cfl1, Chd4, Copa, Copb2, Cot|l, Cox8a, Cul3, Cybb, Dbi, Ddit3, Eef1a1, Eif3f, Eif3i, Fbxo28, Fcer1g, Gpx1, Grb2, H2-M2, H2afz, H3f3a, Ill rn, Inhba, Keap1, Kmt2d, Lhfpl2, Ly6e, March6, Med8, Mtor, Nedd8, Nf1, Nme1, Ogt, Paf1, Plrg1, Pnp, Pparg, Rack1, S100a10, S100a4, S100a6, Sdc4, Sec13, Serf2, Sgk1, Smad2, Smu1, Sqstml, Tab1, Taf3, Trp53, Uhrf1, Wdr43, Wdr61, and Zbtb25;
(h) Gene Set 8 comprising Aamp, Acs|l, Ambra1, Arf4, Arih2, Atf4, Bop1, Cl qb, Calr, Canx, Ccng1, Cdkn1a, Chd4, Cirh1a, Clec2d, Copa, Copb2, Cope, Cpd, Ctss, Cul3, Dad1, Dap, Dcaf13, Ddit3, Ddx41, Dstn, Eif3f, Eif3i, Erp29, Fbxw7, Fth1, Ft|l, Gm9840, Grb2, Gtf3c1, Herpudl, Hif1a, Hnrnpa3, Hsp90bl, Hspa5, Ift20, Keap1, Kmt2d, Krtcap2, Lgals3, Lrr1, Lyz2, Manf, Map3k7, Mthfd2, Mtor, Myc, Naca, Nedd8, Nf1, Noll0, Ostc, P4 hb, Pdia3, Pdia4, Pdia6, Phgdh, Plrg1, Preb, Prpf19, Pten, Ptpn1, Rack1, Rbx1, Rela, Rp12211, Rpn1, Rps19, Rrp9, Sdf2l1, Sec11c, Sec13, Sec22b, Sec31a, Sec61b, Sec61g, Selenos, Serf2, Serp1, Sf3b5, Spcs2, Ssr3, Surf4, Syvn1, Tceal9, Tceb1, Tceb2, Timm13, Tpt1, Tram1, Trp53, Ube2f, Ufm1, Uqcrq, Utp15, Vcp, Vhl, Vprbp, Wdr36, Wdr43, Wdr5, Wdr74, Wdr75, and Xbp1;
(i) Gene Set 9 comprising Acod1, Adam8, Atp5g3, Brap, C3ar1, Cc12, Ccl3, Cc14, Ccl7, Ccnd1, Cd3001d, Cd63, Ch25h, Chd4, Chil3, Crip1, Ctsb, Ctsl, Cul1, Cul3, Cxcl1, Cyp51, Det1, Ear2, Egr2, F10, Fbxo42, Fbxw11, Ffar2, Fpr2, Fyb, Gas7, Gm9840, Gnb2, Gpnmb, Grb2, Gsk3b, Hmgcs1, Huwe1, Ifitm3, Il1f9, Itgam, Jun, Kctd12, Kctd5, Keap1, Klhdc4, Kmt2c, Kmt2d, Lgals1, Lgals3, Lmna, Lmo4, Lrpap1, Ly6c2, Lztr1, Maf, March6, Mcemp1, Mmp12, Mmp13, Mmp8, Msr1, Mtor, Naaa, Naca, Nf1, Nfkbiz, Npc2, Npy, Paf1, Pdpn, Pf4, Plet1, Pparg, Prkcd, Pten, Ptgs2, Ptpn1, Ptpn11, Ptprc, Ptx3, Rbbp5, Rela, Rfwd2, Rheb, Rptor, S100a6, Saa3, Scap, Scd2, Serpinb2, Serpinb6a, Sgk1, Slc7a11, Smad2, Srgn, Syk, Syngri, Timp2, Trem2, Ube2h, Ube2i, Ucp2, Vasp, Vhl, Wdr26, Wfdc21, Ybx1, Zbtb7a, and Zfp3612;
(j) Gene Set 10 comprising Acaca, Ak4, Aldoa, Aldoc, Anapc13, Anxa2, Arih2, Arnt, Basp1, Bnip31, Bsg, C3ar1, Cc19, Cd52, Chil3, Copa, Cul2, Cul3, Cul5, Egr2, Eif3i, Eif4ebpi, Emilin2, Eno1, Ep300, Fam162a, Gapdh, Gbe1, Gpi1, Gsn, Herpudi, Hif1 a, Higd1 a, Hilpda, Hk1, Hk2, Hmox1, Huwe1, ler3, Kctd10, Klk1 b1, Ldha, Lgals3, Lipa, Lmo4, Lpcat2, Lyz2, March6, Mif, Mt1, Mt2, Mtor, Myc, Ndufv3, Nf1, Pdk1, Pfkl, Pgam1, Pgk1, Pgm2, Pkm, Prdx1, Prelidi, Ptpn1, Ptpn11, Rbpj, Rfwd2, Rilpl2, Rnase2a, Sacs, Scd2, Sdc3, Sdc4, Sec13, Slamf9, Slc16a3, Slc2a1, Slc7a2, Smu1, Socs3, Strap, Tarm1, Tceb1, Tceb2, Tgm2, Tlr4, Tpi1, Trf, Ube2f, Vhl, Vim, Wdr43, Wdr82, Wfdc17, and 2010005H1i5Rik;
(k) Gene Set 11 comprising AA467197, Apobeci, Apoe, Ciqa, Ciqb, Ciqc, C3, Car4, Cc122, Ccl3, Ccl4, Ccl6, Cc19, Cd83, Cdc40, Cebpb, Ch25h, Chd4, Copa, Crebbp, Cul1, Ddhd1, Ddx41, Egr2, Eif3f, Eif3i, Ep300, Fam49a, Fbxw11, Fn1, Fnbp11, Gadd45b, Hdac4, Icam1, Icosl, Id2, Ikbkg, Il1a, Il4i1, Inhba, Itgax, Itgb2, Kctd10, Klk1b11, Lpl, Maf, Marcks, Marcksli, Med8, Met, Mmp12, Ms4a6c, Ms4a7, Mt2, Mycbp2, Naca, Nedd8, Nfkbia, Phldai, Plaur, Plrg1, Pparg, Ppfibp2, Prpf19, Ptpn1, Rassf4, Rfwd2, Ring1, Rnase2a, Rpl12, Scimp, Sec13, Skp1 a, Slc43a2, Smu1, Sqstmi, Syk, Syvn1, Taf51, Tceb2, Tmem176a, Tmem176b, Tnfaip2, Traf3, Ufm1, Upp1, Wdr5, Wdr70, Wdr82, Wfdc17, Wfdc21, 0610012G03Rik, Zbtb7a, and Zyx;
(l) Gene Set 12 comprising Ambrai, Aplp2, Atp5g1, Atpifi, B2m, Ccdc88a, Chd4, Copa, Cyba, Ddit3, Ear2, Egr2, Eif3f, Eif3i, Eif5, Fcgrt, Grn, H2-M2, H2-Q₆, Hint1, Id1, Ifi204, Itgal, Kctd12, Laptm5, Lgals3, Ly6e, Mgst1, Mpeg1, Mtdh, Nf1, Nfe212, Nupr1, Paf1, Pparg, Prpf19, Psmb5, Psmb6, Pycard, Rack1, Rnase4, Rp12211, Rpl37a, RplpO, Sart1, Sdc3, Sec61 b, Smad2, Smdt1, Smu1, Spp1, Syvn1, Tab1, Taf5, Taf51, Tagln2, Tmsb10, Traf2, Traf3, Trf, Trp53, Upf1, and Wdr5;
(m) Gene Set 13 comprising Ankfy1, Anxa1, Anxa5, Aph1c, Brap, C3ar1, Ccnd2, Ccr1, Cd300lf, Cd38, Cd68, Cd9, Cdc27, Cdc40, Cebpb, Chd4, Chst11, Clec4e, Creb5, Cul1, Cul3, Cxcl3, Cyba, Dstn, Eif3f, Eif3i, Emp1, Epha4, Fam102b, Fam46a, Fbxw11, Fn1, Foxo3, Ftli, Furin, Gas7, Gdf15, Grb2, H2-K1, Huwe1, Icam1, 117r, Inhba, Keap1, Klhdc4, Klk1b11, Lgals3, Lpl, Ly6c2, Lyz2, March6, Mbnli, Mmp14, Mmp8, Ms4a7, Naca, Neat1, Nf1, Nrp2, Plin2, Plk2, Plrg1, Polr21, Prdx1, Pten, Ptpn1, Rack1, Rasgefib, Rasgrpi, Rela, Rnf20, Rnh1, Rp12211, Rrp9, Saa3, Scd2, Sdc4, Sec13, Selenoh, Serp1, Skp1 a, Slamf7, Slc7a2, Smu1, Spp1, Tab1, Taf5, Ube2d3, Ubr4, Upf1, Vim, Wdr43, Wdr5, Wdr70, Wdr82, and Zbtb25;
(n) Gene Set 14 comprising AC160336.1, Adgrel, Adgre4, Adgrl2, Anxa1, B2m, Clqb, C3, Car4, Ccdc88a, Ccl6, Cd52, Cdc40, Chd4, Chil3, Crip1, Ctsk, Ddx41, Dpf2, Egr2, Eif3i, Ep300, F7, Fcer1g, Fn1, Foxo3, Gpx3, H2-D1, H2-K1, H2-Q₆, H2-Q₇, H3f3b, Hira, Hsp90aal, Hvcn1, Id2, Ifi203, 1118, Illf9, Kdm5c, Klhl6, Lgals1, Lgals3, Ly6e, Malt1, March6, Marcks, Mcub, Med8, Mpc1, Ms4a6d, Msrb1, Mt1, Mt2, Nedd8, Nfe212, Nov, Npc2, Paf1, Pdzk1ip1, Phgdh, Pias1, Pla2g7, Plrg1, Ppic, Ppil2, Ppwd1, Prkcd, Prpf19, Ptges, Rab32, Rbx1, Rela, Rps20, S100a11, Sart1, Selenow, Smu1, St8sia4, Tab1, Taf51, Tceb2, Tmem176a, Tmem176b, Tnip3, Traf2, Tyrobp, Ube2i, Uchl1, Wdr5, Wdr70, Wdr82, Zbtb25, and Zfp106; and
(o) Gene Set 15 comprising AC160336.1, Adgrel, Ahnak, Alcam, Aprt, Bcl2l11, Blvrb, Brap, Bub3, Clqb, Clqc, C3ar1, Cd300c2, Cd33, Cd68, Cdc40, Cebpb, Chchd2, Clec12a, Clec4n, Copa, Csf1r, Ctsz, Cul3, Cul5, Cyba, Ddx41, Dstn, Egr2, Ep300, F7, Fbxw7, Fcer1g, Fcgr2b, Gmfg, Gngt2, Gpr84, Hsp90aal, Huwe1, Igf1, Kat6a, Kctdl2b, Kdm5c, Keap1, Kmt2d, Lst1, Mmp14, Mpeg1, Myc, Naca, P2ry14, Paf1, Pirb, Plrg1, Pou2f2, Pparg, Ppil2, Ppwd1, Prkcd, Prpf19, Prpf4, Ptpn1, Ptpn18, Rack1, Rbbp5, Rnf20, Rnf40, Rnf7, Rps271, Sat1, Serpinb2, Smu1, Socs3, Spp1, Taf5, Tank, Tceb1, Tceb2, Tgm2, Tnfsf15, Traf2, Trem2, Tyrobp, Ufm1, Vcan, Wdr1, Wdr33, Wdr43, Wdr5, Wdr61, Wdr70, Wdr82, Wfdc21, and Ybx1.

For example, the genetically modified isolated cell may comprise (1) alterations in at least two of the genes of Gene Set 1 (e.g., alterations in two, three, four, five, six, seven, eight, nine, ten, or more than ten of the genes of Gene Set 1); (2) alterations in at least two of the genes of Gene Set 2 (e.g., alterations in two, three, four, five, six, seven, eight, nine, ten, or more than ten of the genes of Gene Set 2); (3) alterations in at least two of the genes of Gene Set 3 (e.g., alterations in two, three, four, five, six, seven, eight, nine, ten, or more than ten of the genes of Gene Set 3); (4) alterations in at least two of the genes of Gene Set 4 (e.g., alterations in two, three, four, five, six, seven, eight, nine, ten, or more than ten of the genes of Gene Set 4); (5) alterations in at least two of the genes of Gene Set 5 (e.g., alterations in two, three, four, five, six, seven, eight, nine, ten, or more than ten of the genes of Gene Set 5); (6) alterations in at least two of the genes of Gene Set 6 (e.g., alterations in two, three, four, five, six, seven, eight, nine, ten, or more than ten of the genes of Gene Set 6); (7) alterations in at least two of the genes of Gene Set 7 (e.g., alterations in two, three, four, five, six, seven, eight, nine, ten, or more than ten of the genes of Gene Set 7), (8) alterations in at least two of the genes of Gene Set 8 (e.g., alterations in two, three, four, five, six, seven, eight, nine, ten, or more than ten of the genes of Gene Set 8); (9) alterations in at least two of the genes of Gene Set 9 (e.g., alterations in two, three, four, five, six, seven, eight, nine, ten, or more than ten of the genes of Gene Set 9); (10) alterations in at least two of the genes of Gene Set 10 (e.g., alterations in two, three, four, five, six, seven, eight, nine, ten, or more than ten of the genes of Gene Set 10); (11) alterations in at least two of the genes of Gene Set 11 (e.g., alterations in two, three, four, five, six, seven, eight, nine, ten, or more than ten of the genes of Gene Set 11); (12) alterations in at least two of the genes of Gene Set 12 (e.g., alterations in two, three, four, five, six, seven, eight, nine, ten, or more than ten of the genes of Gene Set 12); (13) alterations in at least two of the genes of Gene Set 13 (e.g., alterations in two, three, four, five, six, seven, eight, nine, ten, or more than ten of the genes of Gene Set 13); (14) alterations in at least two of the genes of Gene Set 14 (e.g., alterations in two, three, four, five, six, seven, eight, nine, ten, or more than ten of the genes of Gene Set 14); or (15) alterations in at least two of the genes of Gene Set 15 (e.g., alterations in two, three, four, five, six, seven, eight, nine, ten, or more than ten of the genes of Gene Set 15).

In some aspects, the genetically modified isolated cell comprises alterations in at least two genes in a first gene set and one or more genes in a second gene set, e.g., comprises alterations in at least two of the genes of Gene Set 1 and at least one of the genes of any one of Gene Sets 2-15.

In some aspects, the genetically modified isolated cell comprises alterations in at least two genes in a first module and at least two genes in a second module, e.g., comprises alterations in at least two of the genes of Gene Set 1 and at least two of the genes of any one of Gene Sets 2-15.

The alterations may be loss-of-function mutations (e.g., mutations that result in reduced or abolished protein function, including deletions) or gain-of-function mutations (e.g., mutations that result in increased gene function, including overexpression and gene duplications). For example, a cell comprising alterations in two of the genes of Module M1 may comprise loss-of-function mutations in both genes; may comprise gain-of-function mutations in both genes; or may comprise a loss-of-function mutation in the first gene and a gain-of-function mutation in the second gene. In some aspects, at least one of the alterations is a loss-of-function alteration. In some aspects, at least one of the alterations is a gain-of-function alteration.

In some aspects, the genetically modified isolated cell is a dendritic cell, a macrophage, a T cell, a TIL, or a NK cell. In some aspects, the genetically modified isolated cell is for use in a cell therapy as described above, e.g., is for use in a dendritic cell therapy, a macrophage cell therapy, an ACT, a TIL therapy, an engineered TCR therapy, a CAR-T therapy, a CAR-Treg therapy, a monocyte or myeloid cell therapy, a NK cell therapy, or a therapy for use in regenerative medicine (e.g., a Müller glia cell therapy or a retinal ganglion cell (RGC) therapy). In some aspects, the cell therapy is for treating a cancer, an inflammatory disease, or an autoimmune disease.

### IX. Pharmaceutical Compositions, Formulations, and Kits

Any of the modulators or agents described herein can be used in pharmaceutical compositions and formulations. Pharmaceutical compositions and formulations of a modulator or agent can be prepared by mixing one, two, three, four, or more than four agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation herein may also contain more than one active ingredients as necessary for the particular indication being treated, preferably those with complementary activities that do not adversely affect each other. For example, it may be desirable to further provide an additional therapeutic agent. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the agent or modulator, which matrices are in the form of shaped articles, for example, films, or microcapsules. The formulations to be used for in vivo administration are generally sterile. Sterility may be readily accomplished, e.g., by filtration through sterile filtration membranes.

In some instances in which the kit contains more than one agent or modulator, the agents or modulators are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent. Suitable containers for the one or more agent include, for example, bottles, vials, bags and syringes.

Any of the agents or modulators described herein may be included in the article of manufacture or kits. Any of the articles of manufacture or kits may include instructions to administer an agent or modulator to a subject in accordance with any of the methods described herein.

In some aspects, the disclosure features a kit comprising a modulator of the interaction between (a) one, two, or all three of Ldb2, Rnf165, and Traf2 and (b) CCR7 for treating an individual having a cancer, an inflammatory disease, or an autoimmune disease according to a method provided in Section V herein. In some aspects, the kit comprises a package insert comprising instructions to administer the modulator to an individual having a cancer, an inflammatory disease, or an autoimmune disease.

In some aspects, the disclosure features a kit comprising (a) an agent that decreases the expression and/or activity of Cebpb; (b) an agent that decreases the expression and/or activity of Traf2; and/or (c) an agent that increases the expression and/or activity of Dido1 for treating an individual having a cancer, an inflammatory disease, or an autoimmune disease according to a method provided in Section VI herein. In some aspects, the kit comprises a package insert comprising instructions to administer the agent to an individual having a cancer, an inflammatory disease, or an autoimmune disease.

In some aspects, the disclosure features a kit comprising (a) an agent that increases the expression and/or activity of Cebpb; (b) an agent that increases the expression and/or activity of Traf2; and/or (c) an agent that decreases the expression and/or activity of Dido1 for treating an individual having an inflammatory disease or an autoimmune disease according to a method provided in Section VI herein.

In some aspects, the kit comprises a package insert comprising instructions to administer the agent to an individual having an inflammatory disease or an autoimmune disease.

In some aspects, the disclosure features a kit comprising a modulator of the interaction between (a) Fbxw11 and (b) Nfkb1 or Nfkb2 for treating an individual having a cancer, an inflammatory disease, or an autoimmune disease according to a method provided in Section VII herein. In some aspects, the kit comprises a package insert comprising instructions to administer the modulator to an individual having a cancer, an inflammatory disease, or an autoimmune disease.

In some aspects, the disclosure features a kit comprising a cell therapy comprising a cell comprising alterations in at least two of the genes in one or more of the following co-functional gene modules provided in Section VIII herein for treating an individual having a cancer, an inflammatory disease, or an autoimmune disease according to a method provided in Section VII herein. In some aspects, the kit comprises a package insert comprising instructions to administer the agent to an individual having a cancer, an inflammatory disease, or an autoimmune disease.

In some aspects, the disclosure features a kit comprising a cell therapy comprising a cell comprising alterations in at least two of the genes in one or more of the following gene sets provided in Section VIII herein for treating an individual having a cancer, an inflammatory disease, or an autoimmune disease according to a method provided in Section VIII herein. In some aspects, the kit comprises a package insert comprising instructions to administer the agent to an individual having a cancer, an inflammatory disease, or an autoimmune disease.

In some aspects, the disclosure features a kit comprising a modulator of the interaction between Rfwd2 and one or more of Wdr82, Ep300, Anapc13, Cul2, Cul5, Huwe1, Crebbp, Skp1 a, Nedd8, Cul1, and Wdr5 for treating an individual having a cancer, an inflammatory disease, or an autoimmune disease according to a method provided in Section III(C) herein. In some aspects, the kit comprises a package insert comprising instructions to administer the modulator to an individual having a cancer, an inflammatory disease, or an autoimmune disease.

In some aspects, the disclosure features a kit comprising a modulator of a gene of Table 1 or Table 2 for treating an individual having a disease or disorder related to APCs and/or inflammation according to a method provided in Section IV herein. In some aspects, the disclosure features a kit comprising a modulator of a gene of Table 1 for treating an individual having a disease or disorder related to APCs and/or inflammation according to a method provided in Section IV herein. In some aspects, the disclosure features a kit comprising a modulator of a gene of Table 2 for treating an individual having a disease or disorder related to APCs and/or inflammation according to a method provided in Section IV herein. In some aspects, the kit comprises a package insert comprising instructions to administer the modulator to an individual having a disease or disorder related to APCs and/or inflammation.

All patent, patent publication, and literature references cited in the present specification are hereby incorporated by reference in their entirety.

### X. Examples

### Example 1. A Systematic Screen of E3 Ligases in Immune Dendritic Cells

The present examples describe a study in which a large gene family, the E3 ligases, and their interacting partners were characterized in the cellular response of primary immune cells to an inflammatory signal. The power of systematic Perturb-Seq to relate different members of one gene family as regulators in distinct co-functional modules, and their impact on individual genes, co-regulated gene programs, and cell state distributions across a mixed population of related cell types, is demonstrated. These examples also show how the modular organization of the regulatory network uncovered by Perturb-seq enables study and prediction of the impact of genetic interactions and relation of in vitro perturbations in a model system to mechanisms underlying disease risk in humans.

No human DC line exists and patient-derived material is limited in scale and accessibility for genetic perturbations. The study therefore screened mouse primary cells, and then related this signal to human genetics signals to prioritize regulators that may also play a large role in human health and disease.

### A. Introduction

Despite systematic efforts in genetics and genomics, our knowledge of the function of many genes remains limited, especially for genes from large gene families, where the general molecular function may be inferred from sequence features, but the specific mechanism, biological process, cellular context and physiological impact of individual genes and their combinations often remain partly or completely unknown. Multiple approaches can help decipher individual gene function, including Genome-Wide Association Studies (GWAS) to relate causal genetic variants to quantitative traits (1000 Genomes Project Consortium, Nature. 526: 68-74, 2015); forward genetic screens followed by phenotypic assessment, including cell viability, images or molecular profiles (Bock et al., Nat. Rev. Methods Primer, 2: 1-23, 2022); and guilt-by-association approaches, based on similarity in molecular patterns between a gene of interest and other genes. Despite their power and utility, each of these approaches has some limitations. Genetic association studies are often limited by the modest effect sizes associated with common variants in human populations (Uffelmann et al., Nat. Rev. Methods Primer, 1; 1-21, 2021); correlative approaches provide suggestive associations but not causal relations; and forward genetic screens have typically had to pre-define the phenotype of interest, such as cell viability (Tsherniak et al., Cell, 170: 564-576e.6, 2017) or a cellular marker (Parnas et al., Cell, 162: 675-686, 2015; Shifrut et al., Cell, 175: 1958-1971.e15, 2018). Finally, all approaches are challenged at deciphering genetic interactions, due to limited statistical power or experimental scale to test exponentially large numbers of combinations.

Large protein families, such as E3 ubiquitin ligases (“E3s”), are an important example of this challenge. The human genome codes for >600 different E3s responsible for catalyzing the ligation of ubiquitin (Ub) to substrates in almost every biochemical pathway (Zhou et al., Nucleic Acids Res, 46: D447-D453, 2018), including many immune functions (Park et al., Adv. Immunol., 124: 17-66, 2014). GWAS have implicated variants in E3 ligase genes in many diseases, including inflammatory and autoimmune diseases (Kattah et al., J. Mol. Biol., 429: 3471-3485, 2017; Senft et al., Nat. Rev. Cancer, 18: 69-88, 2018), but characterizing their specific cellular roles, remains challenging, as is determining their inter-relationships. In particular, dendritic cells (DCs) play a key role in initiating immune responses, including multiple inflammatory and autoimmune diseases (Greb et al., Nat. Rev. Dis. Primer, 2: 1-17, 2016; Lee et al., Science, 343: 1246980, 2014), and heritable variants in multiple genes in DCs contribute to their aberrant inflammatory signaling in disease, including several axes that may be targeted therapeutically (Oikawa et al., Commun. Biol., 3: 1-17, 2020). While previous studies implicate different E3 ligases in the DC inflammatory response to lipopolysaccharide (LPS) (Parnas et al., Cell, 162: 675-686, 2015), relatively little is known about the E3 circuit in these or other primary immune cells, as many studies focus on transformed cancer cell lines.

Recent advances in combining pooled genetic perturbation screens with rich, single-cell readouts, especially single cell RNA-seq (scRNA-seq) in Perturb-Seq assays, open opportunities to dissect the function of large gene families (Adamson et al., Cell, 167: 1867-1882.e21, 2016; Datlinger et al., Nat. Methods, 14: 297-301, 2017; Dixit et al., Cell, 167: 1853-1866.e17, 2016); Frangieh et al., Nat. Genet., 53: 332-341, 2021; Jaitin et al., Cell, 167: 1883-1896e.5, 2016; Jin et al., Science, 370: eaaz6063, 2020; McFaline-Figueroa et al., Nat. Genet., 51: 1389-1398, 2019; Norman et al., Science, 365: 786-793, 2019; Papalexi et al., Nat. Genet., 53: 322-331, 2021; Replogle et al., Nat. Biotechnol., 38: 954-961, 2020; Replogle et al., Cell, 185(14), 2022). In Perturb-Seq screens, the perturbed genes can be partitioned into co-functional modules, based on the similarity of their effects across many genes, and show their impact on co-regulated programs of genes affected similarly across multiple perturbations (Dixit et al., Cell, 167: 1853-1866.e17, 2016); Frangieh et al., Nat. Genet., 53: 332-341, 2021). Moreover, any diversity in cell subsets or processes, such the cell cycle or differentiation, is naturally captured in the screen (Dixit et al., Cell, 167: 1853-1866.e17, 2016)). Most Perturb-Seq studies to date, and especially the very few done in in primary cells, have analyzed up to a few dozen perturbations (Adamson et al., Cell, 167: 1867-1882.e21, 2016; Datlinger et al., Nat. Methods, 14: 297-301, 2017; Dixit et al., Cell, 167: 1853-1866.e17, 2016; Frangieh et al., Nat. Genet., 53: 332-341, 2021; Jaitin et al., Cell, 167: 1883-1896e.5, 2016; Jin et al., Science, 370: eaaz6063, 2020; McFaline-Figueroa et al., Nat. Genet., 51: 1389-1398, 2019; Norman et al., Science, 365: 786-793, 2019; Papalexi et al., Nat. Genet., 53: 322-331, 2021; Replogle et al., Nat. Biotechnol., 38: 954-961, 2020), with a recent notable screen of thousands of genes but only in a transformed cell line (Replogle et al., Cell, 185(14), 2022).

Here, Perturb-seq was used at scale to screen the function of each of 1,130 genes spanning E3 ligases, E3-like proteins and interacting partners, and substrates in the inflammatory response to stimulation with lipopolysaccharide (LPS) in primary mouse bone marrow derived dendritic cells (BMDCs). DCs play a key role in initiating immune responses, and aberrant inflammatory signaling from DCs is a driving force in multiple inflammatory and autoimmune diseases (Greb et al., Nat. Rev. Dis. Primer, 2: 1-17, 2016; Lee et al., Science, 343: 1246980, 2014). The cells in a single experiment spanned DC1, DC2, and migratory DC (mDC) subtypes, a gradient of DC maturation, and a range of gene programs, allowing the role of E3 ligases to be deciphered in multiple contexts simultaneously. A regulatory model distinguished six co-functional modules impacting different eleven programs of co-regulated genes, showing which E3 ligases, adaptors and substrate recognition adaptor proteins regulate each process in DCs, capturing known associations and making many new functional annotations.

Computational integration of the regulatory (genetic) model with physical protein-protein interactions and transcription factor (TF)-target genes relations showed that the regulatory model was congruent with physical mechanistic interactions. E3s and their physically interacting partners were enriched in the same co-functional module, and the programs they regulated were enriched for targets of specific TFs, highlighting multiple paths from E3s and complex members through TFs to different DC processes they regulate. Moreover, the circuit was modular, such that Cullin-RING ligases (the largest subfamily; >200 members) and their known adaptors co-regulated the same processes, but combined with different substrate recognition adaptor proteins to control distinct aspects of the DC life cycle. The circuit was also congruent with human disease biology, with both co-functional modules and their regulated programs enriched in heritability for risk immune and inflammatory diseases. Leveraging the present large screen's design to also randomly sample combinations of perturbations, it was found that intra-module (non-additive) genetic interactions are more prevalent than inter-module ones. The modular architecture was used to devise comβVAE, a new deep learning model that predicts genetic interactions. The present study offers a general scalable approach to dissect gene function, including physiological functions for dozens of E3s and related genes, congruent physical circuits, principles of modularity in the regulatory and molecular architecture, characterization and prediction of genetic interactions, and an overall model of the inflammatory response to help interpret human genetics signal at unprecedented resolution.

### B. Perturb-Seq Screen

To study the role and circuitry of members of the large gene family of E3 ligases in inflammatory responses, a comprehensive set of 1,137 genes encoding E3s and related proteins was curated for screening by Perturb-seq (Table 3).

TABLE 3
Curated E3 gene family list (Symbol, GeneID)

| | | | |
|---|---|---|---|
| Arih1, 23806 | Wdfy1, 69368 | Fbxw8, 231672 | Coro1b, 23789 |
| Rnf212, 671564 | Cntn4, 269784 | Tmem183a, 57439 | Rnf151, 67504 |
| Cbl, 12402 | Crbn, 58799 | Ube3c, 100763 | Kmt2b, 75410 |
| Ube3a, 22215 | Lrsam1, 227738 | Wdr74, 107071 | Tep1, 21745 |
| Pex2, 19302 | Atg16l1, 77040 | Traip, 22036 | Zbtb17, 22642 |
| Paf1, 54624 | Zbtb34, 241311 | Wdr55, 67936 | Lrwd1, 71735 |
| Rnf2, 19821 | Zbtb43, 71834 | Hoxb4, 15412 | Fbxo11, 225055 |
| Cul9, 78309 | Mid2, 23947 | Trim60, 234329 | Wdr1, 22388 |
| Rfpl4, 192658 | Rc3h2, 319817 | Anapc10, 68999 | Kdm5a, 214899 |
| D7Ertd443e, 71007 | Zbtb6, 241322 | Wrap53, 216853 | Rbp2, 19660 |
| Pja2, 224938 | Ift122, 81896 | Zbtb7a, 16969 | Pias4, 59004 |
| Wwp2, 66894 | Zbtb14, 22666 | Coro2b, 235431 | Coro1c, 23790 |
| Mycbp2, 105689 | Kdm5c, 20591 | Kdm2a, 225876 | Hira, 15260 |
| Pam, 18484 | Chfr, 231600 | Fbxl5, 242960 | Fancl, 67030 |
| Syvn1, 74126 | Chd4, 107932 | March4, 381270 | Shkbp1, 192192 |
| Fzr1, 56371 | Dync1i2, 13427 | Spsb2, 14794 | Ciao1, 26371 |
| Lnx1, 16924 | Rnf10, 50849 | Rrp9, 27966 | Rnf215, 71673 |
| Cblb, 208650 | Wsb2, 59043 | Kdm5b, 75605 | Nosip, 66394 |
| Trim39, 79263 | Wdhd1, 218973 | Utp4, 21771 | Btbd2, 208198 |
| Ubr2, 224826 | Klhl20, 226541 | Thoc6, 386612 | Btbd6, 399566 |
| Rnf128, 66889 | Kbtbd4, 67136 | Rnf126, 70294 | Baz1b, 22385 |
| Huwe1, 59026 | Trim50, 215061 | Wdr43, 72515 | Hltf, 20585 |
| Mid1, 17318 | Dtx2, 74198 | Pcgf3, 69587 | Kdm7a, 338523 |
| Trim71, 636931 | Gnb2, 14693 | Wdr92, 103784 | Rmnd5a, 68477 |
| March1, 72925 | Kat6a, 244349 | Pak1ip1, 68083 | Nacc1, 66830 |
| Wwp1, 107568 | Lrr1, 69706 | Phf8, 320595 | Pwp1, 103136 |
| Dcun1d1, 114893 | Wdr76, 241627 | Trim7, 94089 | Klhl10, 66720 |
| Trim25, 217069 | Trim9, 94090 | Fbxw4, 30838 | Rnf32, 56874 |
| Fbxw7, 50754 | Hecw1, 94253 | Rnf113a1, 69942 | Kctd10, 330171 |
| Trim63, 433766 | Ubr5, 70790 | Wdr20rt, 70948 | Kat2b, 18519 |
| Rnf148, 71300 | Patz1, 56218 | Kdm2b, 30841 | Pparg, 19016 |
| Ube4b, 63958 | Znrf3, 407821 | Nol10, 217431 | Bcl6b, 12029 |
| Mib2, 76580 | Eml1, 68519 | Rnf225, 381845 | Prkn, 50873 |
| Fbxl20, 72194 | Fbxo48, 319701 | Wdr70, 545085 | Trim28, 21849 |
| Ufl1, 67490 | Cpne1, 266692 | Ambra1, 228361 | Ube2i, 22196 |
| Rnf41, 67588 | Ppara, 19013 | Btbd7, 238386 | Rnf31, 268749 |
| Ccnb1ip1, 239083 | Map3k1, 26401 | Dtx4, 207521 | Egr2, 13654 |
| Mei4, 75033 | Cdc20b, 238896 | Kmt2c, 231051 | Ubr3, 68795 |
| Peli1, 67245 | Rnf114, 81018 | Wdr18, 216156 | Neurl1a, 18011 |
| Trim11, 94091 | Chd3, 216848 | Cdc40, 71713 | Ube2d1, 216080 |
| Smurf1, 75788 | Zbtb4, 75580 | Mul1, 68350 | Rbck1, 24105 |
| Pml, 18854 | Fbxo27, 233040 | Shprh, 268281 | Rnf144a, 108089 |
| March8, 71779 | Lrrc31, 320352 | Cdc16, 69957 | Ubr7, 66622 |
| Rag1, 19373 | Dpf1, 29861 | Cblc, 80794 | Ube2o, 217342 |
| Trim31, 224762 | Klhl32, 212390 | Dennd3, 105841 | Ube2e1, 22194 |
| Rad18, 58186 | Wdr62, 233064 | Wdtc1, 230796 | Ube2e3, 22193 |
| Trim30a, 20128 | Bach2, 12014 | Asb4, 65255 | Ube2d3, 66105 |
| Rbbp6, 19647 | Ift80, 68259 | Fbxo33, 70611 | Ube2d2a, 56550 |
| Trim12a, 76681 | Trim59, 66949 | Arel1, 68497 | Ube2c, 68612 |
| Siah2, 20439 | Coro2a, 107684 | Wdr45, 54636 | Rnf183, 76072 |
| Btrc, 12234 | Spop, 20747 | Herc1, 235439 | Ube2f, 67921 |
| Trim10, 19824 | Pcif1, 228866 | Zbtb1, 268564 | Cdc34, 216150 |
| Fbxw15, 382105 | Zbtb7b, 22724 | Slitrk5, 75409 | Ube2h, 22214 |
| Trim16, 94092 | Eed, 13626 | Fbxo45, 268882 | Neurl4, 216860 |
| Trim56, 384309 | Plaa, 18786 | Hectd4, 269700 | Ubac1, 98766 |
| Amfr, 23802 | Wdr78, 242584 | Topors, 106021 | Siah3, 380918 |
| Neurl1b, 240055 | Trim34b, 434218 | Pwp2, 110816 | Arih2os, 100038635 |
| Neurl2, 415115 | Trim30c, 434219 | Wdr5b, 69544 | Ube2n, 93765 |
| Neurl3, 214854 | Bptf, 207165 | Rnf13, 24017 | Ranbp2, 19386 |
| Mib1, 225164 | Kctd2, 70382 | Dmxl1, 240283 | Trp53, 22059 |
| Rnf138, 56515 | Rwdd3, 66568 | Asb6, 72323 | Siah1-ps2, 100416386 |
| Fbxo2, 230904 | Coro1a, 12721 | Enc1, 13803 | Siah1-ps1, 20435 |
| Trim41, 211007 | Fus, 233908 | Herc3, 73998 | Gm19741, 100503515 |
| Dcun1d2, 102323 | Trim72, 434246 | Socs5, 56468 | Gm19640, 100503335 |
| Siah1a, 20437 | Rab40b, 217371 | Cpne9, 211232 | Gm12361, 100418366 |
| Stub1, 56424 | Zbtb8b, 215627 | Ppp2r2d, 52432 | Gm19037, 100418151 |
| Sart1, 20227 | Phrf1, 101471 | Trpc4ap, 56407 | Gm18806, 100417751 |
| Cul7, 66515 | Snrnp40, 66585 | Trim46, 360213 | Gm13416, 664938 |
| Rcbtb1, 71330 | Fbxo6, 50762 | Utp15, 105372 | Gm44505, 664779 |
| Trim37, 68729 | Gnb1, 14688 | Ostm1, 14628 | LOC108168312, 108168312 |
| Ltn1, 78913 | Klhl17, 231003 | Klhl4, 237010 | LOC102637711, 102637711 |
| Skp1a, 21402 | Ahi1, 52906 | Rnf39, 386454 | LOC101055995, 101055995 |
| Nedd4, 17999 | Btbd11, 74007 | March9, 216438 | Mkrn3, 22652 |
| Trim21, 20821 | Nle1, 217011 | Nup43, 69912 | Ttll3, 101100 |
| Trim24, 21848 | Pex10, 668173 | Mios, 252875 | Zfp451, 98403 |
| Rchy1, 68098 | Dnaic1, 68922 | Zbtb41, 226470 | Uhrf2, 109113 |
| Lrrc41, 230654 | Rnf130, 59044 | Wdr38, 76646 | Ttll8, 239591 |
| Itch, 16396 | Arnt, 11863 | Wdr60, 217935 | Peli3, 240518 |
| Ubox5, 140629 | March7, 57438 | Atg5, 11793 | Apc, 11789 |
| Ubr1, 22222 | Rbbp4, 19646 | Fbxl4, 269514 | Pten, 19211 |
| Bmi1, 12151 | Eif3i, 54709 | Ibtk, 108837 | Akt1, 11651 |
| Cbx4, 12418 | Cbll1, 104836 | Baz1a, 217578 | Tnf, 21926 |
| Trim8, 93679 | Klhl5, 71778 | Rfwd3, 234736 | Cdkn2a, 12578 |
| Trim15, 69097 | Kbtbd6, 432879 | Zfp91, 109910 | Nfkb1, 18033 |
| Ring1, 19763 | Rnf157, 217340 | Spsb1, 74646 | Tlr4, 21898 |
| Pdlim2, 213019 | Zbtb2, 381990 | Coro7, 78885 | Myc, 17869 |
| Trim27, 19720 | Klhl41, 228003 | Irf2bpl, 238330 | Il10, 16153 |
| Mdm2, 17246 | Nsd1, 18193 | Mylip, 218203 | Hif1a, 15251 |
| Pex12, 103737 | Tdpoz5, 399676 | Zswim2, 71861 | Mex3c, 240396 |
| Pcgf2, 22658 | Gm10697, 100042761 | Dtx3, 80904 | Notch1, 18128 |
| Cdc20, 107995 | Gm4778, 212727 | Stxbp5, 78808 | Stat3, 20848 |
| Traf6, 22034 | Pias1, 56469 | Socs1, 12703 | Jun, 16476 |
| Uhrf1, 18140 | Scap, 235623 | Siah1b, 20438 | Tlr2, 24088 |
| Rnf122, 68867 | Klhl40, 72330 | Fbxo15, 50764 | Mkrn2, 67027 |
| Zbtb44, 235132 | Trim6, 94088 | Rnf167, 70510 | Ctnnb1, 12387 |
| Poc1a, 70235 | Trim5, 667823 | Trim45, 229644 | Birc7, 329581 |
| Upf1, 19704 | Nfx1, 74164 | Klhl35, 72184 | Pax6, 18508 |
| Dcun1d5, 76863 | Rnf38, 73469 | Vps18, 228545 | Fas, 14102 |
| Gnb5, 14697 | Ubr4, 69116 | Hace1, 209462 | Ldlr, 16835 |
| Tle7, 102638837 | Klhl21, 242785 | Trim36, 28105 | Nos2, 18126 |
| Btbd8, 100503185 | Hdac4, 208727 | Rnf139, 75841 | Ifnar1, 15975 |
| Zbtb45, 232879 | Gm9008, 668155 | Wipi2, 74781 | Egfr, 13649 |
| Dcaf15, 212123 | Eml4, 78798 | Asb11, 68854 | Mapk14, 26416 |
| Klhl2, 77113 | Rnf121, 75212 | Wdr48, 67561 | Rnf169, 108937 |
| Rnf223, 100045778 | Skp2, 27401 | Fem1c, 240263 | Rela, 19697 |
| Gm35339, 102638882 | Eml3, 225898 | Fbxw10, 213980 | Gsk3b, 56637 |
| Nup62, 18226 | Tmf1, 232286 | Klhl42, 232539 | Pou5f1, 18999 |
| Crebbp, 12914 | Wdr20, 69641 | Wdr44, 72404 | Stat1, 20846 |
| Rnf40, 233900 | Trim75, 333307 | Vhl, 22346 | Foxp3, 20371 |
| Gtf3c1, 233863 | Fbxw9, 68628 | Trim62, 67525 | Smad3, 17127 |
| Asb3, 65257 | Ccin, 442829 | Eipr1, 380752 | Trim69, 70928 |
| Wdr95, 381693 | Klhl9, 242521 | Phf2, 18676 | Il17a, 16171 |
| Gtf3c2, 71752 | Zbtb49, 75079 | March3, 320253 | Mtor, 56717 |
| Ldb2, 16826 | Sec31a, 69162 | Copb2, 50797 | Foxo1, 56458 |
| Klhl18, 270201 | Zbtb18, 30928 | Ube2cbp, 70348 | Fmr1, 14265 |
| Arpc1b, 11867 | Rnf187, 108660 | Fbxo22, 71999 | Dcst1, 77772 |
| Gm4858, 229571 | Cdc27, 217232 | Phip, 83946 | Ptk2, 14083 |
| Lrba, 80877 | Trim80, 432613 | Fem1b, 14155 | Nlrp3, 216799 |
| Keap1, 50868 | Rnf213, 672511 | Rnf111, 93836 | Cebpb, 12608 |
| Wdr49, 213248 | Zbtb16, 235320 | Cul5, 75717 | Mapk8, 26419 |
| Tbl1xr1, 81004 | Zfp651, 270210 | Wdr59, 319481 | Cdkn1b, 12576 |
| Gm9117, 668346 | Nwd1, 319555 | Klhl36, 234796 | Src, 20779 |
| Gm9125, 668359 | Wdr83, 67836 | Katnb1, 74187 | Cxcr4, 12767 |
| Mynn, 80732 | Taf5l, 102162 | Sh3rf1, 59009 | Ccnd1, 12443 |
| Tdpoz2, 399673 | Rnf43, 207742 | Asb9, 69299 | Fasl, 14103 |
| Dcaf8, 98193 | Smurf2, 66313 | Rbbp7, 245688 | Ar, 11835 |
| Pik3r4, 75669 | Klhl3, 100503085 | Asb12, 70392 | H2, 111364 |
| Rbbp5, 213464 | Gm5773, 436563 | Wdr13, 73447 | Snca, 20617 |
| Birc2, 11797 | Phf21a, 192285 | Trim30d, 209387 | Tnfsf11, 21943 |
| Anapc13, 69010 | Asb14, 142687 | Nsmce1, 67711 | Tcra, 21473 |
| Mdm4, 17248 | March6, 223455 | Kctd13, 233877 | Tcrb, 21577 |
| Eloc, 67923 | Hic2, 58180 | Fbxo17, 50760 | Foxo3, 56484 |
| Trip12, 14897 | Gnb1l, 13972 | Dmwd, 13401 | Smad2, 17126 |
| Fbxl19, 233902 | Kctd17, 72844 | Sec13, 110379 | Nr3c1, 14815 |
| Btbd35f16, 100042159 | Ppp2r2a, 71978 | Abtb1, 80283 | Syk, 20963 |
| Btbd19, 78611 | Kif21a, 16564 | Kbtbd8, 243574 | Ret, 19713 |
| Rnf113a2, 66381 | Tbl1x, 21372 | Fbxl14, 101358 | Tcrd, 110066 |
| Rnf4, 19822 | Dido1, 23856 | Mkrn1, 54484 | Cd28, 12487 |
| Tdpoz1, 207213 | Dio1, 13370 | Herc6, 67138 | Vdr, 22337 |
| Trim26, 22670 | Trim40, 195359 | Cul1, 26965 | Igf1r, 16001 |
| Prpf19, 28000 | Klhl34, 245683 | Asb15, 78910 | Ikbkg, 16151 |
| Rfpl4b, 215919 | Asb18, 208372 | Ing3, 71777 | Msx1, 17701 |
| Hectd1, 207304 | Asb1, 65247 | Arpc1a, 56443 | Nf1, 18015 |
| Dpf3, 70127 | Anapc5, 59008 | Dtx1, 14357 | Ltbr, 17000 |
| Aamp, 227290 | Kctd3, 226823 | Rnf34, 80751 | Sqstm1, 18412 |
| Tle3, 21887 | Traf5, 22033 | Brap, 72399 | Cdk5, 12568 |
| Gm18856, 100417835 | Fbxo9, 71538 | Klhl8, 246293 | Smad1, 17125 |
| Zfp131, 72465 | Msl2, 77853 | Anapc4, 52206 | Nfatc1, 18018 |
| Llgl2, 217325 | Cish, 12700 | Ppp2r2c, 269643 | Rho, 212541 |
| Rnf141, 67150 | Prpf4, 70052 | Wrap73, 59002 | Bid, 12122 |
| Traf7, 224619 | Wdr11, 207425 | Klhl7, 52323 | Flt3, 14255 |
| Zbtb25, 109929 | Bub3, 12237 | Chd5, 269610 | Nr1h3, 22259 |
| Wdfy3, 72145 | Trim68, 101700 | Fbxo42, 213499 | Pgr, 18667 |
| Znrf1, 170737 | Rnf217, 268291 | Zbtb8a, 73680 | Smad7, 17131 |
| Zbtb3, 75291 | Dtx3l, 209200 | Rnf19b, 75234 | Tmem173, 72512 |
| Gemin5, 216766 | Tdpoz3, 399674 | Ipp, 16351 | Ptpn11, 19247 |
| Wdr64, 75820 | Mtf2, 17765 | Rnf220, 66743 | Lif, 16878 |
| Rnf168, 70238 | Wdr91, 101240 | Eloa, 27224 | Irs1, 16367 |
| Rnf14, 56736 | Zc3hc1, 232679 | Rnf11, 29864 | Nanog, 71950 |
| Klhl25, 207952 | Zbtb24, 268294 | Dcaf12, 68970 | Arrb1, 109689 |
| Wdr27, 71682 | Ing2, 69260 | Smu1, 74255 | H2afx, 15270 |
| Kctd6, 71393 | 4930595M18Rik, 245492 | Tle1, 21885 | Map3k14, 53859 |
| Zbtb42, 382639 | Znrf2, 387524 | Rnf20, 109331 | Pink1, 68943 |
| Hectd2, 226098 | Nsmce2, 68501 | Nsmaf, 18201 | Sumo1, 22218 |
| Btbd9, 224671 | Wdr90, 106618 | Rnf115, 67845 | Prdm1, 12142 |
| Wdr25, 212198 | Rnf150, 330812 | Ints12, 71793 | Lrrk2, 66725 |
| Rab40c, 224624 | Cop1, 26374 | Trim33, 94093 | Ntrk1, 18211 |
| Trim67, 330863 | Fbxw11, 103583 | Trim55, 381485 | Ptpn1, 19246 |
| Nbeal2, 235627 | Herc2, 15204 | Zbtb46, 72147 | Nr4a1, 15370 |
| Trim13, 66597 | Abtb2, 99382 | Rae1, 66679 | Irf8, 15900 |
| Rcbtb2, 105670 | Pdzrn3, 55983 | Aurka, 20878 | Dlg4, 13385 |
| Trim30b, 244183 | Tdpoz4, 399675 | Gzf1, 74533 | Raf1, 110157 |
| Rhobtb1, 69288 | Fbxo40, 207215 | Ddb2, 107986 | Map3k7, 26409 |
| Nedd4l, 83814 | Nsd2, 107823 | Fbxo3, 57443 | Cry1, 12952 |
| Trim43c, 666731 | Btbd3, 228662 | Wdsub1, 72137 | Sdc1, 20969 |
| Cul3, 26554 | Trim17, 56631 | Dph7, 67228 | Snai1, 20613 |
| Wdr26, 226757 | Trim58, 216781 | Anapc2, 99152 | Scnn1a, 20276 |
| Zmiz1, 328365 | Preb, 50907 | Traf2, 22030 | Icos, 54167 |
| Cul2, 71745 | Trim38, 214158 | Fbxw2, 30050 | Cryab, 12955 |
| Zbtb37, 240869 | Ube3b, 117146 | Dtl, 76843 | Dab1, 13131 |
| Rnf6, 74132 | Phf1, 21652 | Dcaf6, 74106 | Hes5, 15208 |
| Rc3h1, 381305 | Peli2, 93834 | Copa, 12847 | Plk1, 18817 |
| Exoc5, 105504 | Vps41, 218035 | Ahctf1, 226747 | Mavs, 228607 |
| Gm10696, 100043188 | Wdr7, 104082 | Klhl12, 240756 | Ddx58, 230073 |
| Ecel1, 13599 | Tle6, 114606 | Klhl30, 70788 | Tcrg, 110067 |
| Nbeal1, 269198 | Sh3rf2, 269016 | Ing5, 66262 | Grb2, 14784 |
| Wdr63, 242253 | Cfap57, 68625 | Bard1, 12021 | Trib3, 228775 |
| Rnf123, 84585 | Fbh1, 50755 | Rnf25, 57751 | Vldlr, 22359 |
| Rnf146, 68031 | Rffl, 67338 | Wdr12, 57750 | Isg15, 100038882 |
| Wdr41, 218460 | Mlst8, 56716 | Wdr75, 73674 | Junb, 16477 |
| Gm5286, 383977 | Socs6, 54607 | Thoc3, 73666 | Myh10, 77579 |
| Apaf1, 11783 | Socs4, 67296 | Taf3, 209361 | Mst1r, 19882 |
| Ankfy1, 11736 | Rnf180, 71816 | Jade1, 269424 | Mapk7, 23939 |
| Gnb4, 14696 | Dnaic2, 432611 | Wdr24, 268933 | Glmn, 170823 |
| Dcaf10, 242418 | Cadps2, 320405 | Wdr73, 71968 | Nfatc3, 18021 |
| Zbtb10, 229055 | Rnf144b, 218215 | Bach1, 12013 | Ikbke, 56489 |
| Dcaf17, 75763 | Wdr6, 83669 | Rptor, 74370 | Lrp8, 16975 |
| Brwd1, 93871 | Kcmf1, 74287 | Rnf165, 225743 | Gm7075, 631906 |
| Tbl2, 27368 | Wdr86, 269633 | Fbxl15, 68431 | Atxn3, 110616 |
| Wdr54, 75659 | Ep300, 328572 | Wdr45b, 66840 | Wwtr1, 97064 |
| Zbtb38, 245007 | Kbtbd13, 74492 | Anapc11, 66156 | Npm1, 18148 |
| Kctd9, 105440 | Trim65, 338364 | Btbd1, 83962 | Fasn, 14104 |
| Brwd3, 382236 | Utp18, 217109 | Taf5, 226182 | Acaca, 107476 |
| Plrg1, 53317 | Zbtb48, 100090 | Ddb1, 13194 | Cacybp, 12301 |
| Fbxl12, 30843 | Klhl26, 234378 | Seh1l, 72124 | Nrip1, 268903 |
| Lonrf2, 381338 | March2, 224703 | Ppp2r2b, 72930 | Ybx1, 22608 |
| Tle2, 21886 | Mapkbp1, 26390 | Wdr33, 74320 | Tbk1, 56480 |
| Strn, 268980 | Eml5, 319670 | Elp2, 58523 | Ndn, 17984 |
| Gtf2h2, 23894 | Socs7, 192157 | Phf10, 72057 | Htra2, 64704 |
| Asb8, 78541 | Spag16, 66722 | Cdca3, 14793 | Cyld, 74256 |
| March11, 211147 | Daw1, 71227 | Gnb3, 14695 | Mthfs, 107885 |
| Ing4, 28019 | Lonrf1, 244421 | Phf8-ps, 74042 | Ago2, 239528 |
| Nsd3, 234135 | Rnf222, 320040 | March5, 69104 | Grb10, 14783 |
| Rasd2, 75141 | Strap, 20901 | Ivns1abp, 117198 | Ogt, 108155 |
| Socs2, 216233 | Pias3, 229615 | Kmt2d, 381022 | Cd2ap, 12488 |
| Cdc26, 66440 | Rnf103, 22644 | Chaf1b, 110749 | Dbt, 13171 |
| Btbd18, 100270744 | Gan, 209239 | Klhl24, 75785 | Drd4, 13491 |
| Cdc23, 52563 | Rnf185, 193670 | Wdr53, 68980 | Lifr, 16880 |
| Dpy19l2, 320752 | Sec31b, 240667 | Ppil2, 66053 | Dpysl2, 12934 |
| Spopl, 76857 | Rnf149, 67702 | Lztr1, 66863 | Apbb1, 11785 |
| Grwd1, 101612 | Wdr89, 72338 | Bfar, 67118 | Rapsn, 19400 |
| Gm28043, 106014251 | Rictor, 78757 | Fbxl6, 30840 | Rheb, 19744 |
| Rnf8, 58230 | Kbtbd7, 211255 | Bop1, 12181 | Map2k4, 26398 |
| Kif21b, 16565 | Rnf208, 68846 | Mgrn1, 17237 | Rims1, 116837 |
| Wdfy4, 545030 | Dcaf12l1, 245404 | Bcl6, 12053 | Dars, 226414 |
| Fbxo7, 69754 | Rspry1, 67610 | Rbx1, 56438 | Nedd8, 18002 |
| Rnf207, 433809 | Rnf182, 328234 | Tbc1d31, 210544 | Gmnn, 57441 |
| Fbxo30, 71865 | Dcun1d3, 233805 | Fbxo32, 67731 | Phf7, 71838 |
| Aire, 11634 | Triml1, 244448 | Klhl38, 268807 | Sh3rf3, 237353 |
| Wdr17, 244484 | Rnf24, 51902 | Dcaf13, 223499 | Omd, 27047 |
| Fbxl8, 50788 | Rnf138rt1, 74264 | Rnf19a, 30945 | Det1, 76375 |
| Efcab8, 100504221 | Fbxl7, 448987 | Fbxo4, 106052 | Ndfip1, 65113 |
| Tbl3, 213773 | Trim12c, 319236 | Fbxl3, 50789 | Kdm4a, 230674 |
| Rnf44, 105239 | Fbxo31, 76454 | Trim52, 212085 | Diablo, 66593 |
| Asb7, 117589 | Zbtb7c, 207259 | Klhl1, 93688 | Pa2g4, 18813 |
| Klhl14, 225266 | Eml6, 237711 | Rhobtb2, 246710 | Sept4, 18952 |
| Anapc7, 56317 | Klhl6, 239743 | Trim35, 66854 | Pcgf5, 76073 |
| Wdr66, 269701 | Rnf152, 320311 | Ppwd1, 238831 | Rybp, 56353 |
| Dzip3, 224170 | Dcaf7, 71833 | Trim23, 81003 | Unkl, 74154 |
| Rlim, 19820 | Fbxo44, 230903 | Rhobtb3, 73296 | Tab1, 66513 |
| Kbtbd12, 74589 | Klhl31, 244923 | Traf3, 22031 | Pdcd6ip, 18571 |
| Hecw2, 329152 | Trim3, 55992 | Dcaf4, 73828 | Naca, 17938 |
| Atg16l2, 73683 | Rnf7, 19823 | Asb2, 65256 | Tank, 21353 |
| Wdr31, 71354 | Sag, 20215 | Wdr37, 207615 | Rnf112, 22671 |
| Cfap44, 212517 | Klhl11, 217194 | Mnat1, 17420 | Dda1, 66498 |
| Zbtb9, 474156 | E4f1, 13560 | Klhl28, 66689 | Sh3gl2, 20404 |
| Ssr3, 67437 | Wdr72, 546144 | Cfap52, 71860 | Ube2j2, 140499 |
| G2e3, 217558 | Hic1, 15248 | Coro6, 216961 | Tradd, 71609 |
| Dpf2, 19708 | Spsb4, 211949 | Trim47, 217333 | Smyd1, 12180 |
| Dmxl2, 235380 | Zfp106, 20402 | Pafah1b1, 18472 | Ccdc40, 207607 |
| Ttc3, 22129 | Kdm5d, 20592 | Klhl29, 208439 | 8030462N17Rik, 212163 |
| Ube4a, 140630 | Hya, 109757 | Rnft1, 76892 | Ppp1r11, 76497 |
| Fbxl2, 72179 | Zbtb5, 230119 | Rack1, 14694 | Dok3, 27261 |
| Wdr81, 192652 | Dcaf1, 321006 | Wdr82, 77305 | Ccnb2, 12442 |
| Eml2, 72205 | Trim2, 80890 | Herc4, 67345 | Lrch2, 210297 |
| Dcaf11, 28199 | Ercc8, 71991 | Pex7, 18634 | Stap2, 106766 |
| Klhl22, 224023 | Ing1, 26356 | Nedd1, 17997 | Mthfsl, 100039707 |
| Rnft2, 269695 | Zbtb39, 320080 | Poc1b, 382406 | Maea, 59003 |
| Ahr, 11622 | Dcaf5, 320808 | Traf3ip2, 103213 | Prc1, 233406 |
| Cstf1, 67337 | Kctd21, 622320 | Tmem129, 68366 | Lrrc55, 241528 |
| Rnf186, 66825 | Socs3, 12702 | Rnf145, 74315 | Jade2, 76901 |
| Dync1i1, 13426 | Irf2bp1, 272359 | Med8, 80509 | Zfp598, 213753 |
| Phf14, 75725 | Wdr36, 225348 | Strn4, 97387 | Zfp512b, 269401 |
| Ccnf, 12449 | Rnf216, 108086 | Rnf135, 71956 | Elob, 67673 |
| Rnf133, 386611 | Zbtb22, 81630 | Wsb1, 78889 | Fbxo25, 66822 |
| Klhl13, 67455 | Klhl23, 277396 | Tnfaip1, 21927 | Usp15, 14479 |
| Zbtb33, 56805 | Nhlrc1, 105193 | Traf4, 22032 | Lrrc57, 66606 |
| Cul4b, 72584 | Gm9847, 100043256 | Zpbp2, 69376 | Ufm1, 67890 |
| Xiap, 11798 | Nup37, 69736 | Brca1, 12189 | Eif3f, 66085 |
| Dcaf12l2, 245403 | Llgl1, 16897 | Kctd5, 69259 | Parp9, 80285 |
| Cnot4, 53621 | Fbxo10, 269529 | Cul4a, 99375 | Cgrrf1, 68755 |
| Stxbp5l, 207227 | Znrf4, 20834 | Lonrf3, 74365 | Dazap2, 23994 |
| Pias2, 17344 | Wdr3, 269470 | Rnf5, 54197 | Ddx41, 72935 |
| Zbtb20, 56490 | Tle4, 21888 | Fbxw5, 30839 | Usp28, 235323 |
| Kmt2a, 214162 | Wdr61, 66317 | Stc1, 20855 | Rbx1-ps, 100043674 |
| Kbtbd2, 210973 | Fbxo28, 67948 | Wdfy2, 268752 | Rnf26, 213211 |
| Nacc2, 67991 | Fbxl13, 320118 | Rnf166, 68718 | Armc8, 74125 |
| Rnf181, 66510 | Wdr47, 99512 | Anapc1, 17222 | Triml2, 622117 |
| Pet2, 18630 | Trim32, 69807 | Rnf170, 77733 | March10, 632687 |
| Wdr5, 140858 | Kctd11, 216858 | Birc3, 11796 | Rab1b, 76308 |
| Klhl15, 236904 | Zbtb11, 271377 | Trim54, 58522 | Pdzrn4, 239618 |
| Pja1, 18744 | Hectd3, 76608 | Arih2, 23807 | LOC100861784, 100861784 |
| Zbtb21, 114565 | Rnf125, 67664 | Strn3, 94186 | |
| Wdr34, 71820 | Kbtbd3, 69149 | Zmiz2, 52915 | |
| Gmcl1, 23885 | Zbtb40, 230848 | Wdr77, 70465 | |

All 898 genes annotated as ‘E3 family’ were identified from the Mus musculus species in the integrated annotations for Ubiquitin and Ubiquitin-like Conjugation Database (iUUCD) 2.0 (Zhou et al., Nucleic Acids Res, 46: D447-D453, 2018) in April 2019. The ‘dE3 family’ gene search included members with ‘E3 activity’, ‘E3 adaptor’ and ‘ULD/UBD’ designations in iUUCD. This list was supplemented with 1,054 Mus musculus genes identified by an NCBI Gene search of the term ‘E3 activity’, to a final non-redundant list of 1,137 E3s and interaction partner genes (Table 3). The genes included 382 genes with ‘E3 activity’ designation in the iUUCD (Zhou et al., Nucleic Acids Res, 46: D447-D453, 2018), such as proteins from RING, HECT, U-box, PHD, RBR, and other families; 509 genes with ‘E3 adaptor’ designation in iUUCD, such as those from DWD, BTB, APC, Cullin, BC-box, F-box, DDB1, and other families; 6 genes with an annotated ubiquitin binding domain and one with a ubiquitin-like domain (Rbbp6) (also from iUUCD); and 239 genes based on an NCBI search for ‘E3 activity’, capturing other enzymes in the ubiquitylation cascade (Els, E2s), known E3 substrates (e.g., Tp53, Ikbke, and Cebpb), and members of relevant signaling networks regulated by E3 ligases (e.g., TLR and TNF signaling). 1S Guides were synthesized and screened for targeting 1,130 of the genes (design gRNAs could not confidently be designed for 7 putative pseudogenes).

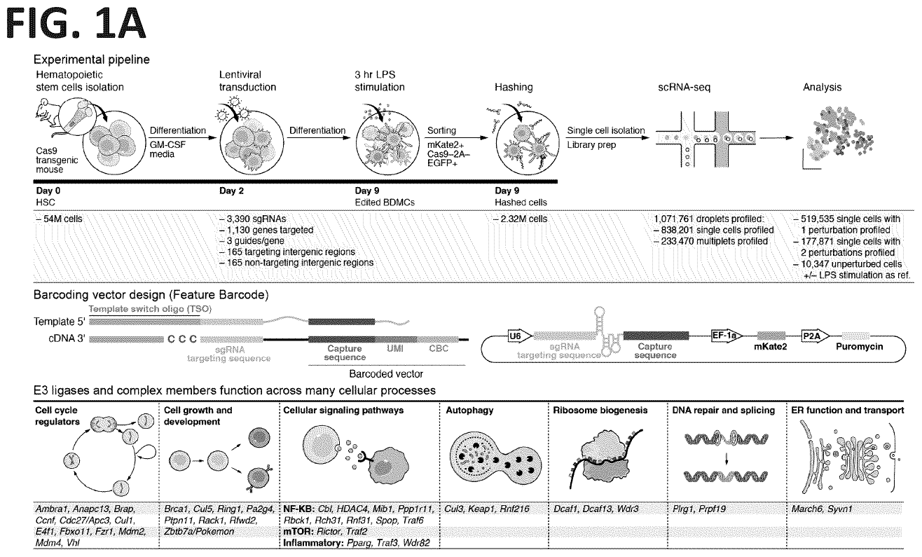

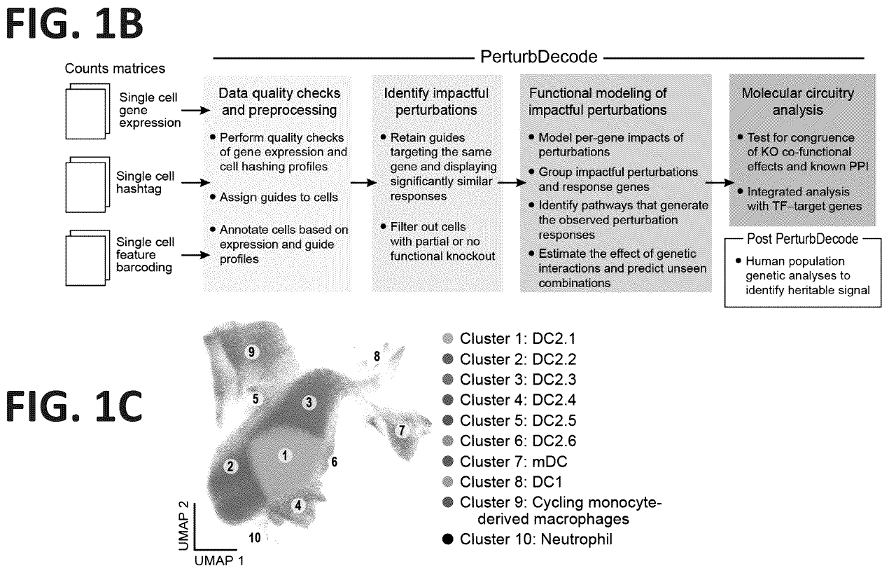

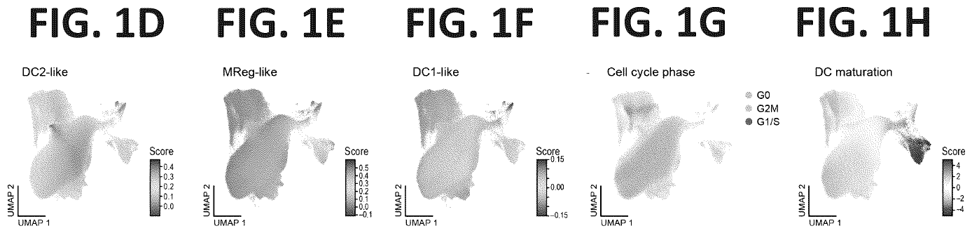

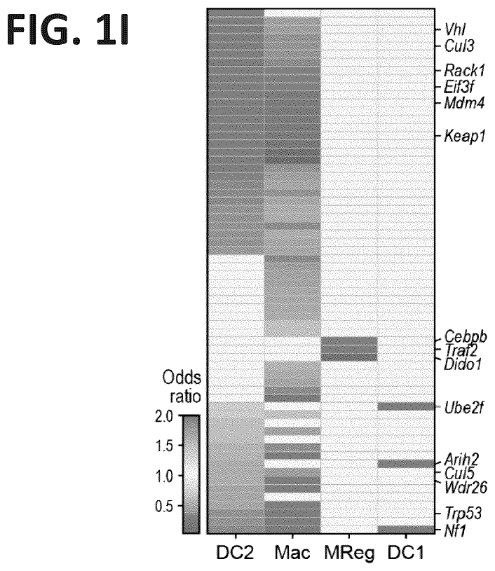

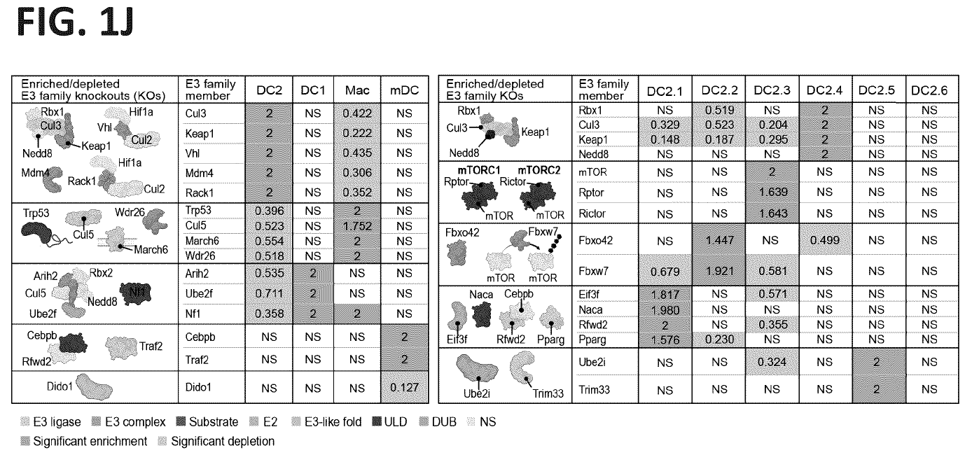

Perturb-seq was optimized for large scale screening and used to screen the 1,130 genes, perturbed by 3,390 targeting guides, profiling 838,201 individual bone marrow-derived dendritic cells (BMDCs), after 3 hours of treatment with lipopolysaccharide (LPS) (FIG. 1A). The 3-hour time point was chosen because the DC transcriptional response to LPS has a single wave, peaking around 3 hours for mature mRNA at both the population and single cell level, and is optimal for observing RNA expression effects (Amit et al., Science, 326: 257-263, 2009; Jovanovic et al., Science, 347: 1259038, 2015; Pearce and Everts, Nat. Rev. Immunol., 15: 18-29, 2015); (Parnas et al., Cell, 162: 675-686, 2015; Rabani et al., CEll, 159: 1698-1710, 2014; Rabani et al., Nat. Biotechnol., 29: 436-442, 2011; Shalek et al., Nature, 498: 236-240, 2013; Shalek et al., Nature, 510: 363-369, 2014), whereas protein level changes occur later (Jovanovic et al., Science, 347: 1259038, 2015). 54 million cells were isolated from the bone marrow of Cas9 transgenic mice (Platt et al., Cell, 159: 440-455, 2014), treated with granulocyte-macrophage colony-stimulating factor (GM-CSF) to differentiate them towards BMDCs, and, on day 2, transduced at a planned multiplicity of infection (MOI) of 0.2 with a pooled lentiviral library of 3,390 sgRNAs targeting the 1,130 genes selected above (3 guides per gene) and 330 control guides (165 targeting intergenic regions and 165 non-targeting).

A new Perturb-Seq vector (pRDA12; FIG. 1A) was designed with a capture sequence appended to the 3′ terminus of the gRNA scaffold sequence to enable direct capture of CRISPR gRNAs for single-cell RNA sequencing (scRNA-seq) (compatible with feature barcoding for droplet-based 3′ scRNA-seq (Replogle et al., Nat. Biotechnol., 38: 954-961, 2020). The FB-LentiGuide-Puro-mKate2 (pRDA12) vector is a lentiviral perturbation vector designed to be compatible with Perturb-seq Feature Barcoding technology (10× Genomics) (pRDA_122). sgRNAs contain a 3′ terminal binding sequence complementary to Feature Capture oligos present on 10× Genomics v3 and v3.1 beads. To enable FACS or puromycin selection of perturbed cells, expression of mKate2-2A-PuroR was driven by the Ef1 a promoter. The designed vector was generated by GenScript.

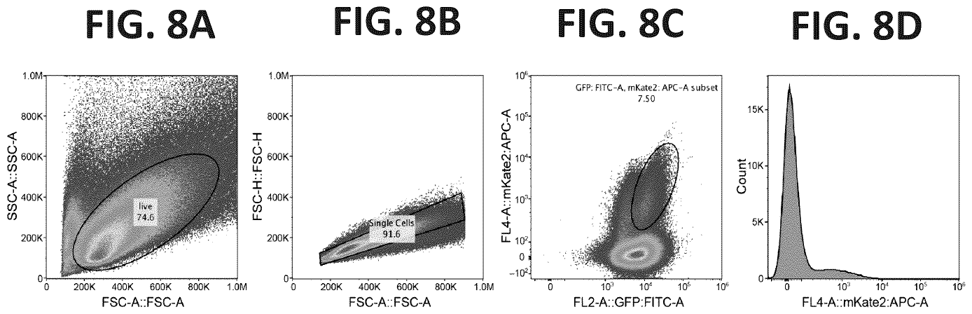

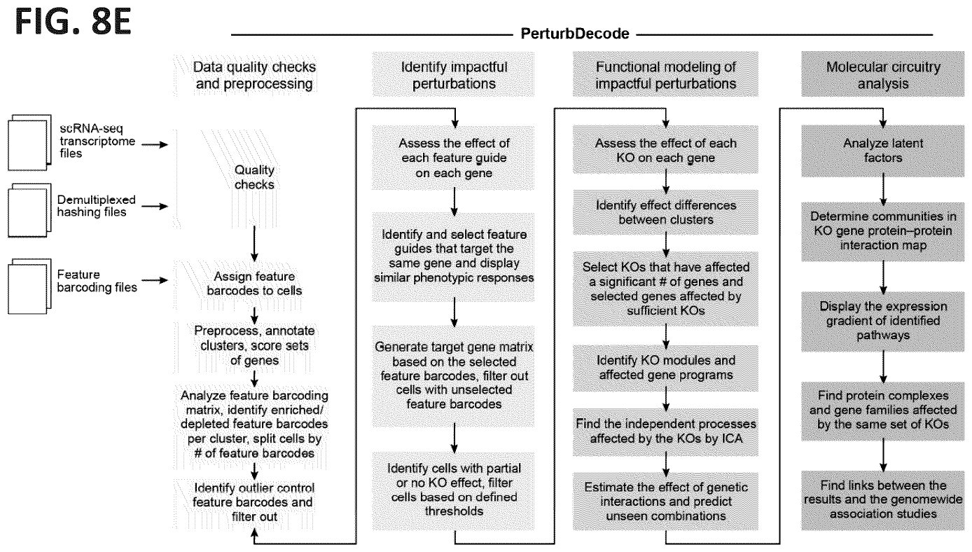

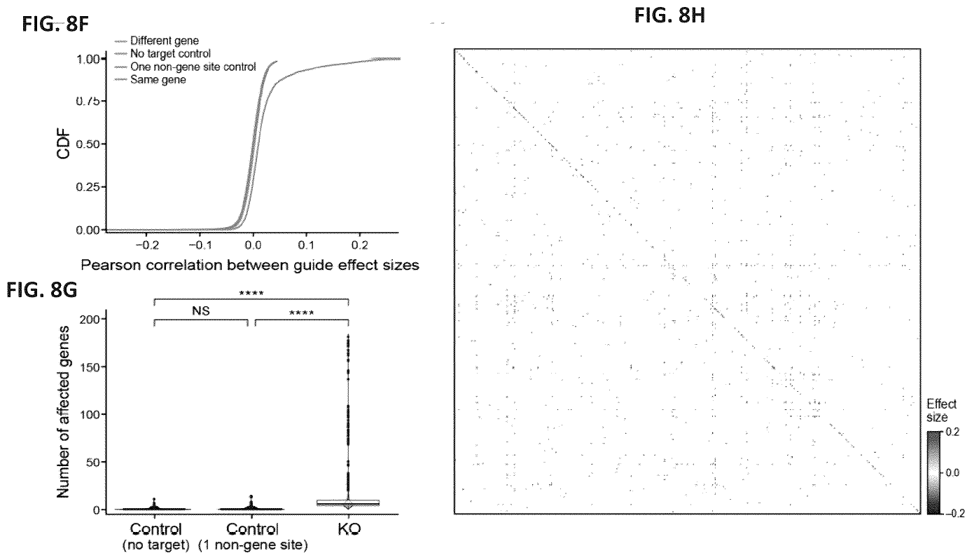

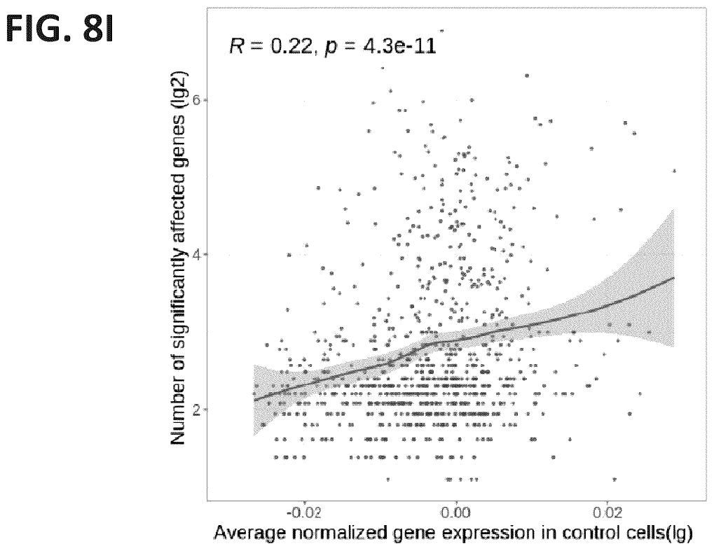

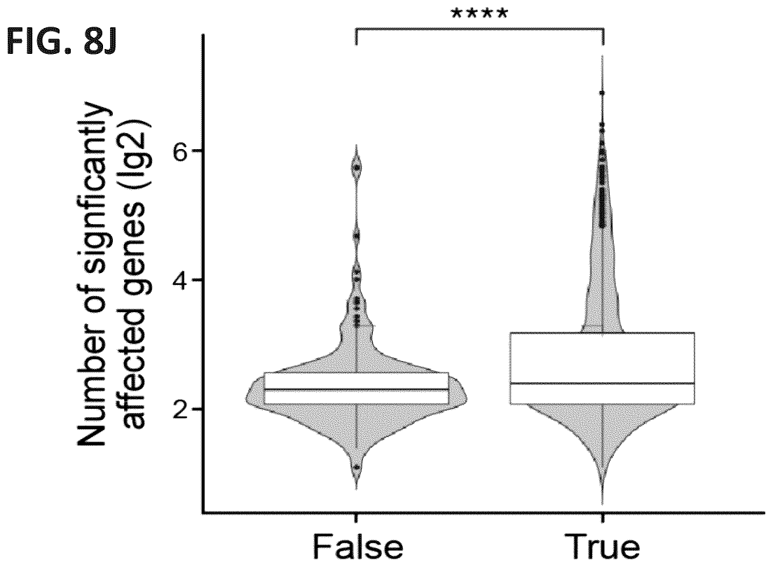

Differentiation of the transduced cells was continued for another 7 days, at which time they are predominantly BMDCs (Amit et al., Science, 326: 257-263, 2009; Chevrier et al., Cell, 147: 853-867, 2011; Garber et al., Mol. Cell, 47: 810-822, 2012; Shalek et al., Nature, 510: 363-369, 2013), and then treated them with LPS. At 3 hours post-LPS treatment, mKate2+Cas9-2A-EGFP+ cells were sorted and 2.32 million cells were loaded onto 46 channels using cell hashing and “super-loading” (Gaublomme et al., Nat. Commun., 10: 2907, 2019; Stoeckius et al., Nat. Methods, 14: 865-868, 2017) (FIGS. 8A-8D), and scRNA-seq was performed. Profiles were obtained from 1,071,671 non-empty droplets (EmptyDrops (Lun et al., Genome Biol., 20: 63, 2019), false discovery rate (FDR)<0.01), containing 838,201 single cells and 233,470 multiplets, followed by dedicated PCR to detect the guide RNA in each cell. After quality control (QC) and guide assignment, 519,535 single cell profiles assigned with one or more gRNA (targeting or control; detected MOI of 1.2) were retained for the main analyses, followed by analysis of 177,871 cells with multiple perturbations with dedicated methods provided below, as well as applying a compressed sensing approach to all the multiplets in a companion study (Cleary companion). As reference, a total of 10,347 unperturbed cells were profiled, encompassing both LPS stimulated and unstimulated cells.

Detailed methods of the Perturb-seq screen are provided in Example 1(C), below.

### C. Perturb-Seq Screen Detailed Methods

### Mice

Six-week to eleven-week-old female, constitutive Cas9-expressing mice were obtained from the Jackson labs (Strain #026179). All experiments conformed to the relevant regulatory standards.

### Bone Marrow-Derived Dendritic Cells

BMDCs were differentiated and perturbed as previously described (Dixit et al., Cell, 167: 1853-1866.e17, 2016). Cells were grown in RPMI media (ThermoFisher 21870-076) supplemented with 10% heat-inactivated fetal bovine serum (FBS) (Invitrogen), 100 U/mL penicillin/streptomycin (GIBCO 15140122), 2 mM L-glutamine (ThermoFisher 25030081), 10 mM HEPES (GIBCO 15630080), 1 mM Na pyruvate (ThermoFisher 11360070), 1× MEM nonessential amino acids (VWR 45000-700), 55 μM (3-mercaptoethanol (GIBCO 21985023), and 20 ng/mL recombinant murine GM-CSF (PeproTech 315-03). On day 0, bone marrow was extracted from mouse femur and tibia by cleaning surrounding tissue and crushing the bones gently via mortar and pestle. Bone marrow was filtered with a 70 μm cell strainer, and red blood cells were lysed in 2 mL RBC lysis buffer (SIGMA R7757) for 10 minutes at room temperature. RBC lysis was quenched with 18 mL media, and cells were spun at 1,500 RPM and resuspended in 25 mL media. Following a final 70 μm filtration, white blood cells were plated in 1,000 mm non-tissue culture-treated plastic dishes with 10 mL media at 200,000 cells/mL. On day 2, cells were fed with 10 mL media and infected with lentivirus (further described below). On day 5, 12 mL media were removed, carefully avoiding nonadherent cells, and 10 mL fresh media was added. On day 7, 5 mL media was added to cultures. On day 8, cells were collected, spun at 1,500 RPM, and resuspended in 10 mL fresh media at 1×10⁶ cells/mL. On day 9, cells were stimulated for 3 hours with 100 ng/mL LPS (Invivogen, tlrl-peklps) and harvested by scraping. Cells then underwent antibody staining for cell hashing (described below), and mKate2+(perturbation vector) and GFP+(Cas9) cells were enriched by Fluorescence Activated Cell Sorting (FACS) (˜8% population) prior to single cell library generation (FIGS. 8A-8E).

### Cloning of Guide Pools

A 3,390 Perturb-Seq guide library was designed with three guides targeting each of the 1,130 genes using the Broad Institute Genetic Perturbation Platform Web sgRNA Designer (available online) (Doench et al., Nat. Biotechnol., 34: 184-191, 2016), along with 330 control guides (165 nontargeting guides and 165 targeting intergenic regions). The pooled CRISPR library was cloned into the FB-LentiGuide-Puro-mKate2 vector backbone as previously described (Frangieh et al., Nat. Genet., 53: 332-341, 2021).

### Cell Hashing and Overloading

BMDCs were washed with PBS and resuspended in 50 μL PBS+2% BSA and 0.1% Tween20 and stained with BioLegend hashing antibodies (BioLegend 155801, 155803, 155805, 155807, 155809, 155811, 155813, 155815) as previously described (McFarland et al., Nat. Commun., 11: 4296, 2020) and mKate2⁺GFP⁺ cells were sorted by FACS (FIG. 8A). 40,000 cells were loaded per channel on 46 3′ v3 channels according to the manufacturer's instructions (10× Genomics, User Guide: Chromium Single Cell 3′ Reagent Kits v3 with Feature Barcoding technology for CRISPR screening, n.d.).

### Single Cell RNA-Seq Library Generation

ScRNA-seq libraries were generated following the manufacturer's instructions (v3 User Guide, 10× User Guide, with Feature Barcoding (10× Genomics, User Guide: Chromium Single Cell 3′ Reagent Kits v3 with Feature Barcoding technology for CRISPR screening, n.d.).

Feature Barcoding (gRNA) and Hashtag Libraries Generation

The cDNA amplification reaction was mixed by adding 5 μL of 2 μM hashtag oligonucleotide (HTO) additive, 15 μL Feature cDNA primer 2000096, 50 μL Amp Mix (10× Genomics, 2000047 or 2000103), and 30 μL cDNA followed by PCR (98° C. for 3 minutes; 98° C. for 15 seconds, 63° C. for 20 seconds, 72° C. for 60 seconds×11 cycles; 72° C. for 1 minute; hold at 4° C.). cDNA supernatant was selected by collecting the eluant from the 0.6× Solid Phase Reversible Immobilization (SPRI) cleanup of the cDNA amplification reaction. Eluant was taken from the 0.6× SPRI cleanup of the cDNA amplification reaction, and another 60 μL of SPRI beads were added to the 150 μL cDNA supernatant. After performing two 80% ethanol washes, elution was performed in 50 μL EB buffer (Qiagen). An additional 1.OX SPRI elution was performed in 30 μL EB buffer. This SPRI-purified cDNA supernatant was used as template for both hashtag and feature barcoding library generation.

To generate feature barcode (single cell gRNA) libraries, 50 μL 10× Amp mix was next mixed with 45 μL Feature SI Primers 1 and 5 μL SPRI-purified cDNA supernatant followed by PCR (98° C. for 45 seconds; 98° C. for 20 seconds, 60° C. for 30 seconds, 72° C. for 20 seconds×15 cycles; 72° C. for 1 minute; hold at 4° C.). Product was purified with 1.OX SPRI beads, eluting in 30 μL EB. Next, the product was run on a 2% TBE agarose gel at 140 volts for 40 minutes and the 250-300 bp gel fragment was purified by adding 800 μL agarose dissolving buffer (Zymo Research) to each sample and incubating at 55° C. at 1,250 RPM for 10 minutes. The dissolved gel was added to a Zymo DNA Clean & Concentrator-5 kit column and columns were spun for 30 seconds at maximum speed, followed by two 200 μL washes with the included wash buffer. Columns were spun for four minutes at max speed to dry, then transferred to a new tube followed by elution in 15 μL water (D4014). 5 μL gel purified product was mixed with 50 μL 10× Amp mix, 35 μL Feature SI Primers 2, and 10 μL Chromium i7 sample indices (PN-120262/PN-220103: Chromium i7 Multiplex Kit) followed by PCR (98° C. for 45 seconds; 98° C. for 20 seconds, 54° C. for 30 seconds, 72° C. for 20 seconds×6 cycles; 72° C. for 1 minute; hold at 4° C.) and the product was purified with 0.7× SPRI and eluted in 30 μL EB.

To generate hashtag libraries, 5 μL SPRI-purified cDNA supernatant was next combined with 25 μL NEBNext 2× MasterMix and mixed with 19 μL water, 0.5 μL 10 μM SI-PCR primer, and 0.5 μL 10 μM K_HTOX primer followed by PCR (98° C. for 10 sec; 98° C. for 2 sec, 72° C. for 15 sec×21 cycles; 72° C. for 1 min; hold at 4° C.). Product was purified with 2.OX SPRI and eluted in 15 μL TE buffer.

### Library Sequencing

Gene expression, hashtag, and feature barcoding libraries were pooled and sequenced together across 46 Illumina HiSeq 2500 lanes (R1 28 11 8 R2 90), yielding on average 15,900 scRNA-Seq reads per cell (R3: 350M reads per lane total of 12 lanes, 78% spike-in), 680 hashing reads per cell (R3: 15M reads, 4% spike-in), and 3,600 feature barcoding reads/cell (R3: 80M reads, 18% spike-in).

### Read Alignment and Demultiplexing

ScRNA-seq reads were mapped to the reference mouse genome mm10_v3.0.0 with Cumulus (Li et al., Nat. Methods, 17: 793-798, 2020) executed through Terra, using CellRanger 3.0.2. Demultiplexing of cell-hashing and feature barcoding data was performed with DemuxEM (Gaublomme et al., Nat. Commun., 10: 2907, 2019) as implemented in Cumulus.

scRNA-Seq Preprocessing

Empty droplets were removed at FDR >0.01 by EmptyDrops (Lun et al., Genome Biol., 20: 63, 2019) with parameters: lower=200, niters=10,000, ignore=10 and retain=1000. Droplets with either <300 detected genes, <1,000 total unique molecular identifiers (UMIs), or >15% mitochondrial reads were removed, retaining 1,071,671 droplet profiles. 13,811 genes expressed in at least 400 of these droplets were retained. 838,201 droplets were categorized as singlets based on hashtags by DemuxEM (Gaublomme et al., Nat. Commun., 10: 2907, 2019)). Cells were assigned a feature (guide) if they had at least 2 feature barcode UMIs, and feature barcode-UMI pairs with <20% of the reads per cell were removed (Dixit et al., Correcting Chimeric Crosstalk in Single Cell RNA-seq Experiments, bioRxiv, 2021), yielding 341,664 cells assigned one barcode and 177,871 cells assigned at least two. 186 targeting guides detected in <20 single perturbed cells were removed from further analysis, retaining 3,204 targeting guides.

### D. An End-to-End Computational Pipeline for Large Perturb-Seq Screens

To analyze large screens, PerturbDecode was developed for end-to-end, automated analysis in four consecutive pillars (FIGS. 1B and 8E): (1) data QC and preprocessing; (2) identification of the effects of perturbations on genes; (3) learning the regulatory topology of perturbed and impacted genes for single and/or combinatorial perturbations; and (4) relating the regulatory (genetic) topology to physical interactions and human genetics. These methods are described in further detail below. Briefly, in pre-processing, PerturbDecode addresses hashing and feature barcoding assignment, detects depleted feature barcodes and cells with multiple guides, and removes outliers. Next, it efficiently identifies impactful guides and impacted cells (Dixit et al., Cell, 167: 1853-1866.ei 7, 2016), by estimating the effect of each guide on each gene with a negative binomial linear regression model, accounting for confounders. It retains impactful guides defined as those with effects that are significantly similar to those of at least one other guide targeting the same gene (vs. a background of all guides), and iteratively identifies and retains impacted cells (Dixit et al., Cell, 167: 1853-1866.e17, 2016). To determine the impact at the level of perturbed genes, PerturbDecode uses a mixed effects negative binomial linear regression model, with cell subsets inferred by initial clustering as random effects and the feature barcode matrix as the set of fixed effects, correcting for confounders. It retains perturbations affecting a significant (FDR <0.1) number of genes, clusters the resulting coefficient matrix to generate co-functional modules and co-regulated programs, and decomposes the matrix by independent component analysis (ICA) to infer latent independent processes that could generate the observed perturbation responses. For combinatorial perturbations, it estimates the effect of genetic interactions and predicts the impact of unseen combinations. Finally, it includes multiple post-hoc analytics to relate the learned model to molecular circuits (protein-protein and TF-target interactions) and to human genetics data.

scRNA-Seq Expression Matrix and Dimensionality Reduction

Single cell expression matrix and feature barcodes were processed in an anndata object format in Scanpy (Wolf et al., Genome Biol., 19: 15, 2018). Raw counts were saved in the ‘counts’ layer for downstream analysis. Expression counts per cell were normalized, to a total of 10⁴ counts per cell, and normalized values were log transformed (natural log), after adding a pseudocount of 1.

A k-nearest neighbor (k=15) cell neighborhood graph was constructed with the first 50 principle components (PCs) of the log normalized expression matrix and clustered with the Leiden algorithm (Traag et al., Sci. Rep., 9; 5233, 2019) (resolution=0.5). Gene signatures were computed as the average expression of the gene set in the cells minus the average expression of a reference set of genes that is randomly sampled from the same expression bins.

Outlier control guides were identified by PCA of the log-normalized expression matrix of the 44,074 control cells with one of 330 control guides, followed by fitting a linear regression model to each of the top 100 PCs with Python statsmodels package (Seabold and Perktold, Proc. 9th Python Sci. Conf.: 92-96, 2010), where in each model one PC was the response and the binary feature barcode matrix of the control guides were the covariates. To identify outliers, the 330×100 coefficients matrix was fitted with each of four algorithms in scikit-learn Python package (Pedregosa et al., Scikit-learn: Machine Learning in Python, 2012): isolation forest (Liu et al., ACM Trans. Knowl. Discov. Data, 6(1): 1-39, 2012), elliptic envelope (Rousseeuw and Driessen, Technometrics, 41: 212-223, 1999), local outlier factor (Breunig et al., ACM S/GMOD Rec., 29: 93-104, 2000), and one-class SVM, and the 9 non-targeting and 22 intergenic guides that were predicted as outliers by at least three methods were removed.

Prediction of Corresponding Gene Expression Clusters in Unperturbed Cells with and without LPS Stimulation

Cluster assignment of LPS unstimulated unperturbed and LPS stimulated unperturbed cells were predicted by a logistic regression model trained on the LPS stimulated perturbed dataset, with the 10 cluster scores of the top 100 marker genes of the clusters as covariates.

### Guide and Knockout Enrichment Analysis

To test guide depletion in the screen vs. the initial guide pool, distributions of the ratio between the number of cells assigned a (single) guide in the screen vs. number of guides in the initial pool were generated for each of the 3,390 targeting and 330 control guides. The ratio distribution of the control guides was taken as background, to calculate an empirical P-value of the depletion of each targeting guide. Targeting guides with ratio of at most 0.08587 were identified as depleted (CDFₙᵤₗₗ (0.08587)=0.0498).

One-sided Fischer's exact tests were used to test the enrichment (separately, depletion) of cells with a particular guide (or guides targeting the same gene) in each cell subset, where the test schema was the tested group versus rest, and a Benjamini-Hochberg FDR was calculated.

### Identification of Congruent Guides Targeting the Same Gene

The effects of each of 3,204 targeting guides on 6,685 genes expressed in at least 5% of the 341,664 singly-perturbed cells were learned using a negative binomial regression model, with control cells as reference, and correcting for the total number of detected genes in a cell, % mitochondrial reads and cell states identified by the initial Leiden clustering. The pairwise Pearson correlation coefficient between the effect size profiles of each pair of guides targeting the same gene were calculated. If no pairs had a positive correlation, all guides were discarded. If all three pairs had r>0.015, all three guides were retained. Otherwise, only the guide pair with the highest positive correlation was retained.

### A Linear Regulatory Model of Knockout Effect

A mixed effects negative binomial linear regression model was fit for each of the 6,685 affected (response) genes, where the gene expression values were the response variable, the cell states identified by Leiden clustering were the random effect covariate, and the knockout (KO) target gene, confounders (number of detected gene/cell, % mitochondrial reads/cell) were the fixed effect covariates, and control cells were the reference. Benjamini-Hochberg FDR was used to correct for multiple hypotheses (6,658 tested genes) with a stringent threshold, such that most regulatory coefficients close to zero were not significant. To generate a background distribution for the number of genes significantly affected due to lentivirus infection or off-target effects, the same model was fit for each of the 299 control guides, testing one control guide against the rest of the control guides. Based on this background distribution, 329 perturbed genes were retained, as ones with significant (FDR <0.1) effect on at least 15 of the 6,685 tested genes.

### Identification of Co-Functional Modules and Co-Regulated Gene Programs

The 329 retained knockouts (perturbed genes) were grouped based on their effects on the 1,041 genes that were affected by at least by 4 perturbations. To this end, the top 50 PCs of the scaled and centered effect size matrix B (Bᵢⱼ=effect size of knockout of gene ion gene J) were used to calculate a k-nearest neighbors graph (k=10) of the knockout (perturbed) genes, and the Leiden algorithm (resolution=0.64) was used to identify the 6 clusters as co-functional modules.

Similarly, to identify co-regulated gene programs, the top 50 PCs of the scaled and centered B^{T}were used to construct a k-NN graph of the 1,041 response genes (k=10), and the Leiden algorithm (resolution=0.8) was used to identify 8 gene programs. Three of these programs were selected for further subclustering upon manual inspection, resulting in 11 gene programs.

Embedding Cells Jointly on KO Module Information and their Gene Expression Profiles

To assess the change in cell distributions across DC2.1, DC2.1 and DC2.3 subsets upon perturbations, supervised-UMAP (Sainburg et al., Neural Comput., 33: 2881-2907, 2021) was used (target_weight=0.5, kNN n_neighbors=18) to embed cells based on their normalized expression profiles and module assignment (M1-M6).

Calculating Wasserstein Distances within and Across Modules

The average population distances between cell subsets perturbed for members of each module (or controls) or between randomly sampled cell subsets perturbed for members of the same modules, were calculated by sampling without replacement 300 cells (100 times), computing Wasserstein distances between pairs of cell populations using the Python Optimal Transport Library (Flamary et al., J. Mach. Learn. Res., 22: 1-8, 2021), and averaging across 100 iterations.

Protein-Protein Interaction Analysis Mus musculus protein-protein interaction network data was downloaded from STRING DB (version 11.5) and interactions with experimental evidence score >0 were selected. Interactions between the 329 knockout targets were used to generate a protein-protein interaction graph. To test for enrichment of intra- and inter-module interactions, 400 random degree-preserving graphs were generated using the BiRewire R package (Gobbi et al., BiRewire: High-performing routines for the randomization of a bipartite graph (or a binary event matrix), undirected and directed signed graph preserving degree distribution (or marginal totals), Bioconductor, 2022) and the distribution of number of intra- and inter-module interactions in these graphs was used as the null distribution to calculate empirical P-values for the corresponding observed number of interactions.

### Inference of KO Effects on TF Factor Activity

Expression scores of high confidence targets (Levels A and B) activated by 123 mouse TFs in DoRothEA (Garcia-Alonso et al., Genome Res., 29:1363-1375, 2019; Holland et al., Biochim. Biophys. Acta, 1863: 194431, 2020, Holland et al., Genome Biol., 21: 36, 2020), were calculated using the R package decoupleR (Badia-i-Mompel et al., Bioinforma. Adv., 2: vbac016, 2022). A linear regression model was used to infer the effects of each 329 KOs on the expression of targets of each of the 123 TFs, where in each model the response variable was the expression score of each TF's target genes, and the covariates were the 1-hot encoded feature barcode matrix (with control cells as the base level) and possible confounders (cell clusters, % mitochondrial reads, number UMIs).

### ICA Module Factorization

Independent components analysis (Herault and Jutten, AIP Conf. Proc., 151: 206-211, 1986; Hyvärinen and Oja, Neural Netw., 13: 411-430, 2000) was used to identify statistically independent factors from the 1041×329 effect size matrix β from the mixed effect linear model (bᵢⱼ=estimated effect of knockout of gene j on expression of gene i). A source of variation sₖ={s₁ₖ,s₂ₖ,s₃ₖ, . . . , S₁₀₄ₖ} is defined as the set of relative weights (i.e., relative expression states) of genes 1, . . . , 1,041, such that the effects of perturbation j on expression of gene i is a weighted sum of the effects over P different sources, written as:

$${b}_{i j} = {a}_{1 j} {s}_{i 1} + {a}_{2 j} {s}_{i 2} + … + {a}_{p j} {s}_{i p}$$

where α₁ⱼ, α₂ⱼ, . . . , αₚⱼ are the mixing weights and αₚⱼ is the overall effect of perturbation j on source p. Thus, in matrix β, each row is an observation of a gene's expression changes due to the varying effects of the knockouts on various pathways (sources of variation) to which the gene belongs.

Identifying the P underlying pathways s₁, . . . , sₚ affected by 329 knockouts is formulated as finding a source matrix S (1,041 by P) and mixing matrix M (P by 329) which are both unknown:

$$B = S M$$

ICA relaxes this factorization problem by assuming that the source signals are independent and requiring that they be non-Gaussian (Hyvärinen and Oja, Neural Netw., 13: 411-430, 2000). Although modeling total perturbation effects as a linear combination of factors may miss nonlinear relationships, the nonlinear separation problem is not identifiable.

ICA decomposition was computed using Information-Maximization (Infomax) (Bell and Sejnowski, Neural Comput., 7: 1129-1159, 1995), as implemented in the ICA package in R (Helwig, ica: Independent Component Analysis, 2022). The optimal number of latent sources, P was determined considering (1) the number of non-Gaussian components estimated by the Ladle estimator (Luo and Li, Biometrika, 103: 875-887, 2016; Nordhausen et al., ICtest: Estimating and Testing the Number of Interesting Components in Linear Dimension Reduction, 2022); (2) reconstruction error of the original matrix from the obtained statistically independent components; and (3) prediction power of the identified factors for the effects of unseen perturbations during fitting. For (2), the ICtest R package (Luo and Li, Biometrika, 103: 875-887, 2016) was used to compute the ladle estimates (gn) for different number of factors, where ‘gn’ is the sum of the vectors giving the measures of variation of the eigenvectors and the normalized eigenvalues of the fourth order blind identification (FOBI) matrix and the estimated number of Gon-gaussian components is the value where gn takes its minimum. For (3), for P between 2 and 30, 80% (263) of the 329 perturbations were randomly sampled 10 times, ICA was fitted each time to the 1,041 by 263 matrix and the effects of each of the remaining 66 perturbations was predicted with a simple linear regression model, where the 1,041 by P matrix S was the covariate matrix.

After selecting P=15, the full 1,041×329 effect size matrix β was decomposed and for each factor sₖ in S, gene i was defined as a prominent gene defining this factor if it had outlier weights sᵢₖ<Q₁(sₖ)−1.5(Q₃(sₖ)−Q₁(sₖ))|sᵢₖ>Q₃(sₖ)+1.5(Q₃(sₖ)−Q₁(sₖ)). Likewise, a KO of gene j was defined as highly affecting factor k if it had outlier weights in component mₖ of the mixing matrix M, mⱼₖ<Q₁(mₖ)−(Q₃(mₖ)−Q₁(mₖ))|mⱼₖ>Q₃(mₖ)+(Q₃ (mₖ)−Q₁(mₖ)).

### Genetic Interaction Analysis

For inter-module interactions, for each of the 1,041 response genes, linear regression models were fit as follows:

$${Y}_{i} = {β}_{0} + {\sum }_{j = 1}^{6} {β}_{j} {M}_{j} + {\sum }_{j = 1}^{6} {\sum }_{𝔱 = ( j + 1 )}^{6} {β}_{jt} {M}_{j} {M}_{t} + ϵ$$

where Yᵢ is the normalized expression level of gene i (corrected for cell cluster, % mitochondrial reads and number UMIs), Mᵢ is a binary covariate denoting if the cell had a perturbation in a gene from module i. The model was fit with single KO cells and inter-module double KO cells. P-values of the β estimates were corrected with Benjamini-Hochberg FDR for the 1,041 tested genes.

For intra-module interactions, for each KO module we randomly partitioned (for 50 times) the KO module Mⱼ into two equal bins in terms of the number of KO genes, M_{j_1} and M_{j_2}, and for each response gene i fit the model:

$${Y}_{i} = {β}_{0} + {β}_{1} {M}_{j \_ 1} + {β}_{2} {M}_{j \_ 2} + {β}_{3} {M}_{j \_ 1} {M}_{j \_ 2} + ϵ$$

where Yᵢ is the normalized expression level of gene i corrected for confounders as above. Benjamini-Hochberg FDR was first calculated for the p-values of the β estimates for each of the 50 iterations, β estimates for which FDR>=0.1 were set to zero, and then the mean of the 50 parameter estimates as taken as the inferred intra-module interaction effect.

### Deep Learning Model to Predict Interactions

To learn models that predict the effect of combinatorial perturbations, conditional VAEs (CVAEs) (Sohn et al., Learning Structured Output Representation using Deep Conditional Generative Models, in: Advances in Neural Information Processing Systems. Curran Associates, Inc., 2015) were used, which model the distribution of a high-dimensional output as a generative model conditioned on the auxiliary covariates. In general, CVAEs aim to learn the marginal likelihood of the data in such a generative process:

$$\begin{matrix}max \\ ϕ , θ\end{matrix} {𝔼}_{{q}_{ϕ} ( z \vert x , a )} [ log {p}_{θ} ( x \vert a , z ) ]$$

where x∈ [symbol] ^{D} is a dataset of samples with labels (conditioned variable) a and generated by ground truth factors z, while ℠, θ parametrize the distributions of the CVAE encoder and the decoder respectively. This can be rewritten as:

$$log {p}_{θ} ( x \vert a , z ) = {D}_{K L} ( q ( z \vert x , a ) \vert \vert p ( z \vert a ) ) + L ( ϕ , θ ; x , a , z )$$

where D_{KL}(∥) is on Wive Kullback-Leibler (KL) divergence between the true and the approximate posterior and [symbol] (φ, θ; x, a, z) is the evidence lower bound (ELBO) on the log-likelihood of the data:

$$log {p}_{θ} ( x \vert a , z ) \geq ℒ ( ϕ , θ ; x , a , z ) = {E}_{{q}_{ϕ} ( z \vert x , a )} [ log {p}_{θ} ( x \vert a , z ) ] - {D}_{K L} ( q ( z \vert x , a ) \vert \vert p ( z \vert a ) )$$

To make optimization tractable in practice, p(z|a) is typically set to the isotropic unit Gaussian [symbol] (0,I) (Kingma and Welling, Found. Trends Mach. Learn., 12: 307-392, 2019). The ELBO for the VAE and CVAE factorizes across the samples (Kingma and Welling, Found. Trends Mach. Learn., 12: 307-392, 2019; Kingma and Welling, Auto-Encoding Variational Bayes, arXiv, 2022; Sohn et al., Learning Structured Output Representation using Deep Conditional Generative Models, in: Advances in Neural Information Processing Systems. Curran Associates, Inc., 2015). Therefore, it is straightforward to apply computationally efficient minibatch based stochastic gradient descent (SGD) and learn the parameters φ of the encoder (q_{φ}(z|x,a)) and θ of the decoder (p_{θ}(x|a,z)) by deep neural networks (Kingma and Welling, Found. Trends Mach. Learn., 12: 307-392, 2019).

In the comβVAE model, (xᵢ,aᵢ) denotes a single data point i, where x∈ [symbol] ^{G} is the G-dimensional observed log-normalized gene expression profiles of G genes in a single cell i and aᵢ∈A=Aᵢ₁×Aᵢ₂× . . . Aᵢₚ representing the P-dimensional auxiliary (independent) discrete covariates of the same cell, such as the knockout(s) perturbing the cell, or confounders (cell subtype or cell cycle phase).

The perturbation covariates in A are assumed to be independent binary covariates and a cell can have multiple perturbations, while other covariates are one-hot encoded. The latent variables zi are generated conditionally on a D dimensional vector v∈ [symbol] ^{D} which are the embeddings of aᵢ learned with a single hidden layer neural network of D units which is jointly trained with the encoder-decoder framework.

An adjustable hyperparameter p is introduced to the original CVAE objective, which was previously shown to result in more disentangled latent representations z in standard VAE models (Burgess et al., Understanding disentangling in $\beta$-VAE, arXiv, 2018; Higgins et al., beta-VAE: Learning Basic Visual Concepts with a Constrained Variational Framework. Presented at the International Conference on Learning Representations, 2022)

$$ℒ ( ϕ , θ ; x , v , z ) = {E}_{{q}_{ϕ} ( z \vert x , v )} [ log {p}_{θ} ( x \vert v , z ) ] - β {D}_{K L} ( q ( z \vert x , v ) \vert \vert p ( z \vert v ) )$$

Assuming the data generating process described above, the objective is to train a model such that, a target counterfactual distribution x′ᵢ of gene expression xᵢ can be generated if cell i had the covariates

$${a}_{i}^{′}$$

instead of aᵢ. For the network architecture after benchmarking the number of hidden units [2,3,4,5,6], and number of units per layer [32, 64, 128, 512, 1024], the encoder and decoder networks were defined with 2 hidden layers, with 512 units in each layer, and ReLU (rectified linear unit) activation function used between the hidden layers. The dimensions [16,32,64,128] were benchmarked for the dimensions of z and v, and both were set to 64. Models were trained and benchmarked with log-transformed normalized gene expression values of 1,041 genes corrected for cell clusters, % mitochondrial reads and total UMI counts to minimize the effect of confounders in evaluating generated vs. observed effects. The model was implemented in Pytorch, and trained with hyperparameters batch_size=1000, max_epochs=8000, optimizer=adam, learning_rate=0.001, weight_decay=0.

### E. Perturb-Seq Screen Yields Impactful Perturbations Consistently Across Guides

Estimating the impact of each of the 3,204 targeting guides (detected in at least 20 cells) on the expression of each of 6,685 genes (expressed in at least 5% of the cells) showed that the correlation in effect sizes between cells with guides targeting the same gene was significantly higher than between guides targeting different genes or between targeting and control guides (P-value <10⁻¹⁶, Kolmogorov-Smirnov (KS) test, FIG. 8F). Focusing on the concordant guides, 2,263 KO guides targeting 1,031 genes were retained for downstream analysis and a model was learned at the targeted gene (rather than guide) level. The average number of genes significantly affected by each perturbed gene (FDR <0.1) was significantly higher than for controls (36.91 vs. 0.78 (non-targeting) and 1.04 (intergenic) on average, P<2.2*10⁻¹⁶, one-sided Wilcoxon rank-sum test, FIG. 8G). Of the 1,031 targeted genes, 544 were also among the 6,685 analyzed genes: the vast majority had 491 had a nominal negative effect on their own expression, 137 of them significantly (FDR<0.1, FIG. 8H), and only four had a significant positive effect. Expressed (detected) E3s affected significantly more genes than undetected ones (P<10⁻⁴, one-sided non-parametric Wilcoxon test, FIG. 8J) and the mean expression of the targeted (KO) gene in unperturbed cells was modestly but positively correlated with the number of genes impacted by their perturbation (Spearman's p=0.22, P<1.5*10⁻¹⁰, FIG. 8I). Overall, these results suggest that CRISPR-induced indels overall caused nonsense mediated decay (NMD) of the respective transcripts, for expressed E3s and family members, as well as with prior observations that some CRISPR-knockout generated indels having poor NMD (Dixit et al., Cell, 167: 1853-1866.e17, 2016; Parnas et al., Cell, 162: 675-686, 2015) or even (futile) transcriptional compensation (El-Brolosy et al., Nature, 568: 193-197, 2019).

### F. DC1-, DC2-, Migratory DC-, and Macrophage-Like Cells are Screened Jointly

BMDC populations are heterogeneous, and previous studies (Helft et al., Immunity, 42: 1197-1211, 2015; Maier et al., Nature, 580: 257-262, 2020; Shalek et al., Nature, 498: 236-240, 2013; Shalek et al., Nature, 510: 363-369, 2014; Villani et al., Science, 356(6335), 2017), including an earlier Perturb-Seq screen in this system (Dixit et al., Cell, 167: 1853-1866.e17, 2016) in this system all highlighted the presence of different subsets, including cells expressing macrophage-like signatures, and “cluster-disrupted” dendritic cells (DCs) (Jiang et al., Immunity, 27: 610-624, 2007; Shalek et al., Nature, 498: 236-240, 2013). Because Perturb-Seq characterizes any cell diversity post hoc, multiple phenotypes are assessed simultaneously (Dixit et al., Cell, 167: 1853-1866.e17, 2016).

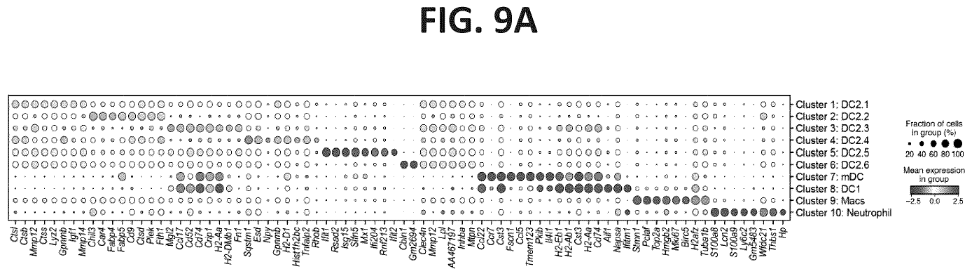

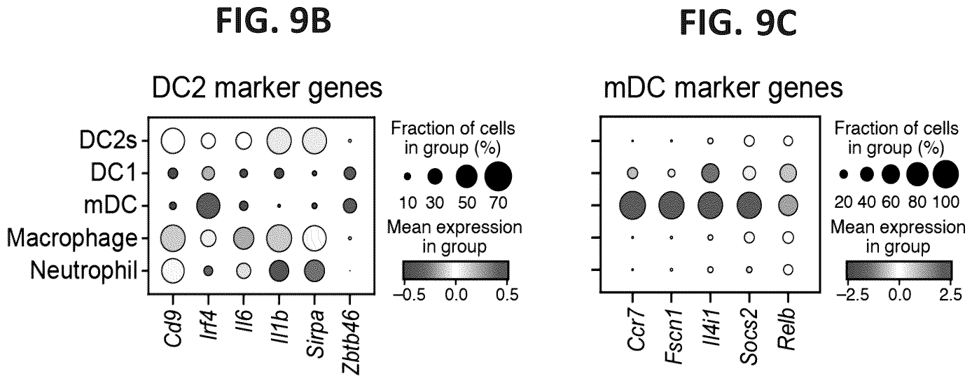

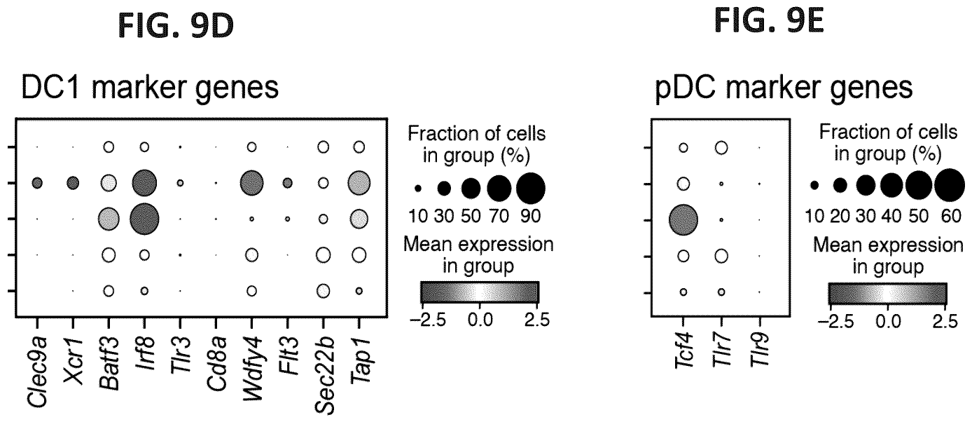

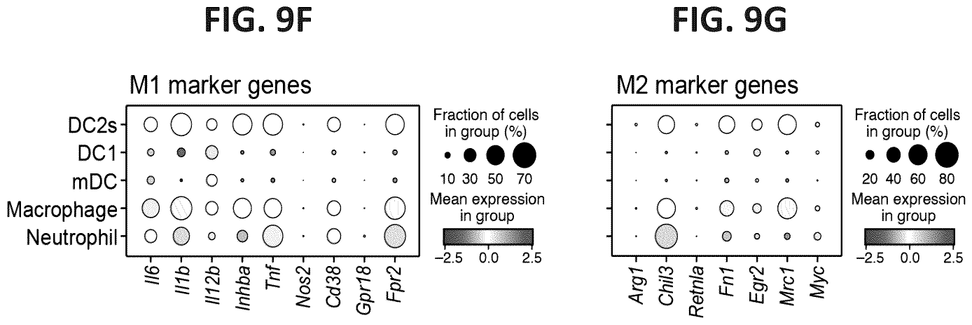

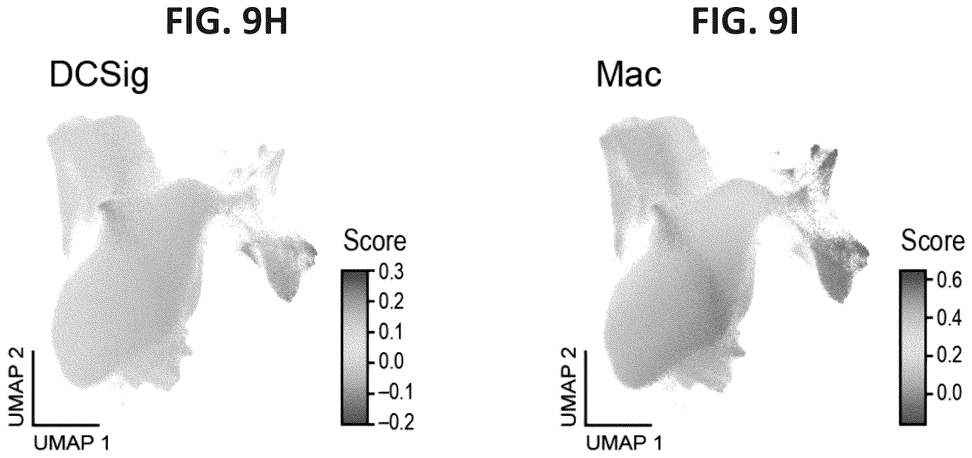

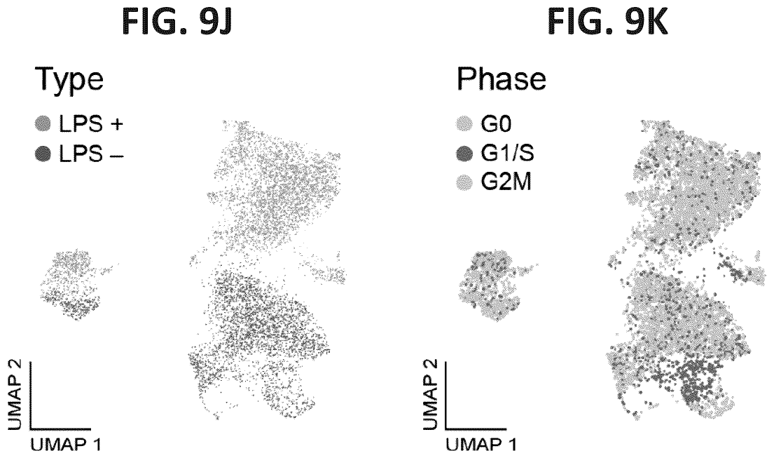

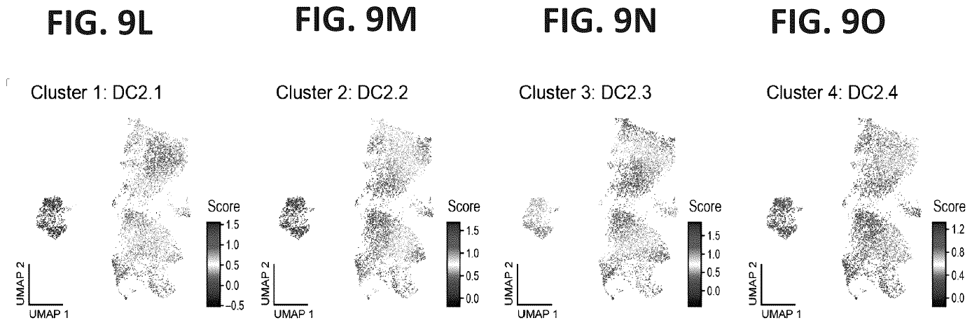

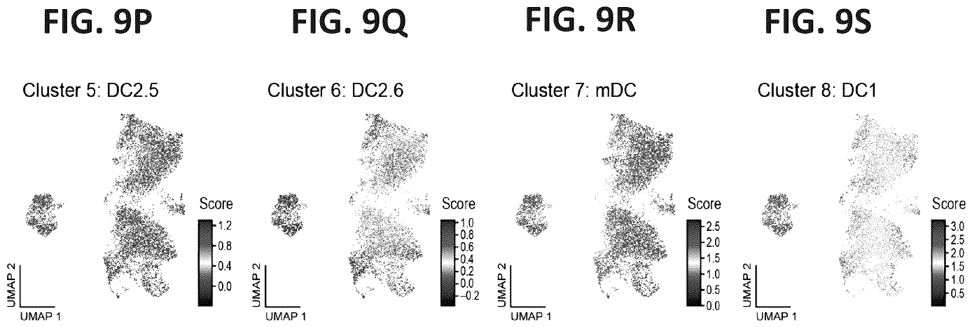

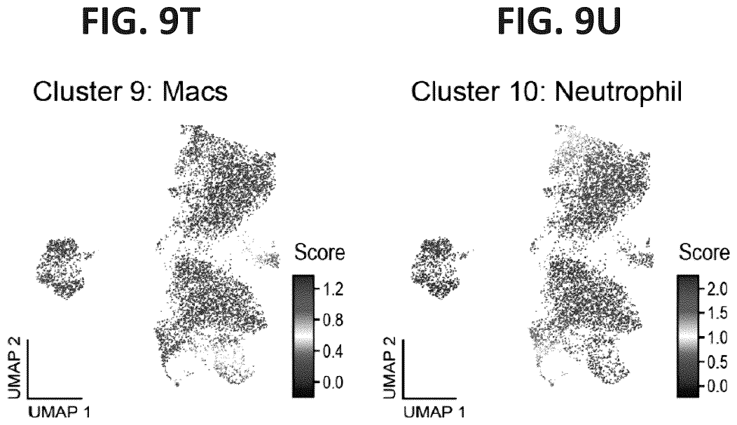

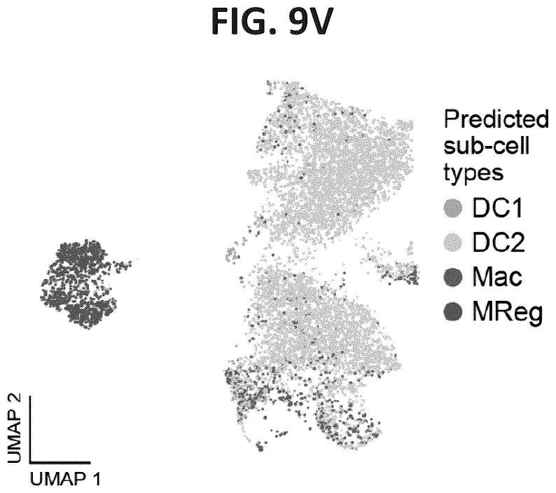

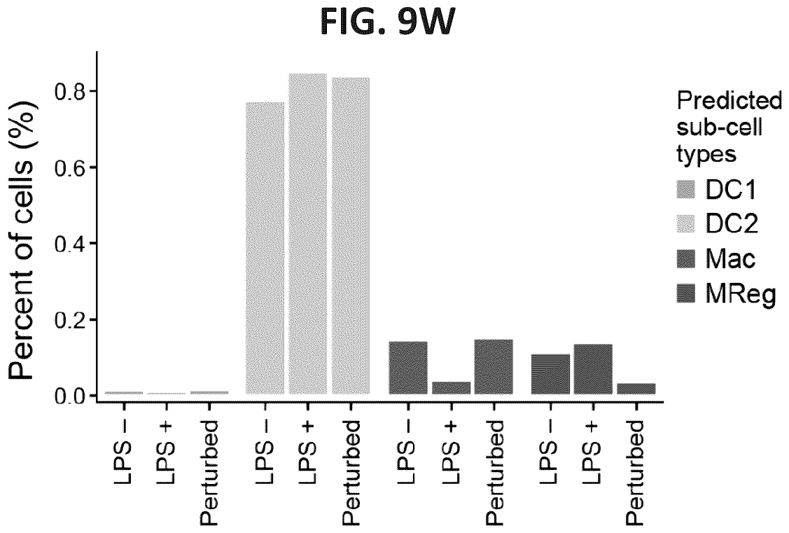

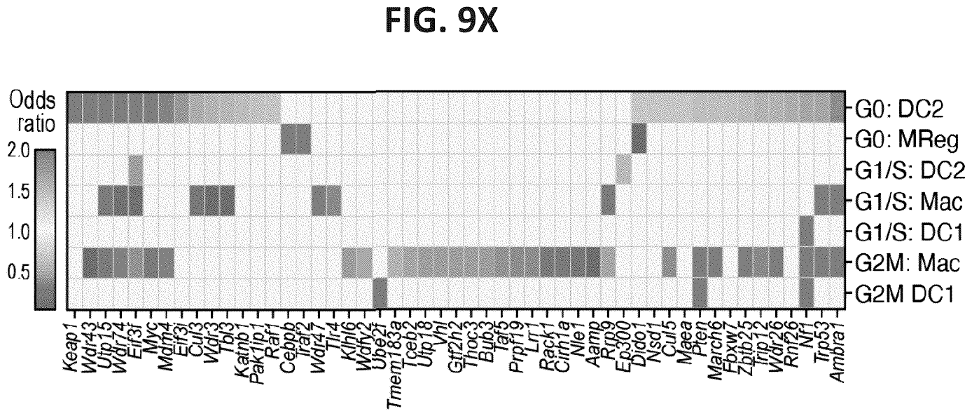

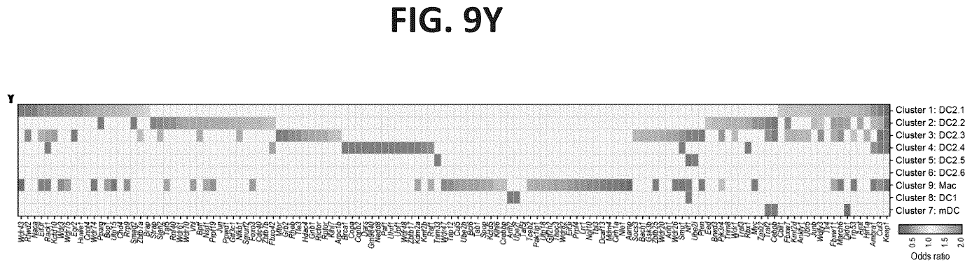

The 519,535 perturbed single cell profiles partitioned into ten clusters (FIGS. 1C-H and 9A and Table 4), which included multiple type 2 dendritic cell (DC2)-like subsets (high expression of Cd9, Ill b, and Sirpa (but also Irf4 and 116); FIGS. 1C and 1D, clusters 1-6; FIGS. 9A and 9B); migratory DC-like (mDC-like) cells (high expression of Ccr7, Fscn1, 114i, Socs2, and Relb (but not Pdl2); cluster 7, FIGS. 1C, 1E, FIGS. 9B, and 9D); DC1-like cells (high expression of Clec9a, Xcr1, Batf3, Irf8, Tap1, Flt3, and Wdfy4, but also Cd8a and Tlr3, and no expression of pDC marker genes (Tcf4, Tlr7, and Tlr9; additionally, Siglec-H was not among detected transcripts); FIGS. 1C, 1F, 9A, 9D, and 9E); and macrophage-like cells (high expression of M1 markers 116, Il1b, and Fpr2 and moderate expression of M2 markers Chil3, Fn1, and Mc1; FIGS. 1C, 9A, 9F, and 9G). The three main DC subsets also followed a gradient of expression from more mature DC-like (most prominent in migratory DCs (mDCs)) to macrophage-like (more prominent in some DC2s) signatures (FIGS. 1H, 9H, and 9I). Each DC2-like cell subset had additional distinguishing markers (FIG. 9A). Cycling cells (FIGS. 1C and 1G, Cluster 9, 13%) expressed signatures of either DC2s or macrophages/monocytes (FIGS. 1D, 1G, 1H, 9H, and 9I). These cycling monocyte-derived macrophages are consistent with previous reports (Erlich et al. Nat. Immunol., 20: 397-406, 2019; Helft et al., Immunity, 42: 1197-1211, 2015). A very small subset (460 cells; 0.08%) expressed a neutrophil signature (FIGS. 1C and 9A, cluster 10). DC2-like, DC1-like, mDC-like, and cycling monocyte-derived macrophages were also present in genetically unperturbed cells, with and without LPS stimulation (FIGS. 9J-9W), but at different proportions, with the fraction of macrophage-like cycling cells lower in genetically unperturbed LPS stimulated cells than in either unperturbed unstimulated DCs or perturbed stimulated DCs (FIG. 9W, P-value <2.2*10⁻¹⁶, one-sided Fisher's exact test), and the fraction of mDCs in unperturbed cells (stimulated and unstimulated) higher than in perturbed stimulated DCs (FIG. 9W, P-value <2.2*10⁻¹⁶, one-sided Fisher's exact test). The increase in monocyte-derived macrophages in genetically perturbed cells (FIG. 9W) suggests that the macrophage-like state is typically repressed by LPS stimulation but remains accessible upon perturbation.

TABLE 4
Top 100 Differentially Expressed Genes (Leiden Clusters)

| Group 0 | | | Group 1 | | | Group 2 | | | Group 3 | | |
|---|---|---|---|---|---|---|---|---|---|---|---|
| name | pvals | pvals_adj | name | pvals | pvals_adj | name | pvals | pvals_adj | name | pvals | pvals_adj |
| Ctsl | 0 | 0 | Chil3 | 0 | 0 | Mgl2 | 0 | 0 | Stmn1 | 0 | 0 |
| Ctsb | 0 | 0 | Car4 | 0 | 0 | Ccl17 | 0 | 0 | Pclaf | 0 | 0 |
| Mmp12 | 0 | 0 | Fabp4 | 0 | 0 | Cd52 | 0 | 0 | Top2a | 0 | 0 |
| Ctss | 0 | 0 | Fabp5 | 0 | 0 | Cd74 | 0 | 0 | Hmgb2 | 0 | 0 |
| Lyz2 | 0 | 0 | Cd9 | 0 | 0 | Crip1 | 0 | 0 | Mki67 | 0 | 0 |
| Gpnmb | 0 | 0 | Ctsd | 0 | 0 | H2-Aa | 0 | 0 | Birc5 | 0 | 0 |
| Igf1 | 0 | 0 | Plek | 0 | 0 | H2-DMb1 | 0 | 0 | H2afz | 0 | 0 |
| Mmp14 | 0 | 0 | Fth1 | 0 | 0 | Fn1 | 0 | 0 | Tuba1b | 0 | 0 |
| Acod1 | 0 | 0 | Wfdc21 | 0 | 0 | H2-Ab1 | 0 | 0 | Smc2 | 0 | 0 |
| Fam20c | 0 | 0 | Sgk1 | 0 | 0 | Ccr2 | 0 | 0 | Ube2c | 0 | 0 |
| Clec4n | 0 | 0 | Fpr1 | 0 | 0 | St3gal5 | 0 | 0 | Rrm1 | 0 | 0 |
| Wfdc17 | 0 | 0 | Atp6v0d2 | 0 | 0 | Dcstamp | 0 | 0 | Tubb5 | 0 | 0 |
| Msr1 | 0 | 0 | Ccl6 | 0 | 0 | AA467197 | 0 | 0 | Hmgb1 | 0 | 0 |
| Pf4 | 0 | 0 | Mt1 | 0 | 0 | Fcrls | 0 | 0 | Ptma | 0 | 0 |
| Abca1 | 0 | 0 | Hebp1 | 0 | 0 | Zyx | 0 | 0 | Ube2s | 0 | 0 |
| Lgmn | 0 | 0 | Ch25h | 0 | 0 | Adam8 | 0 | 0 | Cdca3 | 0 | 0 |
| Cxcl3 | 0 | 0 | Slc7a2 | 0 | 0 | Mmp12 | 0 | 0 | Prc1 | 0 | 0 |
| Gas7 | 0 | 0 | Bhlhe41 | 0 | 0 | H2-Eb1 | 0 | 0 | Cdk1 | 0 | 0 |
| Mmp8 | 0 | 0 | Phgdh | 0 | 0 | Ccr5 | 0 | 0 | Ccna2 | 0 | 0 |
| Cxcl2 | 0 | 0 | Sirpa | 0 | 0 | Vcan | 0 | 0 | Nucks1 | 0 | 0 |
| H2-M2 | 0 | 0 | Spp1 | 0 | 0 | Plxdc2 | 0 | 0 | Tpx2 | 0 | 0 |
| C1qb | 0 | 0 | Cd68 | 0 | 0 | Pfkp | 0 | 0 | Cdca8 | 0 | 0 |
| Nfe2l2 | 0 | 0 | Sdcbp | 0 | 0 | C1qb | 0 | 0 | Racgap1 | 0 | 0 |
| Alas1 | 0 | 0 | Gstm1 | 0 | 0 | Gm2a | 0 | 0 | Tmpo | 0 | 0 |
| Rnf149 | 0 | 0 | Cd93 | 0 | 0 | H2-DMa | 0 | 0 | Nusap1 | 0 | 0 |
| Apoe | 0 | 0 | Il18 | 0 | 0 | S100a6 | 0 | 0 | Cenpa | 0 | 0 |
| Cd38 | 0 | 0 | Ffar2 | 0 | 0 | Btg1 | 0 | 0 | Cenpe | 0 | 0 |
| Inhba | 0 | 0 | Klhdc4 | 0 | 0 | Plbd1 | 0 | 0 | Rrm2 | 0 | 0 |
| Bst1 | 0 | 0 | Mt2 | 0 | 0 | Rbpj | 0 | 0 | Cks1b | 0 | 0 |
| Pla2g7 | 0 | 0 | Mcemp1 | 0 | 0 | Gbp2 | 0 | 0 | Hist1h1b | 0 | 0 |
| Csf1r | 0 | 0 | Cyth3 | 0 | 0 | Id2 | 0 | 0 | Smc4 | 0 | 0 |
| Acpp | 0 | 0 | Bcap31 | 0 | 0 | Mbnl1 | 0 | 0 | Dek | 0 | 0 |
| Fcgr3 | 0 | 0 | Camk1 | 0 | 0 | Clec4n | 0 | 0 | Ccnb1 | 0 | 0 |
| Ccl4 | 0 | 0 | Cebpb | 0 | 0 | Srgn | 0 | 0 | Cenpf | 0 | 0 |
| C1qc | 0 | 0 | Cyfip2 | 0 | 0 | Cfp | 0 | 0 | Lmnb1 | 0 | 0 |
| Itgam | 0 | 0 | S100a8 | 0 | 0 | Sec61b | 0 | 0 | H2afv | 0 | 0 |
| Blvrb | 0 | 0 | Gpr137b | 0 | 0 | Ccnd2 | 0 | 0 | Tacc3 | 0 | 0 |
| Lrpap1 | 0 | 0 | Ppt2 | 0 | 0 | Ccdc80 | 0 | 0 | Selenoh | 0 | 0 |
| Met | 0 | 0 | Sla | 0 | 0 | Cd36 | 0 | 0 | Tubb4b | 0 | 0 |
| Trem2 | 0 | 0 | S100a1 | 0 | 0 | Hemk1 | 0 | 0 | Incenp | 0 | 0 |
| Cers6 | 0 | 0 | Marco | 0 | 0 | Adra2a | 0 | 0 | Spc25 | 0 | 0 |
| Cd14 | 0 | 0 | Sort1 | 0 | 0 | Slc2a6 | 0 | 0 | Cks2 | 0 | 0 |
| Clmp | 0 | 0 | Lox | 0 | 0 | Rnase6 | 0 | 0 | Ccdc34 | 0 | 0 |
| Rnase2a | 0 | 0 | Tlr2 | 0 | 0 | Ccl22 | 0 | 0 | Rad21 | 0 | 0 |
| Timp2 | 0 | 0 | Ndufa4 | 0 | 0 | Itgax | 0 | 0 | Kif23 | 0 | 0 |
| Thbs1 | 0 | 0 | Mrc1 | 0 | 0 | Tns1 | 0 | 0 | Ccnb2 | 0 | 0 |
| Plxdc2 | 0 | 0 | Cd300lf | 0 | 0 | Smdt1 | 0 | 0 | Ran | 0 | 0 |
| Serpinb2 | 0 | 0 | Unc119 | 0 | 0 | Plet1 | 0 | 0 | Kif11 | 0 | 0 |
| Acsl1 | 0 | 0 | Cdc42ep2 | 0 | 0 | Irf4 | 0 | 0 | Atad2 | 0 | 0 |
| Fam102b | 0 | 0 | Ncoa4 | 0 | 0 | Kcnn4 | 0 | 0 | Anp32b | 0 | 0 |
| Sdc1 | 0 | 0 | Hip1 | 0 | 0 | Palld | 0 | 0 | Anln | 0 | 0 |
| Rnf128 | 0 | 0 | Hivep3 | 0 | 0 | Herpud1 | 0 | 0 | Asf1b | 0 | 0 |
| Ccl2 | 0 | 0 | Camk2d | 0 | 0 | Pla2g7 | 0 | 0 | Cbx3 | 0 | 0 |
| Klk1b1 | 0 | 0 | Ly75 | 0 | 0 | Cxcl16 | 0 | 0 | Hmmr | 0 | 0 |
| Saa3 | 0 | 0 | 45178 | 0 | 0 | Mtdh | 0 | 0 | Kif15 | 0 | 0 |
| Fcgr2b | 0 | 0 | Adgre1 | 0 | 0 | Tarm1 | 0 | 0 | Cbx5 | 0 | 0 |
| Emb | 0 | 0 | Pdlim7 | 0 | 0 | Tnip3 | 0 | 0 | Spc24 | 0 | 0 |
| Fn1 | 0 | 0 | Dst | 0 | 0 | Tspan3 | 0 | 0 | Kif20b | 0 | 0 |
| Src | 0 | 0 | Ctsk | 0 | 0 | S100a4 | 0 | 0 | Ranbp1 | 0 | 0 |
| Cmklr1 | 0 | 0 | Gsn | 0 | 0 | Jak2 | 0 | 0 | Tyms | 0 | 0 |
| Acp5 | 0 | 0 | Iqsec1 | 0 | 0 | Rnase2a | 0 | 0 | Plk1 | 0 | 0 |
| Slfn5 | 0 | 0 | Plin2 | 0 | 0 | Clec4b1 | 0 | 0 | Rangap1 | 0 | 0 |
| C3 | 0 | 0 | Pid1 | 0 | 0 | Olfm1 | 0 | 0 | Ckap21 | 0 | 0 |
| Ninj1 | 0 | 0 | Gmpr | 0 | 0 | Pgap2 | 0 | 0 | Lig1 | 0 | 0 |
| Mmp13 | 0 | 0 | Prkar2b | 0 | 0 | Ptx3 | 0 | 0 | Anp32e | 0 | 0 |
| Clec4d | 0 | 0 | Epas1 | 0 | 0 | C1qa | 0 | 0 | Hnrnpab | 0 | 0 |
| Anpep | 0 | 0 | Nr1h3 | 0 | 0 | Irf5 | 0 | 0 | Mcm5 | 0 | 0 |
| C3ar1 | 0 | 0 | Ppp1r12b | 0 | 0 | Gbp3 | 0 | 0 | Fam111a | 0 | 0 |
| C1qa | 0 | 0 | Il17ra | 0 | 0 | Coro1a | 0 | 0 | Cenpq | 0 | 0 |
| Flrt3 | 0 | 0 | Hck | 0 | 0 | H2afy | 0 | 0 | Knl1 | 0 | 0 |
| Igsf6 | 0 | 0 | Fcor | 0 | 0 | C1qc | 0 | 0 | Mcm7 | 0 | 0 |
| Il7r | 0 | 0 | Cd33 | 0 | 0 | Ifi27l2a | 0 | 0 | Nap1l1 | 0 | 0 |
| Cd300c2 | 0 | 0 | Abcc5 | 0 | 0 | Slamf9 | 0 | 0 | Tuba1c | 0 | 0 |
| Adgre1 | 0 | 0 | Gdf15 | 0 | 0 | Sema6d | 0 | 0 | Diaph3 | 0 | 0 |
| Fyb | 0 | 0 | Psen2 | 0 | 0 | Cbfa2t3 | 0 | 0 | Mad2l1 | 0 | 0 |
| Gpr141 | 0 | 0 | Ppfia3 | 0 | 0 | Ltb4r1 | 0 | 0 | H3f3b | 0 | 0 |
| Slamf7 | 0 | 0 | Adipor2 | 0 | 0 | Etv3 | 0 | 0 | Ncapd2 | 0 | 0 |
| Ms4a6d | 0 | 0 | Plscr1 | 0 | 0 | Sema4a | 0 | 0 | Hjurp | 0 | 0 |
| Maf | 0 | 0 | Pparg | 0 | 0 | Itgam | 0 | 0 | Mcm3 | 0 | 0 |
| Pdpn | 0 | 0 | Slc39a2 | 0 | 0 | Cd200r1 | 0 | 0 | Gmnn | 0 | 0 |
| Plk2 | 0 | 0 | Ear1 | 0 | 0 | Pigx | 0 | 0 | Hist1h1e | 0 | 0 |
| C5ar1 | 0 | 0 | Al504432 | 0 | 0 | Clic4 | 0 | 0 | Uhrf1 | 0 | 0 |
| Rhob | 0 | 0 | Gngt2 | 0 | 0 | Zfp36l1 | 0 | 0 | Dut | 0 | 0 |
| Mertk | 0 | 0 | Fam181b | 0 | 0 | Rras2 | 0 | 0 | Fbxo5 | 0 | 0 |
| Rnase4 | 0 | 0 | Mctp1 | 0 | 0 | Pdcd1lg2 | 0 | 0 | Sgo2a | 0 | 0 |
| Ecm1 | 0 | 0 | Selenop | 0 | 0 | Cnn2 | 0 | 0 | Bub1b | 0 | 0 |
| Ms4a6c | 0 | 0 | Ltc4s | 0 | 0 | Ckb | 0 | 0 | Plk4 | 0 | 0 |
| Fam46c | 0 | 0 | Galnt3 | 0 | 0 | Socs6 | 0 | 0 | Bub1 | 0 | 0 |
| Ccl7 | 0 | 0 | Fcgr4 | 0 | 0 | Cd24a | 0 | 0 | Slfn9 | 0 | 0 |
| Itgb5 | 0 | 0 | Agap1 | 0 | 0 | Adam15 | 0 | 0 | Mis18bp1 | 0 | 0 |
| Lpcat2 | 0 | 0 | Acaa2 | 0 | 0 | Vdr | 0 | 0 | Rad51ap1 | 0 | 0 |
| Mcoln2 | 0 | 0 | Perp | 0 | 0 | Gbp7 | 0 | 0 | Nuf2 | 0 | 0 |
| Apbb2 | 0 | 0 | Id1 | 0 | 0 | Blnk | 0 | 0 | Ncapg | 0 | 0 |
| Ifi204 | 0 | 0 | Aldoc | 0 | 0 | Retnla | 0 | 0 | Rpa2 | 0 | 0 |
| Itga1 | 0 | 0 | Serpinb1a | 0 | 0 | Tmem167 | 0 | 0 | Dbf4 | 0 | 0 |
| Cxcl1 | 0 | 0 | B3gnt7 | 0 | 0 | Lrrc32 | 0 | 0 | Ezh2 | 0 | 0 |
| Ppfibp2 | 0 | 0 | Ttyh2 | 0 | 0 | Cyth4 | 0 | 0 | C330027C09Rik | 0 | 0 |
| Arg1 | 0 | 0 | Tbc1d2 | 0 | 0 | Chst7 | 0 | 0 | Aurka | 0 | 0 |
| Ifi211 | 0 | 0 | Syngr1 | 0 | 0 | Mcub | 0 | 0 | Iqgap3 | 0 | 0 |
| Epha4 | 0 | 0 | Lpcat3 | 0 | 0 | Clec2l | 0 | 0 | Kif20a | 0 | 0 |

| | | |
|---|---|---|
| Group 4 | Group 5 | Group 6 |

| name | pvals | pvals_adj | name | pvals | pvals_adj | name |
|---|---|---|---|---|---|---|
| Sqstm1 | 0 | 0 | Ccl22 | 0 | 0 | Ifit1 |
| Esd | 0 | 0 | Ccr7 | 0 | 0 | Rsad2 |
| Npy | 0 | 0 | Cst3 | 0 | 0 | Isg15 |
| Gpnmb | 0 | 0 | Fscn1 | 0 | 0 | Slfn5 |
| H2-D1 | 0 | 0 | Ccl5 | 0 | 0 | Mx1 |
| Hist1h2bc | 0 | 0 | Tmem123 | 0 | 0 | Ifi204 |
| Tnfaip2 | 0 | 0 | Pkib | 0 | 0 | Rnf213 |
| Rhob | 0 | 0 | Il4i1 | 0 | 0 | Ifit2 |
| Prdx1 | 0 | 0 | Rpsa | 0 | 0 | Cmpk2 |
| Grina | 0 | 0 | Serpinb9 | 0 | 0 | Usp18 |
| Clec4d | 0 | 0 | Cd86 | 0 | 0 | Ifi209 |
| Bcl2l11 | 0 | 0 | Serpinb6b | 0 | 0 | Parp14 |
| Cav1 | 0 | 0 | Cacnb3 | 0 | 0 | Trim30a |
| Icam1 | 0 | 0 | Basp1 | 0 | 0 | Ifi47 |
| Creg1 | 0 | 0 | Fau | 0 | 0 | Oasl2 |
| Ampd3 | 0 | 0 | Cd74 | 0 | 0 | Stat1 |
| Ctsl | 0 | 0 | Irf8 | 0 | 0 | Cxcl10 |
| Mgst1 | 0 | 0 | Socs2 | 0 | 0 | Ifi203 |
| Gclm | 0 | 0 | Samsn1 | 0 | 0 | Ifi207 |
| Serpinb6a | 0 | 0 | Tmem176a | 0 | 0 | Gm4951 |
| Ctsb | 0 | 0 | Glipr2 | 0 | 0 | Rtp4 |
| Gpr137b | 0 | 0 | Net1 | 0 | 0 | Ifih1 |
| Ddit3 | 0 | 0 | Rps19 | 0 | 0 | Slfn8 |
| Srxn1 | 0 | 0 | Rpl27a | 0 | 0 | Herc6 |
| Cstb | 0 | 0 | Rps11 | 0 | 0 | Irgm1 |
| Rnf128 | 0 | 0 | Ogfrl1 | 0 | 0 | Ddx58 |
| Ccr12 | 0 | 0 | Wnk1 | 0 | 0 | Gbp5 |
| Aldh2 | 0 | 0 | Tmsb4x | 0 | 0 | Stat2 |
| Atf5 | 0 | 0 | H2-Eb1 | 0 | 0 | Acod1 |
| Gadd45b | 0 | 0 | Rpl15 | 0 | 0 | Pnp |
| Cxcl1 | 0 | 0 | Fam129a | 0 | 0 | Trim30d |
| Ifrd1 | 0 | 0 | Rpl37 | 0 | 0 | Samhd1 |
| Cd68 | 0 | 0 | Lsp1 | 0 | 0 | Znfx1 |
| Ahnak2 | 0 | 0 | Malat1 | 0 | 0 | Gbp2 |
| Csf1 | 0 | 0 | Rps14 | 0 | 0 | Ifitm3 |
| Syngr1 | 0 | 0 | Tmem176b | 0 | 0 | Sp100 |
| Trib3 | 0 | 0 | Rpl34 | 0 | 0 | Daxx |
| Nupr1 | 0 | 0 | Cytip | 0 | 0 | Trim25 |
| Prr13 | 0 | 0 | Marcksl1 | 0 | 0 | Irf7 |
| Smpdl3a | 0 | 0 | Cnn2 | 0 | 0 | Ifi211 |
| Slc3a2 | 0 | 0 | Rpl19 | 0 | 0 | Ifit3 |
| Atp6v0d2 | 0 | 0 | Psmb8 | 0 | 0 | 9930111J21Rik2 |
| H2-Q7 | 0 | 0 | Rps27 | 0 | 0 | Phf11b |
| Clec4e | 0 | 0 | Rps24 | 0 | 0 | Nt5c3 |
| Cdkn1a | 0 | 0 | Rpl26 | 0 | 0 | Eif2ak2 |
| Asns | 0 | 0 | H2-Aa | 0 | 0 | Oas3 |
| Gadd45a | 0 | 0 | Rgs10 | 0 | 0 | Cd69 |
| H2-Q6 | 0 | 0 | Tnfrsf9 | 0 | 0 | Dtx3l |
| Mt2 | 0 | 0 | Ppdpf | 0 | 0 | Gbp7 |
| Atf4 | 0 | 0 | Nudt17 | 0 | 0 | Tap1 |
| Ppp1r15a | 0 | 0 | Bmp2k | 0 | 0 | AW112010 |
| Plin2 | 0 | 0 | Rpl21 | 0 | 0 | Oasl1 |
| Tax1bp1 | 0 | 0 | Gm13546 | 0 | 0 | Ifi44 |
| Scpep1 | 0 | 0 | Rpl28 | 0 | 0 | Mndal |
| Nfkbiz | 0 | 0 | Rps10 | 0 | 0 | Fam46a |
| Plk2 | 0 | 0 | Rpl30 | 0 | 0 | Samd9l |
| Ghitm | 0 | 0 | Rps20 | 0 | 0 | Tor1aip1 |
| Cd300a | 0 | 0 | Rpl18 | 0 | 0 | Xaf1 |
| Bcor | 0 | 0 | Rogdi | 0 | 0 | Slfn1 |
| Il7r | 0 | 0 | Rpl18a | 0 | 0 | Ddx60 |
| Plpp3 | 0 | 0 | Rpl17 | 0 | 0 | Gbp3 |
| Cat | 0 | 0 | Atrx | 0 | 0 | Etnk1 |
| Casp4 | 0 | 0 | Zfp36l1 | 0 | 0 | Bst2 |
| Gabarapl1 | 0 | 0 | Rps15a | 0 | 0 | Zufsp |
| Scarb2 | 0 | 0 | Swap70 | 0 | 0 | Tor3a |
| Slpi | 0 | 0 | Rps7 | 0 | 0 | Irgm2 |
| Cnppd1 | 0 | 0 | Psme2 | 0 | 0 | Ifi206 |
| Ero1l | 0 | 0 | Cd83 | 0 | 0 | Socs1 |
| Ier5 | 0 | 0 | Rpl11 | 0 | 0 | Igtp |
| Hspa9 | 0 | 0 | Tspo | 0 | 0 | Zbp1 |
| Cd83 | 0 | 0 | Srgn | 0 | 0 | Ccl4 |
| Dtx4 | 0 | 0 | Traf1 | 0 | 0 | Parp9 |
| Cox6a2 | 0 | 0 | Zfand6 | 0 | 0 | Cd274 |
| Gns | 0 | 0 | Rpl9 | 0 | 0 | Gpr141 |
| Map1lc3b | 0 | 0 | Rpl23 | 0 | 0 | Lgals9 |
| Txnip | 0 | 0 | Rps5 | 0 | 0 | C1qb |
| Acsl1 | 0 | 0 | Psme1 | 0 | 0 | Zc3hav1 |
| Gdf15 | 0 | 0 | Rasa2 | 0 | 0 | Ifi205 |
| Gla | 0 | 0 | Rpl39 | 0 | 0 | Rasa4 |
| Slc6a8 | 0 | 0 | Ktn1 | 0 | 0 | Parp12 |
| Mylip | 0 | 0 | Actb | 0 | 0 | Tapbp |
| Emp1 | 0 | 0 | Gnb4 | 0 | 0 | Sap30 |
| Rtn4 | 0 | 0 | Rps18 | 0 | 0 | Gbp4 |
| Gstm1 | 0 | 0 | Ikzf4 | 0 | 0 | Irf1 |
| Slc11a1 | 0 | 0 | Pdcd4 | 0 | 0 | Sp140 |
| Rabac1 | 0 | 0 | Rpl35a | 0 | 0 | Themis2 |
| Rhoc | 0 | 0 | Crip1 | 0 | 0 | Helz2 |
| Ypel3 | 0 | 0 | Rabgap1l | 0 | 0 | Trafd1 |
| Maoa | 0 | 0 | Rps23 | 0 | 0 | Tortaip2 |
| Tlcd2 | 0 | 0 | Rps9 | 0 | 0 | Clec2d |
| Procr | 0 | 0 | Rpl24 | 0 | 0 | Cd52 |
| Ednrb | 0 | 0 | Ccl17 | 0 | 0 | Ccl2 |
| Cyb5r1 | 0 | 0 | Gatsl2 | 0 | 0 | C1qc |
| Eif4ebp1 | 0 | 0 | Rpl7 | 0 | 0 | Fcgr1 |
| Arrdc4 | 0 | 0 | Rps4x | 0 | 0 | H2-T22 |
| Gclc | 0 | 0 | Gadd45b | 0 | 0 | Usp25 |
| Aars | 0 | 0 | Gyg | 0 | 0 | Phf11d |
| Chic2 | 0 | 0 | Mir155hg | 0 | 0 | 2810474O19Rik |
| Rragd | 0 | 0 | Rpl10 | 0 | 0 | Ehd4 |
| Gss | 0 | 0 | Cd40 | 0 | 0 | Ccnd2 |

| Group 6 | | Group 7 | | |
|---|---|---|---|---|
| pvals | pvals_ad | name | pvals | pvals_adj |
| 0 | 0 | Cbln1 | 0 | 0 |
| 0 | 0 | Gm2694 | 0 | 0 |
| 0 | 0 | Mmp12 | 1.6E−180 | 5.5E−177 |
| 0 | 0 | Inhba | 1.1E−136 | 1.7E−133 |
| 0 | 0 | Rnase2a | 4.83E−90 | 2.02E−87 |
| 0 | 0 | Cxcl3 | 2.57E−85 | 8.86E−83 |
| 0 | 0 | Dcstamp | 2E−82 | 6.59E−80 |
| 0 | 0 | Plet1 | 1.15E−80 | 3.53E−78 |
| 0 | 0 | H2-M2 | 8.65E−79 | 2.44E−76 |
| 0 | 0 | St3gal5 | 6.6E−76 | 1.69E−73 |
| 0 | 0 | Fn1 | 1.94E−67 | 4.25E−65 |
| 0 | 0 | Cav1 | 1.67E−55 | 2.96E−53 |
| 0 | 0 | Pf4 | 9.09E−52 | 1.34E−49 |
| 0 | 0 | Mag | 3E−16 | 8.28E−15 |
| 0 | 0 | Pcdh7 | 3.16E−13 | 6.88E−12 |
| 0 | 0 | Spink2 | 9.72E−12 | 1.82E−10 |
| 0 | 0 | Fst | 1.38E−11 | 2.54E−10 |
| 0 | 0 | F3 | 1.27E−10 | 2.14E−09 |
| 0 | 0 | Satb2 | 1.18E−06 | 1.27E−05 |
| 0 | 0 | Colec12 | 9.31E−06 | 8.76E−05 |
| 0 | 0 | Galnt9 | 9.55E−06 | 8.95E−05 |
| 0 | 0 | Sobp | 3.55E−05 | 0.000302 |
| 0 | 0 | Gm33251 | 0.000149 | 0.00113 |
| 0 | 0 | 44988 | 0.001008 | 0.006341 |
| 0 | 0 | Mafa | 0.001432 | 0.008619 |
| 0 | 0 | Rhobtb1 | 0.002126 | 0.012198 |
| 0 | 0 | Olfr110 | 0.002322 | 0.013209 |
| 0 | 0 | Cited4 | 0.00474 | 0.024492 |
| 0 | 0 | Rpl39l | 0.010296 | 0.047134 |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 0 | 0 | | | |
| 9.8537140940167E−310 | 1.5E−307 | | | |
| 0 | 2.4E−307 | | | |
| 5.8E−297 | 8.8E−295 | | | |
| 9E−291 | 1.3E−288 | | | |
| 2.1E−288 | 3.1E−286 | | | |
| 1.5E−287 | 2.2E−285 | | | |
| 1E−287 | 1.5E−285 | | | |
| 1.6E−285 | 2.3E−283 | | | |
| 3.7E−280 | 5.2E−278 | | | |
| 4.2E−280 | 5.9E−278 | | | |
| 4.3E−275 | 5.9E−273 | | | |
| 1.2E−275 | 1.7E−273 | | | |
| 3E−275 | 4.1E−273 | | | |
| 1.2E−268 | 1.6E−266 | | | |

| | |
|---|---|
| Group 8 | Group 9 |

| name | pvals | pvals_adj | name | pvals | pvals_adj |
|---|---|---|---|---|---|
| H2-Eb1 | 0 | 0 | S100a8 | 0 | ##### |
| H2-Ab1 | 0 | 0 | Lcn2 | 0 | ##### |
| Cst3 | 0 | 0 | S100a9 | 0 | 4E−92 |
| H2-Aa | 0 | 0 | Ly6c2 | 0 | 2E−89 |
| Cd74 | 0 | 0 | Gm5483 | 0 | 7E−80 |
| Aif1 | 0 | 0 | Wfdc21 | 0 | 3E−80 |
| Napsa | 0 | 0 | Thbs1 | 0 | 2E−71 |
| Ifitm1 | 0 | 0 | Hp | 0 | 1E−52 |
| Rps11 | 0 | 0 | Ifitm1 | 0 | 3E−41 |
| Rpsa | 0 | 0 | 2010005H15Rik | 0 | 5E−41 |
| Ciita | 0 | 0 | F630028O10Rik | 0 | 3E−40 |
| Ccl17 | 0 | 0 | Dgat2 | 0 | 5E−38 |
| Jaml | 0 | 0 | Sod2 | 0 | 7E−41 |
| Ccl22 | 0 | 0 | Gal | 0 | 7E−38 |
| Rps24 | 0 | 0 | Stfa2l1 | 0 | 8E−35 |
| Zfp36l1 | 0 | 0 | Ifitm3 | 0 | 7E−34 |
| Rps8 | 0 | 0 | S100a6 | 0 | 4E−34 |
| Rpl35a | 0 | 0 | Pglyrp1 | 0 | 7E−31 |
| Rpl18a | 0 | 0 | Coro1a | 0 | 6E−33 |
| Rps27a | 0 | 0 | Upp1 | 0 | 5E−31 |
| Rps18 | 0 | 0 | Cebpb | 0 | 2E−30 |
| Rpl27a | 0 | 0 | Lst1 | 0 | 7E−30 |
| Rps15a | 0 | 0 | BC100530 | 0 | 2E−26 |
| Rps7 | 0 | 0 | Il1f9 | 0 | 4E−26 |
| Rps3 | 0 | 0 | Tarm1 | 0 | 2E−26 |
| Rpl21 | 0 | 0 | Ngp | 0 | 1E−24 |
| Fau | 0 | 0 | Vasp | 0 | 3E−26 |
| Rplp0 | 0 | 0 | C3 | 0 | 6E−25 |
| Rps16 | 0 | 0 | Ltb | 0 | 1E−22 |
| Rps6 | 0 | 0 | Ncf1 | 0 | 5E−23 |
| Rps4X | 0 | 0 | Mmp8 | 0 | 1E−22 |
| Rpl9 | 0 | 0 | Mmp9 | 0 | 4E−21 |
| Rpl28 | 0 | 0 | Cd177 | 0 | 1E−20 |
| Rps20 | 0 | 0 | Stfa2 | 0 | 1E−20 |
| Rps3a1 | 0 | 0 | Ffar2 | 0 | 5E−20 |
| Atox1 | 0 | 0 | Mcemp1 | 0 | 7E−20 |
| Adam23 | 0 | 0 | Gsr | 0 | 8E−20 |
| Rps5 | 0 | 0 | Gpr84 | 0 | 4E−19 |
| Rpl13 | 0 | 0 | Stfa3 | 0 | 3E−18 |
| Rpl17 | 0 | 0 | Ckap4 | 0 | 2E−18 |
| Rpl18 | 0 | 0 | H3f3b | 0 | 7E−18 |
| Rps23 | 0 | 0 | Tuba4a | 0 | 1E−17 |
| Rpl32 | 0 | 0 | Arhgdib | 0 | 8E−18 |
| Rpl15 | 0 | 0 | Il1r2 | 0 | 6E−17 |
| Psmb8 | 0 | 0 | Ifitm6 | 0 | 1E−16 |
| Syngr2 | 0 | 0 | Ebi3 | 0 | 4E−17 |
| Tmsb4x | 0 | 0 | Ppp1cb | 0 | 4E−17 |
| Rpl11 | 0 | 0 | Gmfg | 0 | 4E−17 |
| Rps14 | 0 | 0 | Trem3 | 0 | 2E−16 |
| Rps10 | 0 | 0 | Chil1 | 0 | 3E−16 |
| Rpl3 | 0 | 0 | Ms4a3 | 0 | 4E−16 |
| Rpl26 | 0 | 0 | Mxd1 | 0 | 3E−16 |
| Ckb | 0 | 0 | Prdx5 | 0 | 1E−16 |
| Rpl30 | 0 | 0 | Siglece | 0 | 5E−16 |
| Rps19 | 0 | 0 | Ltf | 0 | 3E−15 |
| Rps12 | 0 | 0 | Mgst2 | 0 | 2E−15 |
| Rpl8 | 0 | 0 | Sorl1 | 0 | 2E−15 |
| Rpl12 | 0 | 0 | Spata13 | 0 | 2E−15 |
| H2-DMb2 | 0 | 0 | Plac8 | 0 | 7E−15 |
| Rps13 | 0 | 0 | Slpi | 0 | 3E−15 |
| Rpl14 | 0 | 0 | Mapkapk2 | 0 | 2E−15 |
| Cbfa2t3 | 0 | 0 | Ndufa4 | 0 | 3E−15 |
| Cd52 | 0 | 0 | Il1rn | 0 | 3E−15 |
| Eef1b2 | 0 | 0 | Fpr1 | 0 | 6E−15 |
| Rps25 | 0 | 0 | Fbx15 | 0 | 9E−15 |
| Coro1a | 0 | 0 | Ncf4 | 0 | 2E−14 |
| Cldn1 | 0 | 0 | Camp | 0 | 9E−14 |
| Rpl5 | 0 | 0 | Rac2 | 0 | 4E−14 |
| Rpl23 | 0 | 0 | Ikbke | 0 | 5E−14 |
| Rpl34 | 0 | 0 | Stx11 | 0 | 9E−14 |
| Rpl19 | 0 | 0 | Chil3 | 0 | 5E−14 |
| Irf8 | 0 | 0 | Pou2f2 | 0 | 7E−14 |
| Cd86 | 0 | 0 | Hmgb2 | 0 | 2E−13 |
| Rpl10 | 0 | 0 | Limd2 | 0 | 4E−13 |
| Rpl7 | 0 | 0 | Stfa1 | 0 | 7E−13 |
| Pkib | 0 | 0 | Zfp36l1 | 0 | 3E−13 |
| Etv3 | 0 | 0 | Ptpn6 | 0 | 5E−12 |
| Tpt1 | 0 | 0 | Myl10 | 0 | 1E−11 |
| Plbd1 | 0 | 0 | Id1 | 0 | 8E−12 |
| Malat1 | 0 | 0 | Fcnb | 0 | 2E−11 |
| Rpl10a | 0 | 0 | Napsa | 0 | 8E−12 |
| Rps15 | 0 | 0 | Serpinb1a | 0 | 3E−11 |
| Rpl37 | 0 | 0 | Qsox1 | 0 | 3E−11 |
| Rps27 | 0 | 0 | G0s2 | 0 | 5E−11 |
| Rpl4 | 0 | 0 | Ceacam10 | 0 | 8E−11 |
| S100a11 | 0 | 0 | Mapk13 | 0 | 1E−10 |
| Rack1 | 0 | 0 | Clec4d | 0 | 1E−10 |
| Lsp1 | 0 | 0 | Dach1 | 0 | 2E−10 |
| Rpl39 | 0 | 0 | Klf2 | 0 | 7E−10 |
| Basp1 | 0 | 0 | Csf3r | 0 | 1E−09 |
| Rps29 | 0 | 0 | Dstn | 0 | 8E−10 |
| Nedd4 | 0 | 0 | Fam49b | 0 | 1E−09 |
| Wdfy4 | 0 | 0 | Ly6g | 0 | 3E−09 |
| 2010005H15Rik | 0 | 0 | Gclm | 0 | 2E−09 |
| Ifitm3 | 0 | 0 | Asprv1 | 0 | 3E−09 |
| Sub1 | 0 | 0 | Sgms2 | 0 | 2E−09 |
| Rpl22 | 0 | 0 | Arg2 | 0 | 4E−09 |
| Eef1a1 | 0 | 0 | Pim2 | 0 | 5E−09 |
| Itgae | 0 | 0 | Raf1 | 0 | 5E−09 |
| Il4i1 | 0 | 0 | Tfrc | 0 | 6E−09 |

All p-values listed as 0 are significant.

### G. Conclusions: Scaled Genetic Perturbation and Interactions Screens

Several efficiencies were leveraged to enable Perturb-seq at scale, including hashing and overloading (40,000 cells per droplet channel; 5-fold cost reduction) and shallow sequencing (15,900 reads per cell on average; 2 fold reduction). Future efficiencies could include pre-barcoding (for even higher overloading (Datlinger et al., Nat. Methods, 18: 635-642, 2021)), cheaper sequencing (Simmons et al., Nat. Biotechnol., 1-8, 2022), and further guide-compressed screens (Cleary and Regev, The necessity and power of random, under-sampled experiments in biology, BioRxiv, 2020). For guide compression a larger number of perturbations per cell was initially aimed for, but these have been challenging to achieve in primary cells, and may require cells from CRISPRi (Gemberling et al., Transgenic mice for in vivo epigenome editing with CRISPR-based systems, BioRxiv, 2021) engineered mice.

The large scale of the present screen and the modular organization of the regulatory circuit opened a path to systematically tackle genetic interactions. First, the large-scale screen encompassed a relatively large number of cells with multiple perturbations per cell, but as this was a random sample, any particular combination was present in too few cells to directly estimate their effects. However, because of the organization of the regulators in co-functional modules, genetic interactions could be assessed at the level of modules, testing for the prevalence of significant inter- or intra-module interactions globally, as well as their impacts on individual genes. This analysis showed that intra-module interactions are far more prevalent than inter-module interactions and impact specific target processes. Consistently, the impact of most inter-module combinations of perturbations can be quite well predicted by a naïve additive (linear) model. Moreover, learning the interaction effects was improved by comβVAE, a conditional variational autoencoder that relies on the latent structure of the expression profiles was implemented. This shows the power of combining rich profiles and modular structures to allow prediction of unobserved experiments. Using a higher number of perturbations per cell, implementing ‘compressed screens’ ((Cleary and Regev, The necessity and power of random, under-sampled experiments in biology, BioRxiv, 2020)) and dedicated gene editing tools, such as Cas12 (Zetsche et al., Cell, 163: 759-771, 2015), should help further facilitate the dissection of genetic interactions.

### Example 2. Multiple E3 Ligases Impact Specific DC Cell Subsets or Differentiation

Sixty-five (65) genes assessed in the Perturb-seq screen of Example 1 were targeted by two or more guides that were significantly depleted from BMDCs vs. the input guide library distribution, suggesting that these genes are essential for BMDC survival and proliferation (Dixit et al., Cell, 167: 1853-1866.e17, 2016; Parnas et al., Cell, 162: 675-686, 2015) (P-value <0.05, considering a background of the corresponding change in control guides). Indeed, these were enriched for regulation of cell division (e.g., Aurka, Myc, Plk1, Pou5f1, Prc1, Tle6, Wdr5, Ybx1), including Mdm2 (all three guides depleted), an E3 ligase that ubiquitylates p53 as an active heterodimer with Mdm4 and is essential for cell cycle regulation (Chinnam et al., PLoS Genet., 18: e 1010171, 2022). Some perturbations affected the proportions of all cycling cells of a specific type (FIG. 9X), such as enrichment in cycling macrophages of guides targeting the tumor suppressor and E3 ligase substrate Trp53 and enrichment in cycling DC1-like cells of guides targeting the substrate Nf1, a tumor suppressor in myeloid cells that affects growth sensitivity to GM-CSF (Bollag et al., Nat. Genet., 12: 144-148, 1996) and regulates proliferation regulators (Dasgupta and Gutmann, J. Neurosci., 25: 5584-5594, 2005). Because different subsets were enriched for cycling cells (FIG. 1G), some of these perturbations also shifted subset proportions. For example, consistent with previous reports (Sharma et al., Immunity, 48: 91-106, e6, 2018), Trp53 KO were enriched in cycling monocyte-derived macrophages and depleted in non-cycling DC2s; Trp53's negative regulator E3 ligase Mdm4 had the opposite pattern (FIGS. 1J and 9X).

Perturbation in 64 genes, including 29 E3s and complex members, significantly affected the relative proportion of the main cell subsets, especially the balance of DC2s vs. cycling macrophage-like cells (FIGS. 1I and 1J; FDR <0.15, one-sided Fisher's exact test). Guides depleted in cycling macrophage-like cells vs. DC2s included the cullin E3 ligase Cul3 and its substrate adaptor Keap1, which ubiquitylates Nrf2 regulating redox response and inflammation (Saha et al., Mol. Basel Switz., 25: E5474, 2020; Zhang et al., Mol. Cell Biol., 24: 10941-10953, 2004); Vhl and Rack1, which together complex with Cul2 (Cai and Yang, Cell Div., 11(7), 2016) and ubiquitylate Hif1 a; and Mdm4, which interacts with Mdm2 to stimulate p53 ubiquitylation (Pant et al., Proc. Natl. Acad. Sci., 108: 11995-12000, 2011). Vhl, Cul3, Keap1 and Mdm4 were not previously described as regulators of DC2 differentiation, but deletion of Nrf2 has been shown to impact both tissue resident and BMDC functions (Williams et al., J. Immunol., 181: 4545-4559, 2008) and Rack1 depletion in myeloid cells has been shown to protect against viral infection in mice without affecting the number of F4/80+CD11b+ myeloid cells (Qin et al., J. Immunol., 207(5): 1411-1418, 2021). Conversely, guides enriched in cycling monocyte-derived macrophages vs. DC2s included those targeting Cul5, March6, and Wdr26, E3s not previously recognized as regulators of DC differentiation (FIGS. 1I and 1J).

Other enrichments and depletions further highlight the roles for E3s and related proteins in specific cell subsets. For example, mDCs were enriched for guides targeting the E3 ligase Traf2 and the transcription factor (TF) CEBPB and depleted for guides targeting DIDO. Traf2 plays a role in TNF-mediated NF-KB and MAP kinase signaling, and TNF injection can increase DC trafficking to lymph nodes (Martin-Fontecha et al., J. Exp. Med., 198: 615-621, 2003), suggesting a potential role for Traf2 in regulating DC migration. Enrichment of guides targeting CEBPB in mDCs is consistent with earlier findings (Dixit et al., Cell, 167: 1853-1866.e17, 2016) and CEBPB's natively low level in those cells (FIGS. 1I and 1J). Concomitantly, DC2.2s, where CEBPB expression is higher, are enriched for guides targeting Rfwd2 (COP1), which in turn targets CEBPB for degradation (Ndoja et al., Cell, 182: 1156-1169.e12, 2020). Conversely, guides targeting Dido1, a gene previously characterized only in embryonic stem cell differentiation (Futterer et al., Stem Cell Rep., 8: 1062-1075, 2017; Liu et al., J. Biol. Chem., 289: 4778-4786, 2014), were depleted in mDCs, suggesting a novel role in mDC differentiation (FIGS. 1I and 1J). In another example, guides targeting the E3 ligase Arih2, the E3 ligase substrate Nf1, and the E2 enzyme Ube2f were enriched in DC1s and macrophages and depleted in DC2s (FIGS. 1I and 1J). This is consistent with and expands on their established roles: Arih2 ubiquitylates substrates of Ube2f, the Nedd8 E2 that mediates neddylation of Cul5-Rbx2 (Kostrhon et al., Nat. Chem. Biol., 17: 1075-1083, 2021), causing degradation of the NF-KB inhibitor Ikbb in DCs (Lin et al., Nat. Immunol., 14: 27-33, 2013), and inactivating mutations in Nf1 lead to uncontrolled cell growth and plasmocytoid DC (pDC) neoplasms (Szczepaniak et al., Int. J. Hematol., 110: 102-106, 2019).

Many guides, including those targeting members of the WD-repeat protein subfamily, were specifically enriched in different DC2 subsets, including multiple members of a single complex in the same subset, suggesting that distinct DC2 subsets are controlled by different pathways (FDR <0.15, one-sided Fisher's exact test, FIGS. 1J and 9Y). For example, perturbations of each of the four members of the KEAPI:NEDD8-CUL3:RBX1 complex were specifically enriched in DC2.4s. DC2.3s were enriched for guides targeting E3 ligase substrates and members of the mTOR pathway, including mTORC2 (Mtor and Rictor) and mTORC1 (Mtor and Rptor), consistent with and refining the role of mTor signaling in regulating differentiation and immune functions in DCs (Sukhbaatar et al., Trends Immunol., 37: 778-789, 2016). DC2.2s were enriched for guides targeting Fbox E3 ligase component members, including Fbxo42, a regulator of the TAK1 pathway that activates p38 (Nagler et al., Pigment Cell Melanoma Res., 33: 334-344, 2020) and Fbxw7, a regulator of antiviral immunity in macrophages (Song et al., Nat. Commun., 8: 14654, 2017). DC2.1s were enriched for guides targeting Eif3f, Naca, Rfwd2 (Cop1), and Pparg. In addition to its role as a TF, Pparg is an E3 ligase that targets p65 (Hou et al., Nat. Commun., 3: 1300, 2012) and regulates lipid metabolism in DCs, leading to ferroptosis and impairing maturation (Han et al., Biochem. Biophys. Res. Commun., 576: 33-39, 2021), and DC-T cell interactions in type-2 immunity (Nobs et al., J. Exp. Med., 214: 3015-3035, 2017). DC2.5s were enriched for guides targeting Trim33, an E3 that interacts with Pu.1 (SP11) and regulates the NLRP3 inflammasome, LPS response, and macrophage activation (Gallouet et al., Oncotarget, 8: 5111-5122, 2017; Xue et al., Nat. Commun., 6: 6156, 2015), as well as for guides targeting Ube2i (Ubc9), an E2 SUMO-conjugating enzyme with a role in lupus, regulating sumoylation-based suppression of type I IFN and pDCs (Lu et al., Arthritis Rheumatol., 73: 1467-1477, 2021). Thus, regulators of six DC2 substates were identified, including multiple complex members similarly associated with the same state(s).

### A. Conclusions: E3s Regulate Key Phases of the DC Life Cycle

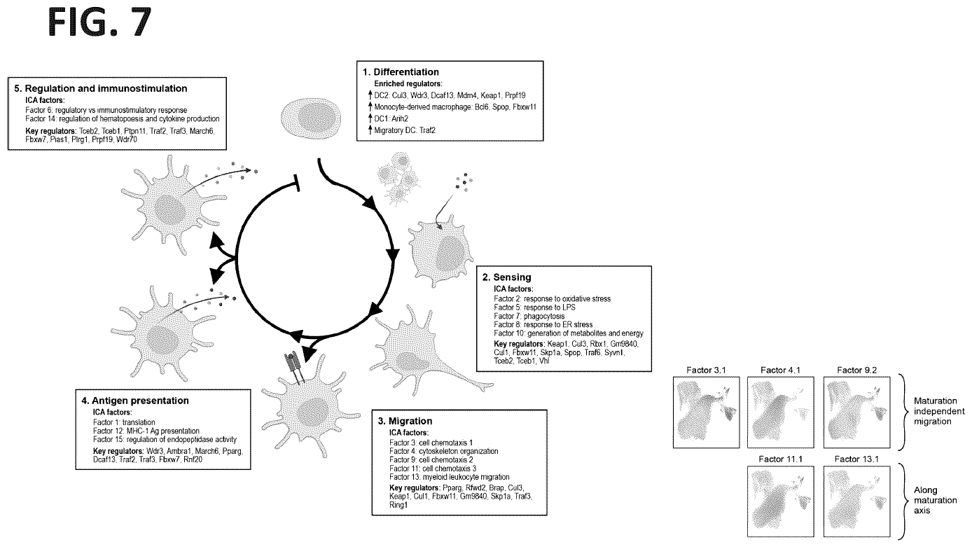

The independent factors and programs of target genes regulated by perturbations in E3s and associated proteins span multiple stages in DC life cycle (FIG. 7), showing the capacity of the present screen to capture many phenotypes and the breadth of roles E3s play in the immune response and the DC life cycle, many of which are novel roles. These include, in order of the lifecycle, regulation of: (1) differentiation towards DC2s (e.g., Cul3-Keap1), DC1 state (e.g., Arih2), and mDCs (e.g., Traf2); (2) sensing, including the response to LPS (factor #5), ER stress (#8), and oxidative stress (#2), ribonucleotide synthesis (#7), and metabolism and energy (#10), regulated by, e.g., the Keap1-Cul3-Rbx1 complex, the Tceb1-Tceb2-Rbxl-Vhl complex, and Traf6; (3) DC migration, including chemotaxis (#3,9,11), cytoskeleton organization (#4), and myeloid migration (#13), regulated by, e.g., Pparg, Rfwd2, Brap, and the Keap1-Cul3-Rbx1 complex; (4) antigen presentation and associated processes (antigen presentation (#12), endopeptidases (#15), and translation (#1)), regulated by, e.g., Ambra1, March6, Traf2, and Traf3; and (5) production of chemokines that promote either a regulatory or immunostimulatory response (#6.1, #6.2, #14), regulated by, e.g., Traf2, Traf3, March6, Pias1, Plgr1, Ptpn11, and Prpf19.

The detailed regulatory model highlights many novel regulatory relations and helps address open questions. For example, it has been unclear whether DC maturation and migration are inextricably linked (Flores-Romo, Immunology, 102: 255-262, 2001; Liu et al., Cell. Mol. Immunol., 18: 2461-2471, 2021). The model shows that while the expression of some migration factors (#11; #13) follow a cell maturation gradient, other factors (#3, #4, and #9) express migration genes independent of DC maturation. While the same regulators are shared across migration and maturation in some factors (e.g., Cul3-Keap1 and the CLR1 complex which co-regulate in Factor #4), they regulate them in opposite ways in others (e.g., Cul3-Keap1 and the CLR1 complex in Factor #9 or #13). This suggests that some, but not all, of the DC migration program is controlled independently of maturation. Several E3s are regulators of multiple programs along the DC lifecycle (e.g., Keap1-Cul3-Rbx1, Fbxw11-Cul1-Skp1 a, Fbxw7, March6), while others play specific roles in key stages (e.g., Rfwd2 (Cop1) in migration; Wdr70 in immunostimulatory vs. immunoregulatory response).

### Example 3. Six Co-Functional Modules of E3 Ligases Regulate Eleven Gene Programs

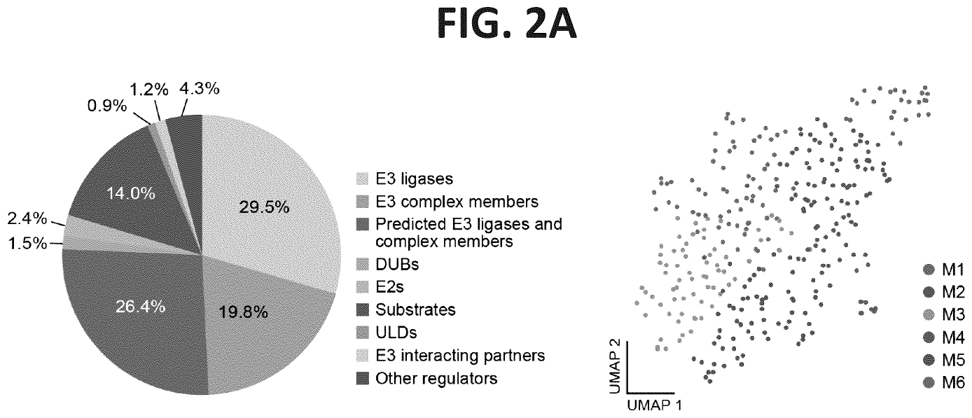

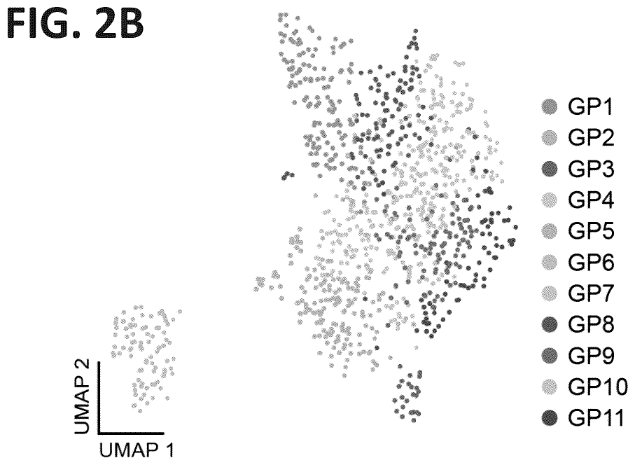

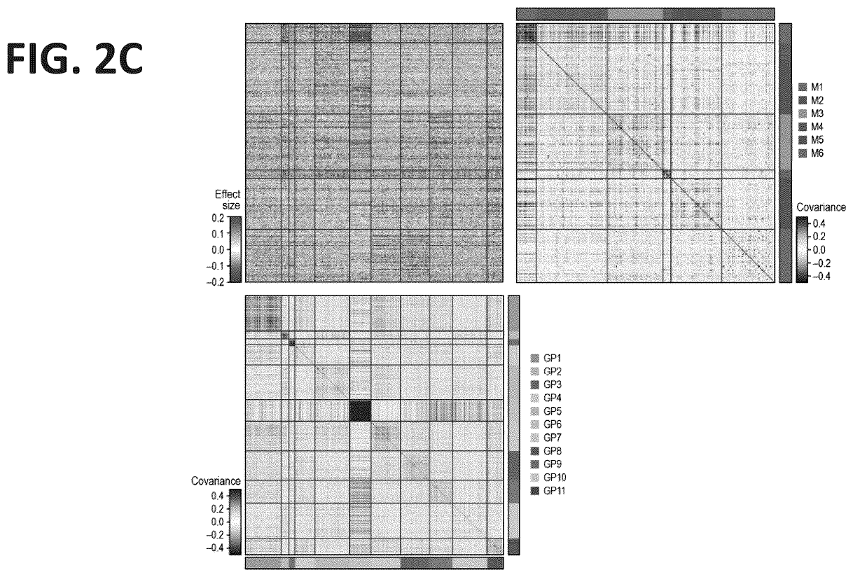

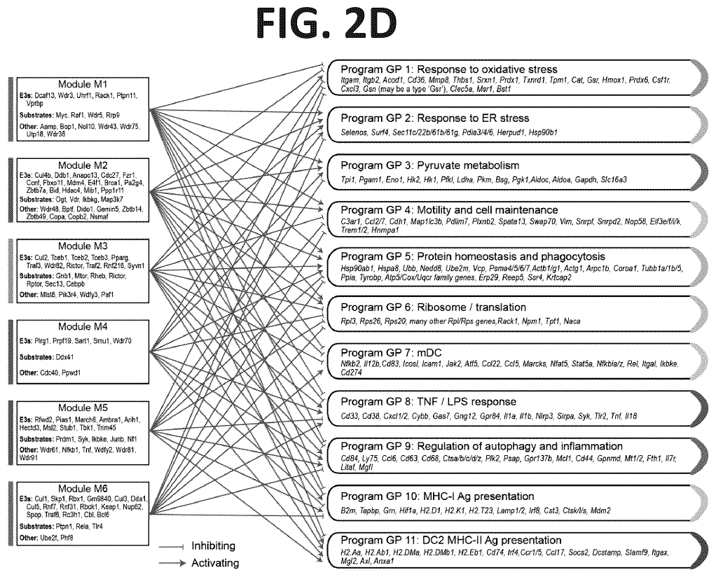

To relate the broad changes identified in the Perturb-seq screen of Example 1 to regulatory mechanisms, a regulatory model associating 329 impactful perturbed genes (affecting the level of at least 15 genes) to 1,041 significantly impacted targets (affected by at least four of the 329 perturbations) was learned and the perturbed genes and impacted targets were clustered into six co-functional gene modules (M1-M6) (Table 5) and eleven co-regulated gene programs (GP1-GP11) (Table 6), respectively (FIGS. 2A-2D).

TABLE 5
Co-functional gene modules M1-M6

| Module | Members (shortlist) | Members (full list) |
|---|---|---|
| Module M1 | E3s: Dcaf13, Wdr3, Uhrf1, Rack1, Ptpn11, | Aamp, Bop1, Cirh1a, Dcaf13, Grb2, |
| | Vprbp | Myc, Nle1, Nol10, Pak1ip1, Ptpn11, |
| | Substrates: Myc, Raf1, Wdr5, Rrp9 | Rack1, Raf1, Rrp9, Taf5, Tbl3, Uhrf1, |
| | Other: Aamp, Bop1, Nol10, Wdr43, Wdr75, | Utp15, Utp18, Vprbp, Wdr3, Wdr36, |
| | Utp18, Wdr36 | Wdr43, Wdr5, Wdr74, Wdr75. |
| Module M2 | E3s: Cul4b, Ddb1, Anapc13, Cdc27, Fzr1, | Ago2, Ahr, Anapc13, Bach1, Baz1a, |
| | Ccnf, Fbxo11, Mdm4, E4f1, Brca1, Pa2g4, | Bid, Bptf, Brca1, Brwd3, Btbd1, Cblc, |
| | Zbtb7a, Bid, Hdac4, Mib1, Ppp1r11 | Ccnf, Cdc27, Cntn4, Copa, Copb2, |
| | Substrates: Ogt, Vdr, Ikbkg, Map3k7 | Coro1a, Cpne9, Cul4b, Ddb1, Dido1, |
| | Other: Wdr48, Bptf, Dido1, Gemin5, Zbtb14, | E4f1, Ecel1, Fbxl14, Fbxl5, Fbxo11, |
| | Zbtb49, Copa, Copb2, Nsmaf | Fbxo42, Fzr1, Gemin5, Gm10697, |
| | | Gm9117, Gtf2h2, Gtf3c1, Hdac4, |
| | | Hectd1, Ift122, Ikbkg, Ing2, Jun, |
| | | Katnb1, Kbtbd13, Kdm2a, Klhl23, |
| | | Klhl3, Kmt2b, LOC100861784, Lrr1, |
| | | Lrrc41, Map3k7, Mdm4, Mib1, Mkrn1, |
| | | Mnat1, Naca, Nsmaf, Ogt, Pa2g4, |
| | | Pcif1, Ppp1r11, Prc1, Ring1, Rnf128, |
| | | Rnf20, Rnf225, Rnf40, Siah1a, Siah2, |
| | | Taf3, Tdpoz2, Tmem183a, Tnfsf11, |
| | | Tradd, Traf3ip2, Trim35, Trim7, Tssc1, |
| | | Ttc3, Ube2n, Ufl1, Unkl, Upf1, Vdr, |
| | | Wdhd1, Wdr48, Wdr95, Wwp1, Ybx1, |
| | | Zbtb14, Zbtb49, Zbtb7a, Zmiz1. |
| Module M3 | E3s: Cul2, Tceb1, Tceb2, Tceb3, Pparg, | Akt1, Ankfy1, Apc, Arpc1b, Birc2, |
| | Traf3, Wdr82, Rictor, Traf2, Rnf216, Syvn1 | Bmi1, Bub3, Cacybp, Cebpb, Chd4, |
| | Substrates: Gnb1, Mtor, Rheb, Rictor, Rptor, | Crebbp, Cul2, Dars, Dcaf10, Dcaf4, |
| | Sec13, Cebpb | Eif3f, Eif3i, Ep300, Fbxl13, Fbxo28, |
| | Other: Mlst8, Pik3r4, Wdfy3, Paf1 | Fbxo3, Fbxw9, Gm13416, Gnb1, |
| | | Gnb2, Grb10, Klhl24, Klhl7, Kmt2c, |
| | | Kmt2d, Mapk14, Med8, Mlst8, Mtor, |
| | | Nosip, Paf1, Pik3r4, Pparg, Ppp2r2a, |
| | | Ppp2r2d, Preb, Rbbp4, Rbbp5, Rheb, |
| | | Rictor, Rnf10, Rnf113a1, Rnf135, |
| | | Rnf216, Rptor, Scap, Sec13, Sec31a, |
| | | Smad2, Syvn1, Taf5l, Traf2, Traf3, |
| | | Traf7, Trim24, Trp53, Ube2e1, |
| | | Ube2e3, Ube3c, Ufm1, Wdfy3, Wdr1, |
| | | Wdr82, Whsc1, Zbtb11. |
| Module M4 | E3s: Plrg1, Prpf19, Sart1, Smu1, Wdr70 | Cdc40, Ddx41, Plrg1, Ppil2, Ppwd1, |
| | Substrates: Ddx41 | Prpf19, Prpf4, Sart1, Smu1, Snrnp40, |
| | Other: Cdc40, Ppwd1 | Wdr70. |
| Module M5 | E3s: Rfwd2, Pias1, March6, Ambra1, Arih1, | Acaca, Ambra1, Amfr, Arih1, Cbll1, |
| | Hectd3, Msl2, Stub1, Tbk1, Trim45 | Cfap57, Cnot4, Cyld, Dcaf7, Det1, |
| | Substrates: Prdm1, Syk, Ikbke, Junb, Nf1 | Dpf2, Eed, Efcab8, Egr2, Fasn, Fbxw7, |
| | Other: Wdr61, Nfkb1, Tnf, Wdfy2, Wdr81, | Foxo3, Gsk3b, Hectd3, Hira, Icos, |
| | Wdr91 | Ifnar1, Ikbke, Ints12, Junb, Kat6a, |
| | | Kctd10, Kctd13, Kctd21, Kctd5, Klhl30, |
| | | Klhl6, Lztr1, March6, Msl2, Nf1, Nfkb1, |
| | | Nsd1, Patz1, Pias1, Prdm1, Pten, |
| | | Rfwd2, Rnf139, Socs3, Spag16, Strap, |
| | | Stub1, Syk, Tab1, Tank, Tbk1, Tnf, |
| | | Trim45, Trip12, Ube2j2, Wdfy2, Wdr61, |
| | | Wdr81, Wdr91, Zbtb25, Zfp106, Zfp91, |
| | | Zmiz2. |
| Module M6 | E3s: Cul1, Skp1, Rbx1, Gm9840, Cul3, | Ahctf1, Anapc11, Arih2, Arnt, Bcl6, |
| | Dda1, Cul5, Rnf7, Rnf31, Rbck1, Keap1, | Brap, Cbl, Cd28, Cstf1, Cul1, Cul3, |
| | Nup62, Spop, Traf6, Rc3h1, Cbl, Bcl6 | Cul5, Dda1, Fbxo33, Fbxw11, Fus, |
| | Substrates: Ptpn1, Rela, Tlr4 | Gm9840, Hif1a, Huwe1, Ing3, Kcmf1, |
| | Other: Ube2f, Phf8 | Kdm5c, Keap1, Maea, Mycbp2, |
| | | Nbeal1, Nedd8, Nup43, Nup62, Phf8, |
| | | Ptpn1, Rae1, Ranbp2, Rbbp6, Rbck1, |
| | | Rbx1, Rc3h1, Rela, Rlim, Rnf144a, |
| | | Rnf31, Rnf7, Seh1l, Skp1a, Spop, |
| | | Ssr3, Tbl1xr1, Tceb1, Tceb2, Tceb3, |
| | | Tdpoz5, Thoc3, Tlr4, Traf6, Trim28, |
| | | Trim33, Ube2d3, Ube2f, Ube2h, Ube2i, |
| | | Ubr4, Ubr5, Vhl, Wdr20, Wdr26, |
| | | Wdr33, Zbtb17, Zbtb7b. |

TABLE 6
Co-regulated gene programs GP1-GP11

| Program | Description | Members |
|---|---|---|
| Program GP1 | Response to oxidative stress | Itgam, Itgb2, Acod1, Cd36, Mmp8, Thbs1, |
| | | Srxn1, Prdx1, Txnrd1, Tpm1, Cat, Gsr, |
| | | Hmox1, Prdx6, Csf1r, Cxcl3, Gsn (may be a |
| | | type ‘Gsr’), Clec5a, Msr1, Bst1 |
| Program GP2 | Response to endoplasmic | Selenos, Surf4, Sec11c/22b/61b/61g, |
| | reticulum (ER) stress | Pdia3/4/6, Herpud1, Hsp90b1 |
| Program GP3 | Pyruvate metabolism | Tpi1, Pgam1, Eno1, Hk2, Hk1, Pfkl, Ldha, |
| | | Pkm, Bsg, Pgk1, Aldoc, Aldoa, Gapdh, |
| | | Slc16a3 |
| Program GP4 | Motility and cell maintenance | C3ar1, Ccl2/7, Cdh1, Map1lc3b, Pdlim7, |
| | | Plxnb2, Spata13, Swap70, Vim, Snrpf, |
| | | Snrpd2, Nop58, Eif3e/f/j/k, Trem1/2, Hnrnpa1 |
| Program GP5 | Protein homeostasis and | Hsp90ab1, Hspa8, Ubb, Nedd8, Ube2m, Vcp, |
| | phagocytosis | Psma4/5/6/7, Actb1/g1, Actg1, Arpc1b, |
| | | Coroa1, Tubb1a/1b/5, Ppia, Tyrobp, |
| | | Atp5/Cox/Uqcr family genes, Erp29, Reep5, |
| | | Ssr4, Krtcap2 |
| Program GP6 | Ribosome/translation | Rpl3, Rps26, Rps20, many other Rpl/Rps |
| | | genes, Rack1, Npm1, Tpt1, Naca |
| Program GP7 | Migratory dendritic cells (mDCs) | Nfkb2, Il12b, Cd83, Icosl, Icam1, Jak2, Atf5, |
| | | Ccl22, Ccl5, Marcks, Nfat5, Stat5a, Nfkbia/z, |
| | | Rel, Itgal, Ikbke, Cd274 |
| Program GP8 | TNF/LPS response | Cd33, Cd38, Cxcl1/2, Cybb, Gas7, Gng12, |
| | | Gpr84, Il1a, Il1b, Nlrp3, Sirpa, Syk, Tlr2, |
| | | Tnf, Il18 |
| Program GP9 | Regulation of autophagy and | Cd84, Ly75, Ccl6, Cd63, Cd68, Ctsa/b/c/d/z, |
| | inflammation | Plk2, Psap, Gpr137b, Mcl1, Cd44, Gpnmd, |
| | | Mt1/2, Fth1, Il7r, Litaf, Mgll |
| Program GP10 | MHC-I antigen (Ag) presentation | B2m, Tapbp, Grn, Hif1a, H2.D1, H2.K1, |
| | | H2.T23, Lamp1/2, Irf8, Cst3, Ctsk/l/s, Mdm2 |
| Program GP11 | DC2 MHC-II Ag presentation | H2.Aa, H2.Ab1, H2.DMa, H2.DMb1, H2.Eb1, |
| | | Cd74, Irf4, Ccr1/5, Ccl17, Socs2, Dcstamp, |
| | | Slamf9, Itgax, Mgl2, Axl, Anxa1 |

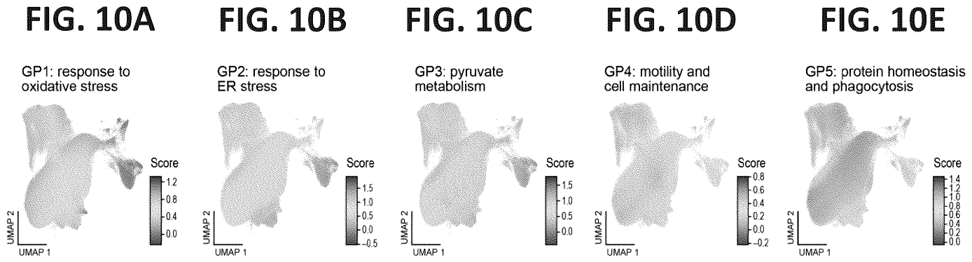

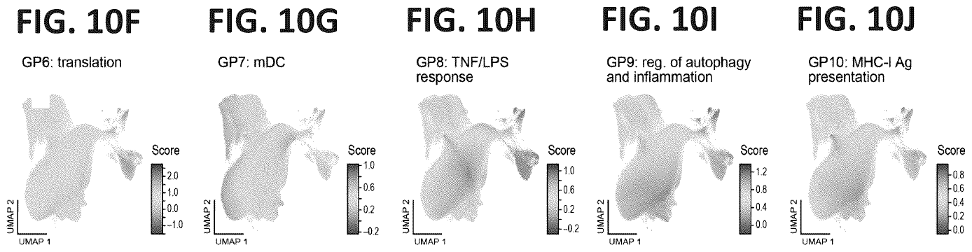

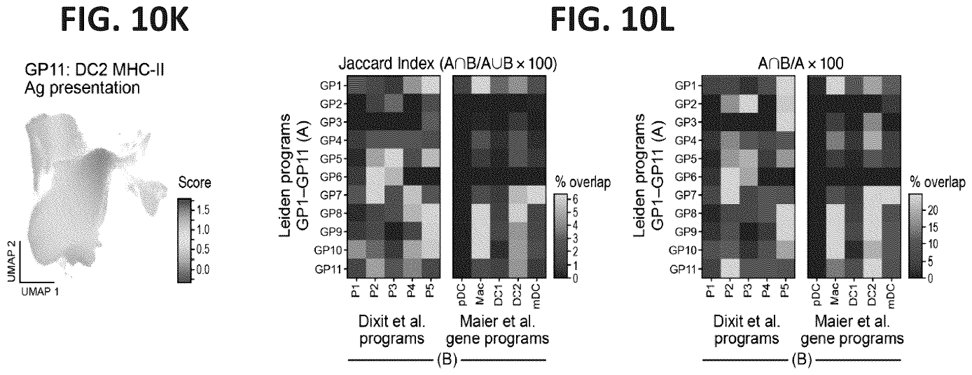

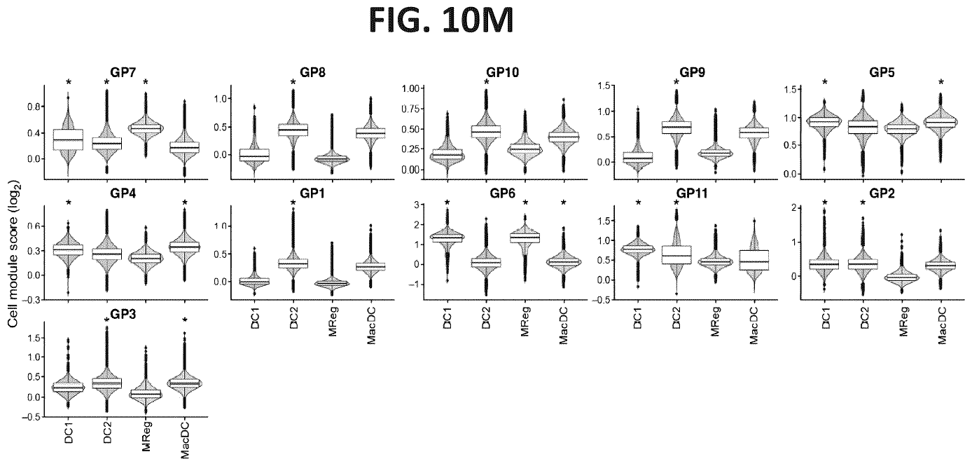

The eleven programs (FIGS. 2C, 2D, and 10A-1 OK; Table 6) consisted of genes strongly co-regulated across the perturbations and were enriched for different immune and cellular processes. The programs were annotated by their enrichment in functional categories and previously described signatures as response to oxidative stress (GP1); endoplasmic reticulum (ER) stress response (GP2); pyruvate metabolism (GP3); motility and cell maintenance (GP4); protein homeostasis and phagocytosis (GP5); translation (high in mDCs and DC1s) (GP6); mDCs (GP7); TNF/LPS response (GP8); autophagy regulation and inflammation (GP9); MHC-I antigen presentation (GP10); and DC2 and MHC-II antigen presentation (GP11) (Maier et al., Nature, 580: 257-262, 2020). Some programs are expressed broadly across all cell subsets (e.g., GP5, FIGS. 10E and 10M), while others are quite specific (e.g., GP6 in DC1s and mDCs; FIGS. 10F and 10M). The programs were not necessarily independent of each other: some were regulated in anti-correlated ways(e.g., opposite regulatory effects on GP6 (DC1/mDC expressed translation) vs. GP9 (autophagy and inflammation) and 11 (DC2 and MHC-II presentation)), while others were regulated in similar (though not identical) ways (e.g., GP4 (motility and cell maintenance), GP5 (protein homeostasis and phagocytosis), and GP6 (translation); FIG. 2C).

The programs refined ones that were previously defined in the same system under perturbation of 24 TFs (Dixit et al., Cell, 167: 1853-1866.e17, 2016) (e.g., P2 genes partition into mDC (GP7) and translation (GP6)) and uncovered new functional groupings (e.g., DC2 MHC-II antigen presentation (GP11), response to ER stress (GP2), and pyruvate metabolism (GP3)) (FIG. 10L), showing that the expanded scope and nature of perturbations could reveal additional regulatory processes.

### A. Regulatory Patterns and Targets Reveal the Functional Roles and Co-Associations of E3s

The six co-functional modules (comprising 329 regulators) each impacted a different subset of programs (FIGS. 2C and 2D), such that modules M1 and M2 were generally positively correlated with each other and negatively correlated with modules M3, M4, and M5, reflecting opposing functions.

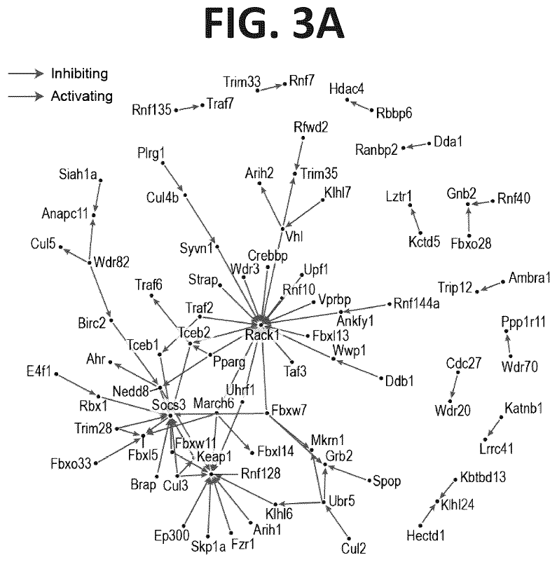

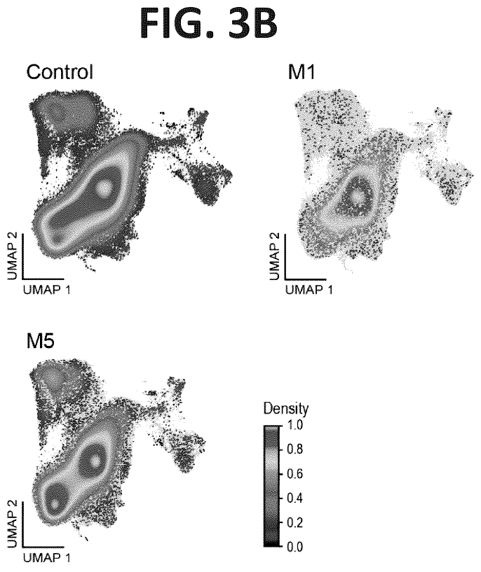

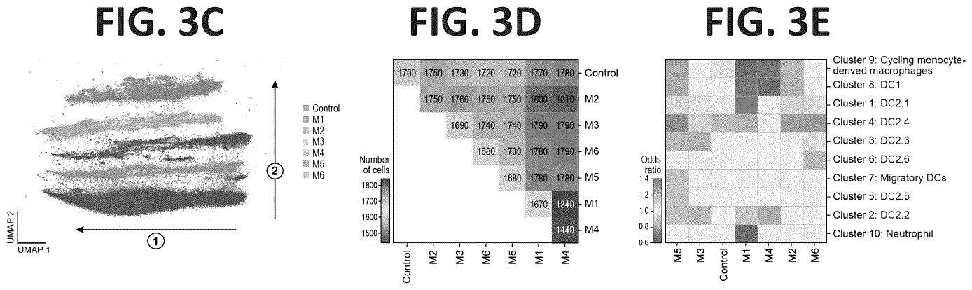

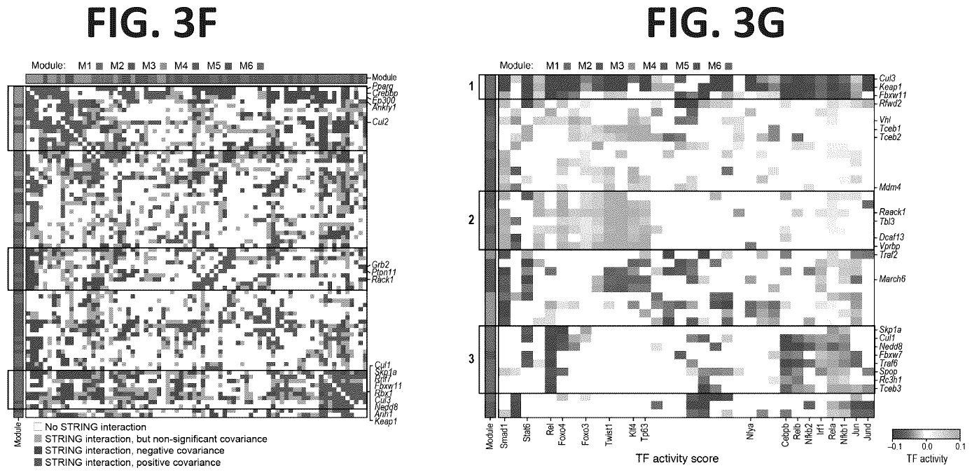

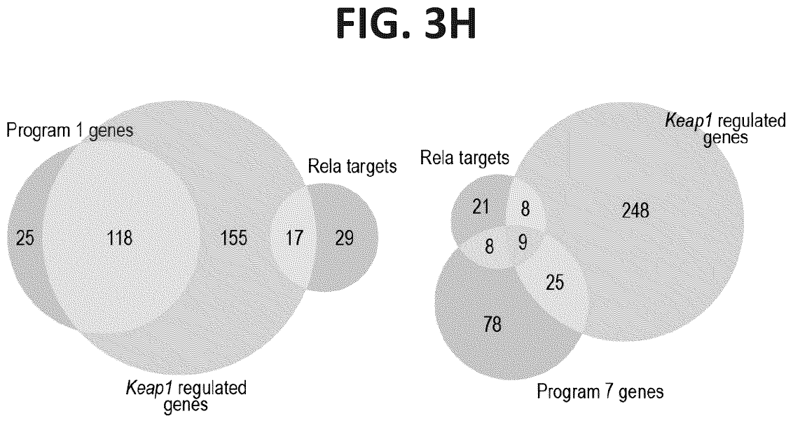

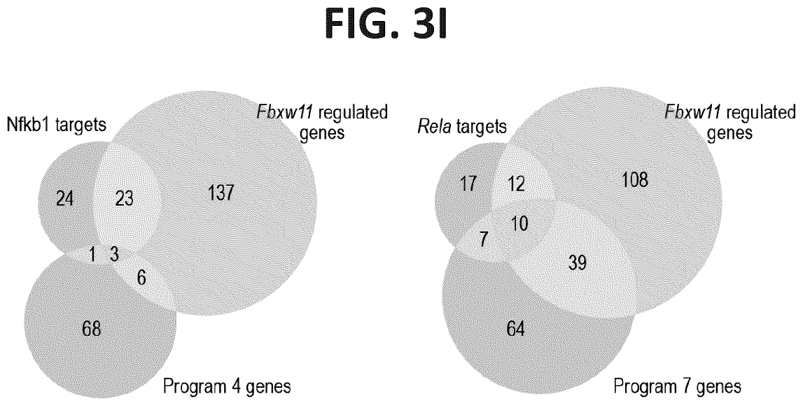

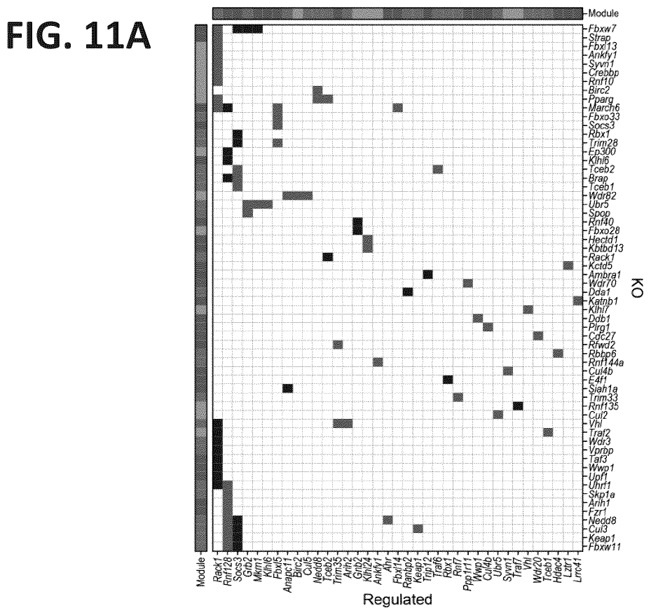

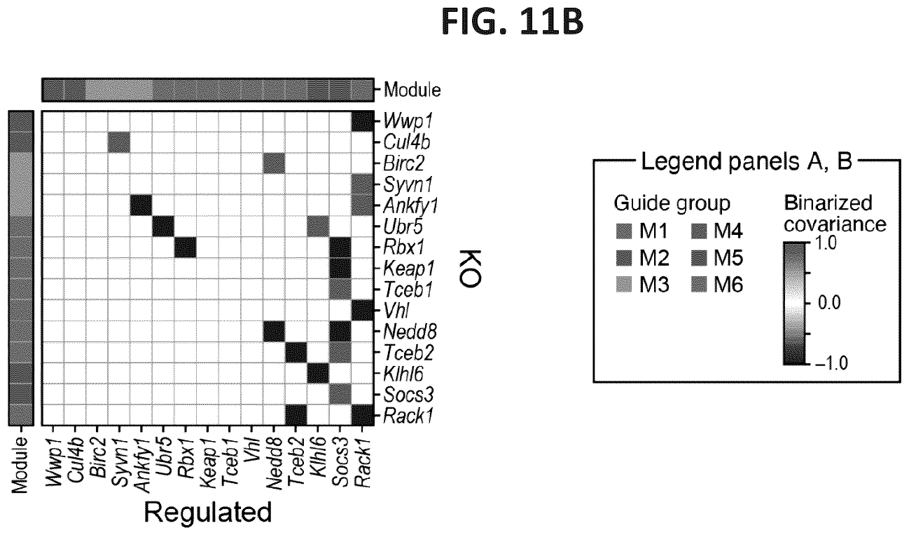

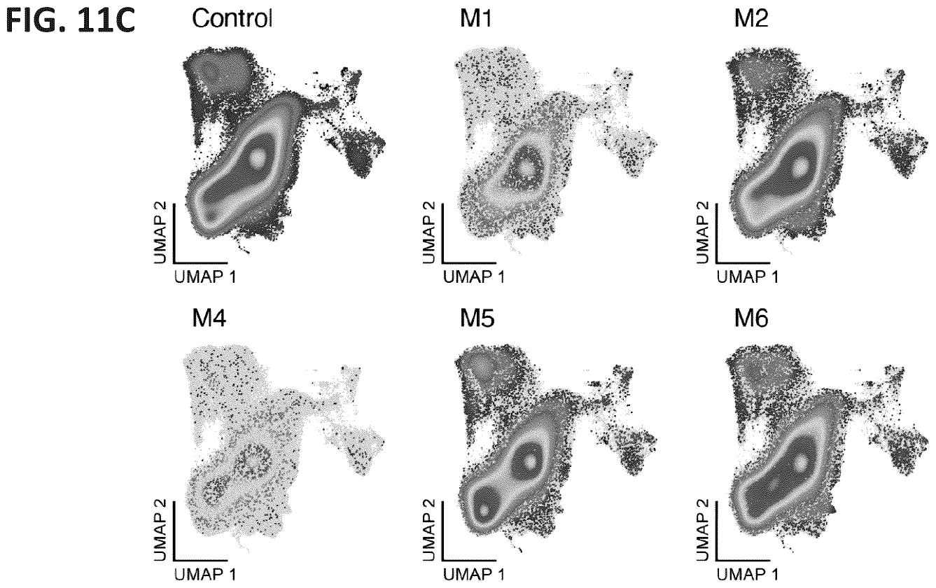

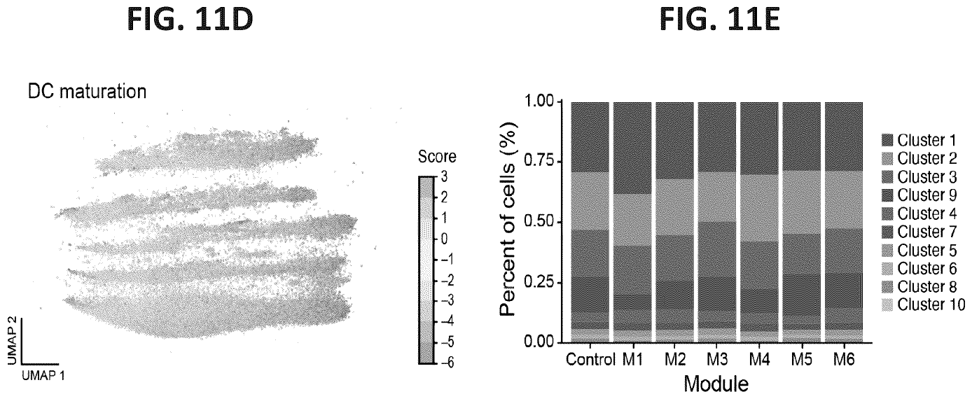

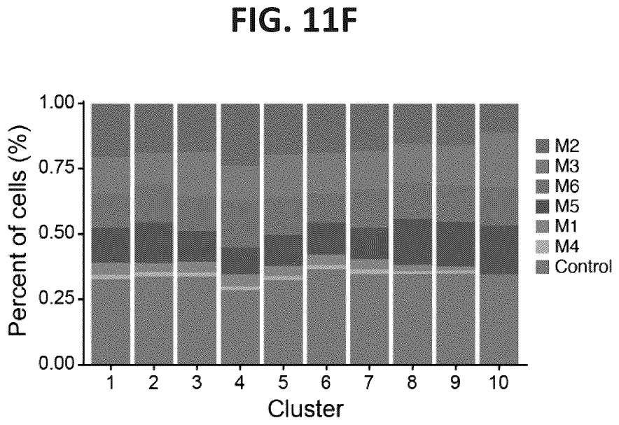

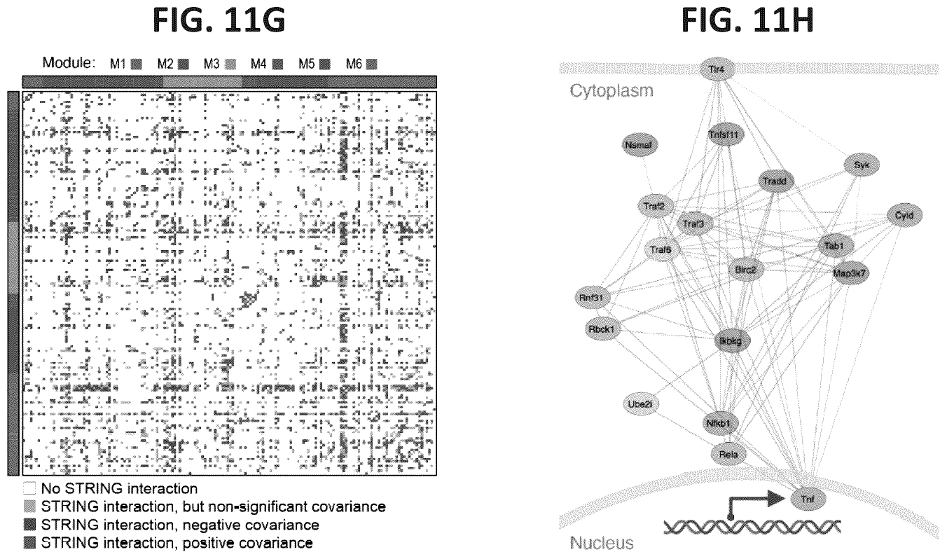

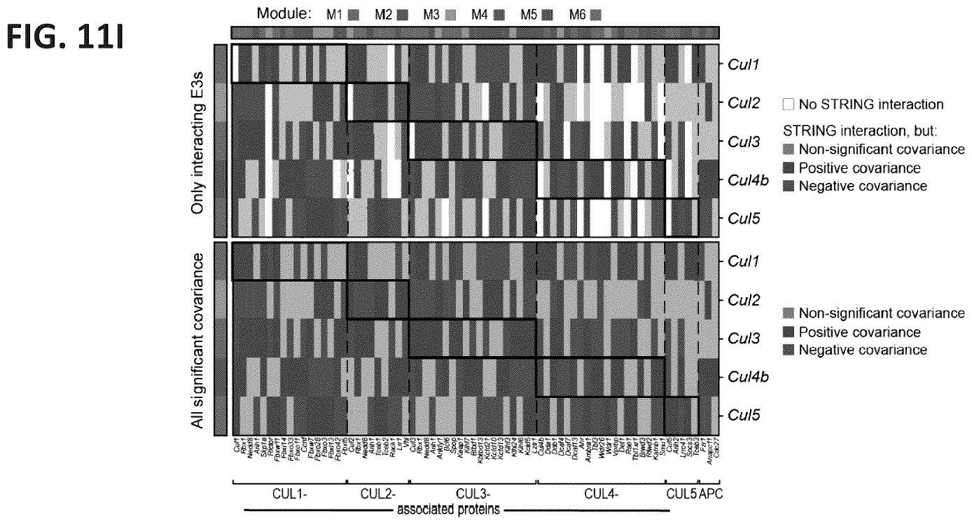

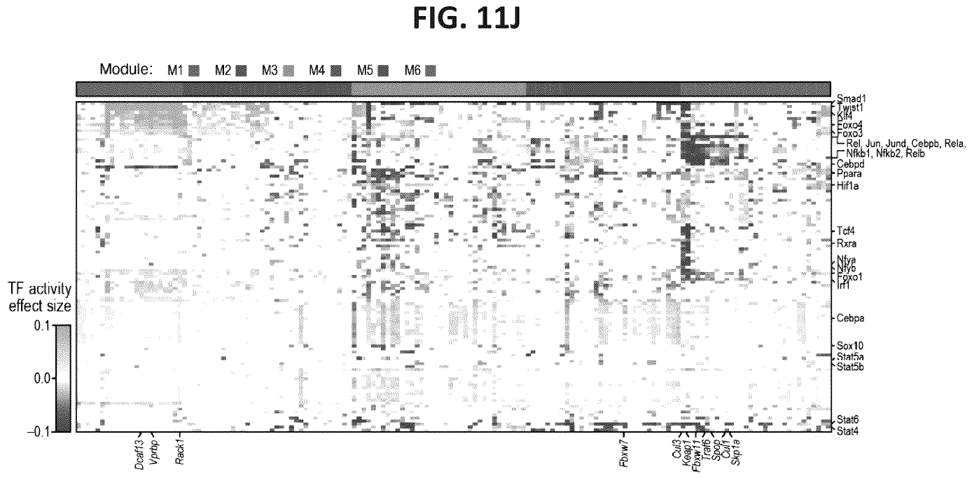

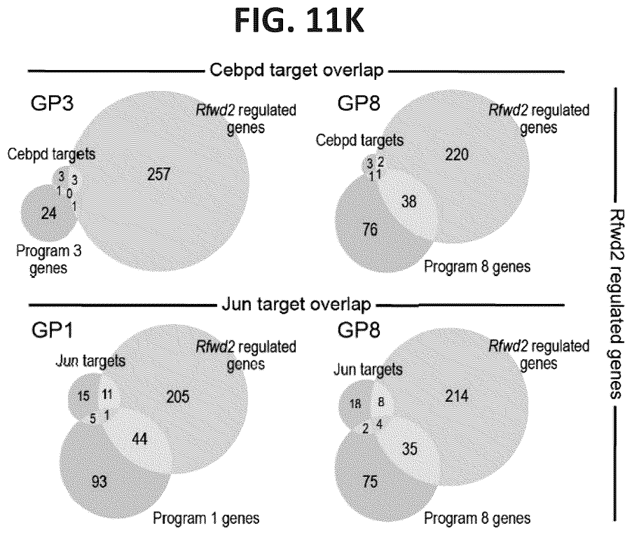

The co-functional modules included 97 E3 ligases, 65 E3 complex members, and 3 ubiquitin-like domains. For 85 of these genes, there was no prior literature evidence for roles in DCs or inflammation; the present findings can thus be deemed as novel functional annotations (Table 1 or 2), based on these genes' co-membership and their target programs (FIG. 2D). Three of the E3 ligase or complex members in the model are “authorities” in the E3 circuit, as their expression is impacted (positively and negatively) by a particularly high number of other E3s (FIGS. 3A, 11A, and 11B): E3s Rack1 (which targets Hif1 a and BimEL (Cai and Yang, Cell Div., 11(7), 2016; Corsini et al., Oncotarget, 6: 6524-6534, 2015; Qin et al., J. Immunol., 207: 1411-1418, 2021)) and Rnf128 (Grail; regulates Tbk1 and interferon and antiviral response (Nurieva et al., Immunity, 32: 670-680, 2010; Song et al., Nat. Immunol., 17: 1342-1351, 2016; Su et al., J. Immunol., 183: 438-444, 2009)), and E3 complex member Socs3 (regulates Jak2 and gp130 and response to cytokines). All three have known roles in DC biology or inflammation: Rack1 suppresses type 1 IFN responses rendering mice more susceptible to viral infection (Qin et al., J. Immunol., 207: 1411-1418, 2021), Rnf128 targets the DC maturation marker Cd83 for degradation (Su et al., J. Immunol., 183: 438-444, 2009), and Socs3 regulates JAK/STAT signaling, inflammation and macrophage polarization (McCormick and Heller, Front. Immunol., 6: 549, 2015). Notably, only 14 of 329 regulators were previously identified in a genome-wide CRISPR screen for regulators of TNF protein expression in the same system (Parnas et al., Cell, 162: 675-686, 2015), and these were further distinguished by our model into different modules, with most (9 of 14) in M6 (e.g., Ube2f, Rbck1, Rnf31, Spop, Traf6, and Nedd8 (positive regulators), and Rc3 h1 (negative regulator)). This shows the expanded power of functional discovery based on comprehensive expression profiles compared to one highly validated reporter/marker.

### M1 Regulators

M1 regulators strongly activated ER stress (GP2), protein homeostasis and phagocytosis (GP5), and translation (GP6) through the action of regulators of ribosome biogenesis (predicted E3s Bop1, Wdr36 and Noll 0, Cul4 substrate receptor Dcaf11 (VprBP); processome members E3 adaptors Dcaf13 and Wdr33), including those involved in the stress response (predicted E3s Wdr43, Wdr75, and Utp18) and cellular migration (E3 Aamp, predicted E3 Bop1) (Tables 1 and 2). This is consistent with the enrichment of ribosome biogenesis and stress genes in the translation (GP6) and ER stress (GP2) programs. Additionally, M1 regulators repressed the mDC (GP7), TNF/LPS response (GP8), and MHC-I antigen presentation (GP10) programs.

### M2 Regulators

M2 regulators had generally similar impacts to those of M1, repressing MHC-I antigen presentation (GP10) and the TNF/LPS response (GP8) (albeit more weakly) and activating protein homeostasis and phagocytosis (GP5) and DC1/mDC expressed translation (GP6). Consistently, they included multiple negative regulators of NFkB and interferon signaling (E3 complex member Bid, CopA and Copb2, Ikbkg, the SUMO E3 HDAC4 and E3 Mib1 (which prevent or signal Ikba degradation, respectively), predicted E3 Nsmaf, E3 Ppp1 r11, and the substrate Map3k7) and known regulators of cell differentiation in general (Bptf, Dido1, Gemin5, E3 and TF Zbtb7a) and of DC differentiation and maturation in particular (Ogt, Vdr (regulated by Mdm2), and TFs and predicted E3s Zbtb14 and Zbtb49), as well as the cell cycle and cell growth E3s APC (Anapc13, Cdc27, and Fzr1), CCNF, E4f1, Ring1, and Brca1; E3 adaptor Ddb1; E3 complex members Mdm4 and Pa2g4; DUB Wdr48, and Fbxo11. Module members included multiple components of one complex and pathway, such as Cul4b and Ddb1 from the same cullin E3 complex (below).

### M3 Regulators

M3 regulators had largely opposite effects to M2 and M1 regulators: M3 regulators activated the TNF/LPS response (GP8) and autophagy and inflammation (GP9), and repressed translation (GP6) and mDCs (GP7). Module members included many known regulators of inflammation (substrate Gnb1, the E3 and TF Pparg; E3s Traf3 and Wdr82 (which regulates TRAF3)), mTOR signaling (E3s Rictor and Traf2, E3 interacting partner Mlst8; substrates Mtor, Rheb and Rptor) (Lalani et al., J. Immunol., 194: 334-348, 2015; Murakami et al., J. Immunol., 202:1942-1947, 2019; Ricote and Glass, Biochim. Biophys. Acta, 1771: 926-935, 2007; Zhu et al., J. Immunol., 195: 5358-5366, 2015), autophagy (E3 Rnf216 (Triad3); predicted E3s Pik3r4 and Wdfy3), and ER transport (E3 Syvn1, substrates Sec13 and Sec31 a). In particular, the module includes Traf2, Pparg and Cebpb. CEBPB is known to activate DC2s and repress mDCs (Dixit et al., Cell, 167: 1853-1866.e17, 2016), and guides targeting Traf2 and CEBPB were consistently enriched in mDCs. CEBPB and PPARG physically interact (Lefterova et al., Genes Dev., 22: 2941-2952, 2008) and their targeting guides were enriched in CD2.2 cells. Other regulators include PAF1, which induced Tnf expression and inflammatory signaling (FIG. 2D), and regulators of ER transport (Sec13, Sec31 a, and Syvn1), all previously identified and validated as positive regulators of TNF expression in this system (Parnas et al., Cell, 162: 675-686, 2015). The present model now further relates them to mTOR pathway and autophagy regulators and to repressing mDC-like states.

### M4 Regulators

M4 regulators activated ER stress (GP2), pyruvate metabolism (GP3), protein homeostasis and phagocytosis (GP5), TNF/LPS response (GP8), and DC2 MHC-II antigen presentation (GP11) and repressed oxidative stress (GP1) and translation (GP6). They included DNA repair and splicing regulators, such as the E3s Plrg1 and Prpf1 and the predicted E3 Cdc40 (Prp17), suggesting a link between DNA repair and splicing and metabolism/translational control.

### M5 Regulators

M5 regulators activated autophagy and inflammation (GP9), MHC-I antigen presentation (GP10), and DC2 MHC-II antigen presentation (GP11), and repressed pyruvate metabolism (GP3), motility and cell maintenance (GP4), translation (GP6), and LPS/TNF response (GP8), through the action of regulators of antigen presentation or inflammation (SUMO E3 Pias1 and its substrate Prdm1 (negative regulators of MHC-II) and the substrates Syk, Nfkb1, and Tnf), endocytosis/trafficking (predicted E3s Wdfy2, Wdr81 and Wdr91, E3 March6), and negative regulators of CEBPB, the key DC2 TF (Rfwd2 (Cop1) and Det1 of the Cul4-Rfwd2-Det1 complex) (Lau and Deng, Trends Plant Sci., 17: 584-593, 2012; Ndoja et al., Cell, 182: 1156-1169.e12, 2020; Yoshida et al., Blood, 122: 1750-1760, 2013; Wertz et al., Science, 303: 1371-1374, 2004).

### M6 Regulators

Finally, M6 regulators activated mDCs (GP7) and TNF/LPS response (GP8) and repressed phagocytosis and granulation (GP1) and pyruvate metabolism (GP3) through the action of multiple cytokines and positive regulators of NF-kb (LUBAC E3s Rbck1 and Rnf31 (whose substrate Ikbkg is in “opposing module” M2), the substrate Rela; E3s Spop, Rc3 h1, Traf6 and Cbl; Tlr4; and E3 complex member Bcl6), positive regulators of cytoskeleton organization and migration (RING-like Phf8 (Asensio-Juan et al., Nucleic Acids Res., 40: 9429-9440, 2012; Gu et al., PLoS One, 11: e0146645, 2016; Zhou et al., J. Exp. Clin. Cancer Res., CR 37: 215, 2018), Ub and SUMO substrate Ptpn1 (Martin-Granados et al., J. Mol. Cell Biol., 7: 517-528, 2015)), and E3 Keap1, a negative regulator of redox and stress that targets Nrf2 (Bellezza et al., Biochim. Biophys. Acta Mol. Cell Res., 1865: 721-733, 2018; Hammer et al., Front. Immunol., 8: 1922, 2017; Rojo de la Vega et al., Cancer Cell, 34: 21-43, 2018; Sihvola and Levonen, Arch. Biochem. Biophys., 617: 94-100, 2017). M6 members also included multiple cullin and RING-like ligases (Cull, Cul3, Cul5, Keap1, Rnf31, Rbck1, Brap, Arih2, Traf6), and help assign putative roles to other E3s, such as Brap, a GWAS gene for psoriasis and carotid atherosclerosis, which activated inflammatory responses in human aortic smooth muscle cells (Lc et al., Nat. Commun., 8: 2017; Liao et al., Mol. Med., 17: 1065-1074, 2011) but did not have a previously known cellular role in immune cells.

The co-functional modules also impacted global shifts in cell state/type distributions, consistently with the programs they regulate. For example, cells perturbed for M5 regulators were associated with a shift from naïve DCs to macrophage-like cells, consistent with module's role as an activator of the DC2/MHC-II presentation (GP11) program, while perturbation to M1 and M2 regulators had the opposite effect (FIGS. 3B, 3E, 11C, 11E, and 11 F; FDR <0.1, Fisher's exact test). Across DC2s, one axis of variation distinguished between cells with perturbations from different modules (FIGS. 3C and 3D, direction 1). The co-functional modules also impacted distributions within one cell state/type. For example, different modules had distinct effects on the cells within DC2.1, DC2.2 and DC2.3 subsets, (FIGS. 3C and 3D, direction 2), orthogonally to the maturation gradient (FIG. 3C direction 1, FIG. 11D).

### B. Co-Functional Modules are Enriched for Physically Interacting E3s

It was next asked if the genetic regulatory network could be aligned and consistent with molecular mechanisms, such as co-complex membership, physical interactions and impact of E3s on TFs that regulate gene expression directly. First, to relate the co-functionality of E3s and complex members to shared molecular mechanisms, known protein interactions between each pair of regulators were searched for in the STRING database (Szklarczyk et al., Nucleic Acids Res., 49: D605-D612, 2021)) and these findings were compared to their module membership (FIG. 11G).

There was a significant enrichment of physical interactions between module members for four of the six co-functional modules (M1, M2, M4, and M6), as well as between one pair of modules (M3 and M5) (P<0.05, degree preserving permutation test), suggesting that co-functional effects are congruent with joint underlying molecular mechanisms. As expected, physical interactions between members of the same module were generally associated with positive correlation in functional effects, and physical interactions between members of different modules were generally associated with negative correlation in functional effects (FIGS. 3F and 11G). Such interactions include Gbr2, Ptpn11, and Rack1 (M1); Pparg, Crebbp, Ep300, Ankfy1, and Cul2 (M3); and Cul1, Skp1a, Rnf7, Fbxw1, Rbx1, Cul3, Nedd8, Arih1, and Keap1 (M6). Within components of the NFKB signaling pathway (FIG. 11H), multiple known TNF activators (Rela, Rbck1, Rnf31; all from M6) both physically interact with TNF and have positively correlated effects, whereas several TLR/NFKB signaling inhibitors (Nfkb1 (Yang et al., J. Immunol., 186: 1989-1996, 2011), Cyld (Yoshida et al., J. Biol. Chem., 280: 41111-41121, 2005), and Tab1 (Yang et al., EMBO Rep., 18: 205-216, 2017); all in M5) have physical interactions but negatively correlated effects, showing consistency between the genetic model and molecular mechanisms.

This analysis highlighted basic “rules” of co-regulation between E3 cullin, adaptor, and substrate recognition adaptor proteins, wherein multiple components of each Cullin complex grouped together in the same module (except the common interactor Rbx1), while the Cullin substrate recognition adaptor proteins were in other modules, consistent with their specializing or directing Cullin E3 complexes to different substrates and pathways (FIG. 11I). For example, the SCF core complex members Cul1, Skp1 and Rbx1 are members of M6, as is Cul3, but Cul1-Skp1-Rbx1 substrate-specific adaptors or Cul3-Rbx1 adaptors are partitioned to multiple other modules (e.g., Cull-associated adaptors Fbxll4 and Fbxl5 in M2; Fbxll3 and Fbx03 in M3; Fbxw7 in M5; Fbxo33 and Fbxw11 in M6; Cul3-associated adaptors Klhl3, Klhl24, Klhl6 in in M2, M3, and M5, respectively; and Cul5-associated adaptor Socs3 in M5; FIG. 11I). Similarly, Cul4b and its adaptor Ddb1 are part of M2, but Dda1, which recruits and organizes substrate receptors with both Cul3 and Cul4 complexes, is in M6. Furthermore, each of the interacting pairs of Rbx1 and Arih1 and Cul5 and Arih2 are in M6, consistent with their physical interaction and known functional roles in forming highly specific neddylated CRLs (Kostrhon et al., Nat. Chem. Biol., 17: 1075-1083, 2021), but their expected adaptors were not (e.g., most F-box proteins for Cull and Arih1; except Fbxw7 and 11). This highlights the versatility and modularity of the ubiquitin system, whereby different adaptors/receptors target different substrates regulating different aspects of DC lifecycle, and how a systematic Perturb-Seq screen and computational analysis can decipher this organization.

### C. Conclusions: Congruent Genetic Effects and Physical Interactions Relate Complex Members and Characterize E3 Partners and Substrates

Screening both E3s and their interacting partners and substrates allowed relation of functional (genetic) effects to physical interactions and molecular mechanisms. Co-functional modules of regulators are enriched for physical protein-protein interactions and members from the same E3 complex. While multiple components of each Cullin complex grouped together in the same module (except the common interactor Rbx1), the Cullin substrate recognition adaptor proteins were in other modules, highlighting their specializing or directing Cullin E3 complexes to different substrates and pathways. Because programs may not be independent, and regulators are strictly partitioned to separate modules, when regulators have multiple roles, this partitioning can mask their full set of relationships. For example, in the CUL4-DDB1-RBX1-DET1-RFWD2 E3 cullin complex, Cul4b and its adaptor Ddb1 are both members of M2, and Rbx1, Cull/3/5, and Det1 are in M5. This challenge is addressed when considering independent factors associated with overlapping regulators: Rfwd2 (Cop1) co-regulated the response to LPS with Rbx1 (factor #5), the response to oxidative stress with Ddb1 (#2), and chemotaxis with Det1 (#9). Thus, the ICA factors allow us to relate different adaptors with partly overlapping effects (e.g., Rbx1, Det1, and Ddb1) and the way in which they combine with different substrate recognition adaptor proteins.

Combining co-functional genetic profiles with physical interactions showed congruence between genetic relations and molecular mechanisms, and helped suggest new interactions between E3s and putative adaptors. For example, surprisingly, Rfwd2, but not other members of the CUL4-DDB1-RBX1-DET1-RFWD2 complex, is a regulator of factors 3, 10, and 11, suggesting that it may interact with other complexes to ubiquitylate targets. Other regulators of these factors that have strong physical interactions with Rfwd2 and could be such candidates include: Ptpn11 (Shp2) (factor 3 and 10 regulator) that acts as an adaptor with p38-pRfwd2 to bind and catalyze Ub-mediated degradation of FASN (Muramatsu et al., Blood, 115: 1969-1975, 2010); Wdr82 and Ep300 (factor 10 and 11); Anapc13 (Schwickart et al., Mol. Cell. Biol., 24: 2004, p. 1), Cul2, Cul5, and Huwe1 (factor 10); and Crebbp, Skp1 a, Nedd8, Cul1, and Wdr5 (factor 11).

Because knockouts of E3s and their substrates should have opposite effects when the substrate is targeted for degradation, and similar effects when Ub modification is activating, the directionality of protein level regulation can be predicted by the correlation between expression profiles of E3-substrate pairs in the screen. For example, the regulatory profile of Cebpb is negatively correlated with that of Rfwd2 in all ICA factors where both are regulators (#3,5,11), consistent with the targeting of CEBPB for degradation by the CUL4-DDB1-RBX1-DET1-RFWD2 complex. Fbxw11 KO leads to repression of (inferred) activity of all of NFKB1 (p105/p50 precursor), NFKB2 (p100/p52 precursor), Rela (p65) and Relb. Because the targets of these transcriptional activators are repressed both by the TFs' own KO and by Cul3, Keap1 and Fxbw11 KO, it is unlikely that this effect is mediated by the TF's degradation. For Cul3 and Keap1, the effect is likely through direct CUL3-KEAP1 ubiquitin modification and subsequent degradation of IKBKB (Lee et al., Mol. Cell, 36: 131-140, 2009). As for Fxbw11, it is reported to directly bind with NFKB1 and NFKB2 (Heissmeyer et al., Mol. Cell Biol., 21: 1024-1035, 2001), and this binding is enhanced in the presence of a proteasome inhibitor (Kim et al., Mol. Cell Biol., 35: 167-181, 2015). While the current model suggests that full-length Nfkb1 is processed constitutively and Fbxw11 (also known as BTrCP2) targets Nfkb2 for complete degradation upon stimulation such as LPS activation (Heissmeyer et al., Mol. Cell Biol., 21: 1024-1035, 200; Zhang et al., Cell, 168: 37-57, 2017), the data provided herein may not be fully consistent with such a model. It is hypothesized that Fbxw11 could be part of the system that interacts with the proteasome to process p105 (Nfkb1) and p100 (Nfkb2) to generate active p50 and p52, respectively. These analyses may be particularly helpful for multi-subunit E3 Ligase complexes reusing core scaffolds and adaptors with different substrate recognition adaptor proteins.

### Example 4. E3 Perturbation Effects on Gene Programs can be Explained by Modulation of TF Activities

To better understand how the E3 ligases may propagate to the transcriptional level, the above-described genetic model was combined with a model associating transcription factors (TFs) to their physical targets, thus allowing inference of TFs whose activity (as inferred from the expression of their known targets (Badia-i-Mompel et al., Bioinforma. Adv., 2: vbac016, 2022; Garcia-Alonso et al., Genome Res., 29: 1363-1375, 2019)) is impacted by each perturbation.

Overall, the 329 knockout (KO) perturbations were significantly associated with inferred changes in activity of 123 TFs (FIGS. 3G and 11J)|, with 32 TFs with the most prominent effects from 41 E3 ligase and complex members (FIG. 3G). Most notably, Cul3 and Keap1 perturbations decreased the activity of many TFs, including Nfkb1, Nfkb2, Rela, Relb, Jund, Irf1, Cebpb, Nfya, Klf4, Foxo3, and Foxo4 and increased the (inferred) activity of just four of the 32 highly regulated TFs: Atf6, Wt1, Srf, and Pax6 (FIG. 3G, box 1). Importantly, it is shown Cul3 and Keap1 perturbations increased (inferred) activity of the well-known CUL3-KEAP1 substrate and master regulator of oxidative stress, Nrf2 (FIG. 11J). The set of TFs whose activity was impacted by Cul3 and Keap1 perturbations was further partitioned to two subsets based on their opposing regulation by different adaptors and receptors. For example, Rack1, Dcaf13 and Vprbp perturbations increased the (inferred) activity of Foxo3/4, Klf4, Twist1, and Smad1 (FIG. 3G, box 2), while perturbations of M6 E3s, including Spop, Traf6, Fbxw7, Fbxw11, and Cull decreased the (inferred) activity of Irf1, Nfkb2, Nfkb1, Rela, Relb, Rel, and Jun (FIG. 3G, box 3).

The present analysis detects established links between E3s and regulated TFs, supporting its validity. For example, Hif1 a activity increased following KO of any member of the E3 ligase complex VHL-TCEB1-TCEB2, which binds and ubiquitylates Hif1 a for degradation (Haase, Curr. Pharm Des., 15: 3895-3903, 2009) (FIG. 11J). Cebpb and Jun's inferred activity increased in cells with KO of the E3 ligase Rfwd2 (FIG. 3G), a member of the CUL4-DDB1-DET1-RFWD2 complex (that targets Cebp family TFs and Jun for ubiquitination and degradation (Wertz et al., Science, 303: 1371-1374, 2004). Indeed, Rfwd2 represses programs that are activated by Cebpb (GP3 and GP8) or by Jun1 (GP1 and GP8) and are enriched for their bound targets (FIG. 11K). Because Rfwd2 knockout similarly increases the (inferred) activity of Foxl2 and JunD, these could be additional Rfwd2 substrates.

By relating joint TF targets, E3 targets and E3 regulated programs, E3s impact on different programs through different mediating TFs is explained. For example, by this analysis, Rela mediates Cul3 and Keap1 effects on GP7 and GP8, but not on GP1 (FIG. 3H), and E3 Fbxwl l's impacts on GP7 (but not GP4) (FIG. 3I). Notably, Cul3, Keap1, Fxbw11 and Rela itself are all members of M6, and the expression of Rela's targets in GP7 is decreased when any of these regulators is perturbed (KO) in the PerturbSeq screen (FIGS. 2C and 2D). However, because KO of Cul1, Keap1 and Fbxw11 leads to (inferred) reduction in activity of Rela, it is unlikely that Cul3, Keap1 or Fbxw11's effects is through targeting of Rela for degradation.

### A. Perturbed E3 Ligases Impact Multiple Statistically-Independent Pathways

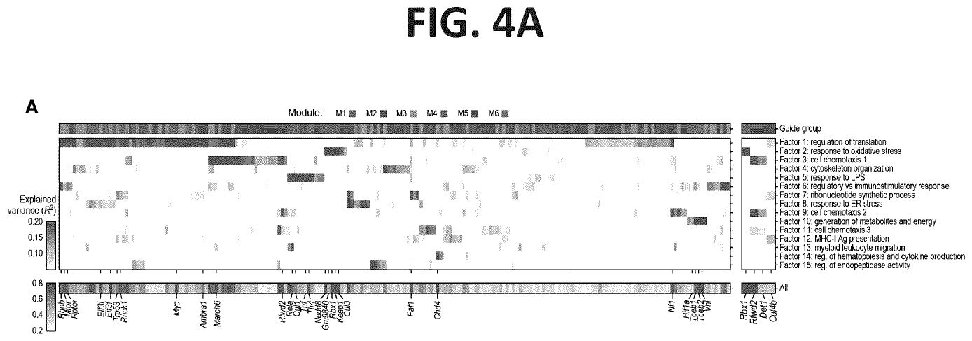

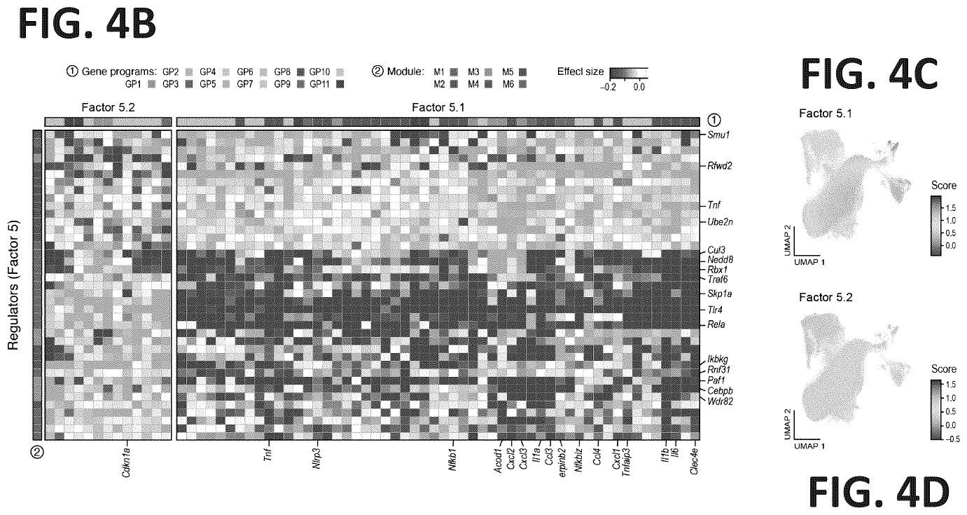

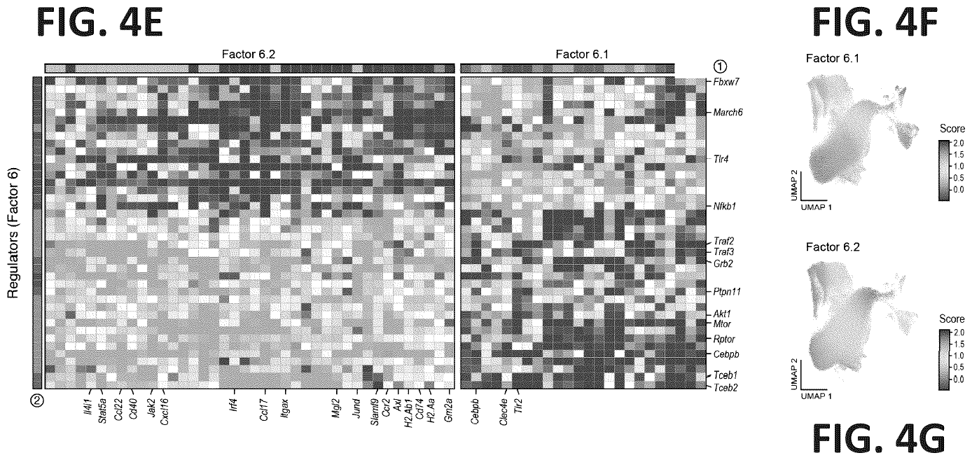

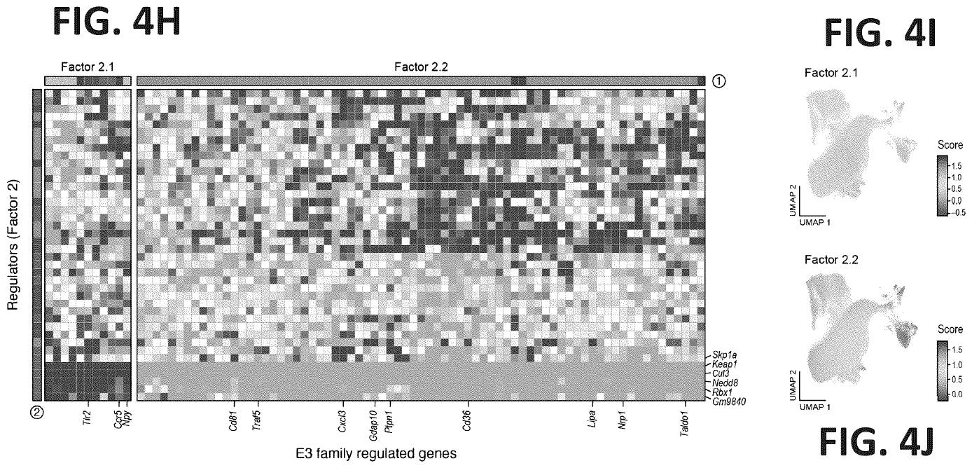

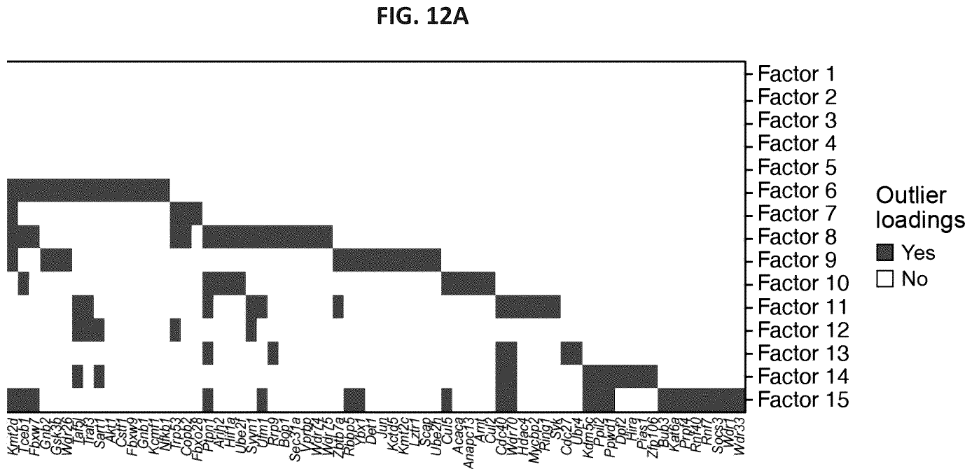

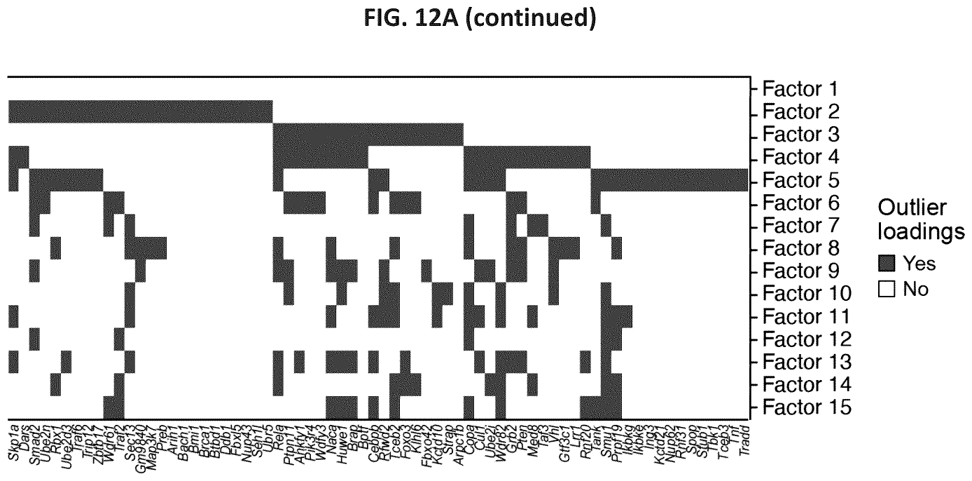

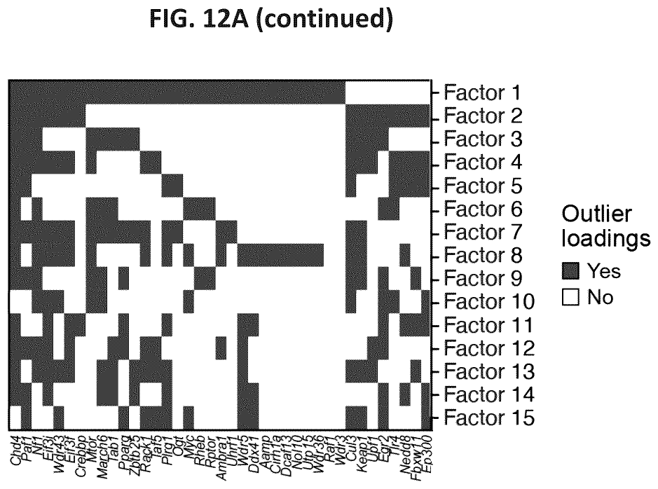

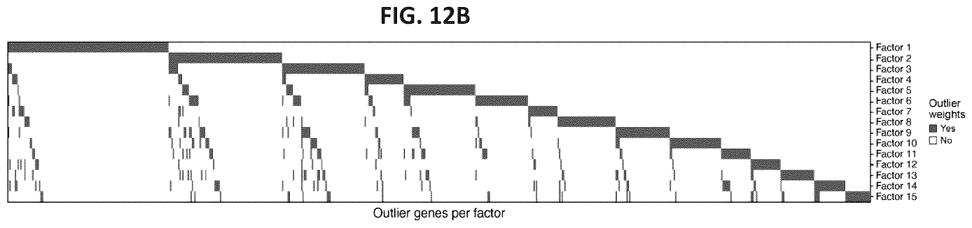

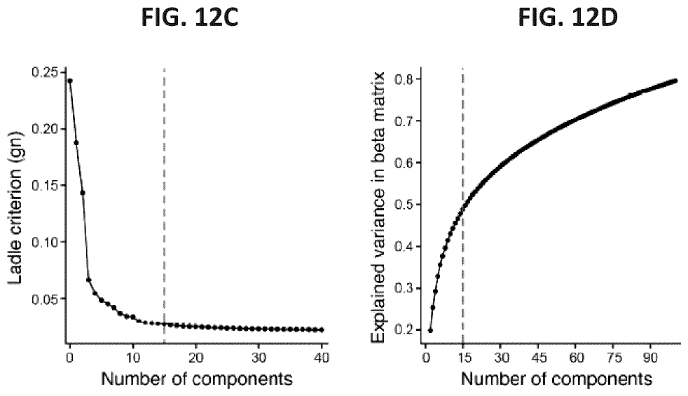

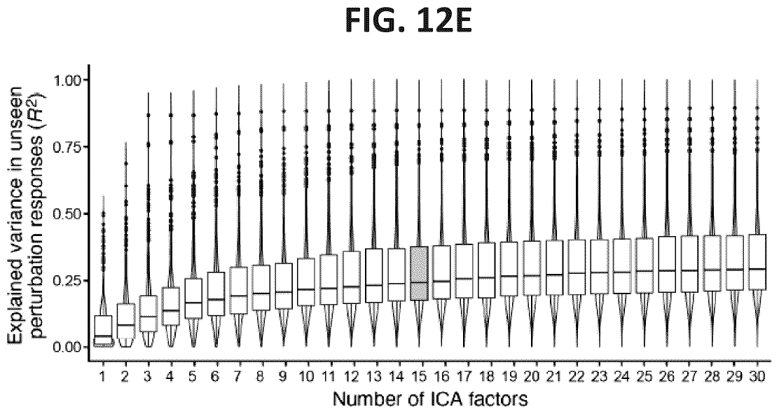

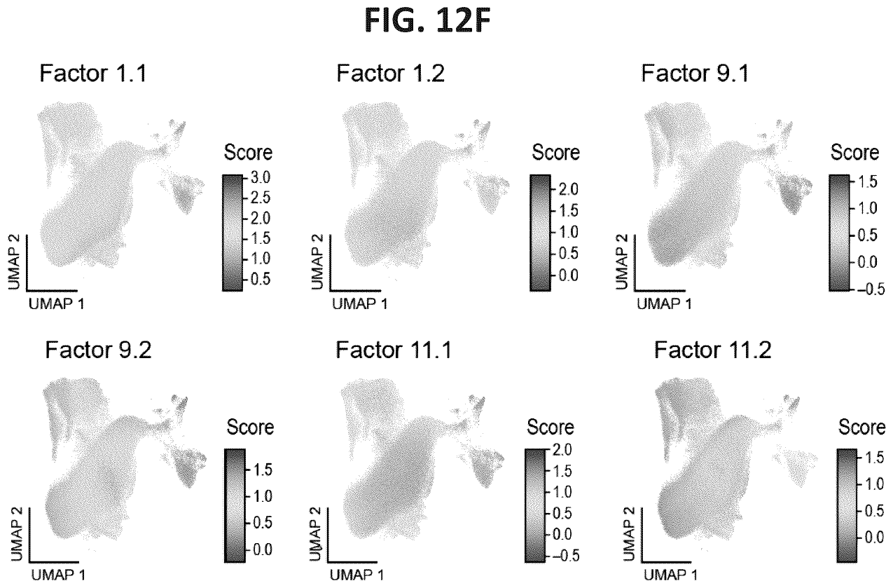

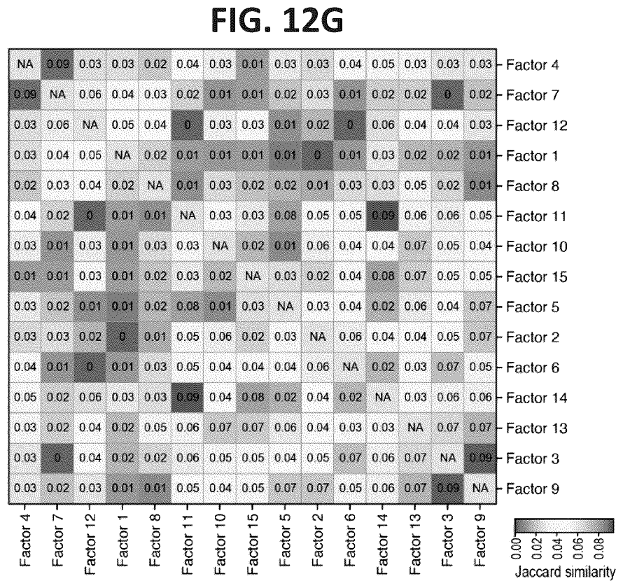

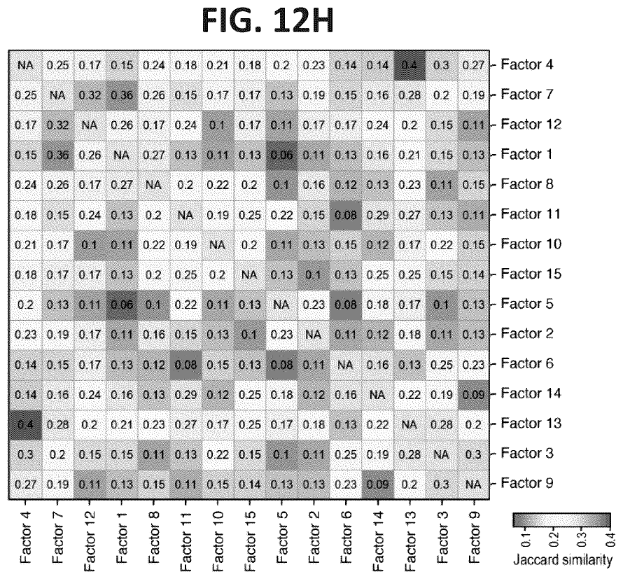

The effect of perturbing one gene on another gene's expression can be due to various pathways with direct or indirect dependencies, and indeed, pairs of programs in the present regulatory model are dependent, as reflected by their pairwise positive and negative correlations (FIG. 20). To decompose the observed regulatory effects into a set of statistically-independent factors, Independent Components Analysis (ICA) (Hyvärinen and Oja, Neural Netw., 13: 411-430, 2000) was performed on the regulatory matrix, recovering 15 independent latent factors whose weighted sums optimally explained the perturbation effects (FIGS. 12A-12E), and these factors were annotated by enrichment in functional gene sets and known marker genes (Table 7). On average, the factors explained 35% of the variance of observed effects of the 329 impactful regulators (27 explained well (>70%); 72 explained poorly (<20%)) (FIG. 4A, bottom). The latent factors have little to no correlation (maximal Spearman ρ=0.14) and little overlap in highly loading genes (FIGS. 12B and 12G; maximum Jaccard similarity index=0.09), but perturbed genes can affect multiple independent factors (FIGS. 12A and 121H; maximum Jaccard similarity index=0.4).

TABLE 7
ICA Factors and Genes

| Factor 1 gene group 1 | Factor 1 gene group 2 | Factor 1 guide group 1 | Factor 1 guide group 2 |
|---|---|---|---|
| Eef1a1, Tpt1, Rps27, | Ms4a7, Sgk1, Lhfpl2, | Rack1, Utp15, Wdr43, | Ddx41, Plrg1, Chd4, |
| Rplp0, Eif3f, Eif3e, | Il1rn, Alcam, Psap, | Raf1, Uhrf1, Myc, | Tab1, Zbtb25, |
| Eif3k, Eif1, Rpl3, Rpl14, | Ctsb, Ccdc88a, H2.M2, | Wdr3, Aamp, Dcaf13, | March6, Crebbp, |
| Rpl10a, Rps6, Rps27a, | B2m, Laptm5, H2.K1, | Cirh1a, Wdr36, Taf5, | Pparg, Nf1, Ambra1, |
| Rpl8, Rpl32, Rpl13, | H2.D1. | Nol10, Ogt, Wdr5. | Eif3f, Eif3i, Rptor, Mtor, |
| Rps8, Rps18, Rpl11, | | | Rheb, Paf1. |
| Rps17, Eef1g, Rpl12, | | | |
| Rps2, Rps12, Rplp1, | | | |
| Rpl13a, Naca, Npm1, | | | |
| Btf3, Rpl38, Rpl7a, | | | |
| Rack1, Rpl35, Rpl31, | | | |
| Rpl36, Rpl22l1, Rplp2, | | | |
| Rpsa, Rps19, Rps16, | | | |
| Rps25, Rpl41, Rps29, | | | |
| Rps9, Rps28, Eef1b2, | | | |
| Rpl24, Rpl37a, Rps15, | | | |
| Rps26, Eef2, Rpl10, | | | |
| Rpl23a, Rps21, Rpl39, | | | |
| Rps4x, Rpl36a, Rpl26, | | | |
| Rpl17, Rps24, Rpl23, | | | |
| Rpl5, Rps7, Rpl7, | | | |
| Rps14, Rps20, Fau, | | | |
| Rps13, Rpl34, Rps3, | | | |
| Rpl27a, Rps23, Rps11, | | | |
| Rpl21, Rpl18, Rpl15, | | | |
| Rpl9, Rpl29, Rpl6, | | | |
| Rps15a, Rpl22, Rpl4, | | | |
| Rpl30, Rpl35a, Rps5, | | | |
| Rpl18a, Rps3a1, | | | |
| Rps10, Rpl37, Rpl28, | | | |
| Rpl19, Gapdh, | | | |
| Cox7a2l, Hspa5, Aprt, | | | |
| Hspa8, Atp5e, | | | |
| Hsp90ab1, Cdh1, | | | |
| Uqcrh, Cox4i1, Ncl, | | | |
| Ssr4, Pabpc1, Anxa2, | | | |
| Actb. | | | |
| Factor 2 gene group 1 | Factor 2 gene group 2 | Factor 2 guide group 1 | Factor 2 guide group 2 |
| Ccr5, H3f3b, Tlr2, | Mmp8, Thbs1, Acod1, | Wdr43, Map3k7, Brca1, | Btbd1, Upf1, Tlr4, |
| Sdc4, Lmo4, Ddit3, | Slc7a11, Cxcl3, Cd81, | Traf2, Ube2n, Bach1, | Nup43, Wdr61, Trip12, |
| Nupr1, Mcl1, Npy, | Traf5, Tma16, Gdap10, | Fbxl5, Ddb1, Skp1a, | Seh1l, Preb, Egr2, |
| Inhba, Btg1. | Rnf128, Cd53, Plet1, | Gm9840, Nedd8, Rbx1, | Sec13, Chd4, Bmi1, |
| | Mmp12, Prkcb, | Arih1, Cul3, Keap1, | Dars, Ep300, Smad2, |
| | Slc48a1, Creg1, Cd36, | Fbxw11, Traf6, | Ubr5, Crebbp, Nf1, |
| | Rhob, Lipa, Hvcn1, | Ube2d3, Zbtb17. | Eif3f, Eif3i, Paf1. |
| | Met, Alas1, Pf4, Tarm1, | | |
| | Tpm4, Il1f9, Alox5ap, | | |
| | Raf1, Slpi, Mgst2, | | |
| | Phlda1, Hmox1, Cd52, | | |
| | Zyx, Uchl1, Mmp19, | | |
| | Esd, Tubb2a, Akr1a1, | | |
| | Runx2, Al314180, | | |
| | Gbe1, Slc43a2, Mylip, | | |
| | Ptpn1, Gstm1, Nampt, | | |
| | Pla2g7, Cat, Txnrd1, | | |
| | Ppfibp2, Dap, Mcoln2, | | |
| | Chpf2, Prdx1, Cyb5a, | | |
| | Nrp1, Srxn1, Gclm, | | |
| | Prdx6, Gss, Ptgr1, | | |
| | Taldo1, | | |
| | B430306N03Rik, | | |
| | Rnasel, Dck, Ass1, | | |
| | Clec4n, Procr, Ampd3, | | |
| | Blvrb, Pgd, Abcc1. | | |
| Factor 3 gene group 1 | Factor 3 gene group 2 | Factor 3 guide group 1 | Factor 3 guide group 2 |
| Stap1, | Lgals1, Naaa, Plet1, | Naca, Fbxo42, Cul3, | Upf1, Bptf, Ptpn11, |
| X2010005H15Rik, | Creg1, Cd36, Lipa, | Keap1, Rfwd2, Kctd10, | Rela, Huwe1, Brap, |
| Pid1, Spata13, Trem1, | Fabp5, Ftl1, Lgals3, | Egr2, Chd4, Tab1, | Cebpb, Tceb2. |
| Metrnl, Upp1, Id1, Glrx, | Rab3il1, Ctsz, Mt1, | Zbtb25, Foxo3, Klhl6, | |
| Thbs1, Emb, Tgfbi, | Sdc3, Cd63, Lgmn, | March6, Pparg, Nf1, | |
| S100a8, | Vat1, Myof, Psap, | Arpc1b, Wdfy3, Ankfy1, | |
| F630028O10Rik, | Gpnmb, Dnmt3a, Pld3, | Pik3r4, Mtor, Paf1, | |
| Pou2f2, C5ar1, Ly6c2, | Atp6v0d2, Ctsb, Mgll, | Strap. | |
| Gpr84, Clec5a, Fpr1, | S100a1, Slamf7, Plk2, | | |
| Clec4d, S100a11, Fn1, | Fam46c, Ccdc88a, | | |
| Il1f9, Phlda1, Icam1, | Rnase2a, Acp5, | | |
| Gadd45a, Cd14, | Ddhd1. | | |
| Lpcat2, Cpd, Sod2, | | | |
| Lcn2, Saa3, Prdx5, | | | |
| Fcgr2b, Serpinb2, | | | |
| Cebpb, Snx18, | | | |
| Wfdc21. | | | |
| Factor 4 gene group 1 | Factor 4 gene group 2 | Factor 4 guide group 1 | Factor 4 guide group 2 |
| Ccr5, Ccr2, Cotl1, | Ubb, Sdc4, Marcksl1, | Rack1, Wdr43, Taf5, | Skp1a, Nedd8, Cul3, |
| Ifi27l2a, Lgals1, Cfl1, | Malat1, Ddit3, Bri3, | Naca, Copa, Taf3, | Keap1, Cul1, Fbxw11, |
| Ptma, AC160336.1, | Klk1b1, Mgll, Wfdc17, | Rnf20, Gtf3c1, Upf1, | Rela, Tlr4, Chd4, Pten, |
| Flna, Tmsb4x, Coro1a, | Gbp5, Gbp2, Mndal, | Bptf, Lrr1, Grb2, | Nf1, Wdfy3, Ankfy1, |
| Arhgdib, Ear2, Lpl, | Ddhd1, H2.K1, H2.D1, | Ptpn11, Vhl, Huwe1, | Pik3r4. |
| Actb, Actg1, Crip1, | Cdkn1a, Cd274. | Brap, Ube2i, Wdr82, | |
| Lsp1, Pfn1, Il1rn, | | Med8, Dars, Ep300, | |
| Lcp1, Clec4a2. | | Eif3f, Eif3i, Mtor, Paf1. | |
| Factor 5 gene group 1 | Factor 5 gene group 2 | Factor 5 guide group 1 | Factor 5 guide group 2 |
| Bcl2a1d, Bcl2a1b, | Mafb, Malat1, Klf4, | Copa, Smu1, Ube2n, | Ogt, Ikbkg, Rnf31, |
| Cxcl2, Gpr84, Acod1, | Abcg1, Gda, Cd300lf, | Tradd, Rfwd2, Trip12, | Skp1a, Nedd8, Rbx1, |
| Slc7a11, Rassf4, | Cd200r1, Socs6, | Tank, Tbk1, Tnf, Stub1, | Cul3, Cul1, Fbxw11, |
| Marco, Cxcl3, Met, | Nabp1, Laptm5, Txnip, | Ikbke, Kctd21, Ube2i, | Rela, Tlr4, Traf6, Spop, |
| Nfkbiz, Cxcl1, Cd14, | Hist1h1c, Cdkn1a. | Zbtb17, Ep300. | Ing3, Ube2d3, Nup62, |
| Marcksl1, Il12b, Nfkb1, | | | Tceb3, Cebpb, Prpf19, |
| Ehd1, Ptgs2, Slco3a1, | | | Plrg1, Wdr82, Chd4, |
| Il1b, Il6, Il1a, Nlrp3, | | | Smad2, Paf1. |
| Tnf, Cpd, Sod2, Slfn2, | | | |
| Fam20c, Ccl3, Ccl4, | | | |
| Serpinb2, Hivep3, | | | |
| Malt1, Ccl17, Ptx3, | | | |
| Tnfsf15, AA467197, | | | |
| Mtpn, Clic4, Nrp2, | | | |
| Cpeb4, Fam102b, | | | |
| Zc3h12c, Ppfia3, | | | |
| Sh3bp5, AW112010, | | | |
| Cflar, Tnfaip3, Slc7a2, | | | |
| Clec4e, Plek, Cav1, | | | |
| Inhba, Marcks. | | | |
| Factor 6 gene group 1 | Factor 6 gene group 2 | Factor 6 guide group 1 | Factor 6 guide group 2 |
| Pid1, Lgals1, Ndufa4, | Ccr2, Zfp36l1, Cst3, | Myc, Gnb1, Gnb2, | Wdr26, Tlr4, Cstf1, |
| S100a8, Fpr1, Igf1, | Plet1, Itgax, Dcstamp, | Akt1, Traf2, Ube2n, | Kcmf1, Sart1, Wdr61, |
| Hmox1, Ptpn1, Tlr2, | Mmp12, Cd52, Clec4n, | Grb2, Ptpn11, Tank, | Nfkb1, Egr2, Pten, |
| Sgk1, Sdcbp, Fam20c, | Ccl22, Stat5a, Cd40, | Traf3, Fbxw9, Cebpb, | Smad2, Tab1, Foxo3, |
| Rilpl2, Pmp22, Cebpb, | Srgn, Il4i1, Mgl2, | Tceb1, Tceb2, Kmt2d, | Klhl6, March6, Nf1, |
| Fabp5, Fabp4, Ftl1, | Sema4a, Grk3, Vcan, | Taf5l, Chd4, Wdfy3, | Fbxw7, Gsk3b. |
| Ctsd, Bhlhe41, Lyz2, | Scimp, Ahr, Slamf9, | Ankfy1, Pik3r4, Rptor, | |
| Fth1, Ctsb, Clec4e. | Gbp5, Gbp2, Cxcl16, | Mtor, Rheb. | |
| | Jak2, Etv3, Bhlhe40, | | |
| | Ccl17, Irf4, Pfkp, | | |
| | Nectin2, Gm2a, Axl, | | |
| | Cd74, H2.Ab1, H2.Aa, | | |
| | AA467197, Jund, Clic4, | | |
| | Btg1. | | |
| Factor 7 gene group 1 | Factor 7 gene group 2 | Factor 7 guide group 1 | Factor 7 guide group 2 |
| Eef1a1, Ddit3, Atf4, | S100a6, Bcl2a1b, | Rack1, Wdr43, Uhrf1, | Taf3, Ogt, Cul3, Keap1, |
| Sgk1, Lhfpl2, Il1rn, | Fcer1g, H3f3a, | Copa, Copb2, Smu1, | Wdr61, Med8, |
| Sqstm1, H2.M2, | S100a4, Cox8a, Cotl1, | Grb2, Kmt2d, Plrg1, | Smad2, Tab1, Zbtb25, |
| Cybb, Abca1, Inhba. | Atp5j2, Serf2, Atp5g1, | Sec13, Chd4, Eif3f, | March6, Pparg, Nf1, |
| | Nme1, Calm1, Cfl1, | Eif3i, Mtor. | Ambra1, Trp53, Paf1, |
| | H2afz, Dbi, S100a10, | | Fbxo28. |
| | Nedd8, Atp5j, Ly6e, | | |
| | Sdc4, Pnp, Actb, Gpx1. | | |
| Factor 8 gene group 1 | Factor 8 gene group 2 | Factor 8 guide group 1 | Factor 8 guide group 2 |
| Tpt1, Rpl22l1, Rps19, | Dstn, Lyz2, Fth1, Ctss, | Rack1, Utp15, Wdr43, | Copa, Copb2, Gm9840, |
| Tceal9, Arf4, Ift20, | Acsl1, Ccng1, | Rrp9, Myc, Aamp, | Nedd8, Rbx1, Cul3, |
| Mthfd2, Herpud1, | Clec2d, Cdkn1a. | Dcaf13, Cirh1a, Wdr36, | Keap1, Rela, Hif1a, |
| Uqcrq, Phgdh, C1qb, | | Vprbp, Wdr74, Nol10, | Ufm1, Tceb1, Tceb2, |
| Hspa5, P4hb, Tram1, | | Wdr75, Map3k7, Naca, | Kmt2d, Sec31a, Preb, |
| Pdia3, Calr, Pdia4, | | Gtf3c1, Bop1, Lrr1, | Sec13, Pten, Ube2f, |
| Rpn1, Hsp90b1, | | Grb2, Vhl, Wdr5, | Arih2, Ptpn1, Nf1, |
| Pdia6, Vcp, Erp29, | | Prpf19, Ddx41, Plrg1, | Ambra1, Trp53, Syvn1, |
| Sec11c, Serf2, Ostc, | | Chd4, Eif3f, Eif3i, Mtor. | Fbxw7. |
| Manf, Sdf2l1, Sec61b, | | | |
| Dad1, Sec61g, Sf3b5, | | | |
| Cope, Spcs2, Timm13, | | | |
| Krtcap2, Dap, Ddit3, | | | |
| Atf4, Cpd, Ftl1, Lgals3, | | | |
| Hnrnpa3, Selenos, | | | |
| Ssr3, Canx, Sec22b, | | | |
| Surf4, Serp1, Xbp1. | | | |
| Factor 9 gene group 1 | Factor 9 gene group 2 | Factor 9 guide group 1 | Factor 9 guide group 2 |
| Chil3, Klhdc4, Atp5g3, | Ch25h, S100a6, Fyb, | Naca, Vhl, Ube2h, | Fbxo42, Jun, Gnb2, |
| Mcemp1, Ffar2, Ly6c2, | Ifitm3, Maf, Lgals1, | Wdr26, Gm9840, Cul1, | Grb2, Ptpn11, Cul3, |
| Slc7a11, Fpr2, Ear2, | Lmna, Ccl2, Ccl7, | Fbxw11, Rela, Zbtb7a, | Keap1, Ybx1, Kmt2d, |
| Naaa, Ucp2, Plet1, | Mmp8, Acod1, Kctd12, | Rfwd2, Det1, Huwe1, | Kmt2c, Rbbp5, March6, |
| Il1f9, Scd2, Ptpn1, | Msr1, Itgam, Mmp12, | Brap, Ube2i, Egr2, | Rptor, Mtor, Rheb. |
| Lmo4, F10, Vasp, | Pf4, Nfkbiz, Cxcl1, | Chd4, Pten, Kctd5, | |
| Sgk1, Zfp3612, Cyp51, | Npc2, Srgn, Ptgs2, | Lztr1, Smad2, Pparg, | |
| Hmgcs1, Wfdc21, | Saa3, Gas7, Ccl3, | Nf1, Paf1, Scap, | |
| Prkcd, Ptprc, Syk. | Ccl4, Serpinb2, Pdpn, | Gsk3b. | |
| | Npy, C3ar1, Syngr1, | | |
| | Cond1, Lgals3, Mmp13, | | |
| | Lrpap1, Crip1, Adam8, | | |
| | Timp2, Cd300ld, | | |
| | Trem2, Ctsl, Serpinb6a, | | |
| | Cd63, Gpnmb, Ctsb, | | |
| | Ptx3. | | |
| Factor 10 gene group 1 | Factor 10 gene group 2 | Factor 10 guide group 1 | Factor 10 guide group 2 |
| Chil3, Ak4, Bnip3l, Pfkl, | Herpud1, Lipa, Hmox1, | Wdr43, Myc, Copa, | Vhl, Cul3, Tlr4, Rfwd2, |
| Pdk1, Slc16a3, Hilpda, | Cd52, Prdx1, Lmo4, | Smu1, Anapc13, | Cul2, Tceb1, Tceb2, |
| Hk2, Bsg, Slc2a1, | Lpcat2, Rilpl2, Slamf9, | Ptpn11, Arnt, Hif1a, | Wdr82, Kctd10, Egr2, |
| Aldoc, Pgm2, Hk1, | Lyz2, Wfdc17, Ccl9. | Huwe1, Sec13, Eif3i, | Ep300, Ube2f, Cul5, |
| Pkm, Pgk1, Gpi1, | | Mtor. | Arih2, Ptpn1, March6, |
| Aldoa, Prelid1, Eno1, | | | Nf1, Strap, Acaca. |
| Gapdh, Higd1a, | | | |
| Fam162a, Eif4ebp1, | | | |
| Mif, Tpi1, Ldha, Sacs, | | | |
| X2010005H15Rik, Ier3, | | | |
| Trf, Vim, Emilin2, Gsn, | | | |
| Tarm1, Scd2, Gbe1, | | | |
| Sdc4, Socs3, Rbpj, | | | |
| C3ar1, Lgals3, Anxa2, | | | |
| Pgam1, Basp1, Klk1b1, | | | |
| Ndufv3, Mt1, Sdc3, | | | |
| Tgm2, Mt2, Rnase2a, | | | |
| Slc7a2. | | | |
| Factor 11 gene group 1 | Factor 11 gene group 2 | Factor 11 guide group 1 | Factor 11 guide group 2 |
| Rpl12, Ch25h, Car4, | X0610012G03Rik, | Ring1, Naca, Copa, | Hdac4, Mycbp2, Ikbkg, |
| Upp1, Fn1, Lpl, Itgax, | Klk1b11, Ms4a6c, Maf, | Smu1, Wdr5, Wdr70, | Skp1a, Nedd8, Cul1, |
| Mmp12, Itgb2, Met, | Apoe, Tmem176b, | Zbtb7a, Ufm1, Traf3, | Fbxw11, Rfwd2, Tceb2, |
| Phlda1, Zyx, Il1a, Ccl3, | Tmem176a, C1qa, | Cebpb, Cdc40, Prpf19, | Ptpn1. |
| Ccl4, Plaur, Scimp, | C1qb, C1qc, Rassf4, | Ddx41, Plrg1, Wdr82, | |
| Wfdc21, Ccl6, Id2, | Slc43a2, Ppfibp2, | Kctd10, Egr2, Syk, | |
| Wfdc17, Ccl9, Mt2, | Icam1, Tnfaip2, Nfkbia, | Sec13, Taf5l, Med8, | |
| Rnase2a, AA467197, | Marcksl1, Icosl, Ms4a7, | Chd4, Ep300, Crebbp, | |
| Inhba. | Fam49a, Ccl22, | Pparg, Eif3f, Eif3i, | |
| | Gadd45b, Cd83, Il4i1, | Syvn1. | |
| | Fnbp1l, C3, Apobec1, | | |
| | Sqstm1, Marcks, | | |
| | Ddhd1. | | |
| Factor 12 gene group 1 | Factor 12 gene group 2 | Factor 12 guide group 1 | Factor 12 guide group 2 |
| Rplp0, Rpl22l1, Rpl37a, | Atpif1, Atp5g1, Pycard, | Rack1, Taf5, Smu1, | Copa, Traf2, Upf1, |
| Trf, Fcgrt, Id1, Ly6e, | Cyba, Smdt1, Psmb5, | Sart1, Prpf19, Egr2, | Wdr5, Traf3, Smad2, |
| Mgst1, Kctd12, Ear2, | Hint1, Sec61b, Psmb6, | Taf5l, Chd4, Paf1. | Tab1, Pparg, Nf1, |
| Mpeg1, Itgal, Rnase4, | Ddit3, Nupr1, Eif5, | | Ambra1, Trp53, Eif3f, |
| Spp1, Sdc3, Aplp2, | H2.Q6, Mtdh, Tagln2, | | Eif3i, Syvn1. |
| Ifi204, B2m, Laptm5, | Lgals3, Tmsb10, | | |
| Grn, Nfe2l2. | Ccdc88a, H2.M2. | | |
| Factor 13 gene group 1 | Factor 13 gene group 2 | Factor 13 guide group 1 | Factor 13 guide group 2 |
| Rpl22l1, Klhdc4, Polr2l, | Cd38, Cxcl3, Fn1, | Rack1, Wdr43, Rrp9, | Smu1, Cdc27, Skp1a, |
| Klk1b11, Ccr1, Cyba, | Mbnl1, Lpl, Ptpn1, | Taf5, Naca, Rnf20, | Ubr4, Cul1, Fbxw11, |
| Emp1, Vim, Furin, | Icam1, Sdc4, Epha4, | Upf1, Grb2, Wdr5, | Rela, Wdr70, Ube2d3, |
| Anxa5, Rnh1, Selenoh, | Fam46a, Saa3, Gas7, | Cul3, Keap1, Wdr82, | Huwe1, Brap, Cebpb, |
| Mmp8, Ly6c2, Scd2, | Serp1, Dstn, Lyz2, | Sec13, Chd4, Ankfy1, | Cdc40, Plrg1, Pten, |
| Prdx1, Ms4a7, Cd9, | Cond2, Nrp2, | Eif3f, Eif3i. | Ptpn1, Tab1, Zbtb25, |
| Cd68, Plin2, C3ar1, | Fam102b, Mmp14, | | Foxo3, March6, Nf1. |
| Gdf15, Cd300lf, Ftl1, | Rasgrp1, Slc7a2, | | |
| Neat1, Lgals3, Anxa1, | Clec4e, Inhba, H2.K1. | | |
| Chst11, Creb5, Spp1, | | | |
| Il7r, Aph1c, Slamf7, | | | |
| Plk2, Rasgef1b. | | | |
| Factor 14 gene group 1 | Factor 14 gene group 2 | Factor 14 guide group 1 | Factor 14 guide group 2 |
| Rps20, Chil3, Gpx3, | Mcub, | Smu1, Nedd8, | Traf2, Wdr5, Kdm5c, |
| St8sia4, Adgrl2, Car4, | C1qb, S100a11, Fn1, | Rbx1, Rela, Dpf2, | Hira, Pias1, Zfp106, |
| Ppic, Selenow, Phgdh, | Hvcn1, Cd52, Pla2g7, | Wdr70, Ppwd1, Sart1, | Ube2i, Tceb2, Wdr82, |
| Il18, Ptges, Ms4a6d, | Lgals3, Anxa1, Crip1, | Cdc40, Prpf19, Ddx41, | Egr2, Taf5l, Med8, |
| H3f3b, Tmem176b, | Ccl6, Id2, Malt1, Tnip3, | Plrg1, Ppil2. | Chd4, Ep300, Tab1, |
| Tmem176a, Fcer1g, | Prkcd, Nfe2l2, Marcks. | | Zbtb25, Foxo3, Klhl6, |
| Mpc1, Lgals1, | | | March6, Eif3i, Paf1. |
| AC160336.1, | | | |
| Hsp90aa1, Ly6e, | | | |
| Adgre1, Il1f9, Uchl1, | | | |
| Rab32, Pdzk1ip1, | | | |
| Npc2, H2.Q7, H2.Q6, | | | |
| Ifi203, C3, Adgre4, | | | |
| Ctsk, Msrb1, Nov, | | | |
| Tyrobp, F7, Mt1, Mt2, | | | |
| Ccdc88a, B2m, H2.K1, | | | |
| H2.D1. | | | |
| Factor 15 gene group 1 | Factor 15 gene group 2 | Factor 15 guide group 1 | Factor 15 guide group 2 |
| Ybx1, Rps27l, | Fcer1g, C1qb, C1qc, | Rack1, Myc, Taf5, | Wdr43, Cul3, Keap1, |
| AC160336.1, | Aprt, Cyba, Ptpn18, | Naca, Copa, Prpf4, | Kdm5c, Kat6a, Ybx1, |
| Hsp90aa1, Chchd2, | Pou2f2, Gpr84, | Smu1, Rnf20, Rnf40, | Ufm1, Socs3, Huwe1, |
| Bcl2l11, Vcan, | Gngt2, Adgre1, Gmfg, | Traf2, Wdr5, Wdr70, | Brap, Egr2, Ep300, |
| Serpinb2, Wfdc21, | Lst1, Igf1, Csf1r, | Ppwd1, Wdr61, Tank, | Pparg. |
| Spp1, Alcam, Dstn, | Mpeg1, Ptpn1, Clec4n, | Cebpb, Tceb1, Tceb2, | |
| Kctd12b, Tnfsf15, Sat1, | Blvrb, Fcgr2b, Cd68, | Wdr1, Kmt2d, Cdc40, | |
| Ahnak. | C3ar1, Trem2, Ctsz, | Prpf19, Ddx41, Plrg1, | |
| | Tyrobp, Cd300c2, | Bub3, Wdr82, Ppil2, | |
| | F7, Pirb, Clec12a, | Rbbp5, Wdr33, Cul5, | |
| | Cd33, Egr2, P2ry14, | Rnf7, Ptpn1, Paf1, | |
| | Tgm2, Prkcd, Mmp14. | Fbxw7. | |

Each factor simultaneously captures both induced and repressed genes along with diametrically opposed regulators. For example, the LPS response factor (#5) (FIGS. 4B-4D) includes activation of LPS and TNF response genes (and repression of genes more highly expressed in DC2s vs. mDCs and DC1s; FIGS. 4C and 4D). It is positively regulated by well-established activators of TNF and the LPS response (e.g., Rnf31, Traf6, Paf1, Ikbkg, Rela, Cebpb) (Parnas et al., Cell, 162: 675-686, 2015), and repressed by known negative regulators (e.g., Rfwd2 (Ndoja et al., Cell, 182: 1156-1169.e12, 2020)), as well as by previously-uncharacterized regulators, both positively (E3 adaptor Skp1a) and negatively (E2 Ube2n; E3 substrate adaptor Smu1). The DC immune control factor (#6) (FIGS. 4E-4G) consists of inflammation and cytokine response genes in two opposing patterns, capturing the maturation gradient from immunostimulatory monocyte derived macrophages and DC2s to immunomodulatory mDCs genes and its corresponding, diametrically-opposed regulation by E3 Traf2 and E3 adapter Ptpn11 vs. March6 and Fbxw7, respectively.

Because one regulator can be associated with multiple ICA factors, this decomposition groups together different subsets of multi-subunit E3 complex members. For example, all four components of the Cul3-Skp1 a-Rbx1-Nedd8 complex are associated as negative regulators of the response to oxidative stress factor (#2) (FIGS. 4H-4J). Conversely, different combinations of subunits of one complex are associated with different ICA factors, predicting how interactions of one core complex with different substrate adaptor proteins can drive a variety of gene regulation programs. For example, in the Rfwd2/Cul4a/Ddb1/Rbx1/Det1 complex, Rfwd2 (Cop1), the E3 substrate recognition adaptor protein that forms an active E3 complex, has outlier loadings indicating regulation of ICA Factors 3, 5, 9, 10, and 11 (Table 7). Other members of this complex regulate other factors, not impacted by Rfwd2 perturbation (e.g., Ddb1 regulates Factor 2, Table 7). Interestingly, while Rfwd2 and Det1 knockout are both similarly strongly associated with the same factors (#3, 9, and 11), other complex member knockouts (Rbx1, Cul4b) have only weak associations in those same factors (FIG. 4A, right panel). Thus, Rfwd2 or Det1 may interact with other E3 or cullin complex members, just as the Cul4 complex interacts with other substrate recognition adaptor proteins.

### B. Intra-Module Genetic Interactions and Inter-Module Additivity in Combinatorially-Perturbed Cells

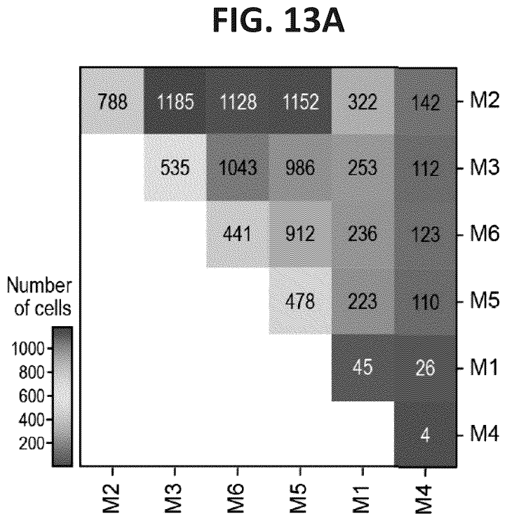

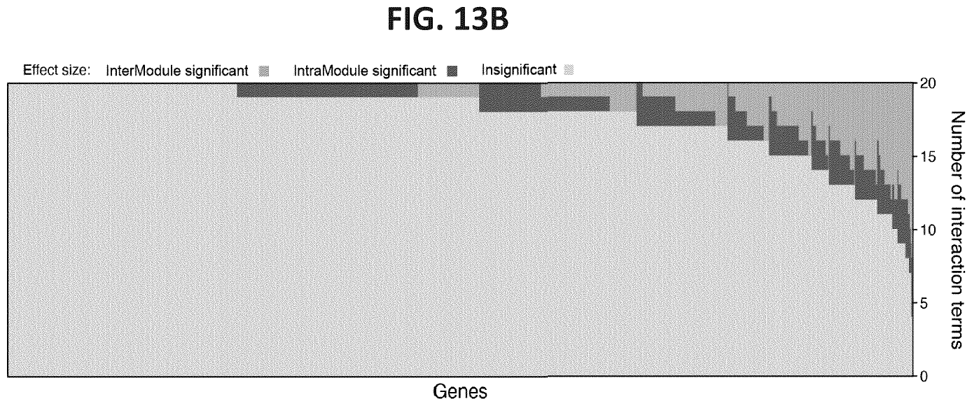

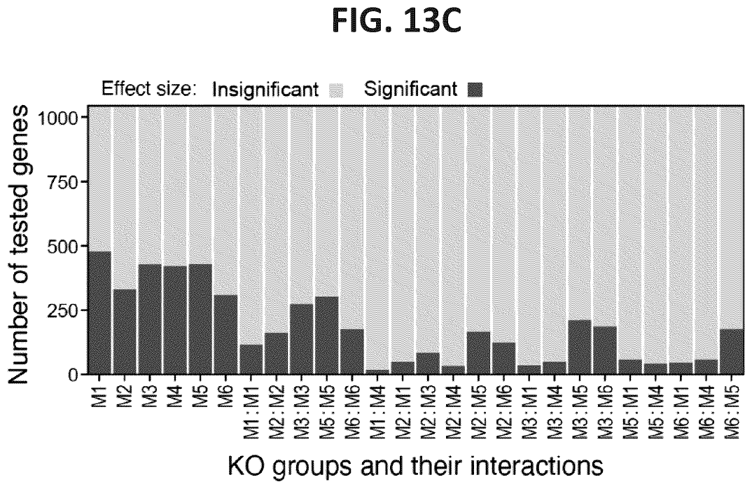

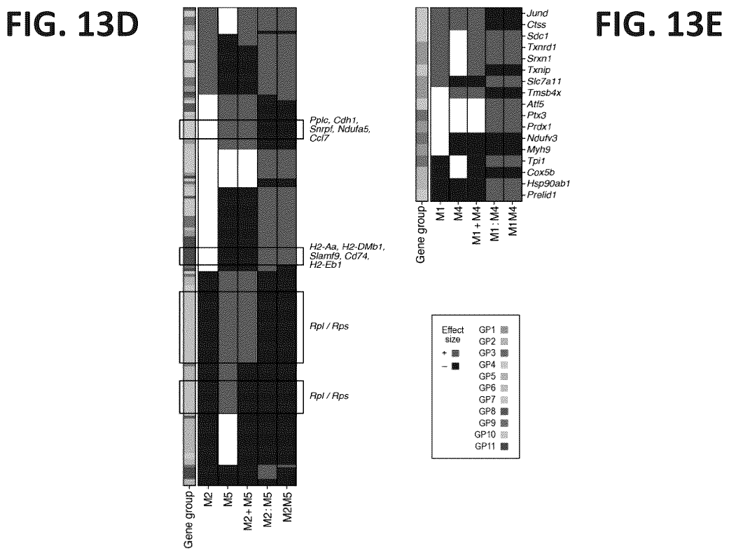

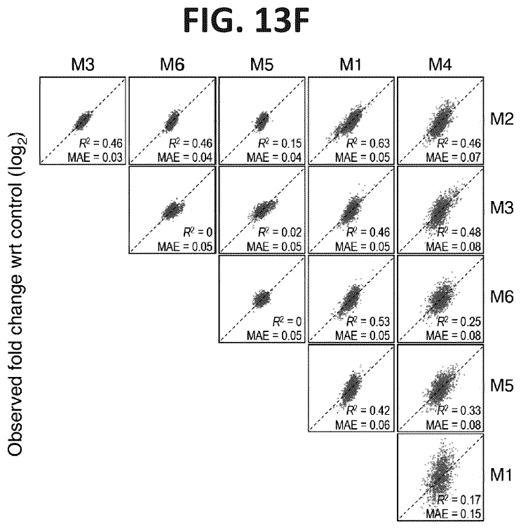

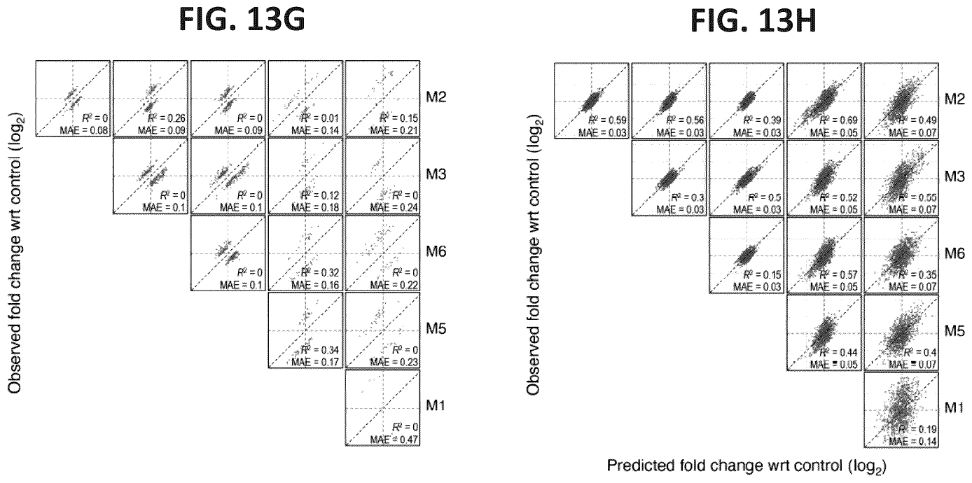

In the regulatory network, many of the E3s and other regulators impact the same genes and processes when perturbed individually, but, given the possible non-additivity of biological interactions, determining their effect if perturbed jointly (combinatorial perturbation) requires an additional experiment. In the large screen, 177,871 cells had more than one guide assigned (with 10,244 cells with guides targeting two or more of the 329 singly-impactful regulators; FIG. 13A), opening the way to test for such genetic interactions. However, because of random sampling from an enormous number of possible combinations, few to no cells were profiled for any specific combination, such that the joint effect of any individual combination could not be determined with confidence. Instead, it was reasoned that the co-functional modules could be leveraged to group all cells perturbed by a pair of guides from a given pair of modules (including the same module) to gain statistical power to decipher genetic interactions between or within modules, rather than between their individual constituent genes. Double perturbations were thus analyzed in a setting where cells are assigned to perturbed modules instead of perturbed genes. (The few detected triply perturbed cells were removed prior to analysis, as were module 4 perturbations given the very small number of cells.)

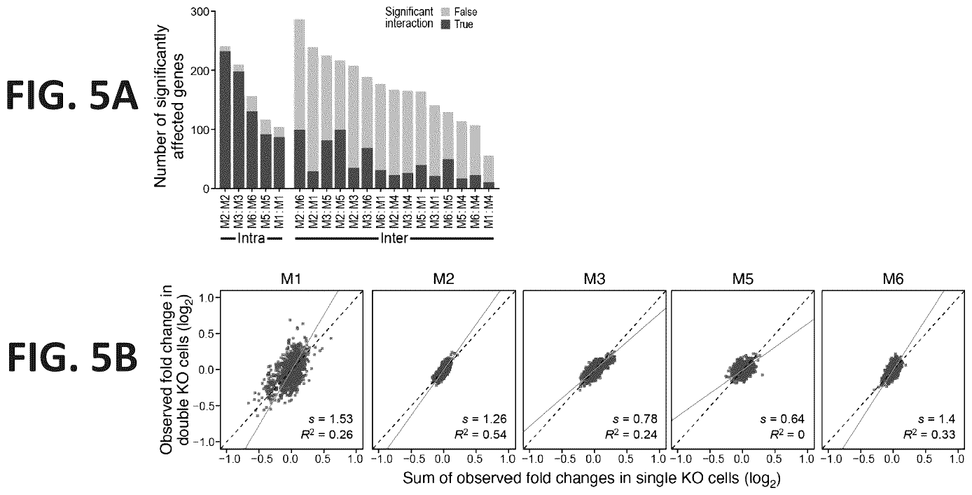

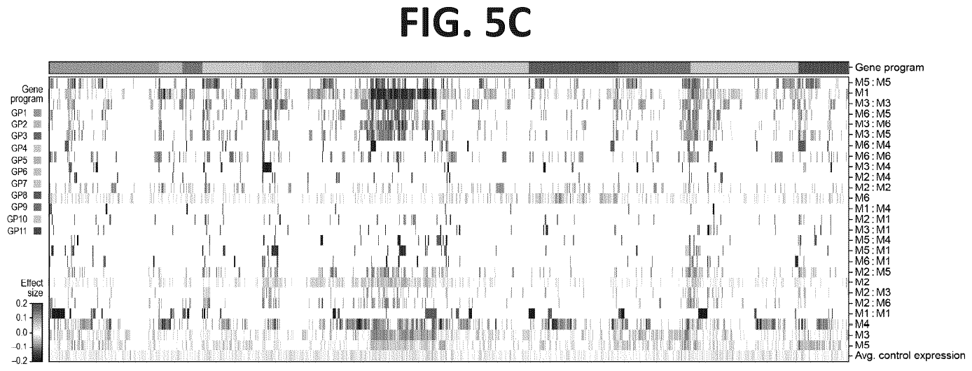

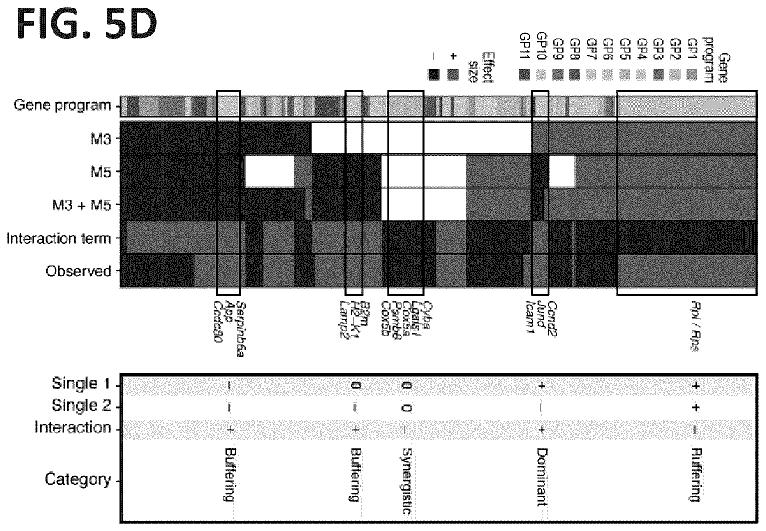

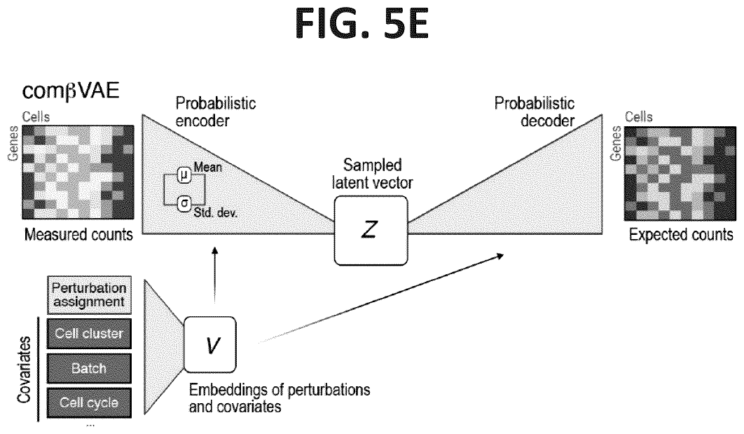

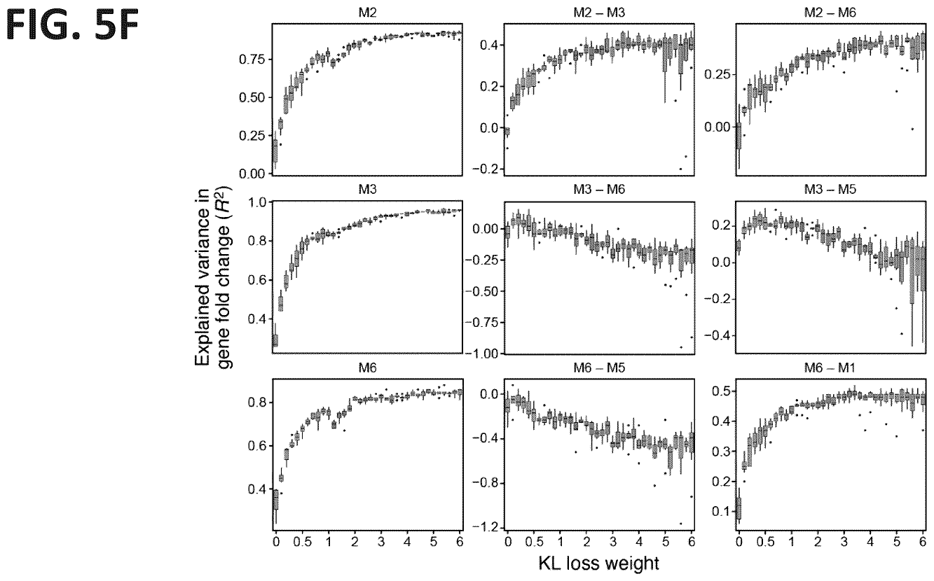

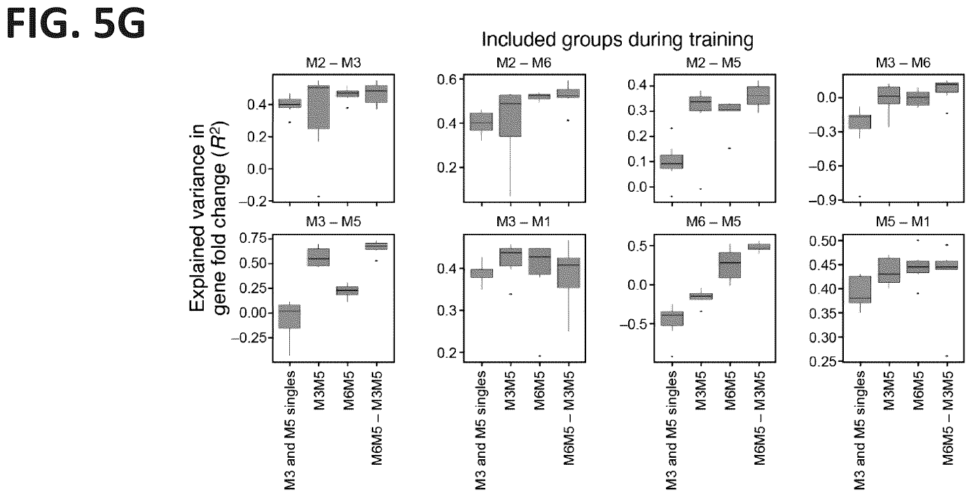

The proportion of genes whose expression has a significant interaction term due to a combinatorial perturbation was much greater in intra- vs. inter-module combinations (FIG. 5A). Thus, when two genes within the same module (intra-module combination) were perturbed in the same cell, the combined effects on the expression of genes were often different than the sum, with a super-linear relationship in M1, M2, and M6 (FIGS. 5A and 5B). Conversely, when two genes from different modules were perturbed in the same cell (inter-module combinations), the impact on most genes was additive (FIG. 5A).

At the level of expression of the individual affected genes, a range of patterns was found, with almost half (490 of 1,041 tested genes) with at least one significant interaction term (either positive (synergistic) or negative (antagonistic)) as a result of at least one of the 15 inter-module KO pair groups, and 650 with interaction terms in the five intra-module perturbation pairs (FDR <0.1, FIGS. 5C, 5D, 13B, 13D, and 13E). Genes vary in the extent of genetic interactions on their expression (FIG. 13B). Module pairs vary in the extent of interactions (FIG. 13C). In particular, a joint perturbation of an M3 and M5 regulator yielded non-additive effects in many genes (FIG. 5D), enriched for biosynthetic, translation, cytokine production, and inflammatory response genes. These included buffering for translation (GP6) and MHC-I presentation (GP10) program genes; synergy for protein homeostasis and phagocytosis (GP5) program genes, and dominance for mDC (GP7) program genes (FIG. 5D). Notably, the impacts of single perturbations in M3 and M5 regulators are often correlated (FIGS. 2C and 5D). Thus, similarly to the joint perturbation of two regulators from the same module, non-additive effects may be more prevalent for regulators from different modules but with similar effects on gene programs when individually perturbed.

### C. ComβVAE Predict Combinatorial Perturbations within and Across Modules

Next, it was determined how well the effects of the double knockouts can be predicted from profiles of single knockout cells. As a baseline, a simple linear model was first used to assess the overall effects of each of the six co-functional modules on the 1,041 response genes, and predict the log 2 fold changes of these genes in 20 pairwise module combinations by adding the individual KO group effects. As expected from the above analysis, additive effects explained most of the intra-module interactions quite poorly (FIG. 5B), while a substantial fraction of the variance in fold change was explained for some of the inter-module pair combinations (FIGS. 13F-13H). Inter-module groups where the additive model performed worse involved pairs of perturbations where more target genes show significant interactions, such as M2-M5 and M3-M5 (FIGS. 5A, 13C, and 13F-13H), or those with fewer double KO cells, like M1-M4 (FIGS. 13A and 13F-13H), which may have affected the estimation of the ground truth values. For genes with significant interaction terms, the additive model lost its predictive power in most of the module pairs (FIGS. 13F-13H), including a change in effect direction for many genes between prediction and observation.

It was hypothesized that better prediction performance could be gained by learning the interaction effects based on the latent structure of the gene expression profiles. To test this hypothesis, comβVAE, a conditional variational autoencoder (cVAE) (Lopez et al., Nat. Methods, 15: 1053-1058, 2018; Sohn et al., Learning Structured Output Representation using Deep Conditional Generative Models, in: Advances in Neural Information Processing Systems. Curran Associates, Inc., 2015) was developed, wherein the latent variables of the observed data are distributed conditioned on input data labels (FIG. 5E). The model was trained with 89,463 single KO cells (of 329 impactful KOs, 80,189 cells for training, 9,274 cells for validation) and a random sample of 70% of control cells, conditioning on 7 groups (controls+6 regulator modules). With the remaining 30% of control cells, the trained model was used to generate profiles based on the counterfactual questions “What would be the profile of this control cell if it had a single knockout from module x?” and “What would be the profile of this control cell if it had a double knockout, one from module x and another from module y?” Note that this model only addresses inter-module interactions, as the conditioning is done per KO module. Finally, the expression fold changes were calculated between these generated cells and the population of control cells.

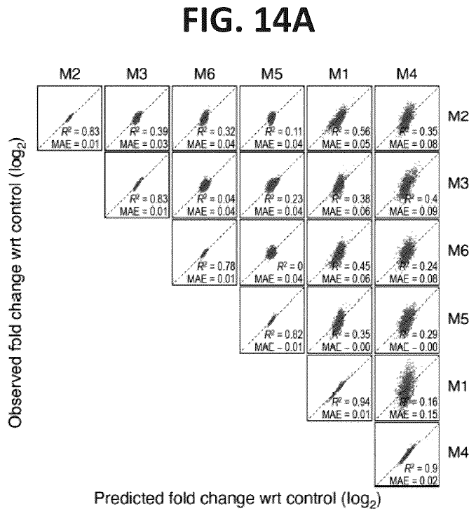

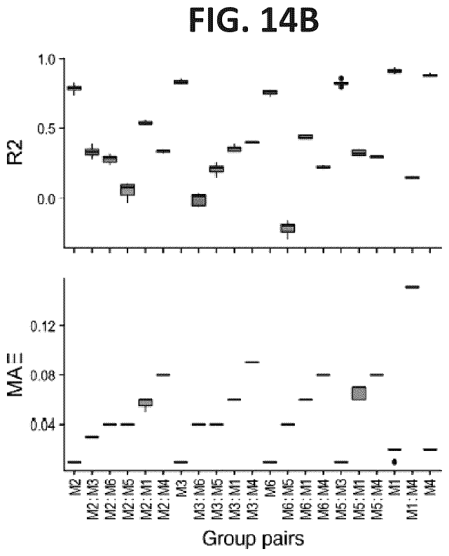

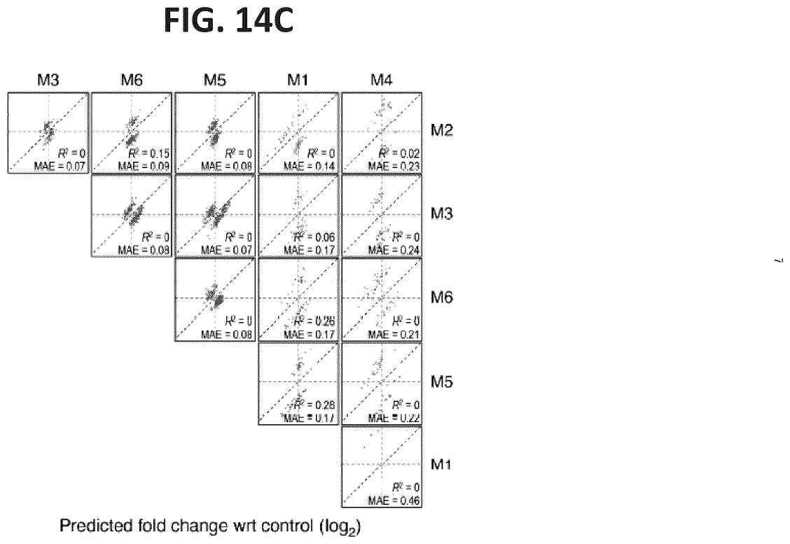

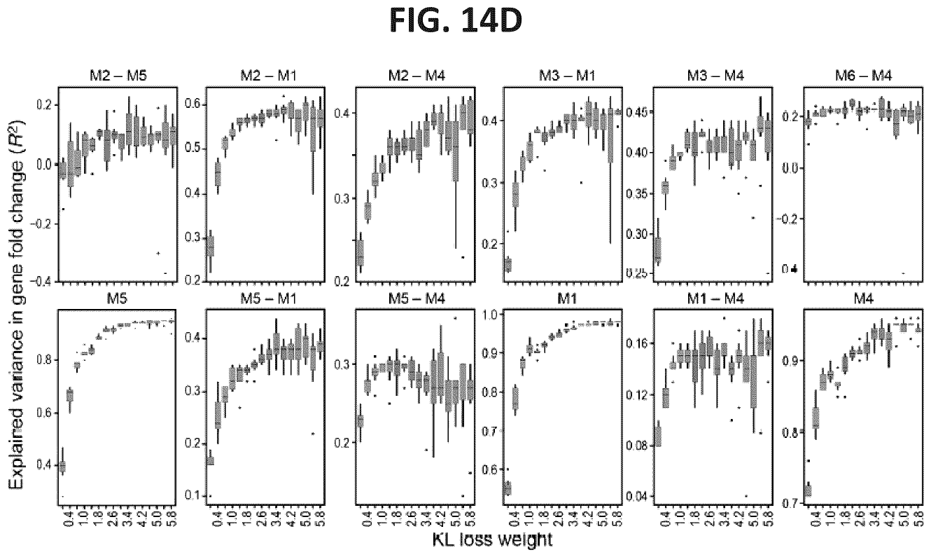

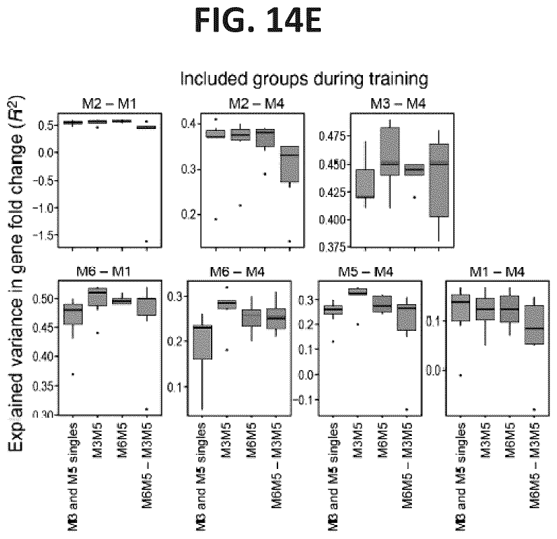

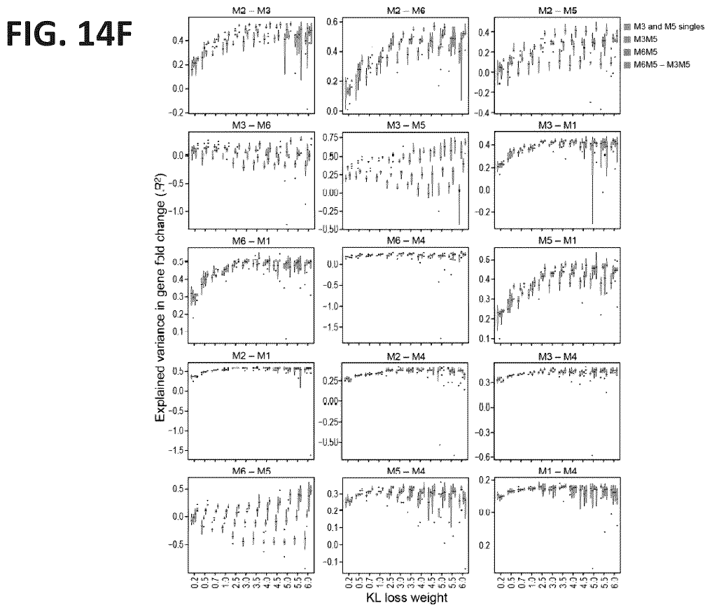

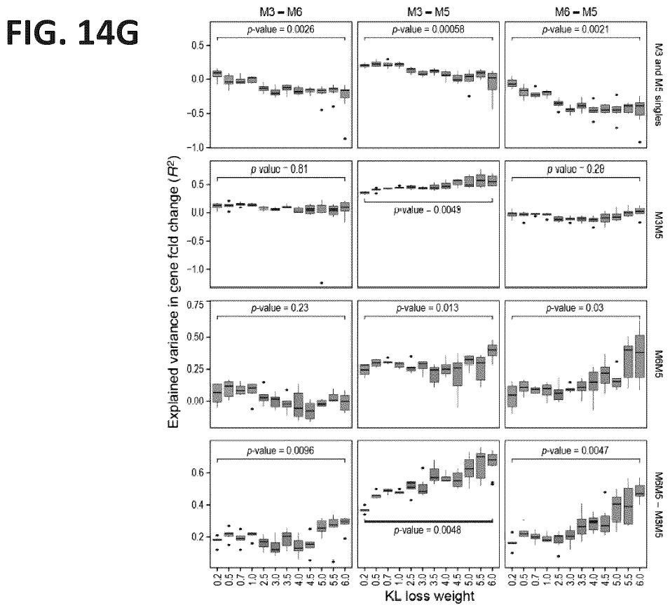

While the explained variance in the expression fold changes for generated KO profiles of single genes from the groups observed during training (single knockout modules) was quite high (0.77<r²<0.95, mean 0.85), estimates for double knockouts varied based on the module pair (FIGS. 14A and 14B). The profiles of cells with pairs of KOs from M3*M5, the module pair with the highest number of significant inter-module interactions (FIG. 13C), were estimated far better by the comβVAE (r²=0.23) than by the additive model (r²=0.02) (FIGS. 13F, 14A, and 14B). Moreover, the comβVAE had smaller mean absolute errors than the additive model when predicting the fold changes of genes with significant interaction terms (FDR<0.1), especially in module pairs with the highest number of genes with significant interactions (M3*M5, M3*M6 and M6*M5; FIGS. 13G and 14C). Higher values of Beta, the hyperparameter that changes the weight of the Kullback-Leibler (KL) loss term (which acts as a regularizer on the latent space distribution of the gene expression embeddings as well as the KO embeddings), increased the explained variance in single KOs modules and in double KO module pairs with fewer genes with interaction terms, but reduced it for pairs with more genes with interaction terms (FIGS. 5F, 13C, and 1D). Thus, greater latent space disentanglement (i.e., larger regularization parameter Beta) leads to better conditioning on the individual single KO groups, and better learning of additive effects at the cost of non-additive ones.

To evaluate how the predictions change when some double KO cells are included during training, the model was trained with the training set of the singly perturbed cells and the double KO cells of either M3M5, M5M6, or both. Interestingly, including double KO cells from one pair of modules increased the prediction performance of other unseen double KO groups (FIGS. 5G and 14E). Furthermore, when double KO cells were included in the training, higher beta values increased the prediction performance of inter-module groups with more interaction terms (FIGS. 14F and 14G). Thus, including some combinatorial perturbations during training along with better latent space disentanglement helps the model to learn both the generative factors and connections between them, leading to better prediction of unseen combinations.

### Example 5. In Vitro Perturbation-Defined Regulators and Programs are Associated with Genetic Risk in Inflammatory Diseases

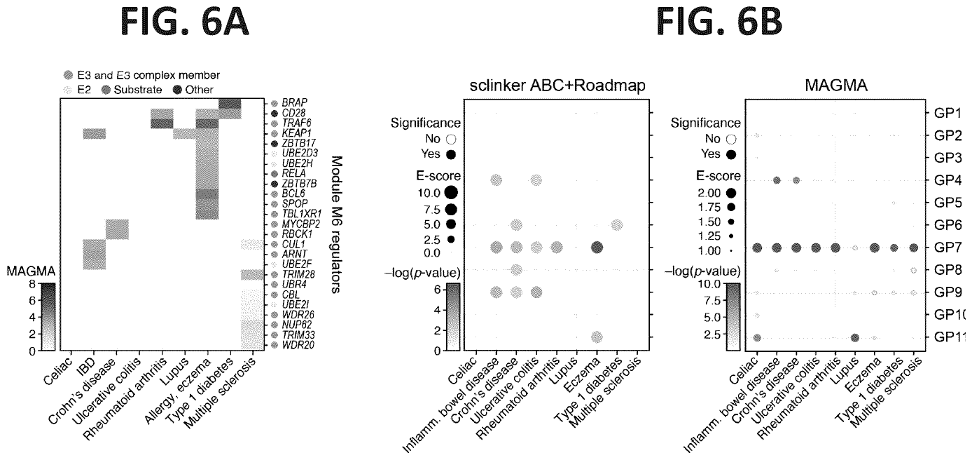

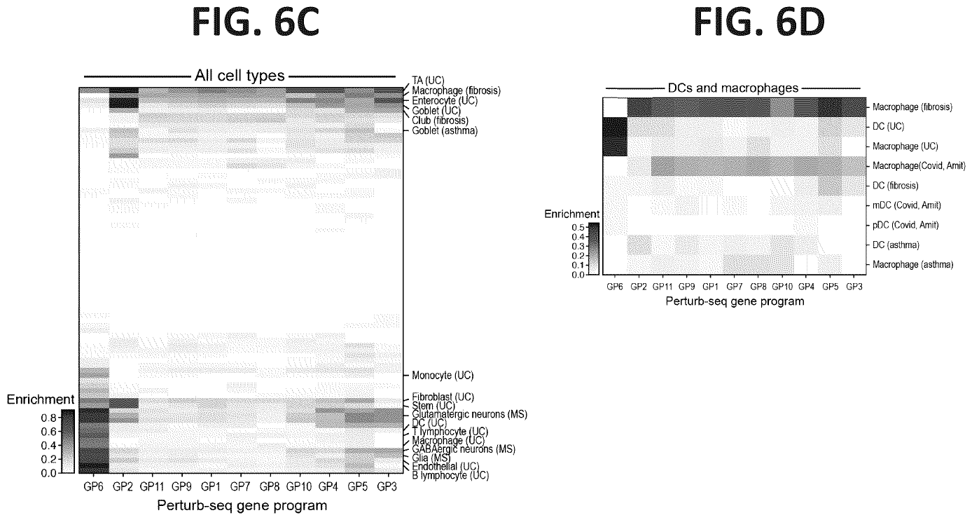

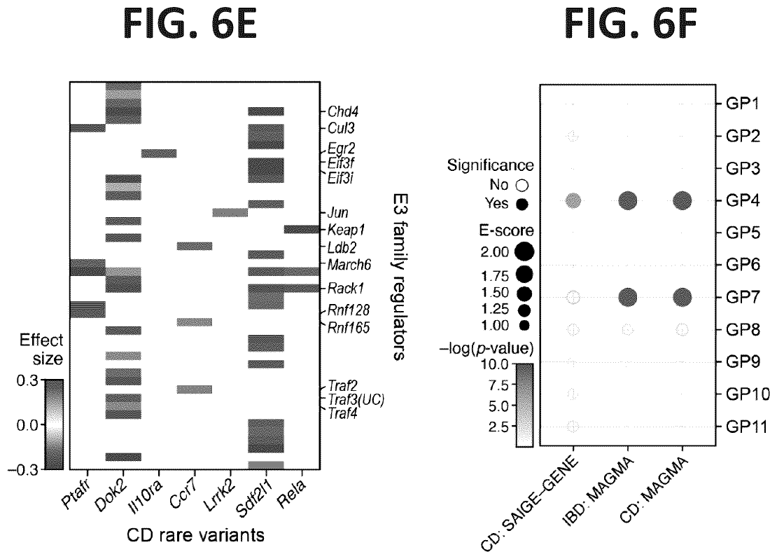

To determine whether the presently uncovered regulatory network contributes to or is active in human disease, it was next asked whether impactful regulators in the modules or the programs they regulate are also likely to be causal in human disease, based on either heritability signals, regulation in vivo during disease progression, or both. The modules and programs were thus tested for enrichment in common-variant driven disease associations using sc-linker (Jagadeesh et al., Nat. Genet., 54: 1479-249, 2022) and MAGMA (de Leeuw et al., PLoS Comput. Biol., 11: e1004219, 2015) with GWAS summary statistics from nine immune-related diseases (average N=79.5K, (FIGS. 6A and 6B and Tables 8-11). Each gene program was also compared each gene program to cell-type-specific disease progression programs induced in disease vs. healthy tissue by scRNA-Seq (Jagadeesh et al., Nat. Genet., 54: 1479-249, 2022) (FIGS. 6C and 6D). Support for both relationships was found.

Among the co-functional modules, common and low frequency variants in genes in module M1 were enriched for heritability across all traits tested (1.62-fold on average, P=1.52×10⁻⁵), and especially immune-related traits (1.94 fold; P=2×10⁻⁴), compared to genes constituting all the modules (Tables 8 and 9).

TABLE 8
Co-functional gene module enrichment in common and
low frequency variant driven disease associations

| | Meta.enr | Meta.senr | Meta.penr |
|---|---|---|---|
| M1 | 1.62400298 | 0.12115438 | 1.52E−05 |
| M2 | 1.17302285 | 0.22026348 | 0.74024796 |
| M3 | 1.23893256 | 0.13213048 | 0.29303818 |
| M4 | 1.46882215 | 0.14637968 | 0.01174794 |
| M5 | 1.33736482 | 0.10260705 | 0.02070405 |
| M6 | 1.27651003 | 0.13788682 | 0.20050713 |

TABLE 9
Co-functional gene module enrichment in
immune-related disease associations

| | Meta.enr | Meta.senr | Meta.penr |
|---|---|---|---|
| M1 | 1.93850134 | 0.22599601 | 0.00020706 |
| M2 | 0.99209897 | 0.66496014 | 0.87109566 |
| M3 | 1.13188089 | 0.41906365 | 0.93935825 |
| M4 | 1.18156712 | 0.57568017 | 0.88732626 |
| M5 | 1.37567614 | 0.2590584 | 0.28726225 |
| M6 | 1.25121401 | 0.29928731 | 0.61338537 |

TABLE 10
Extended sc-linker program heritability (Groups GP11-GP6)

| rownames(EE1) | GP11 | GP10 | GP9 | GP8 | GP7 | GP6 |
|---|---|---|---|---|---|---|
| RBC.distwidth | 12.7641477 | 8.22071113 | 6.05984675 | 9.03808447 | 8.77586731 | 13.3174905 |
| RBC.count | 1.16932398 | 5.38127414 | 5.1663828 | 10.9324617 | 12.5179383 | 13.815879 |
| WBC.count | 10.0540809 | 11.8840318 | 10.2150494 | 16.5887246 | 15.1130735 | 11.1618018 |
| Platelet.count | 4.72385062 | 10.4359016 | 11.6103838 | 12.1335607 | 12.6045983 | 13.3911148 |
| Eosino.count | 36.5423687 | 8.53152937 | 17.3678461 | 14.4823455 | 24.7671456 | 10.2102068 |
| ALL_Inflammatory | 1.12888595 | 18.4162306 | 19.5525127 | 20.0396061 | 40.2214402 | 11.8301463 |
| UC | 15.0229255 | 10.6254562 | 17.6881968 | 14.0004604 | 32.3690438 | 9.05599019 |
| CD | 5.82916049 | 1 | 19.4838837 | 10.7634305 | 30.167724 | 22.5925505 |
| RA | 15.4196766 | 6.54735437 | 16.9907595 | 8.3280765 | 34.8210878 | 27.3734254 |
| Celiac | 35.5722339 | 3.21400785 | 7.73962127 | 15.5642192 | 21.4830136 | 24.3827696 |
| Lupus | 23.5875939 | 22.2065529 | 33.7717889 | 4.27136291 | 28.5906626 | 33.3865803 |
| T1D | 19.5737502 | 17.0693866 | 34.316359 | 21.0234151 | 14.8895476 | 35.5389734 |
| IBD | 4.15884735 | 9.42461494 | 17.2029875 | 11.7461769 | 32.1663709 | 14.9935369 |
| PBC | 4.22033902 | 9.97301243 | 30.5110037 | 8.2474934 | 29.4115241 | 21.5001848 |
| BMIZ | 1 | 1.29338772 | 1 | 1.60104299 | 2.36320731 | 1 |
| Edu.years | 1 | 1.9358139 | 1 | 1.30162276 | 2.68777595 | 1 |
| Lung.FVCSmoke | 2.30733523 | 2.1684352 | 2.46046294 | 1 | 1.76802229 | 7.40624519 |
| Intelligence | 1 | 1.25053046 | 1 | 1.19229003 | 4.26395538 | 3.62880076 |
| Neuroticism | 1.18015873 | 1.45414384 | 1 | 1.28346925 | 2.89652955 | 5.72051208 |
| Diastolic.bp | 1 | 3.51159247 | 9.44945137 | 2.73204983 | 3.23841895 | 6.23177563 |
| Height | 1.72181197 | 3.83896432 | 1 | 1 | 1 | 11.0575994 |
| Morning.Person | 1 | 2.68738478 | 1 | 2.71082645 | 3.029779 | 3.24481502 |
| Menarche.Age | 1 | 1.06381772 | 1.29034943 | 1.02299678 | 1 | 2.76111962 |
| Total.protein | 15.0815949 | 14.0576374 | 11.9737746 | 11.2634719 | 17.5164085 | 21.4867378 |
| WHRadjBMI | 1.93536989 | 1.6065755 | 3.55259291 | 3.11610373 | 1.27785186 | 9.08226226 |
| Lung.FEV1FVCSmoke | 3.04110827 | 1.34640338 | 1.22748693 | 1.80171351 | 2.55571596 | 7.62239326 |
| Reaction.Time | 1 | 2.3004183 | 1 | 2.76897491 | 1 | 6.07812801 |
| Creatinine | 1.99065971 | 2.8745415 | 1.74558759 | 1 | 3.35655067 | 3.4414001 |
| BD_SCZ | 1 | 2.63703997 | 1.11420156 | 1 | 2.27830773 | 1.59747343 |
| Heel.Tscore | 2.65438734 | 2.99230615 | 1.79958227 | 3.43642849 | 1 | 6.94436515 |
| Sleep.Duration | 1 | 4.71988119 | 3.75118905 | 2.15741161 | 3.66661837 | 3.1363174 |
| IGF1 | 1 | 1 | 1.23037876 | 1 | 6.1722672 | 1 |
| General.RiskTolerance | 2.53342258 | 1 | 2.07968207 | 1 | 2.21973824 | 1 |
| AspartateAminotransferase | 1 | 9.10084846 | 4.9694543 | 10.9263759 | 17.9053041 | 7.17891476 |
| Insomnia | 1 | 1 | 1 | 1.39834241 | 1 | 3.03315439 |
| Num.Children | 9.50996069 | 1 | 3.14452331 | 1 | 1 | 1.50379487 |
| Atrial.Fibrillation | 2.79042069 | 7.55744811 | 3.52739956 | 1 | 5.41361762 | 2.32486328 |
| Testosterone.Male | 1.66113166 | 2.15507853 | 1 | 2.58455432 | 3.51104807 | 1.34748862 |
| BMI1 | 1.11815815 | 1 | 1 | 1.13105749 | 2.60258683 | 3.73121068 |
| Hypothyroidism | 20.2345808 | 12.5036604 | 16.5869101 | 7.94689372 | 12.4566862 | 21.9170645 |
| Balding | 8.51989233 | 1 | 2.85926346 | 4.10067368 | 1 | 4.60583179 |
| Alkalinephosphatase | 7.86661216 | 1 | 3.93644232 | 6.62369942 | 8.88885635 | 22.9462117 |
| Allergy.Eczema | 10.3520281 | 4.48785358 | 12.2790625 | 5.63420148 | 15.3993784 | 14.8324925 |
| Drinks.perWeek | 1 | 1.65759151 | 1.00400382 | 2.52924906 | 1.80723851 | 4.39522125 |
| Phosphate | 2.74278177 | 1 | 1 | 2.84899465 | 1 | 1 |
| Medication.Use | 1 | 1.61641123 | 1 | 3.76792735 | 1.5113645 | 1.65950596 |
| BRCA | 1 | 1 | 2.98502521 | 1 | 2.623579 | 6.2855886 |
| VitaminD | 1 | 2.83195971 | 1.23706134 | 1 | 9.07438389 | 1 |
| MDD | 1 | 8.27733884 | 2.46166036 | 1 | 2.73110446 | 1 |
| Cholesterol | 2.28988681 | 11.8117501 | 14.3069803 | 6.9280058 | 23.8331367 | 3.99724661 |
| ADHD | 2.43693501 | 1 | 1 | 1 | 1 | 4.10461972 |
| TotalBilirubin | 3.37395225 | 20.5005443 | 5.33989302 | 5.90477721 | 1 | 1 |
| Menopause.Age | 1 | 1.03202652 | 2.44933147 | 9.01961885 | 1 | 13.1847611 |
| Pigment.Sunburn | 66.1338018 | 1 | 1 | 3.75526399 | 1 | 28.858243 |
| SCZvsBD | 1 | 1 | 3.26338793 | 1 | 1 | 2.84055811 |
| HDL | 32.9434926 | 12.3149349 | 24.8705918 | 22.275372 | 23.3265705 | 5.76274975 |
| LDL | 1 | 9.21471064 | 15.9779426 | 1.91414962 | 10.4774717 | 19.9152348 |
| Cigarettes.Perday | 1 | 2.57150891 | 1 | 5.35587815 | 1 | 12.4910632 |
| Ischemic.Stroke | 7.94768239 | 1 | 6.9430171 | 4.64571272 | 1 | 17.2886927 |
| Anorexia | 6.82460991 | 4.64332226 | 1 | 5.69833729 | 1 | 3.11382934 |
| MS | 9.81484051 | 14.443894 | 10.0832086 | 26.0408443 | 37.5081148 | 1 |
| Ever.smoked | 5.88888773 | 9.70646303 | 8.10477582 | 1 | 1 | 1 |
| PRCA | 1 | 1.79471424 | 1 | 7.32965503 | 2.97777604 | 4.31123283 |

TABLE 11
Extended sc-linker program heritability (Groups GP5-GP1)

| rownames(EE1) | GP5 | GP4 | GP3 | GP2 | GP1 |
|---|---|---|---|---|---|
| RBC.distwidth | 5.34284283 | 11.4772729 | 8.73773473 | 14.7479905 | 6.42135885 |
| RBC.count | 9.39698387 | 10.6784839 | 9.18095298 | 10.2179087 | 5.30901992 |
| WBC.count | 15.7824926 | 20.8949271 | 16.7041011 | 10.167312 | 8.71624132 |
| Platelet.count | 14.5441782 | 24.9330053 | 1 | 1 | 10.4432113 |
| Eosino.count | 24.6653314 | 21.4201646 | 23.5757785 | 20.2736869 | 4.12439287 |
| ALL_Inflammatory | 16.353471 | 12.0919112 | 12.8434167 | 4.83771078 | 17.2194373 |
| UC | 13.2760007 | 34.1715668 | 37.7277022 | 1 | 5.15384325 |
| CD | 15.1314595 | 23.2944062 | 18.6579287 | 16.7336972 | 1 |
| RA | 1.65719858 | 15.1823046 | 11.0800237 | 1 | 14.0956214 |
| Celiac | 44.9966068 | 6.07541195 | 22.1804601 | 16.6552348 | 3.06790426 |
| Lupus | 19.1708961 | 17.4779691 | 23.7575657 | 36.3121133 | 16.4771551 |
| T1D | 25.7817247 | 19.6674869 | 26.2665003 | 1.93665482 | 5.43534483 |
| IBD | 13.8404187 | 29.7325361 | 6.97240364 | 7.36221805 | 2.10445187 |
| PBC | 17.8206935 | 27.8982596 | 32.3878258 | 11.3973644 | 1 |
| BMIZ | 1.77409539 | 1.82421485 | 1 | 1 | 1.5429169 |
| Edu.years | 1.60159183 | 4.45664902 | 1 | 2.52689046 | 1.59215421 |
| Lung.FVCSmoke | 1 | 2.21948124 | 1 | 1 | 1 |
| Intelligence | 1 | 6.36877088 | 1 | 1.31300607 | 1 |
| Neuroticism | 4.0168829 | 5.33199159 | 1 | 1 | 1 |
| Diastolic.bp | 7.16754356 | 11.2576446 | 1.29714546 | 2.24291493 | 2.02820578 |
| Height | 2.24813992 | 2.35777964 | 1 | 6.49066215 | 1.2754737 |
| Morning.Person | 1 | 1 | 1 | 7.31925722 | 1 |
| Menarche.Age | 5.10674027 | 1 | 1 | 4.28816583 | 1 |
| Total.protein | 19.2279783 | 13.0622541 | 1.96871278 | 1.20563714 | 6.96773756 |
| WHRadjBMI | 1 | 6.59499358 | 5.55265549 | 3.57702493 | 1 |
| Lung.FEV1FVCSmoke | 4.82054013 | 2.57653088 | 1 | 2.41608609 | 2.26590403 |
| Reaction.Time | 2.79013565 | 1 | 4.19608725 | 3.50824469 | 1 |
| Creatinine | 3.70952931 | 10.2268195 | 1 | 1 | 2.02831472 |
| BD_SCZ | 6.9926378 | 6.07569451 | 1 | 1 | 1 |
| Heel.Tscore | 2.45006436 | 6.5092453 | 1 | 4.56678873 | 1 |
| Sleep.Duration | 3.80401542 | 3.94951564 | 2.35249621 | 2.2902215 | 3.2529019 |
| IGF1 | 1 | 3.27218711 | 1 | 1 | 1.47481127 |
| General.RiskTolerance | 1 | 4.59698804 | 1 | 1 | 3.18204276 |
| AspartateAminotransferase | 6.82100431 | 15.8080743 | 1 | 4.27080742 | 4.00673018 |
| Insomnia | 1 | 1 | 4.60940748 | 1 | 1 |
| Num.Children | 2.34371265 | 1 | 1 | 7.94573708 | 1.21675023 |
| Atrial.Fibrillation | 2.65332063 | 1 | 1.16050306 | 1.26831963 | 1 |
| Testosterone.Male | 9.97438485 | 2.316242 | 1 | 1 | 1 |
| BMI1 | 1 | 1.78254077 | 1 | 4.50974966 | 1 |
| Hypothyroidism | 13.4911763 | 13.0721704 | 2.85652547 | 8.54638469 | 2.9048437 |
| Balding | 2.12200309 | 9.31132847 | 1 | 6.30620966 | 1 |
| Alkalinephosphatase | 17.5634009 | 19.4295013 | 1 | 13.2989696 | 4.27830456 |
| Allergy.Eczema | 6.90242331 | 7.88384981 | 22.659494 | 4.40869025 | 7.2903012 |
| Drinks.perWeek | 2.38703208 | 3.13614622 | 2.83694827 | 1 | 1.77482252 |
| Phosphate | 7.52099476 | 12.0061971 | 23.1966867 | 1 | 3.54297185 |
| Medication.Use | 4.95143446 | 1 | 3.83221035 | 1 | 1 |
| BRCA | 19.3210059 | 15.2904066 | 2.05012427 | 3.1591539 | 2.1565718 |
| VitaminD | 2.63882863 | 1.37705554 | 1 | 9.88325763 | 1.50852837 |
| MDD | 7.81811421 | 2.11107863 | 1.35499617 | 9.39522573 | 5.85477498 |
| Cholesterol | 5.16444244 | 4.00258426 | 1 | 36.9135436 | 5.41752518 |
| ADHD | 1 | 4.36532563 | 2.73173681 | 8.62737823 | 1.22162185 |
| TotalBilirubin | 7.87429263 | 7.27152564 | 7.32022175 | 5.41102647 | 2.43550941 |
| Menopause.Age | 2.2467961 | 3.01604938 | 6.33546753 | 3.19051839 | 1 |
| Pigment.Sunburn | 8.96305481 | 9.34159773 | 1 | 1.01953952 | 4.69614097 |
| SCZvsBD | 6.02029728 | 1 | 14.9949518 | 1 | 1 |
| HDL | 1 | 1 | 1 | 84.5842967 | 5.71394213 |
| LDL | 10.3216551 | 1 | 1 | 8.68379126 | 9.15289609 |
| Cigarettes.Perday | 1.66147865 | 1 | 1.2936421 | 1 | 1 |
| Ischemic.Stroke | 6.19034194 | 9.37170138 | 12.8108573 | 1 | 1.11070733 |
| Anorexia | 3.6568682 | 4.27347082 | 1.59538415 | 12.915337 | 1 |
| MS | 1.9137627 | 1 | 1 | 21.4411644 | 10.9378224 |
| Ever.smoked | 3.99913675 | 9.25778724 | 17.8726895 | 1 | 1 |
| PRCA | 3.04132986 | 5.2049059 | 42.5175335 | 23.1419771 | 1 |

In particular, M1 member genes and the predicted E3 WDR36 have a significant MAGMA score in allergy/eczema and blood traits, and the E3 adapter PTPN11 has a significant MAGMA score in type 1 diabetes (T1 D) and blood traits (Z-scored per-trait MAGMA scores, Bonferroni correction α=0.1). Notably, perturbing module M1 activates the mDC (GP7), TNF/LPS response (GP8), and MHC-I Ag presentation (GP10) programs and represses ER stress (GP2), protein homeostasis and phagocytosis (GP5) and translation (GP6) (FIGS. 2C and 2D; Tables 10 and 11), consistent with the association of variants in this regulating module with inflammatory disease. Additionally, many E3s and E3 complex members in M6 are associated with risk of immune-related traits (FIG. 6A), including Traf6 (eczema and RA), Rbck1 (CD), Bcl6 (eczema), Keap1 (eczema, IBD, and lupus), Brap (T1 D), and Cull (IBD) (Z-scored per-trait MAGMA scores, Bonferroni MTC a=0.1; FIG. 6A).

Several co-regulated gene programs also showed heritability enrichment in immune diseases. These included the mDC program (GP7) (FIG. 6B, highest score by both sc-linker and MAGMA), especially REL (rank 1 average MAGMA scores across immune diseases), JAK2 (rank 3) and STATSA (rank 8); and the motility and cell maintenance (GP4) program in IBD and related traits (FIG. 6B), with top driving genes CCL2 (rank 2) and CCL7 (rank 9) (Carson et al., Cell. Immunol., 314: 63-72, 2017; Morris et al., Front. Immunol., 9: 2018).

Concomitantly, several of the perturbation-affected programs were enriched (relative to all genes expressed in this cell type) in disease progression programs from multiple cell types and diseases, especially those of three key cellular processes that are also implicated in modulating immune responses—mitochondrial metabolism (Jovanovic et al., Science, 347: 1259038, 2015; Pearce and Everts, Nat. Rev. Immunol., 15: 18-29, 2015), ER stress (Cubillos-Ruiz et al., Cell, 161: 1527-1538, 2015) and translation/antigen presentation. This is consistent with a model where processes affected by heritable variation in regulators lead to dysregulation of their target programs. For example, translation program genes (GP6) were enriched in disease progression programs of immune and non-immune cells in inflammatory diseases, including ulcerative colitis (UC) and multiple sclerosis (MS) (FIGS. 6C and 6D); ER stress response genes (GP2) were enriched in disease progression programs in epithelial cells in UC, lung fibrosis and asthma (FIG. 6C); and disease progression programs in macrophages and DCs in UC are enriched for the translation program (GP6) (FIG. 6D). These programs are all regulated by module M1, which is itself enriched for heritability of disease risk, as noted above. Thus, this analysis suggests that at least two modules (M1 and M6) may contribute to disease risk through their respective activation or repression of disease-induced programs including ER stress (GP2), motility and cell maintenance (GP4), and translation (GP_6), including by E3 risk genes Wdr36 (M1) and Keap1, Cul1, and Rbck1 (M6). In addition, the GP4 program is enriched for genes with rare-variant driven association in Crohn's disease (Sazonovs et al., 2022), including COX4/1, POLD4 and NPY(ranked 1, 2 and 4; FIG. 6F).

Interaction between NPY neurons and immune cells in the enteric nervous system has previously been implicated in IBD pathogenesis, and NPY expression changes occur in animal models of IBD (Chandrasekharan et al., Am. J. Physiol. Gastrointest. Liver Physiol., 304: G949-957, 2019; El-Salhy and Hausken, Neuropeptides, 55:137-144, 2016).

Finally, the present model was applied as a “look up” resource for proposing regulators of new rare risk variants that were recently identified for Crohn's disease (Sazonovs et al., Nat. Genet., 54: 1275-1283, 2022). The present model suggests that II10ra expression is repressed by Egr2 and that expression of Ccr7, a chemokine receptor regulating many aspects of DC function and guiding DCs to lymph nodes (Rodriguez-Ferndndez and Criado-Garcia, Front. Immunol., 11: 528, 2020), is repressed by Ldb2, Traf2, and Rnf165 (FIG. 6E). While Traf2 deletion impacts inflammation in both DCs and keratinocytes (Etemadi et al., eLife, 4: e10592, 2015), Traf2-based regulation of Ccr7 has not been previously reported. Increased Ccr7 expression could be an important mechanism in increased T cell infiltration and inflammation controlled by Traf2.

Thus, multiple components of the present model, including regulatory modules and their impacted programs, are congruent with both risk genes for human immune and inflammatory disease and the dysregulated expression programs observed in patients. This highlights the relevance of this in vitro screen to human disease.

### A. Detailed Methods Relating to Disease Progression Gene Programs

Disease progression programs were defined as previously described (Jagadeesh et al., Nat. Genet., 54: 1479-249, 2022) using publicly available scRNA-seq datasets, processed, annotated, and analyzed as previously described (Jagadeesh et al., Nat. Genet., 54: 1479-249, 2022). A gene-level non-parametric Wilcoxon rank sum differential expression test was performed between cells from healthy and disease tissues of the same cell type as previously described (Jagadeesh et al., Nat. Genet., 54: 1479-249, 2022).

### B. Detailed Methods Relating to Identification of Heritability Signal

Gene programs and co-functional modules were tested for enrichment in heritability signal using both scLinker (Jagadeesh et al., Nat. Genet., 54: 1479-249, 2022) and MAGMA (de Leeuw et al., PLoS Comput. Biol., 11: e1004219, 2015) over a set of 60 relatively independent diseases and traits (average N=297K) (Jagadeesh et al., Nat. Genet., 54: 1479-249, 2022).

In sclinker, each program or module was combined with enhancer-gene linking strategies defined by either SNPs in enhancers linked to genes based on the Roadmap (Liu et al., Genome Biol., 18: 193, 2017) and Activity-By-Contact (ABC) SNP-To-Gene (S2G) strategies either aggregated across all biosamples related to blood (RoadmapUABC-Blood). For each gene score X and S2G strategy Y, a combined annotation X×Y was defined by assigning to each SNP the maximum gene score among genes linked to that SNP (or 0 for SNPs with no linked genes); this generalizes the standard approach of constructing annotations from gene scores using window-based strategies (Finucane et al., Nat. Genet., 50: 621-629, 2018; Zhu and Stephens, Nat. Commun., 9: 4361, 2018). Heritability analysis of these sclinker annotations was performed using stratified LD score regression (Bulik-Sullivan et al., Nat. Genet., 47: 291-295, 2015; Finucane et al., Nat. Genet., 47: 1228-1235, 2015) conditional on a set of 86 baseline coding, conserved and LD-related annotations (baseline-LDv2.1 (Gazal et al., Nat. Genet., 49: 1421-1427, 2017)). The Enrichment Score (Escore) metric (Jagadeesh et al., Nat. Genet., 54: 1479-249, 2022) reported was derived from heritability enrichment analysis and its corresponding p-values.

For MAGMA analysis, the MAGMA z-score was computed for each gene module or program using a 0 kb window based strategy for linking SNPs to genes (de Leeuw et al., PLoS Comput. Biol., 11: e1004219, 2015) and then a gene set enrichment analysis of the MAGMA z-scores was performed for each with respect to a set of 1,000 sets of same size of randomly selected genes from across all perturbation programs (using the fgsea software (Korotkevich et al., Fast gene set enrichment analysis, bioRxiv, 2021)).

### C. Conclusions: Leveraging Cell Screens to Decipher Human Genetics

The reverse genetic approach of Perturb-Seq screens can complement and help interpret the results of forward genetic studies in humans, such as GWAS, especially in systems like primary DCs, where human cell models are lacking. By considering both regulators and regulated programs from the model in the context of associations from human GWAS and single cell profiles from relevant human disease tissues, it was found that both common and low frequency variants in regulators in module M1 are enriched for heritability in immune-related traits, including the in predicted E3 WDR36 9 (in allergy/eczema and blood traits) and E3 adapter PTPN11 in T1 D and blood traits. Moreover, two of the programs affected by perturbations in module M1 regulators—ER stress (GP2) and translation (GP6)-are differentially expressed in relevant cell types in immune disease, including macrophages and DCs in UC (GP2) and fibrosis and asthma (GP6).

### Example 6. Cell Therapies

The present findings show that multiple perturbations of genes within different knockout modules (Examples 3 and 4; Table 5) are more often nonlinear, impacting a larger number of regulated genes, whereas combinatorial perturbations in which the two perturbations were members of two different gene modules had effects that were more often additive. These findings are used to explore the search space of combinatorial perturbations used to improve cellular therapies, including design of enhanced chimeric antigen receptor T cell (CAR-T) therapies, T cell receptor-engineered T cell (TCR-T) therapies (autologous or induced pluripotent stem cell (IPSC)-derived), monocyte/myeloid cell therapies, or a therapy for use in regenerative medicine (e.g., a Müller glia cell therapy or a retinal ganglion cell (RGC) therapy).

Basic experiments are performed to identify the core modules in each cell type. It is predicted that the linearity (or non-linearity) of combinatorial perturbations of multiple genes across modules and within modules would remain consistent across different cell types.

In one example, a dendritic cell (DC) cancer immunotherapy that increases killing of cancer cells by activated T cells is developed by engineering progenitor cells (autologous or stem-cell derived) with several perturbations to improve DC priming and activation of T cells. Engineering at least two perturbations from the same knockout module (Table 5) is expected to have a larger impact (more nonlinearities across a larger number of impacted genes) than engineering two perturbations from two different modules (where the effect was most commonly additive).

In a further example, DC cancer immunotherapies are designed to activate programs GP9-11 (“Regulation of autophagy and inflammation,” “MHC-I Ag presentation,” and “MHC-II Ag presentation”) as presented in Table 6 to improve presentation and cross-presentation. Module M1 contains genes that are repressors of GP9-11 (FIG. 2D), and there is a strong negative M1:M1 perturbation interaction term for a subset of genes in the GP10 gene program. Selecting two M1 module genes to perturb combinatorially in a DC therapy could yield a better-presenting DC that would lead to more tumor cell killing by T cells.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating a cancer, an inflammatory disease, or an autoimmune disease in an individual, the method comprising administering to the individual an effective amount of a modulator of the interaction between (a) one, two, or all three of LIM domain-binding protein 2 (Ldb2), Ring finger protein 165 (Rnf165), and TNF receptor-associated factor 2 (Traf2) and (b) chemokine receptor type 7 (CCR7).

2. The method of claim 1, wherein the individual has a cancer and the modulator is an agent that decreases the expression and/or activity of one, two, or all three of Ldb2, Rnf165, and Traf2.

3. The method of claim 1, wherein the individual has an inflammatory disease or an autoimmune disease and the modulator is an agent that increases the expression and/or activity of one, two, or all three of Ldb2, Rnf165, and Traf2.

4. A method for;
(a) increasing expression of chemokine receptor type 7 (CCR7) in an antigen-presenting cell (APC);
(b) increasing APC migration to a tumor and/or a lymph node in an individual; and/or
(c) increasing T cell homing to a tumor in an individual, the method comprising contacting the APC with or administering to the individual an effective amount of an agent that decreases the expression and/or activity of one, two, or all three of LIM domain-binding protein 2 (Ldb2), Ring finger protein 165 (Rnf165), and TNF receptor-associated factor 2 (Traf2).

5. The method of claim 4, wherein the APC is in an individual.

6. (canceled)

7. The method of claim 4, wherein:
(a) CCR7 expression in the APC is increased by at least 10% relative to expression in the absence of the agent;
(b) APC migration to the tumor and/or lymph node in the individual is increased by at least 10% relative to migration in the absence of the agent; or
(c) T cell homing to the tumor in the individual is increased by at least 10% relative to T cell homing in the absence of the agent.

8. (canceled)

9. The method of claim 4, wherein the individual has a cancer.

10. (canceled)

11. The method of claim 4, wherein the APC is a dendritic cell (DC), a macrophage, or a glial cell.

12.-15. (canceled)

16. The method of claim 1, wherein the inflammatory disease or autoimmune disease is a neurodegenerative disease, arthritis, allergy, eczema, fibrosis, asthma, lupus erythematosus, an inflammatory bowel disease, ulcerative colitis, or Crohn's disease.

17.-18. (canceled)

19. The method of claim 4, wherein the agent is a proteolysis targeting chimera (PROTAC), a small molecule, an antibody or antigen-binding fragment thereof, a peptide, a mimic, or an inhibitory nucleic acid.

20. The method of claim 19, wherein:
(a) the inhibitory nucleic acid is an ASO or an siRNA; or
(b) the antigen-binding fragment is a bis-Fab, an Fv, a Fab, a Fab′-SH, a F(ab′)₂, a diabody, a linear antibody, an scFv, an scFab, a VH domain, or a VHH domain.

21. (canceled)

22. The method of claim 19, wherein the antibody or antigen-binding fragment thereof binds:
(a) Ldb2, Rnf165, or Traf2; or
(b) CCR7.

23. (canceled)

24. The method of claim 20, wherein the agent is a bispecific antibody comprising an antigen-binding domain that targets the tumor microenvironment.

25. The method of claim 4, wherein the method further comprises administering to the individual or contacting the APC with one or more additional agents.

26. The method of claim 4, wherein the method further comprises administering to the individual or contacting the APC with one or more agents that modulate the expression of one or more of Akt1, Ankfy1, Apc, Arpc1 b, Birc2, Bmi1, Bub3, Cacybp, Cebpb, Chd4, Crebbp, Cul2, Dars, Dcaf10, Dcaf4, Eif3f, Eif3i, Ep300, Fbxl13, Fbxo28, Fbxo3, Fbxw9, Gm13416, Gnb1, Gnb2, Grb10, Klhl24, Klhl7, Kmt2c, Kmt2d, Mapk14, Med8, Mlst8, Mtor, Nosip, Paf1, Pik3r4, Pparg, Ppp2r2a, Ppp2r2d, Preb, Rbbp4, Rbbp5, Rheb, Rictor, Rnf10, Rnf113a1, Rnf135, Rnf216, Rptor, Scap, Sec13, Sec31a, Smad2, Syvn1, Taf51, Traf2, Traf3, Traf7, Trim24, Trp53, Ube2e1, Ube2e3, Ube3c, Ufm1, Wdfy3, Wdr1, Wdr82, Whsc1, and Zbtb11.

27. A kit comprising a modulator of the interaction between (a) one, two, or all three of Ldb2, Rnf165, and Traf2 and (b) CCR7 for treating an individual having a cancer, an inflammatory disease, or an autoimmune disease according to the method of claim 1.

28. (canceled)

29. A method of monitoring the response of an individual having a cancer, an inflammatory disease, or an autoimmune disease to treatment with a modulator of the interaction between (a) one, two, or all three of Ldb2, Rnf165, and Traf2 and (b) CCR7 and treating the individual, the method comprising:
(a) (i) determining, in a biological sample obtained from the individual at a time point following administration of the modulator, the expression level of one or more of Ldb2, Rnf165, and Traf2; and
(b) (ii) comparing the expression level of the one or more genes in the biological sample with a reference level, thereby monitoring the response in the individual to treatment with the modulator, wherein:
(a) the individual has a cancer, the expression level of the one or more genes is increased in the biological sample obtained from the individual relative to the reference level, and the method further comprises administering to the individual one or more additional doses of the modulator, wherein the modulator is an agent that decreases the expression and/or activity of one, two, or all three of Ldb2, Rnf165, and Traf2; or
(b) the individual has an inflammatory disease or an autoimmune disease, the expression level of the one or more genes is decreased in the biological sample obtained from the individual relative to the reference level, and the method further comprises administering to the individual one or more additional doses of the modulator; wherein the modulator is an agent that increases the expression and/or activity of one, two, or all three of Ldb2, Rnf165, and Traf2.

30.-32. (canceled)

33. A method for increasing the proportion of migratory dendritic cells (mDCs) in an individual; increasing anti-tumor immunity in an individual; or treating a cancer, an inflammatory disease, an autoimmune disease, or an infectious disease in an individual, the method comprising administering to the individual an effective amount of:
(a) an agent that decreases the expression and/or activity of CCAAT/enhancer-binding protein beta (Cebpb);
(b) an agent that decreases the expression and/or activity of TNF receptor-associated factor 2 (Traf2); and/or
(c) an agent that increases the expression and/or activity of Death-inducer obliterator 1 (Dido1).

34.-35. (canceled)

36. A method for decreasing the proportion of migratory dendritic cells (mDCs) in an individual; decreasing autoimmune activity in an individual; or treating an inflammatory disease, an autoimmune disease, or an infectious disease in an individual, the method comprising administering to the individual an effective amount of:
(a) an agent that increases the expression and/or activity of CCAAT/enhancer-binding protein beta (Cebpb);
(b) an agent that increases the expression and/or activity of TNF receptor-associated factor 2 (Traf2); and/or
(c) an agent that decreases the expression and/or activity of Death-inducer obliterator 1 (Dido1).

37.-63. (canceled)

64. A method for treating a cancer, an inflammatory disease, or an autoimmune disease in an individual, the method comprising administering to the individual an effective amount of a modulator of the interaction between (a) F-box and WD repeat domain containing 11 (Fbxw11) and (b) nuclear factor kappa B subunit 1 (Nfkb1) or nuclear factor kappa B subunit 2 (Nfkb2).

65.-66. (canceled)

67. A method for;
(a) increasing processing of Nfkb1 and/or Nfkb2 into an active form, the method comprising contacting a cell capable of expressing Fbxw11 with an agent that increases expression and/or activity of Fbxw11;
(b) decreasing processing of Nfkb1 and/or Nfkb2 into an active form, the method comprising contacting a cell capable of expressing Fbxw11 with an agent that decreases expression and/or activity of Fbxw11; or
(c) modulating an immune response directed by Nfkb1 and/or Nfkb2 in an individual, the method comprising administering to the individual an effective amount of:
(i) an agent that increases expression and/or activity of Fbxw11; or
(ii) an agent that decreases expression and/or activity of Fbxw11.

68.-155. (canceled)

156. A method for preventing or treating a disease or disorder related to antigen-presenting cells (APCs) and/or inflammation in an individual, the method comprising administering to the individual an effective amount of a modulator of a gene of Table 1, thereby treating the individual.

157.-178. (canceled)
